(19) United States
(12) Patent Application Publication
(10) Pub. No.: US 2022/0289794 A1
(43) Pub. Date: Sep. 15, 2022

(54) SYNTHETIC ANTIMICROBIAL PEPTIDES

(71) Applicant: AJK BIOPHARMACEUTICAL, LLC, Irvine (US)

(72) Inventors: Keykavous PARANG, Irvine, CA (US); Rakesh TIWARI, Irvine, CA (US); Sandeep LOHAN, Lake Forest, CA (US); Eman MOHAMMED, Menoufia (EG); Dindyal MANDAL, Lake Forest, CA (US)

(21) Appl. No.: 17/639,082

(22) PCT Filed: Aug. 31, 2020
(86) PCT No.: PCT/US2020/048761

§ 371 Date: Feb. 28, 2022

Related U.S. Application Data
(60) Provisional application No. 62/893,633, filed on Aug. 29, 2019.

(51) Int. Cl. C07K 7/64; A61P 31/04; C07K 7/06; C07K 7/08; A01N 25/26; A61K 9/51; A61K 9/00; A61K 38/12; A61K 38/08; A61K 38/10; A01N 37/46; A61L 12/14; A61L 12/08

(52) U.S. Cl. CPC C07K 7/64; A61P 31/04; C07K 7/06; C07K 7/08; A01N 25/26; A61K 9/5115; A61K 9/0019; A61K 9/0014; A61K 9/007; A61K 38/12; A61K 38/08; A61K 38/10; A01N 37/46; A61L 12/14; A61L 12/088; A61K 38/00

(57) ABSTRACT

Synthetic peptides comprising a sequence of amino acids XnVm, wherein X represents positively charged amino acid, Y represents hydrophobic amino acid, and both n and m are greater than 2 are disclosed. In accordance with the purposes of the disclosed compositions and methods, as embodied and broadly described herein, the disclosed subject matter relates to synthetic antimicrobial peptides and methods of making and using same.

### CROSS REFERENCE TO RELATED APPLICATIONS

This application claims the benefit of priority to U.S. Provisional Application 62/893,633, filed Aug. 29, 2019, which is incorporated by reference herein in its entirety.

### FIELD

The field of the invention is antibiotics, in particular peptides with antibiotic properties.

### BACKGROUND

Antimicrobial resistance is an emerging issue in the 21st century due to antibiotic overuse. The Centers for Disease Control and Prevention (CDC) estimates that each year in the US, 23,000 people die due to bacterial resistance out of 2 million infected people. Furthermore, a recent global report estimates that ˜10 million people will die every year by 2050 due to antimicrobial resistance. Clinically reported pathogenic microbes include Enterococcus faecium, Staphylococcus aureus, Klebsiella pneumoniae, Acinetobacter baumanni, Pseudomonas aeruginosa, and Escherichia coli, (ESKAPE), that cause infectious disease in humans and animals (El-Mahallawy et al., 2016). They have adapted and grown in the presence of several classes of antibiotics, which resulted in the phenomenon known as Antimicrobial Resistance (AMR) (Nordstrom and Malmsten 2017).

This problem led to a national effort by the White House in 2014 for combating Antibiotic-Resistant Bacteria. The increasing prevalence of drug-resistant pathogens and toxicities associated with some frontline antibiotics, such as vancomycin for Gram-positive bacteria, and carbapenems and colistin for Gram-negatives, are occurring during a period of decline in the discovery and development of novel anti-infective agents. Thus, the development of novel compounds with activity against multidrug-resistant bacteria (MDRB) is urgently required to address this immediate public health concern.

Multidrug microbial resistance poses major challenges to the management of infection. The increase in the prevalence of drug-resistant pathogens is occurring at a time when the discovery and development of new anti-infective agents are slowing down dramatically. To regain the upper hand against resistant infections, modern antibiotic discovery programs should have at the forefront a goal of developing new antimicrobial agents that limit the emergence of resistance. Furthermore, the delivery of a number of commercially available antibiotics is challenging due to their serious side effects, the presence of an active efflux mechanism by bacteria, and/or limited uptake by bacteria because of a permeability barrier.

Antimicrobial peptides (AMPs) are a class of antibacterial agents that are widely produced in many organisms as host antimicrobial peptides and inflammatory agents in response to microorganisms' invasion (Ageitos et al. 2017). They have been isolated from various organisms, such as micro-organisms, plants, frogs, crustaceans, and mammals (Robert et al., 2008). In nature, there are lipopeptide AMPs, for example, polymyxins, and daptomycin which were approved by the FDA as an antibacterial peptide for clinical usage. For example, daptomycin, the first approved lipopeptide antibiotic approved for the treatment of Gram-positive bacteria pathogen originated from Streptomyces roseosporus. Vancomycin a branched tricyclic glycosylated peptide acts on enterococcus bacteria from a site different from β-lactam antibiotics penicillin and cephalosporin is obtained from Streptomyces Orientalis (Domhan et al., 2018). Often, AMPs exist in nature as prodrugs and are stored in the host as non-toxic compounds, but they are released as lethal weapons on the invading parasitic microorganisms (Seo et al., 2012.).

There are, however, drawbacks in the clinical usage and application of AMP as an antibacterial agent due to the following challenges. First, many peptides are unstable in the serum, especially when exposed to proteolytic enzymes and various salts that are found in the serum (Knappe et al., 2010). Second, they may exhibit a high level of cytotoxicity and hemolytic effect on red blood cells (De Smet et al., 2005). Also, several AMPs have a narrow spectrum of antibacterial activity. For example, vancomycin is active against Gram-positive bacteria and is considered as first-line drug treatment for methicillin-resistant Staphylococcus aureus (MRSA) and has no activity against Gram-negative bacteria. On the other hand, meropenem is a drug of choice for the treatment of multi-drug resistant Gram-negative bacteria such as Pseudomonas aeruginosa. Likewise, Gram-positive and Gram-negative bacteria may develop resistance to AMPs by changing the net charges and permeability of the cell surface, thereby decreasing the attraction of positively charged peptides to the cell wall (Kumar et al., 2018). What are thus needed are new antimicrobial peptides and methods of making and using same. The compositions and methods disclosed herein address these and other needs.

### SUMMARY

In accordance with the purposes of the disclosed compositions and methods, as embodied and broadly described herein, the disclosed subject matter relates to synthetic antimicrobial peptides and methods of making and using same. In specific examples, the disclosed subject matter relates to a synthetic peptide comprising a sequence of amino acids XₙYₘ, wherein X represents positively charged amino acid, Y represents hydrophobic amino acid, and both n and m are greater than 2. Also disclosed are antimicrobial compositions comprising a synthetic peptide of one of claims a nanoparticle, wherein the synthetic peptide is combined with a nanoparticle. Still further, disclosed are method of inhibiting or halting microbial growth, and use for treating infections, with the synthetic peptides and antimicrobial compositions disclosed herein. Also disclosed are formulations comprising a synthetic peptide as disclosed herein and a pharmaceutical acceptable carrier. Still further, disclosed are pegylated forms of the disclosed synthetic peptides. In yet further aspects, disclosed herein are compositions comprising a synthetic peptide, wherein the synthetic peptide comprises a non-peptide bond coupling two adjacent amino acids of the peptide. Also disclosed are kits comprising a synthetic peptide as disclosed herein; and instructions for applying the synthetic peptide in a manner effective to inhibit or halt microbial growth.

Additional advantages of the disclosed process will be set forth in part in the description which follows, and in part will be obvious from the description, or can be learned by practice of the disclosed process. The advantages of the disclosed process will be realized and attained by means of the elements and combinations particularly pointed out in the appended claims. It is to be understood that both the foregoing general description and the following detailed description are exemplary and explanatory only and are not restrictive of the disclosed process, as claimed.

### BRIEF DESCRIPTION OF THE FIGURES

The accompanying figures, which are incorporated in and constitute a part of this specification, illustrate several aspects of the disclosure, and together with the description, serve to explain the principles of the disclosure.

FIG. 1. [R₄W₄] (1), R₄W₄ (2), [R₄W₃] (3), and R₄W₃ (4) previously reported by us (Oh et al., 2014).

FIG. 2. Peptides containing arginine residues and unnatural hydrophobic residues with an equal number of arginine and hydrophobic residues.

FIG. 3. Peptides containing arginine residues and unnatural hydrophobic residues with four arginine residues and three hydrophobic residues.

FIG. 4. Examples of peptides containing arginine residues and two unnatural hydrophobic 3,3-diphenyl-L-alanine residues combined with one tryptophan at different positions.

FIG. 5. Examples of peptides containing arginine residues and two unnatural hydrophobic 3-(2-naphthyl)-1-alanine residues combined with one tryptophan at different positions.

FIG. 6. Examples of linear and cyclic peptides with broad-spectrum antibacterial activity.

FIG. 7. Antimicrobial Peptide Conjugates with antibiotics.

FIG. 8. MIC results of Tetracycline with peptides [R₅W₄] (IFX-301), [R₅W₄K] (IFX-315), [R₆W₄] (IFX-318), IFX-031 and IFX-067-1 with 11 commercially available antibiotics.

FIG. 9. MIC results of tetracycline with peptides [R₅W₄] (IFX-301), [R₅W₄K] (IFX-315), [R₆W₄] (IFX-318), and [IFX135].

FIG. 10. MIC results of tobramycin with peptides [R₅W₄] (IFX-301), [R₅W₄K] (IFX-315), [R₆W₄] (IFX-318), and [IFX135].

FIG. 11. MIC results of levofloxacin with peptides [R₅W₄], (IFX-301), [R₅W₄K] (IFX-315), [R₆W₄] (IFX-318), IFX-031, and IFX-067-1.

FIG. 12. MIC results of levofloxacin with peptides [R₅W₄] (IFX-301), [R₅W₄K] (IFX-315), [R₆W₄] (IFX-318), and [IFX135].

FIG. 13. MIC results of ciprofloxacin with peptides[R₅W₄] (IFX-301), [R₅W₄K] (IFX-315), [R₆W₄] (IFX-318), IFX-031, and IFX-067-1.

FIG. 14. MIC results of ciprofloxacin with peptides R₅W₄] (IFX-301), [R₅W₄K] (IFX-315), [R₆W₄] (IFX-318), and [IFX135].

FIG. 15. MIC results of clindamycin with peptides [R₅W₄] (IFX-301), [R₅W₄K] (IFX-315), [R₆W₄] (IFX-318), IFX-031, and IFX-067-1.

FIG. 16. MIC results of clindamycin with peptides [R₅W₄] (IFX-301), [R₅W₄K] (IFX-315), [R₆W₄] (IFX-318), and [IFX135].

FIG. 17. MIC results of daptomycin with peptides[R₅W₄] (IFX-301), [R₅W₄K] (IFX-315), [R₆W₄] (IFX-318), IFX-031, and IFX-067-1.

FIG. 18. MIC results of Daptomycin with peptides [R₅W₄] (IFX-301), [R₅W₄K] (IFX-315), [R₆W₄] (IFX-318), and [IFX135].

FIG. 19. MIC results of polymyxin with peptides [R₅W₄] (IFX-301), [R₅W₄K] (IFX-315), [R₆W₄] (IFX-318), IFX-031, and IFX-067-1.

FIG. 20. MIC results of polymyxin with peptides [R₅W₄] (IFX-301), [R₅W₄K] (IFX-315), [R₆W₄] (IFX-318), and [IFX135].

FIG. 21. MIC results of Kanamycin with peptides [R₅W₄] (IFX-301), [R₅W₄K] (IFX-315), [R₆W₄] (IFX-318), IFX-031, and IFX-067-1.

FIG. 22. MIC results of Kanamycin with peptides [R₅W₄] (IFX-301), [R₅W₄K] (IFX-315), [R₆W₄] (IFX-318), and [IFX135].

FIG. 23. MIC results of meropenem with peptides [R₅W₄], (IFX-301), [R₅W₄K] (IFX-315), [R₆W₄] (IFX-318), IFX-031, and IFX-067-1.

FIG. 24. MIC results of Meropenem with peptides [R₅W₄] (IFX-301), [R₅W₄K] (IFX-315), [R₆W₄] (IFX-318), and [IFX135].

FIG. 25. MIC results of vancomycin with peptides [R₅W₄], (IFX-301), [R₅W₄K] (IFX-315), [R₆W₄] (IFX-318), IFX-031, and IFX-067-1.

FIG. 26. MIC results of Vancomycin with peptides R₅W₄] (IFX-301), [R₅W₄K] (IFX-315), [R₆W₄] (IFX-318), and [IFX135].

FIG. 27. MIC results of metronidazole with peptides [R₅W₄], (IFX-301), [R₅W₄K] (IFX-315), [R₆W₄] (IFX-318), IFX-031, and IFX-067-1.

FIG. 28. MIC results of Meropenem-conjugate conjugate with [R₅W₄K] IFX-315.

FIG. 29A-29B. Inhibition of MRSA 33952 biofilm formation by (FIG. 29A) IFX-031, IFX-031-1, and IFX-111; (FIG. 29B) vancomycin.

FIG. 31. Inhibition of Klebsiella pneumoniae BAA-2470 biofilm formation by IFX-031, IFX-031-1, and IFX-111.

FIG. 32. Inhibition of Pseudomonas aeruginosa 47085 biofilm formation by ciprofloxacin.

FIG. 33. Inhibition of Pseudomonas aeruginosa 47085 biofilm formation by IFX-031, IFX-031-1, and IFX-111.

FIG. 34. Inhibition of Escherichia coli BAA-2471 biofilm formation by tigecycline.

FIG. 35. Prevention of Escherichia coli BAA-2471 biofilm formation by IFX-031, IFX-031-1, and IFX-111.

FIG. 36. Cytotoxicity of peptides in hepatic cell line (HepaRG, ThermoFisher HRPGC10).

FIG. 37. Cytotoxicity of peptides in hepatic cell line (HepaRG, ThermoFisher HRPGC10).

FIG. 38. Cytotoxicity of peptides in human skin fibroblast cell line (HeKa, ATCC PCS-200-011).

FIG. 39. Cytotoxicity of peptides in heart/myocardium cells (H9C2, ATCC No. CRL 1446).

FIG. 40. Cytotoxicity of peptides in heart/myocardium cells (H9C2, ATCC No. CRL 1446).

FIG. 41. Cytotoxicity of peptides in heart/myocardium cells (H9C2, ATCC No. CRL 1446).

FIG. 42. Cytotoxicity of peptides in heart/myocardium cells (H9C2, ATCC No. CRL 1446).

FIG. 43. Cytotoxicity of peptides in human lung fibroblast cells (MRC-5, ATCC CCL-171).

FIG. 44. Cytotoxicity of peptides in human lung fibroblast cells (MRC-5, ATCC CCL-171).

FIG. 45. Cytotoxicity of peptides in human lung fibroblast cells (MRC-5, ATCC CCL-171).

FIG. 46. Cytotoxicity of peptides in human lung fibroblast cells (MRC-5, ATCC CCL-171).

FIG. 47. Generation of gold nanoparticles by peptides determined by UV.

FIG. 48. Generation of gold nanoparticles by peptides determined by UV.

FIG. 49. Physical mixture MIC determination of combination between [R₅W₄] (IFX-301)-Au-NP with tetracycline. MIC results of tetracycline with [R₅W₄] Au-NP shows additive effect against PSA and E. coli.

FIG. 50. MIC results of tobramycin with peptide [R₅W₄]Au-NP show additive effect against MRSA and KPC.

FIG. 51. MIC results of meropenem with peptide [R₅W₄]Au-NP showed no enhancement.

FIG. 52. MIC results of Levofloxacin with peptide [R₅W₄]Au-NP shows an additive effect against E. coli.

FIG. 53. MIC results of ciprofloxacin with peptide [R₅W₄]Au-NP showed an additive effect against PSA and E. coli.

FIG. 54. MIC results of clindamycin with peptide [R₅W₄]Au-NP show an additive effect against E. coli.

FIG. 55. MIC results of kanamycin with peptide [R₅W₄]Au-NP showed no enhancement.

FIG. 56. MIC results of polymyxin with peptide [R₅W₄]Au-NP show no enhancement.

FIG. 57. MIC results of daptomycin with peptide [R₅W₄]Au-NP showed no enhancement.

FIG. 58. MIC results of vancomycin with peptide [R₅W₄]Au-NP showed no enhancement.

FIG. 59. The mixture of peptide [R₅W₄] (IFX-301) with antibiotics (1:1 ratio) was then used in the synthesis of Au-NP ratio. MIC results of tetracycline with peptide [R₅W₄]Au-NP showed an additive effect against PSA and E. coli and KPC.

FIG. 60. The mixture of peptide [R₅W₄] (IFX-301) with antibiotics (1:1 ratio) was then used in the synthesis of Au-NP ratio. MIC results of tobramycin with peptide [R₅W₄]Au-NP showed significant enhancement against MRSA, KPC and E. coli.

FIG. 61. The mixture of peptide [R₅W₄] (IFX-301) with antibiotics (1:1 ratio) was then used in the synthesis of Au-NP ratio. MIC results of meropenem with peptide [R₅W₄]Au-NP showed significant enhancement against MRSA and PSA and additive effect against KPC and E. coli.

FIG. 62. The mixture of peptide [R₅W₄] (IFX-301) with antibiotics (1:1 ratio) was then used in the synthesis of Au-NP ratio. MIC results of levofloxacin with peptide [R₅W₄]Au-NP showed significant enhancement with MRSA, PSA, and E. coli and additive effect against KPC.

FIG. 63. The mixture of peptide [R₅W₄] (IFX-301) with antibiotics (1:1 ratio) was then used in the synthesis of Au-NP ratio. MIC results of ciprofloxacin with peptide [R₅W₄]Au-NP showed significant enhancement against MRSA and PSA, and additive effect against KPC and E. coli.

FIG. 64. The mixture of peptide [R₅W₄] (IFX-301) with antibiotics (1:1 ratio) was then used in the synthesis of the Au-NP ratio. MIC results of Clindamycin with peptide [R₅W₄]Au-NP showed significant enhancement against KPC, PSA, and E. coli.

FIG. 65. The mixture of peptide [R₅W₄] (IFX-301) with antibiotics (1:1 ratio) was then used in the synthesis of Au-NP ratio. MIC results of kanamycin with peptide [R₅W₄]Au-NP showed significant enhancement with PSA and E. coli, and additive effect with MRSA and KPC.

FIG. 66. The mixture of peptide [R₅W₄] (IFX-301) with antibiotics (1:1 ratio) was then used in the synthesis of Au-NP ratio. MIC results of polymyxin with peptide [R₅W₄]Au-NP showed significant enhancement against MRSA and E. coli, and additive against KPC and PSA.

FIG. 67. The mixture of peptide [R₅W₄] (IFX-301) with antibiotics (1:1 ratio) was then used in the synthesis of Au-NP ratio. MIC results of daptomycin with peptide [R₅W₄]Au-NP showed significant enhancement against MRSA and PSA, and additive effect with KPC and E. coli.

FIG. 68. The mixture of peptide [R₅W₄] (IFX-301) with antibiotics (1:1 ratio) was then used in the synthesis of Au-NP ratio. MIC results of vancomycin with [R₅W₄]Au-NP showed significant enhancement against MRSA and E. coli, and additive effect against KPC and PSA.

FIG. 69. The antiviral activity of peptides alone and in combination with remdesivir against human coronavirus 229E (HCoV-229E) demonstrating significant synergistic activity.

FIG. 70. Hemolytic Assay result of cyclic peptide [W₄R₄] (IFX-326) against human red blood cells using 0.2% Triton X and PBS buffer pH 7.4 as positive and negative controls respectively

FIG. 71. The time-dependent survival rate of G. mellonella, which were treated with peptide [W₄R₄] (IFX-326) and tetracycline.

### DETAILED DESCRIPTION

The materials, compounds, compositions, articles, and methods described herein can be understood more readily by reference to the following detailed description of specific aspects of the disclosed subject matter and the Examples and Figures included therein.

Before the present materials, compounds, compositions, articles, devices, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refer to at least one of something selected from the group consisting of A, B, C and N, the text should be interpreted as requiring only one element from the group, not A plus N, or B plus N, etc.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified, thus fulfilling the written description of all Markush groups used in the appended claims.

Thus, there is still a need for effective antimicrobial compounds, particularly antimicrobial compounds that are safe and effective when used against microbes resistant to conventional antibiotics.

In embodiments of the inventive concept, synthetic peptides that incorporate both hydrophobic and positively charged amino acids at opposite sides are provided that have broad-spectrum antibacterial activity against Gram-positive and Gram-negative bacteria, including their antibiotic-resistant strains. Amino acids of such peptides can be naturally occurring or non-naturally occurring and can be present as either D or L isomers. In some embodiments the synthetic peptides are conjugated to and/or used in combination with other compounds, such as antibiotics, metal nanoparticles, and antibiotics in addition to the metal nanoparticles that enhance or add to their antibacterial action. Preferred peptide compound(s) prevented or reduced bacterial biofilm generation.

One should appreciate that the disclosed techniques provide many advantageous technical effects, including safe and effective treatment of infections that are resistant to prior art antibiotic therapy.

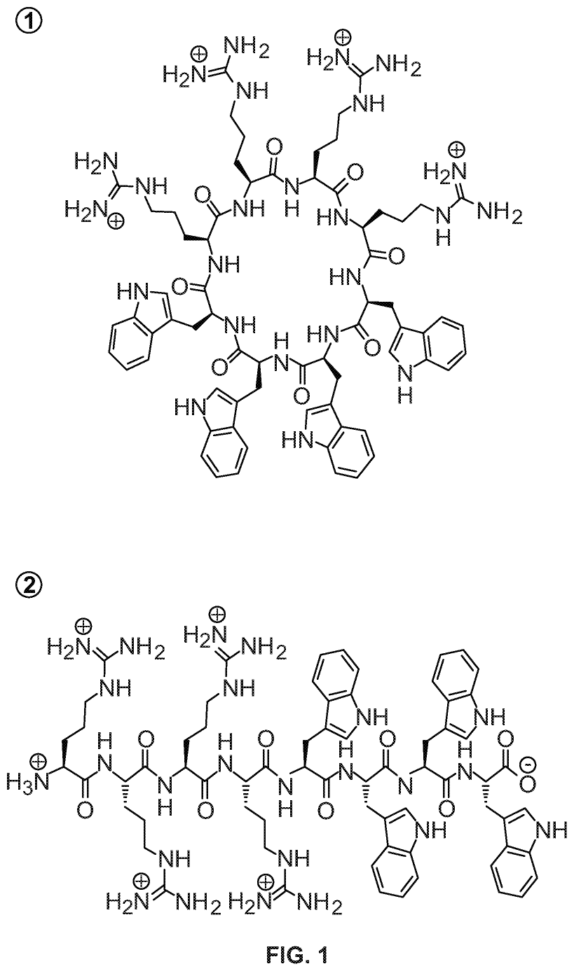

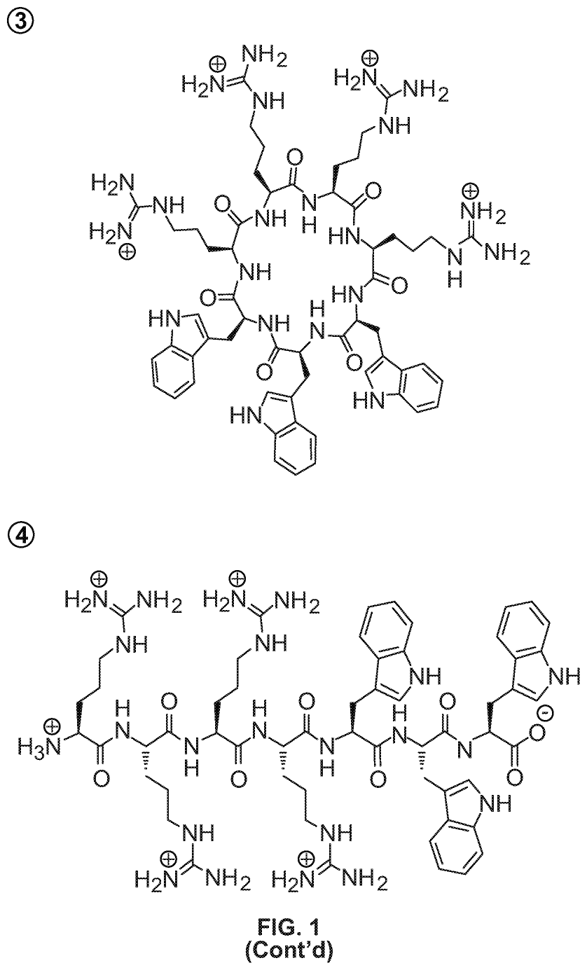

Compositions and methods of the inventive concept include the linear and cyclic peptides containing natural and/or unnatural positively-charged amino acids and hydrophobic residues as antibacterial agents. We have previously synthesized and evaluated several cyclic and linear peptides (FIG. 1) that demonstrated antibacterial activity against Methicillin-Resistant Staphylococcus aureus (MRSA) and Pseudomonas aeruginosa (PA01) (Oh et al. 2014). Cyclic peptide [W₄R₄] containing positively-charged arginine (R) and hydrophobic tryptophan (W) residues was previously shown by us to have antibacterial activities. Cyclic peptide [W₄R₄] (1, FIG. 1) had MIC 2.67 μg/mL (1.95 μM) and 42.8 μg/mL (31.3 μM) against MRSA and Pseudomonas aeruginosa, respectively.

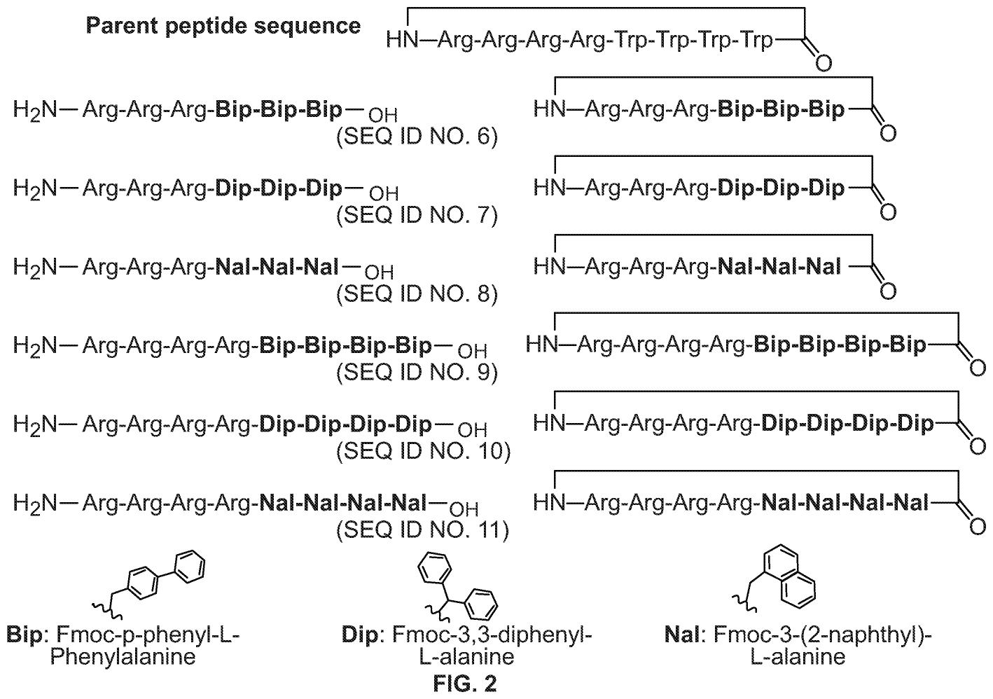

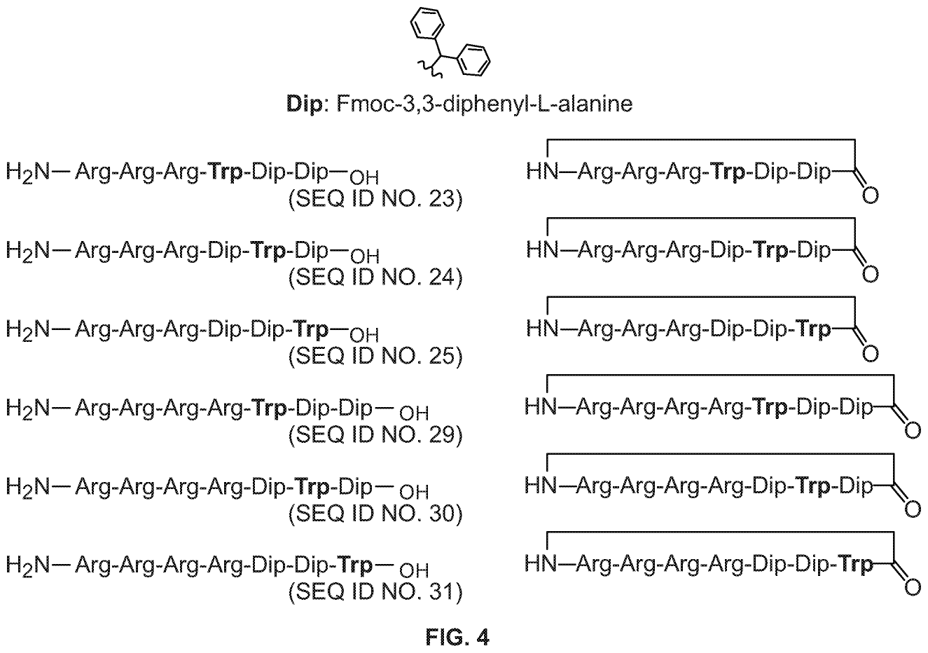

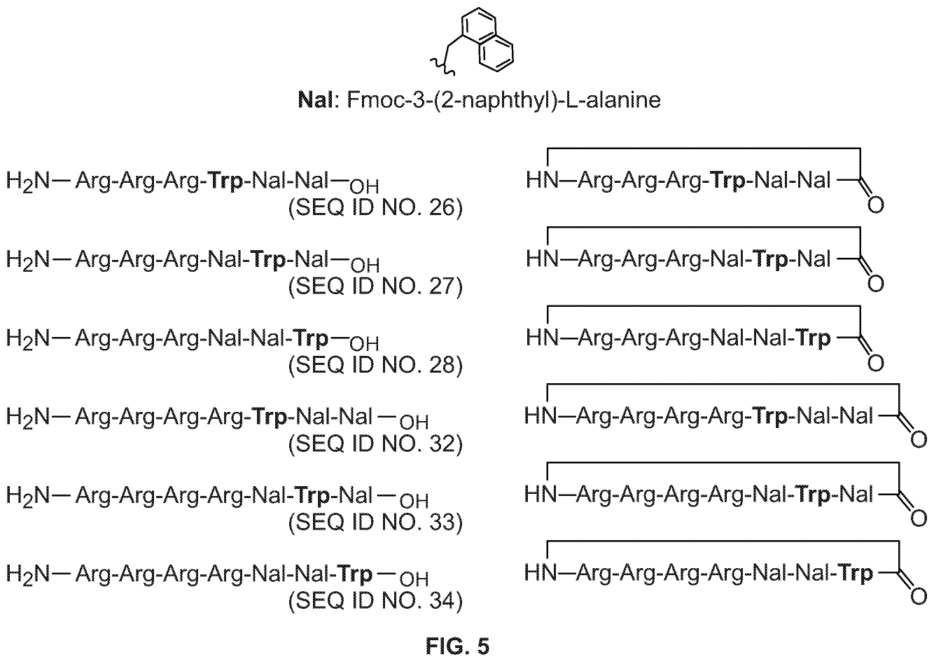

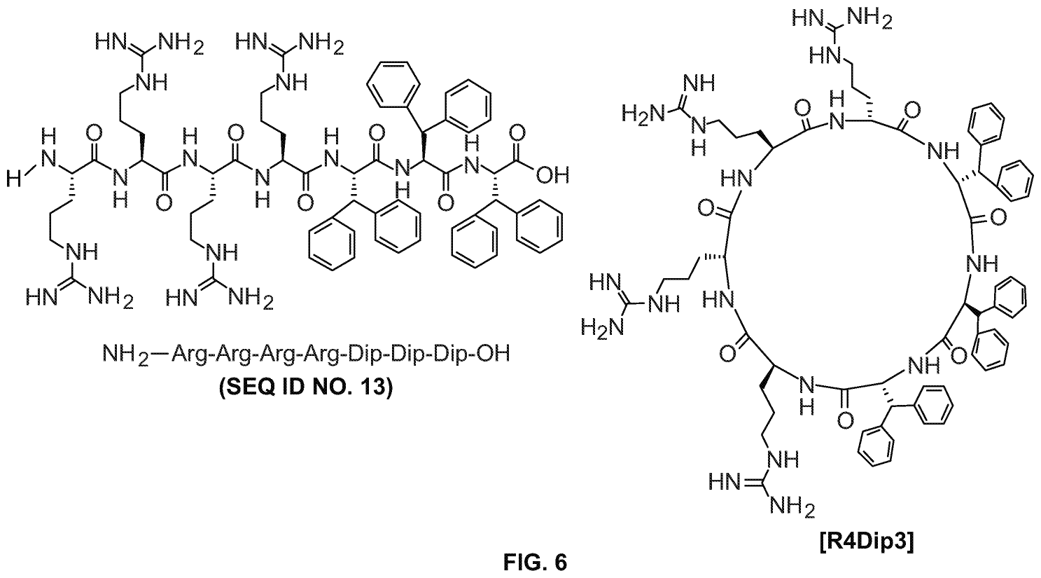

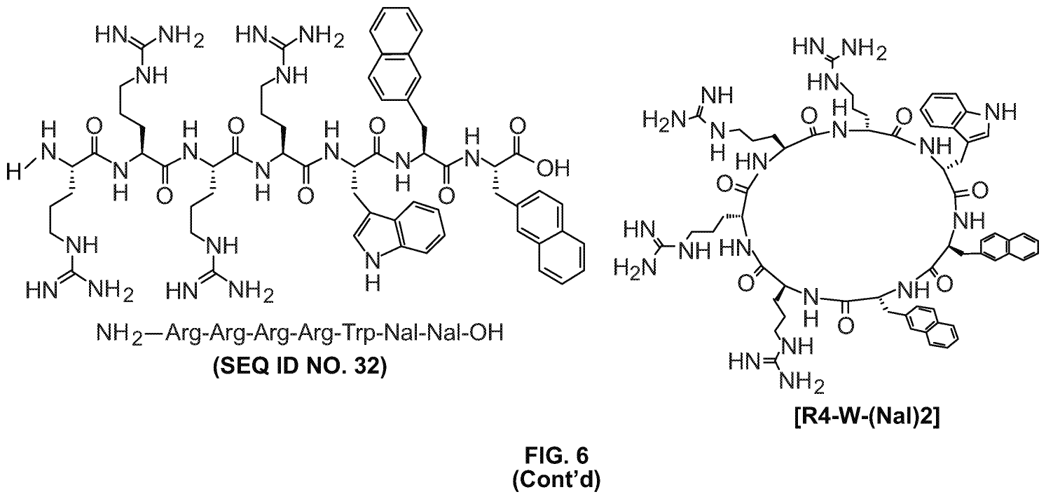

This invention is distinct from our previous work since it includes chemical modifications, such as the substitution of L-amino acid with D-amino acids to avoid proteolytic enzymes, the substitution of positively charge arginine or hydrophobic residues with non-natural amino acids, and generation of sequences that have not been discovered and have significantly higher broad-spectrum activity against both Gram-positive and Gram-negative bacteria and multidrug-resistant strains. Some of the sequences are shown in FIGS. 2-6.

We synthesized linear and cyclic peptides that have hydrophobic and positively-charged residues as the amino acids sequence in its basic structure. We varied the amino acid constituents in the structure to determine antibacterial activity effectiveness of derived compounds and establish the structure-activity relationship. The strategy was to vary net hydrophobicity and the positive charges of derived compounds based on structure-activity relationship and evaluate antibacterial potentials. We observed that the appropriate positively-charged and hydrophobic residues enhanced the inherent penetrating properties of the cyclic peptide with a resultant increase in antibacterial activities. Furthermore, the combination or conjugation of a cyclic or linear peptide with antibiotics, gold nanoparticles, or antibiotics in addition to gold nanoparticles generated a wide spectrum of activity against Gram-positive and Gram-negative multidrug-resistant clinically reported bacteria pathogens.

Preferred compound(s) could be used as a stand-alone therapy to treat bacterial infections. Compound(s) can also be used in combination with current antibacterial drugs and/or gold or silver nanoparticles to provide potent therapies for treating infections. Our data support use to treat both Gram-positive and Gram-negative infections. These compounds represent a new class of antibacterial agents. The structures of these series of compounds are different than those of current antibacterial drugs. Therefore, these compounds will likely not be compromised by existing mechanisms of drug resistance. The additive and synergistic nature of these peptides, in combination with antibiotics, gold nanoparticles, or antibiotics in addition to the gold nanoparticles, suggest a potential for co-administration to fight bacterial infections. The used amino acids, peptide sequence, examples of their structures, in vitro antibacterial activities, hemolytic assay, and preliminary in vivo activities are summarized here.

Preferred peptide compound(s) could be physically mixed with antibiotics and antiviral for generation of synergistic antibacterial and antiviral activities.

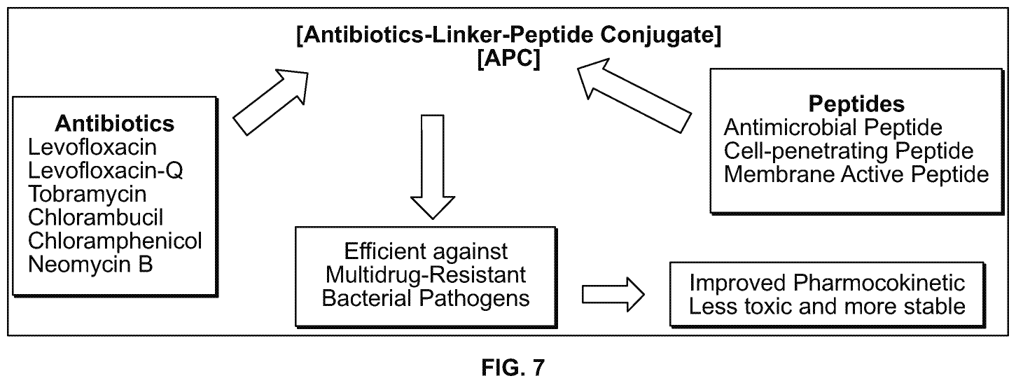

Combination or Conjugation with Antibiotics. These peptides can be used alone or in combination with current clinical antibiotics to provide enhanced treatments of bacterial infections. The peptides can be physically mixed with the antibiotics or can be conjugated with antibiotics as Antibiotics-Peptide Conjugates (APC) (FIG. 7).

Antimicrobial peptides that can be used to improve the delivery of the antibiotics through the bacteria membrane, to minimize their toxicity against normal cells, and to overcome the bacterial resistance. The combination or conjugation with antibiotics will provide synergistic activities and bypass the efflux mechanism. Some antimicrobial peptides were found to have molecular transporter properties, which would potentially aid in the delivery of other antibiotics such as Meropenem, Ciprofloxacin, Tedizolid, and Levofloxacin, which might suffer from several limitations such as efflux, resistance, toxicity, and stability. Some of these peptides are shown to possess additive and synergistic activity in in-vitro models when combined with tetracycline. For example, [R₄W₄] acted synergistically with tetracycline against methicillin-resistant Staphylococcus aureus (MRSA) and E. coli in time-kill assays (Oh et al., 2014). Combined therapy of [R₄W₄] and tetracycline was more effective than either drug alone when tested in-vivo for the survival of Galleria mellonella infected with MRSA. This is clinically and scientifically significant; MRSA and E. coli are the two most commonly isolated bacteria in hospital and community-associated infections. At 4 h of incubation, tetracycline and [R₄W₄] in combination are consistently more active than either agent alone (with the exception of 8× against MRSA). Antagonism is observed at 4× against E. coli. The combination is markedly more effective against MRSA than E. coli at 4 h, perhaps because the compound was more able to penetrate the gram-positive cell wall (Oh et al., 2014).

At 24 h of incubation, tetracycline and [R₄W₄] in combination remained consistently more than or equally as active as either agent alone, with the exception of 8× against E. coli. Although synergy as defined by a ≥2 log 10 CFU/mL decrease was only observed at 1×, the MIC of tetracycline against E. coli, decreases as high as 1.98 log 10 CFU/mL and 1.73 log 10 CFU/mL were observed at 2× the MIC of tetracycline against both MRSA and E. coli, respectively (Oh et al., 2014). A similar pattern was observed with lead peptides described in this invention. We have shown here the synergistic activity of peptides with 11 antibiotics and Remdesivir (an antiviral drug). We have also shown here the synergistic effect of peptides with antibiotics in addition to gold nanoparticles. Preferred peptide compound(s) prevented or reduced bacterial biofilm generation.

These pathogens are responsible for significant morbidity and mortality in the United States and globally. Other peptides in this class will act the same way in synergistic or additive antibacterial activity. Examples of antibiotics are Meropenem, Ciprofloxacin, Tedizolid, and Levofloxacin, Imipenem, Tobramycin, and Clindamycin.

Preferred peptide compound(s) could be used directly for the generation of gold nanoparticles and silver nanoparticles with improved antibacterial properties. The peptides can be used alone or in combination with nanoparticles and peptide-capped nanoparticles. Examples of nanoparticles are gold and silver nanoparticles that can be used along with peptides and antibiotics to improve the activity against multidrug-resistant bacteria. Cell-penetrating peptide-capped nanoparticles with antimicrobial properties will be preferentially taken up by bacteria, where they gradually release their cargo antibiotics resulting in sustained local antibacterial effect by a double-barreled mechanism without causing significant toxicity to normal cells. Peptide-capped metal nanoparticles have antimicrobial and cell-penetrating properties by perturbing bacterial membranes and becoming membrane permeabilizers, respectively. Cell-penetrating peptides with intrinsic antibacterial activity entrap and enhance the uptake of antibiotics across the membrane when they cap the metal nanoparticles.

Preferred peptide compound(s) could be physically mixed with antibiotics first and then be used for the generation of gold and silver nanoparticles to afford synergistic antibacterial activities

Preferred peptide compound(s) could be use directly for the generation of gold nanoparticles and silver nanoparticles and then physically mixed with antibiotics for generation improved antibacterial activities.

Amino acids. Examples of positively-charged amino acids in the linear and cyclic peptides are L-arginine, L-lysine, l-histidine, d-histidine, D-arginine, D-lysine. Furthermore, positively-charged amino acids ornithine, L- or D-arginine residues with shorter or longer side chains (e.g., C3-Arginine (Agp), C4-Arginine (Agb)), diaminopropionic acid (Dap) and diaminobutyric acid (Dab), amino acids containing free side-chain amino or guanidine groups, and modified arginine and lysine residues.

Examples of hydrophobic residues in the linear and cyclic peptides are L-tryptophan, D-tryptophan, L-phenylalanine, d-phenylalanine, L-isoleucine, d-isoleucine, p-phenyl-L-phenylalanine (Bip), 3,3-diphenyl-L-alanine (Dip), 3,3-diphenyl-D-alanine (dip), 3(2-naphthyl)-L-alanine (NaI), 3(2-naphthyl)-D-alanine (naI), 6-amino-2-naphthoic acid, 3-amino-2-naphthoic acid, 1,2,3,4-tetrahydronorharmane-3-carboxylic acid, 1,2,3,4-tetrahydro-3-isoquinolinecarboxylic acid (Tic-OH), 1,2,3,4-tetrahydro-3-isoquinolinecarboxylic acid, modified d- or 1-tryptophan residues like N-alkyl or N-aryl tryptophan, substituted d- or L-tryptophan residues (e.g., 5-hydroxy-L-tryptophan, 5-methoxy-L-tryptophan, 6-chloro-L-tryptophan), other N-heteroaromatic and hydrophobic amino acids, and fatty amino acids NH₂—(CH₂)ₓ—COOH (x=1-20) or NH₂—(CH₂)ₓ—(CH═CH)_{y}—COOH (x=1-15, y=1-15, Z or E configuration).

Sequence. A preferred sequence of these peptides includes linear (XₙYₘ) or cyclic [XₙYₘ] or hybrid peptides (cyclic-linear) [Xₙ]Yₘ, or Xₙ[Yₘ], where x is a positively-charged amino acid, Y is a hydrophobic residue, and n and m=2-9. In specific examples, n, can be 2, 3, 4, 5, 6, 7, 8, or 9. In other examples, m can be 2, 3, 4, 5, 6, 7, 8 or 9. Other amino acids can be inserted between positively charged, between hydrophobic residues, or between positively-charged and hydrophobic residues, while multiple positively charged residues or multiple hydrophobic amino acids are next to each other creating a positively charged component in one side and a hydrophobic component in the other side. Cyclic peptides with above formula include those formed through N- to C-terminal cyclization, disulfide cyclization, stapled method, click cyclization and any other cyclization method. Cyclic peptides include bicyclic peptides with [X]ₙ[Y]ₘ, where one cyclic peptide contains positively-charged amino acids and the other cyclic peptide contains hydrophobic amino acids. The cyclic peptides may be connected directly through an amino acid or an appropriate linker. Similar or different positively charged or hydrophobic residues may be in the same peptide. In other words, positively charged amino acids can be the same or different. Similarly, hydrophobic amino acids in the same sequence can be the same or different. The peptides can have hybrid structures with cyclic peptides contain positively-charged residues or hydrophobic residues attached to linear hydrophobic or positively-charged residues, respectively. Some of the sequences are shown in Tables 1 and 2 and FIGS. 2-6.

In another aspect, the peptides in this invention may have antiviral activity against coronaviruses or other viruses as stand-alone or in combination with other antiviral agents.

The peptides have synergistic activity with current antivirals like Remdesivir that is used against SARS-CoV-2.

In another aspect, the peptides of may be in the form of a composition that may be used to treat or prevent infection, transmission, or acquisition of COVID-19 and other coronaviruses-related diseases.

Synthesized compounds are active against SARS-CoV-2 and other coronaviruses and may have potential activity as antiviral agents.

Inventors believe that compounds of the inventive concept can exhibit antiviral activity against a broad range of viruses, in particular enveloped viruses. Examples of suitable DNA viruses include (but are not limited to) Herpesviruses, Poxviruses, Hepadnaviruses, and Asfarviridae. Suitable RNA viruses include (but are not limited to) Flavivirus, Alphavirus, Togavirus, Coronavirus, Hepatitis D, Orthomyxovirus, Paramyxovirus, Rhabdovirus, Bunyavirus, Filovirus, Retroviruses, and Retroviruses. In particular, the Applicant believes that compounds of the inventive concept can be effective against disease caused by a coronavirus, such as COVID-19.

In another aspect, the synthesized peptides may be chemically linked to another compound to provide a composition of matter and may contain a carrier or excipient, and may be used in a method for treating, preventing, or reducing bacterial diseases by delivering the composition of matter in injectable, solid or semi-solid forms, such as a tablet, film, gel, cream, ointment, pessary, or the like.

Compounds of the inventive concept can be provided to an individual in need of treatment by any suitable route. Suitable routes include injection, infusion, topical application to skin, topical application to a mucus membrane (e.g. oral, nasal, vaginal, and/or rectal mucosa), application to the ocular surface, introduction to the gastrointestinal tract, and/or inhalation. Modes of application can vary depending on the bacterial disease being treated, the stage of the bacterial disease, and/or characteristics of the individual being treated. In some embodiments, the manner of application of the drug can change over the course of treatment. For example, an individual presenting with acute symptoms may initially be treated by injection or infusion in order to rapidly provide useful concentrations of the drug, then moved to ingestion (for example, of a pill or tablet) to maintain such useful concentrations over time.

Accordingly, formulations that include a drug of the inventive concept can be provided in different forms and with different excipients. For example, formulations provided for ingestion can be provided as a liquid, a powder that is dissolved in a liquid prior to consumption, a pill, a tablet, or a capsule. Solid forms provided for ingestion can be provided with enteric coatings or similar features that provide release of the drug in a selected portion of the gastrointestinal tract (e.g. the small intestine) and/or provide sustained release of the drug over time. Formulations intended for topical application can be provided as a liquid, a gel, a paste, an ointment, and/or a powder. Such formulations can be provided as part of a dressing, film, or similar appliance that is placed on a body surface. Formulations intended for injection (e.g. subcutaneous, intramuscular, intraocular, intraperitoneal, intravenous, etc.) or infusion can be provided as a liquid or as a dry form (such as a powder) that is dissolved or suspended in liquid prior to use. Formulations intended for inhalation can similarly be provided in a liquid form or a dray form that is suspended or dissolved in liquid prior to use, or as a dry powder of particle size suitable for inhalation. Such inhaled formulations can be provided as an atomized spray or subjected to nebulization to generate a liquid droplet suspension in air or other suitable gas vehicle for inhalation.

Liquid formulations can be in the form of a solution, a suspension, a micellar suspension, and/or an emulsion. Similarly, dry, or granular formulations can be provided as lyophilized or spray-dried particulates, which in some embodiments can be individually encapsulated.

Compounds of the inventive concept can be provided in any amount that provides a suitably effective antibacterial effect. It should be appreciated that this can vary for a given compound depending upon the route of administration, the bacteria being treated, and the characteristics of the individual being treated. Suitable doses can range from 0.1 μg/kg to 100 mg/kg body weight, or from 0.01 μg/mL to 100 mg/mL w/w/concentration.

Dosing schedules applied to a compound of the inventive concept can vary depending upon the bacteria being treated, the mode of application, the severity of the disease state, and the characteristics of the individual. In some embodiments, the application of the drug can be essentially constant, for example, through infusion, incorporation into ongoing intravenous therapy, and/or inhalation. In other embodiments, a compound of the inventive concept can be applied once. In still other embodiments, a compound of the inventive concept can be provided periodically over a suitable period. For example, a compound of the inventive concept can be provided every 2 hours, every 3 hours, every 4 hours, every 6 hours, every 8 hours, every 12 hours, daily, on alternating days, twice a week, weekly, every two weeks, monthly, every 2 months, every 3 months, every 6 months, or yearly.

As noted above, formulation, dose, and dosing schedule for a compound of the inventive concept can vary depending on the state of the bacterial disease. In some embodiments, such a compound can be provided to an individual in need of prophylactic treatment, for example, to an uninfected individual in order to prevent the establishment of infection by a bacteria or virus following exposure. In other embodiments, a compound of the inventive concept can be provided to an individual who is infected with a bacteria or virus but is asymptomatic. In still other concepts, a compound of the inventive concept can be provided to an individual that is infected with a bacteria or virus and is symptomatic. As noted above, dosing, route, and dosing schedule of the compound can be adjusted as symptoms of an active viral infection change.

In some embodiments, a compound as described above can be used in combination with one or more other active companion compounds. Suitable companion compounds include antibacterial compounds, antiviral compounds, antifungal compounds, anti-inflammatory compounds, bronchodilators, and compounds that treat pain. The Inventor anticipates that synergistic (i.e. greater than additive effects) can result from such combinations regarding antibacterial or antiviral effect, reduction in disease time course, reduction in the severity of symptoms, and/or morbidity.

Similarly, in some embodiments, two or more compounds as described above can be used in combination. The Inventor anticipates that synergistic (i.e. greater than additive effects) can result from such combinations regarding antibacterial effect, reduction in disease time course, reduction in the severity of symptoms, and/or morbidity.

The antimicrobial peptides have both antimicrobial properties and molecular transporters of antibiotics. The peptides have antimicrobial and cell-penetrating properties by perturbing bacterial membranes and becoming membrane permeabilizers, respectively. Cell-penetrating peptides with intrinsic antibacterial activity can entrap and enhance the uptake of antibiotics across the membrane. Antimicrobial properties will be preferentially taken up by bacteria, where they gradually release their cargo antibiotics resulting in sustained local antibacterial effect by a double-barreled mechanism without causing significant toxicity to normal cells. The peptides have synergistic activity with current antibiotics.

Bacterial strains. Bacteria include Gram-positive and Gram-negative bacteria and biofilm resulted from any of these bacterial strains. Some examples of bacteria are Methicillin-resistant Staphylococcus aureus (MRSA), Acinetobacter baumannii, Enterococcus faecalis, Clostridium difficile, Klebsiella pneumonia, Escherichia coli, Staphylococcus epidermidis, Streptococcus pneumoniae, Streptococcus mutans, Streptococcus pyogenes, Pseudomonas aeruginosa, Mycobacterium tuberculosis, Carbapenem-resistant Enterobacteriaceae (CRE) gut bacteria, and Neisseria Gonorrhea. Table 3 shows some examples of bacteria.

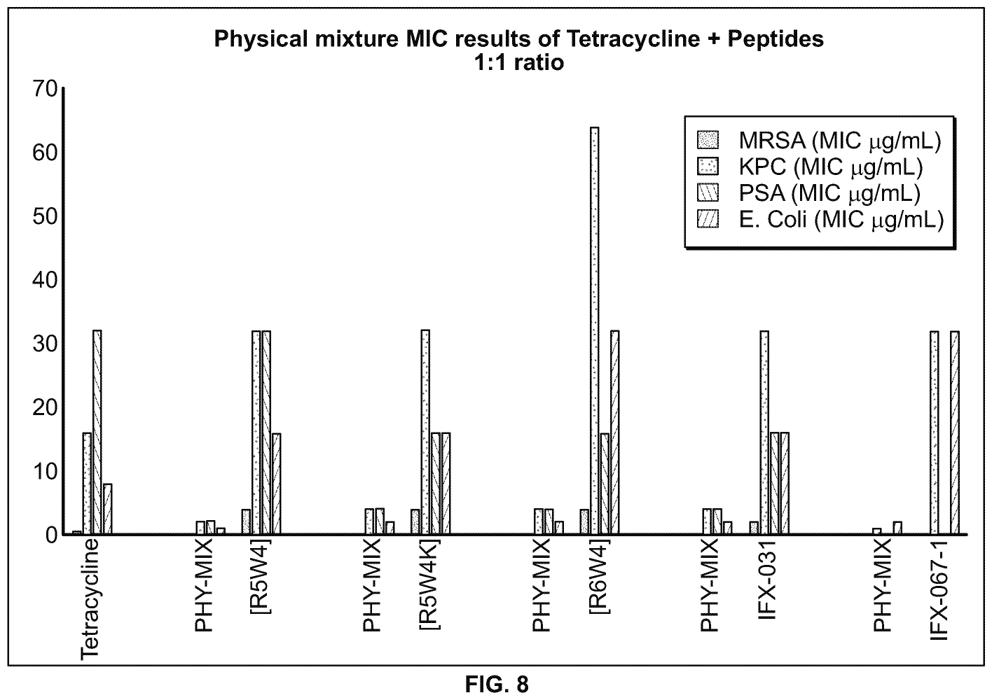

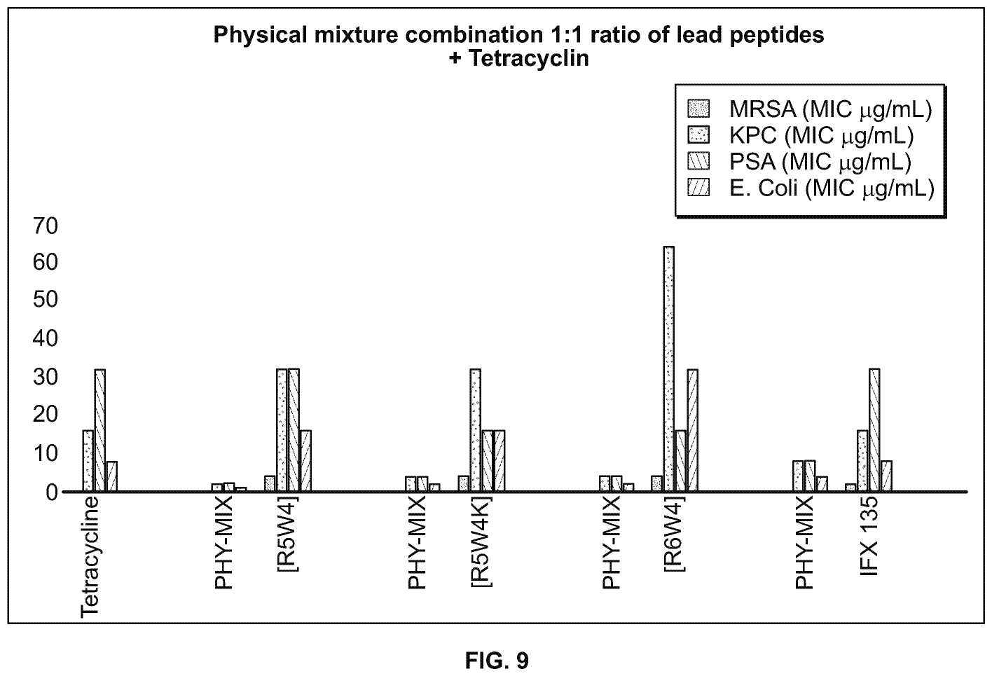

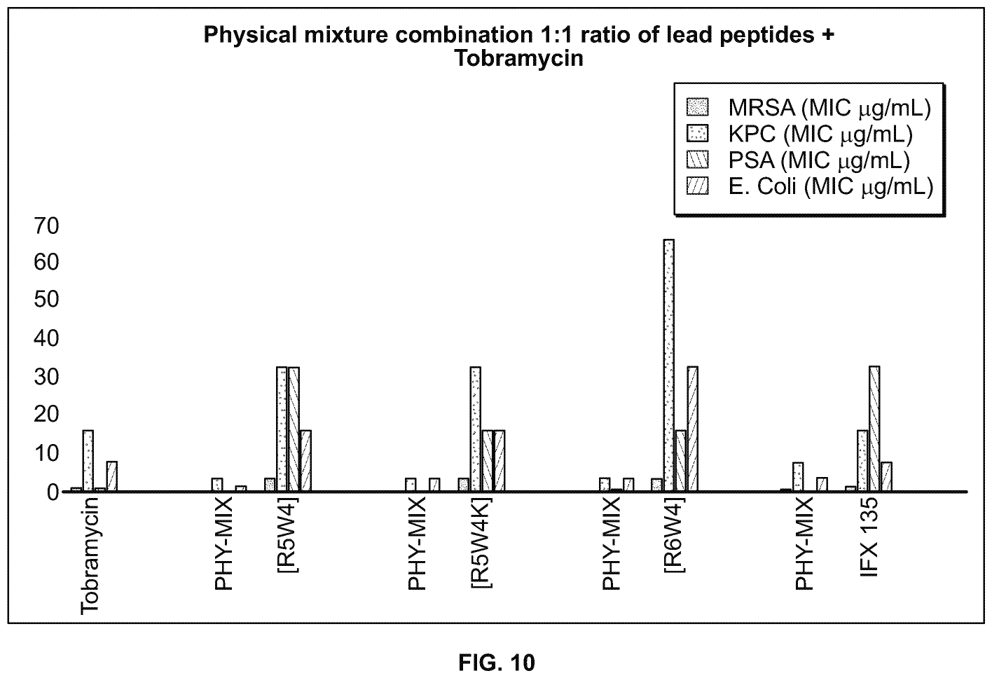

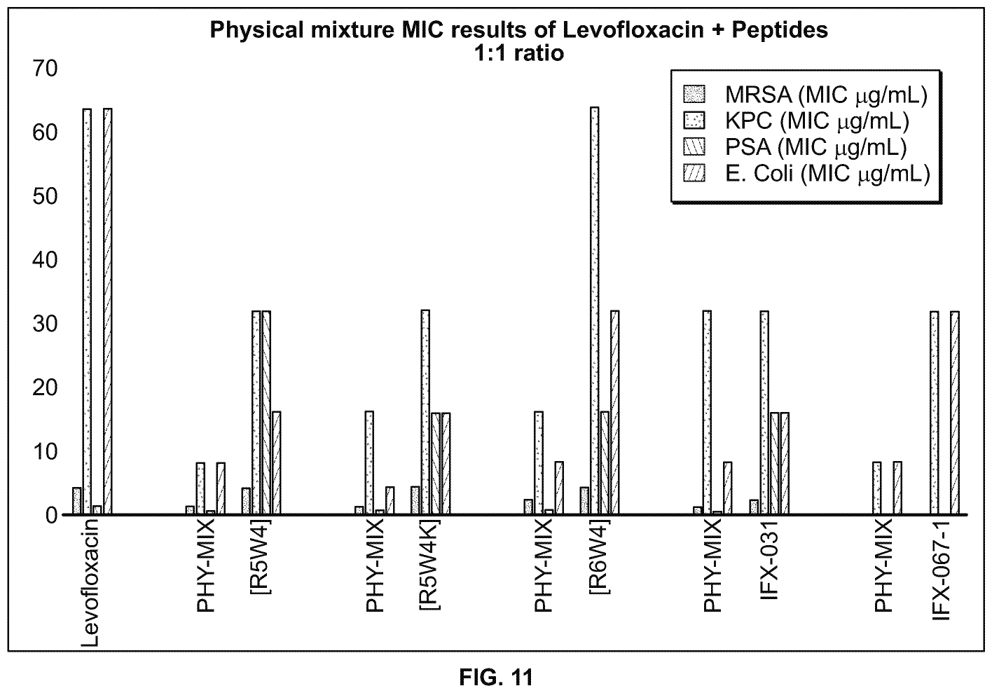

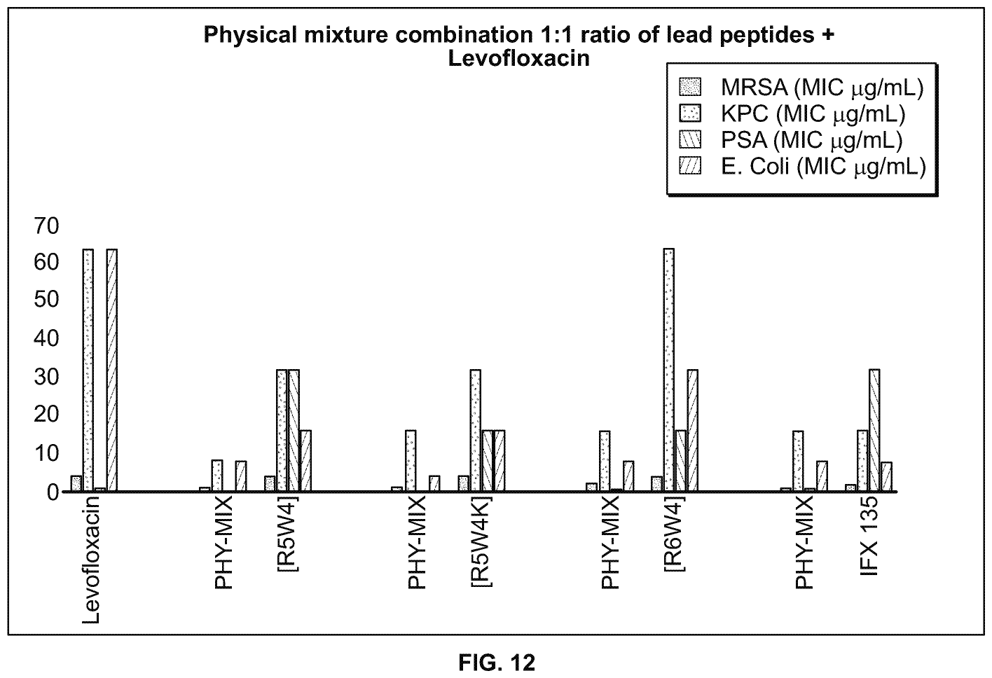

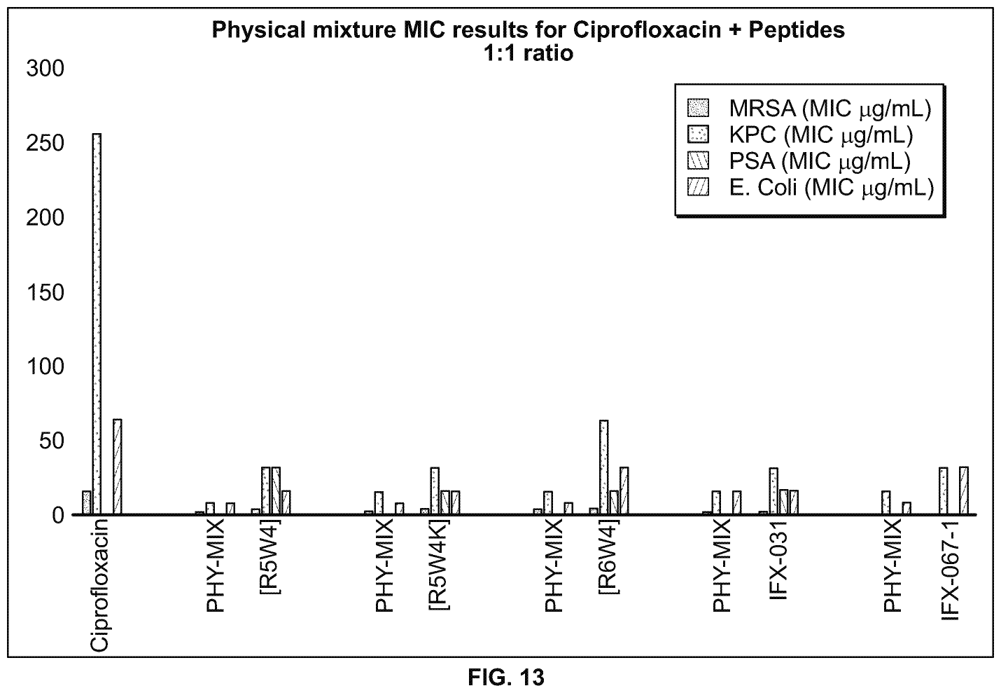

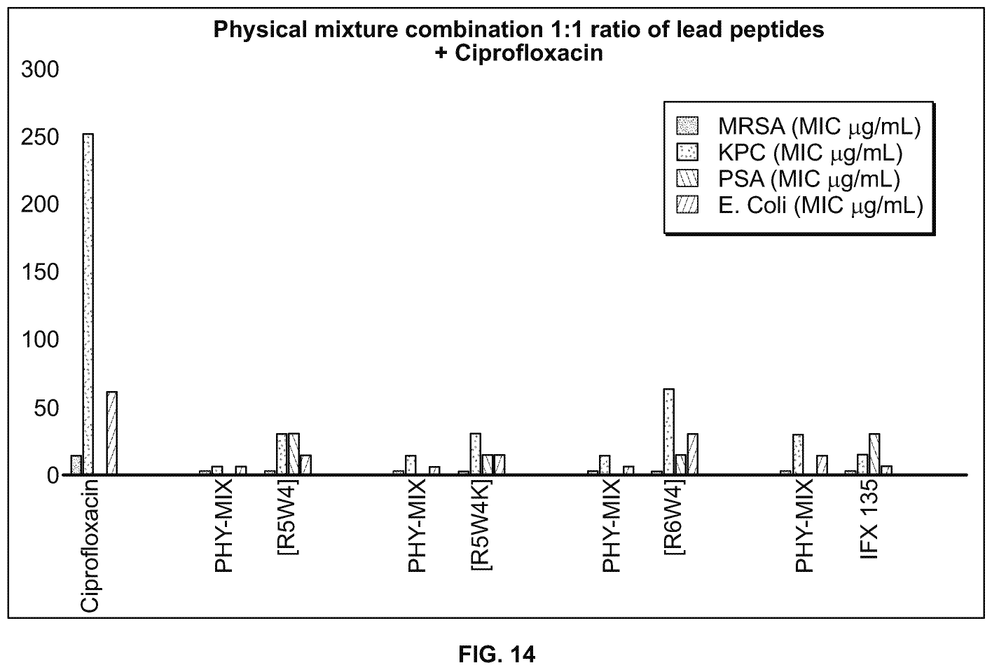

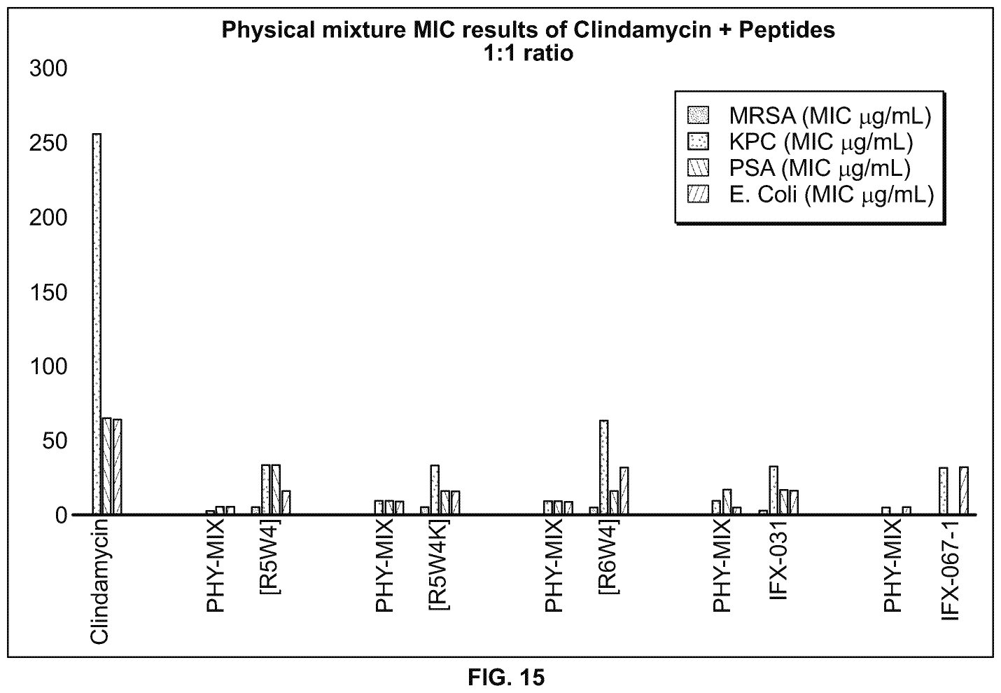

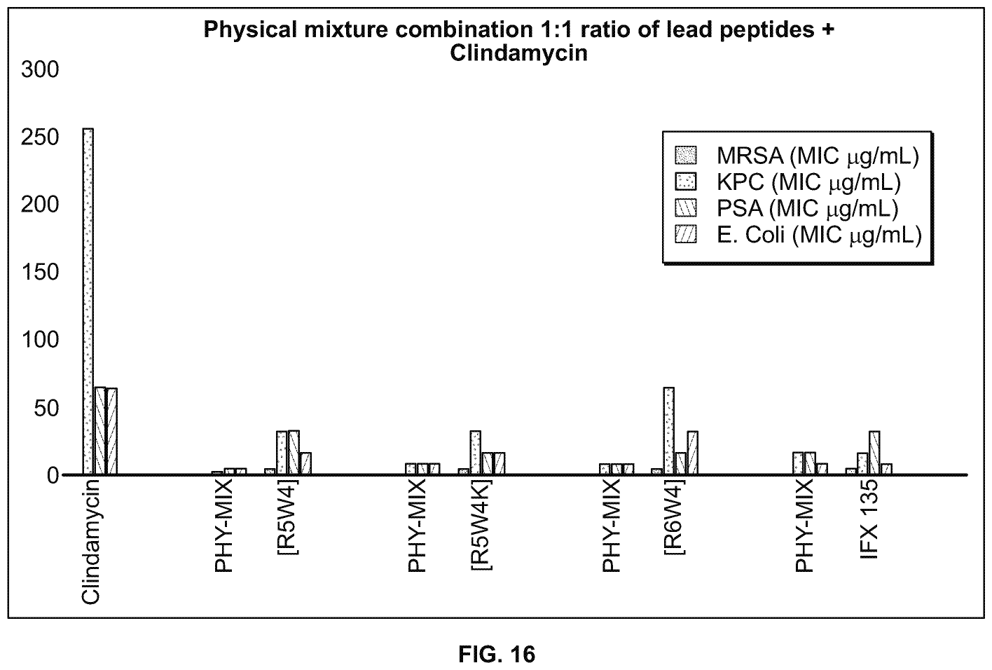

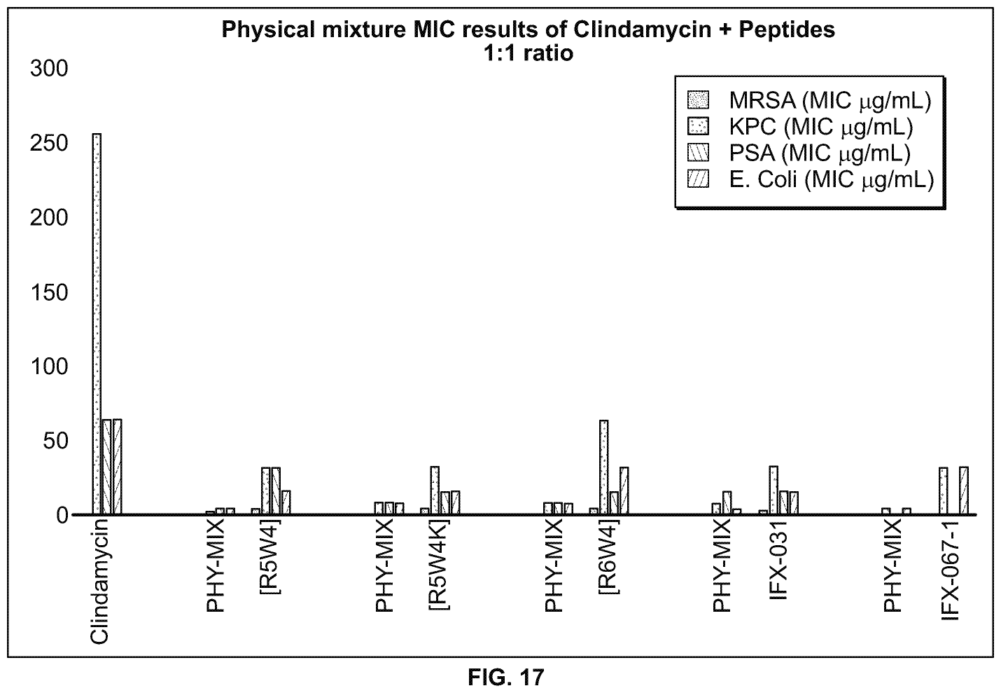

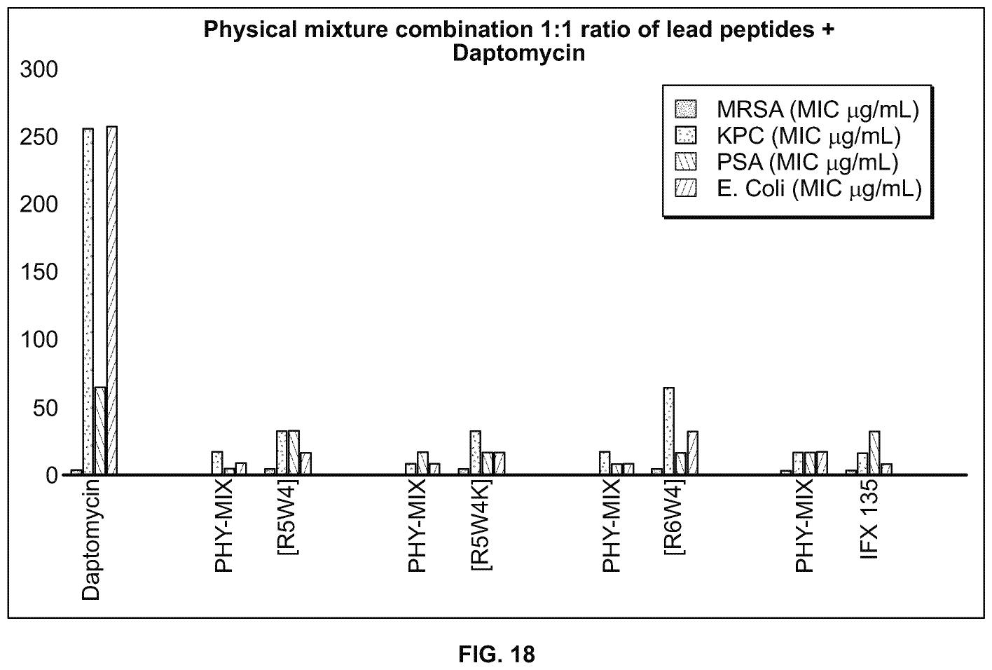

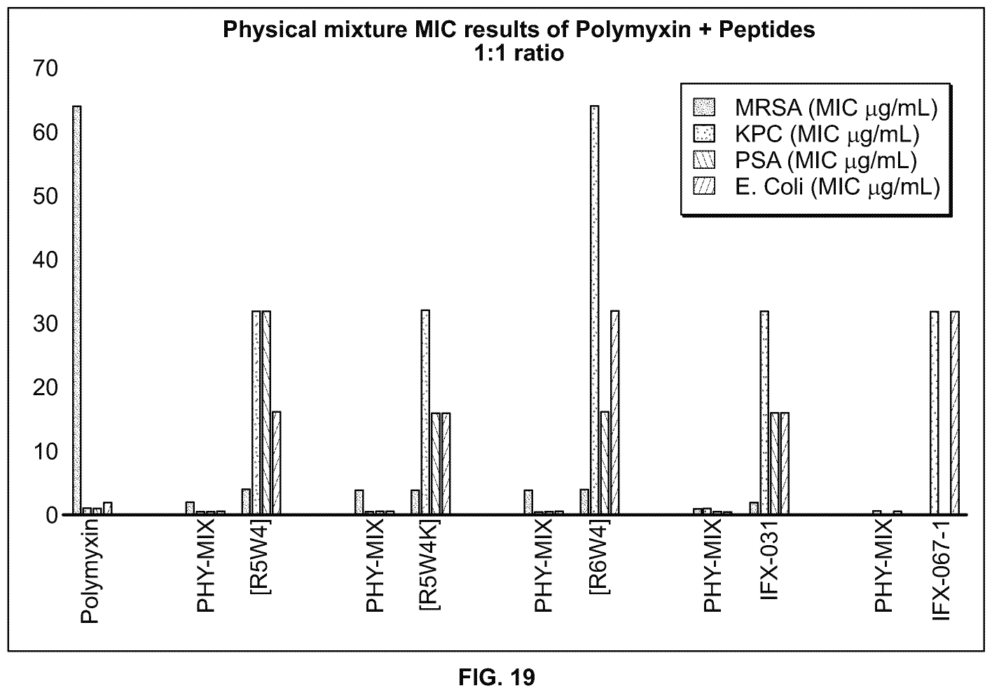

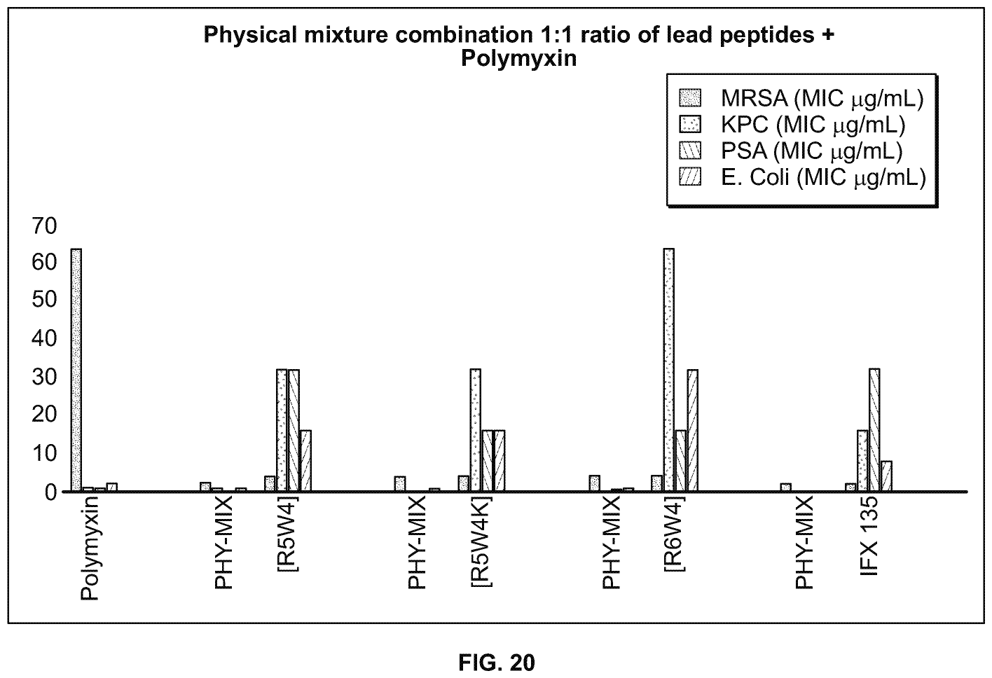

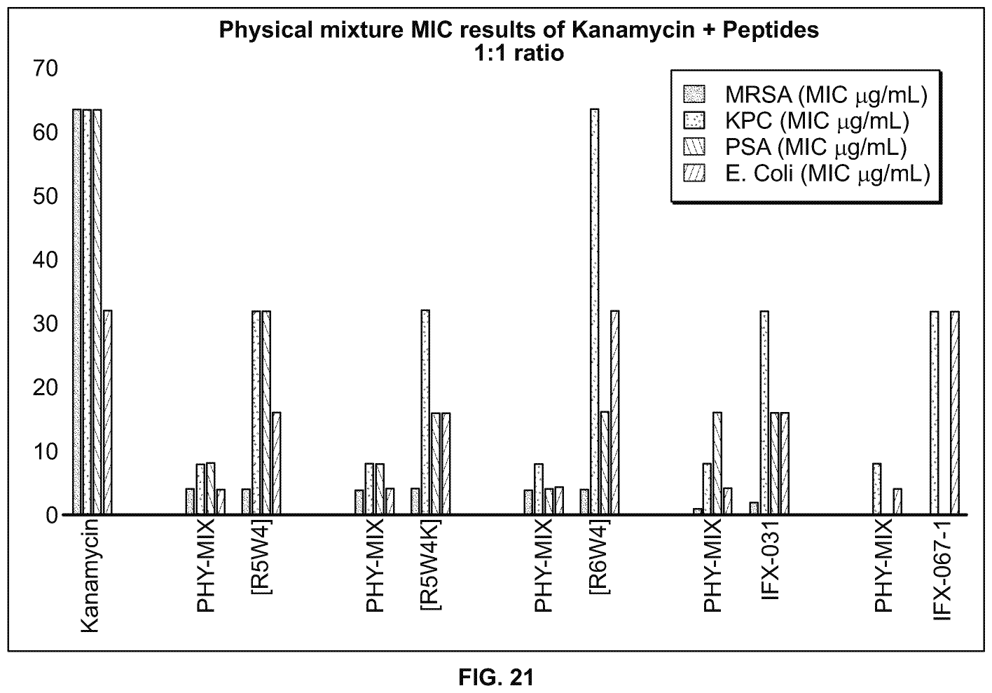

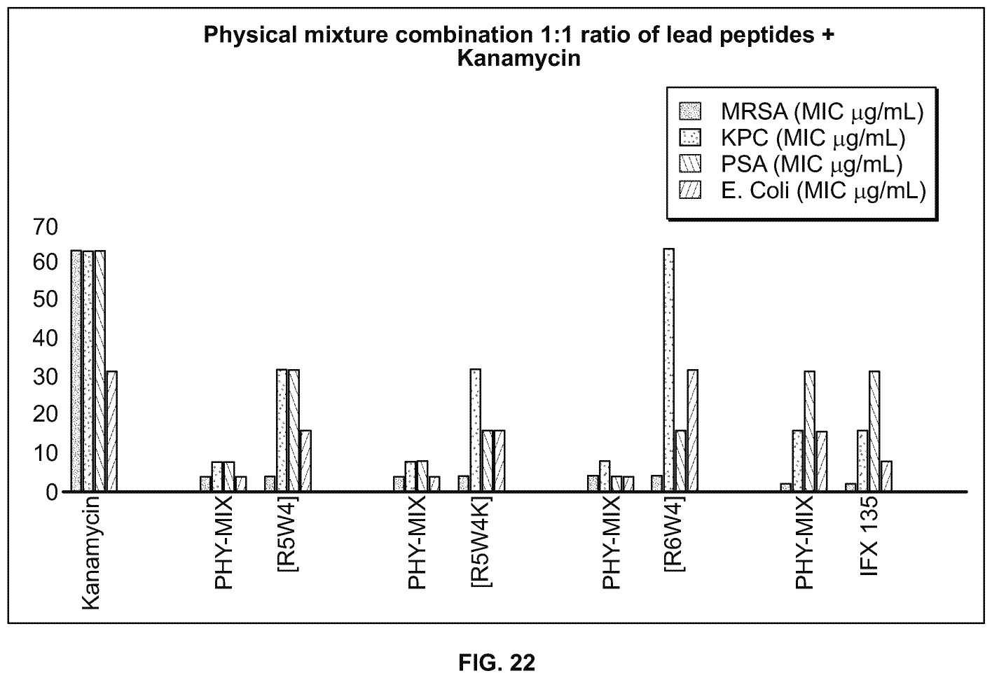

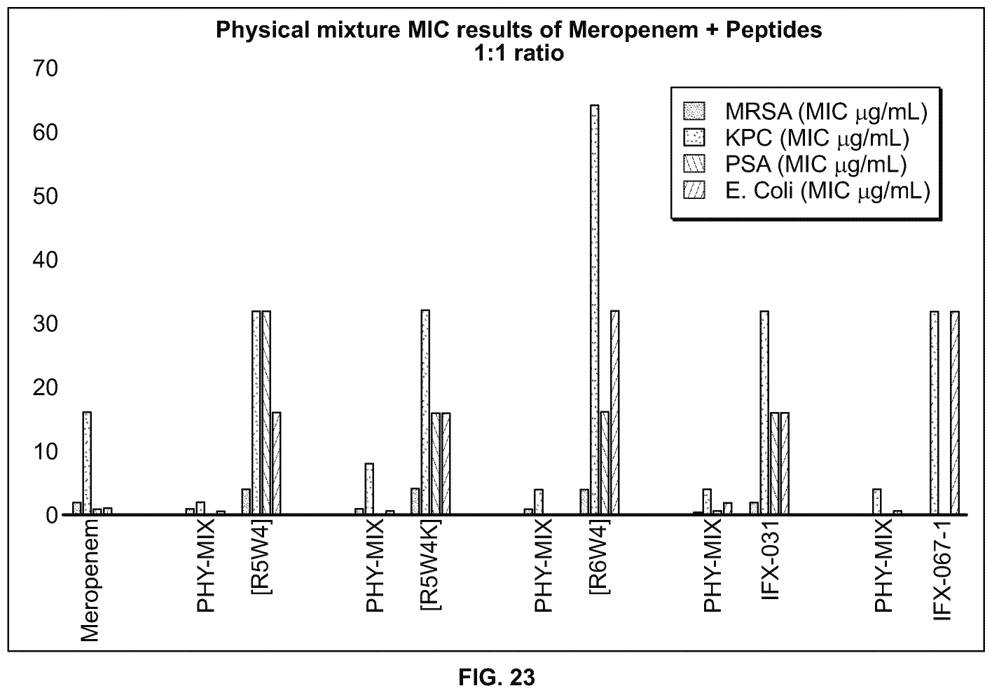

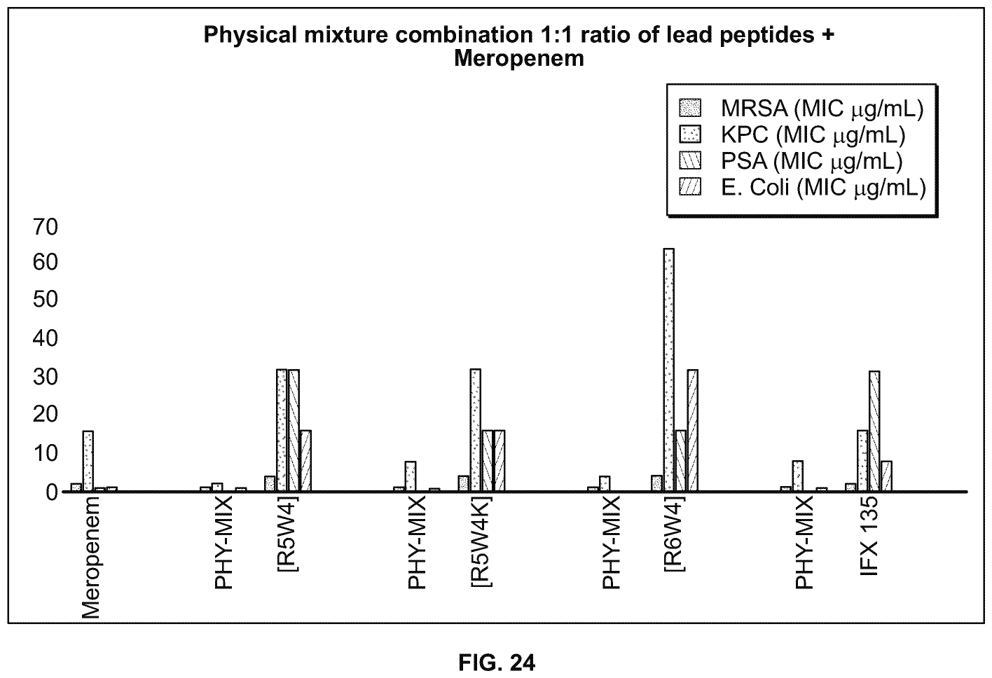

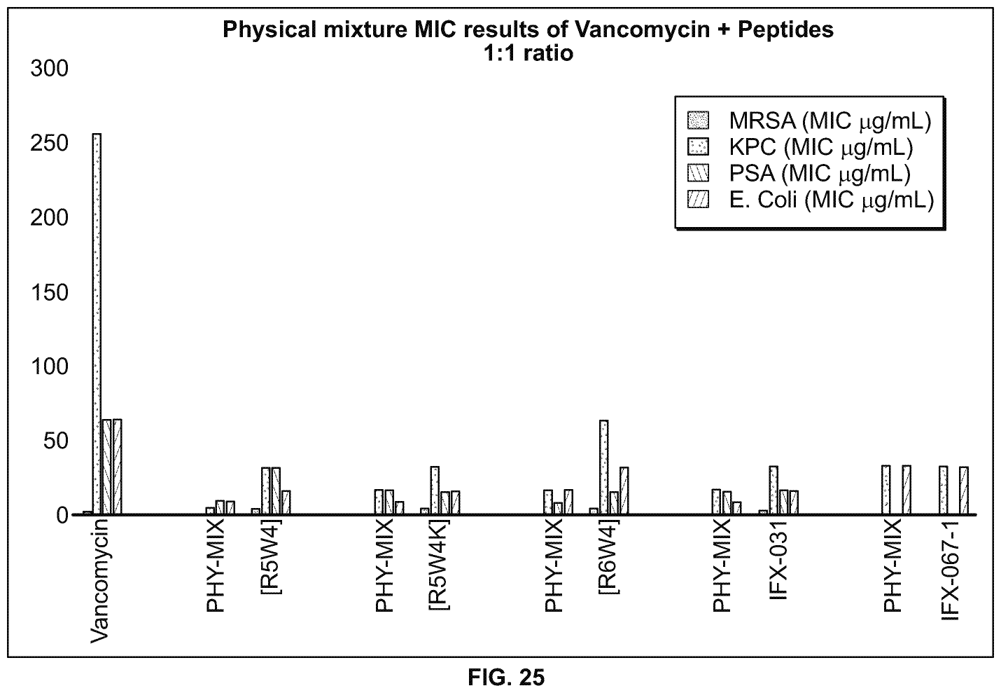

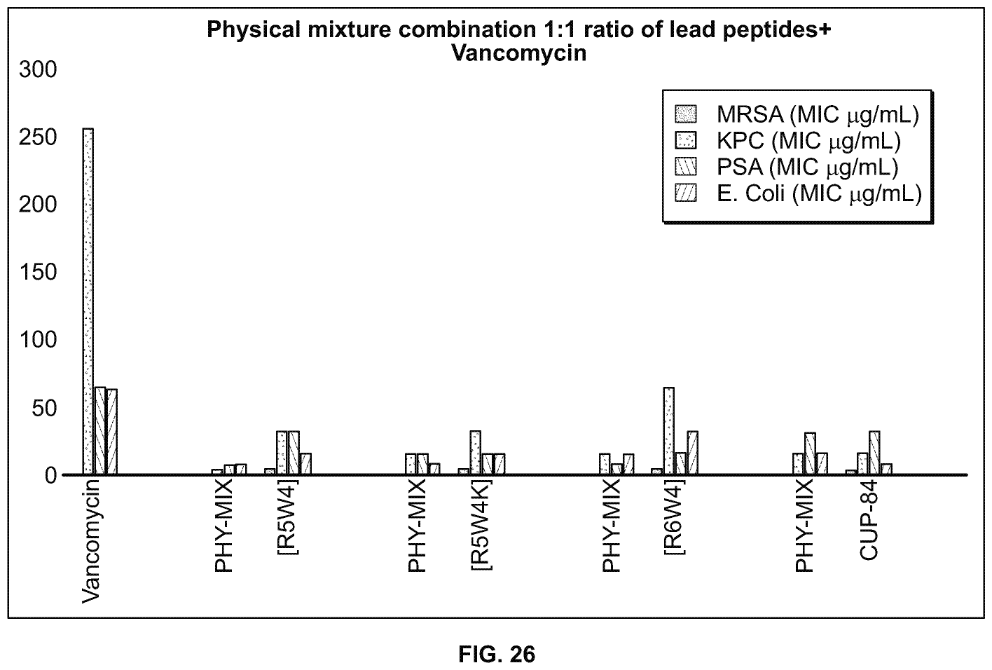

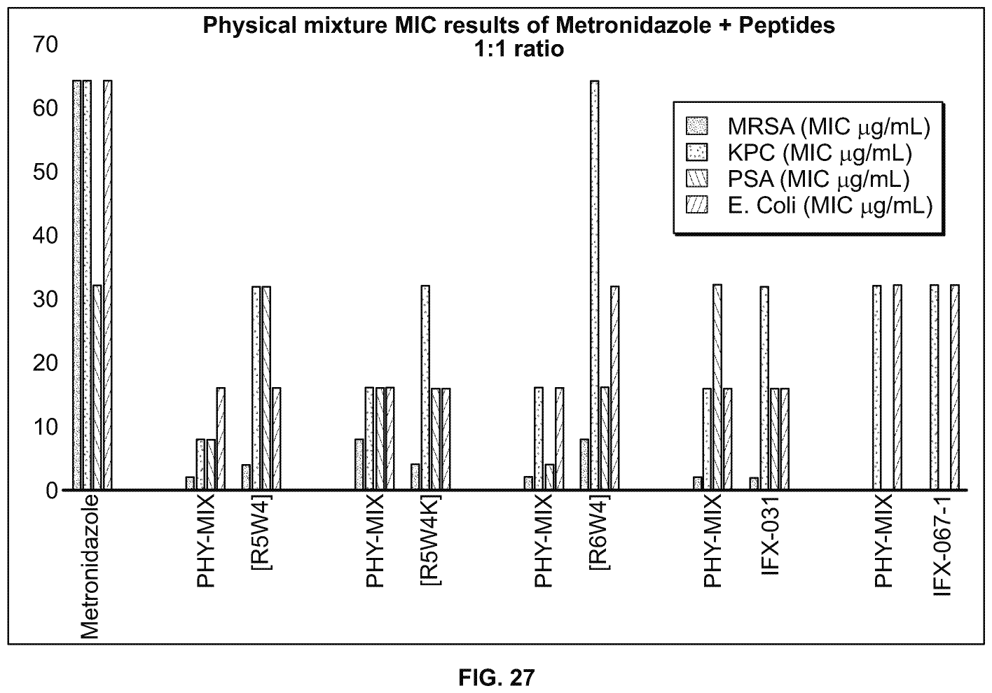

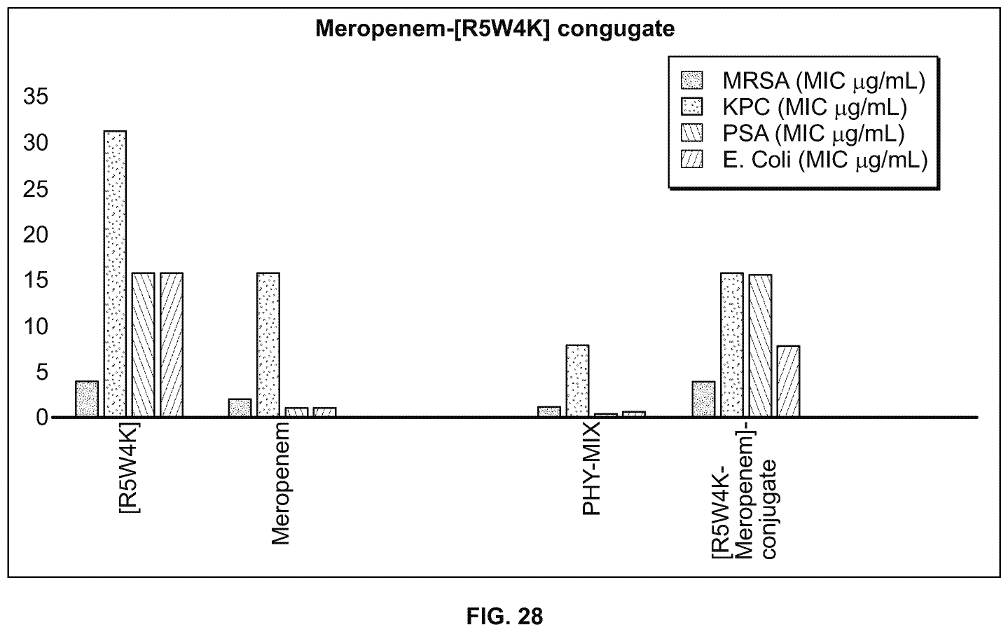

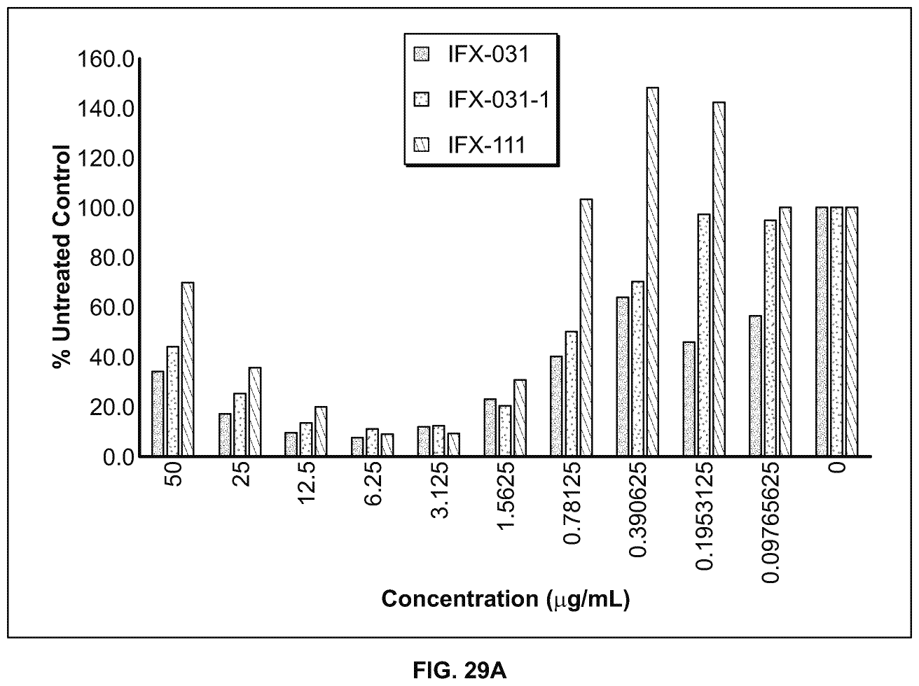

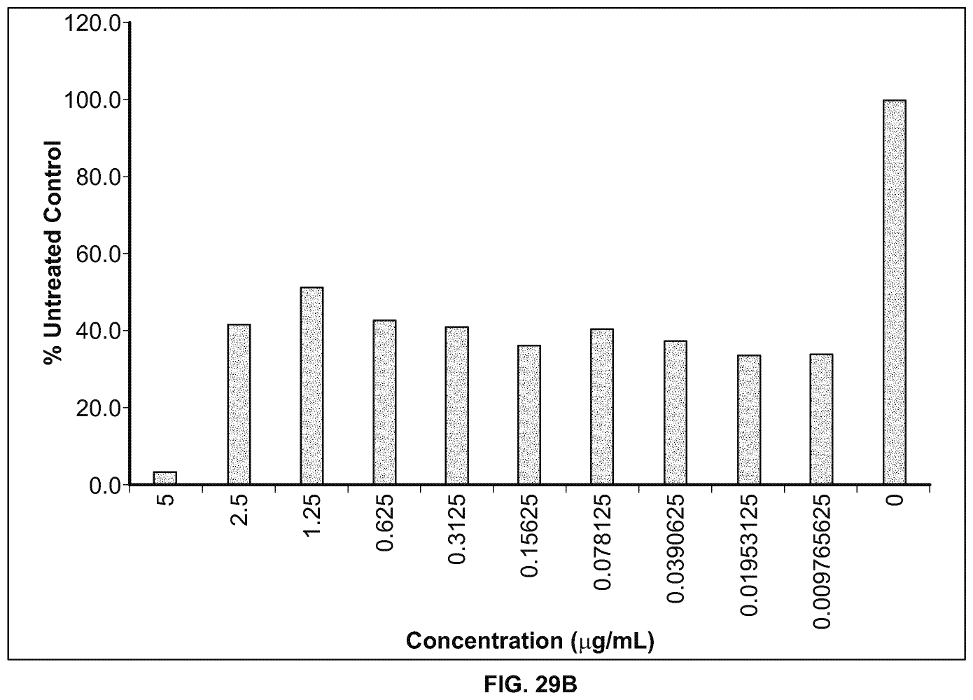

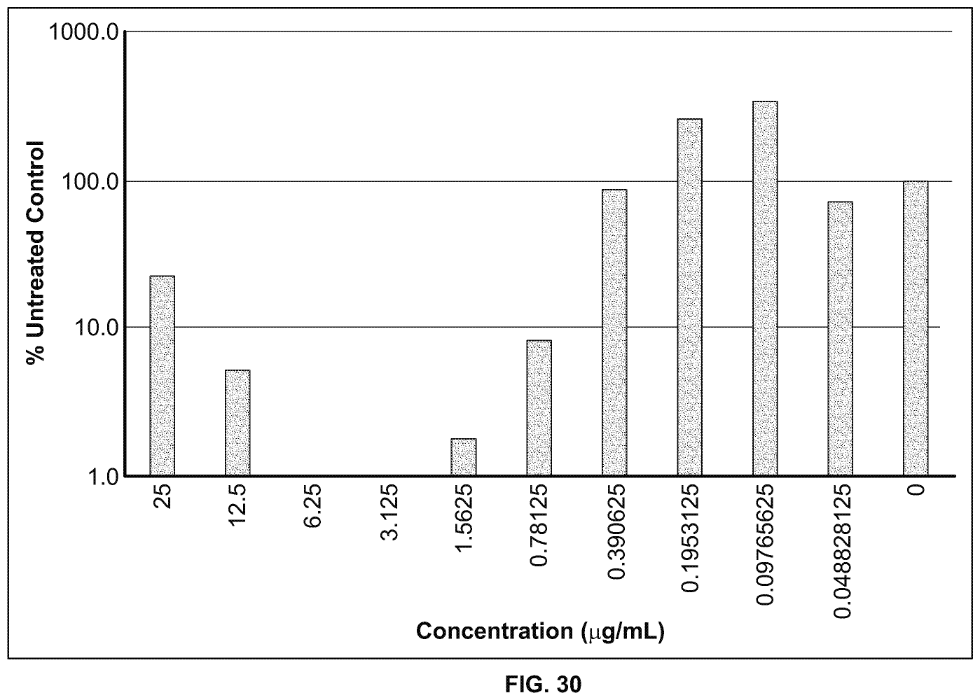

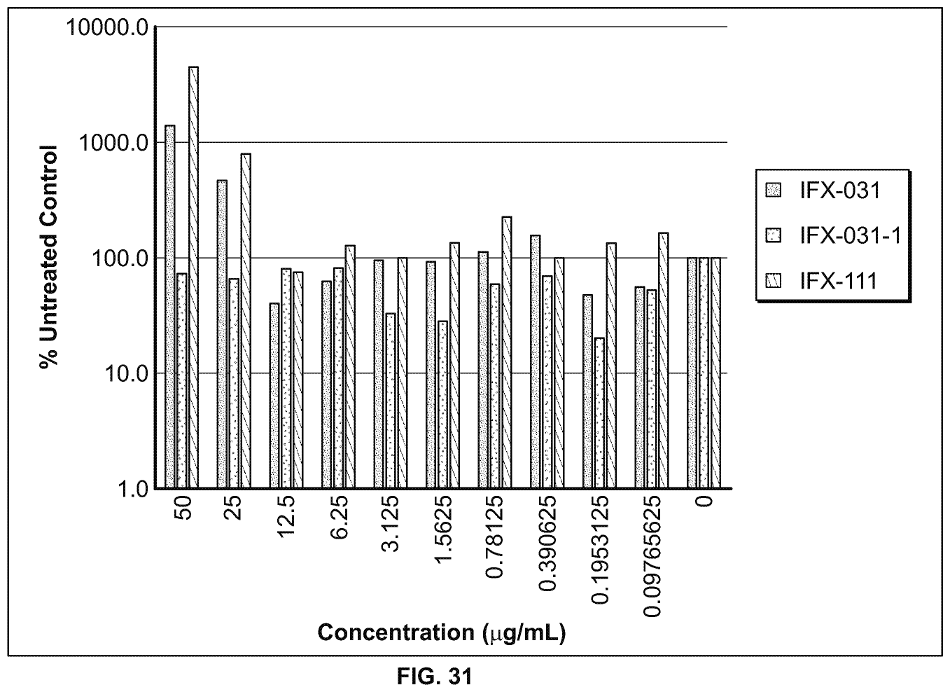

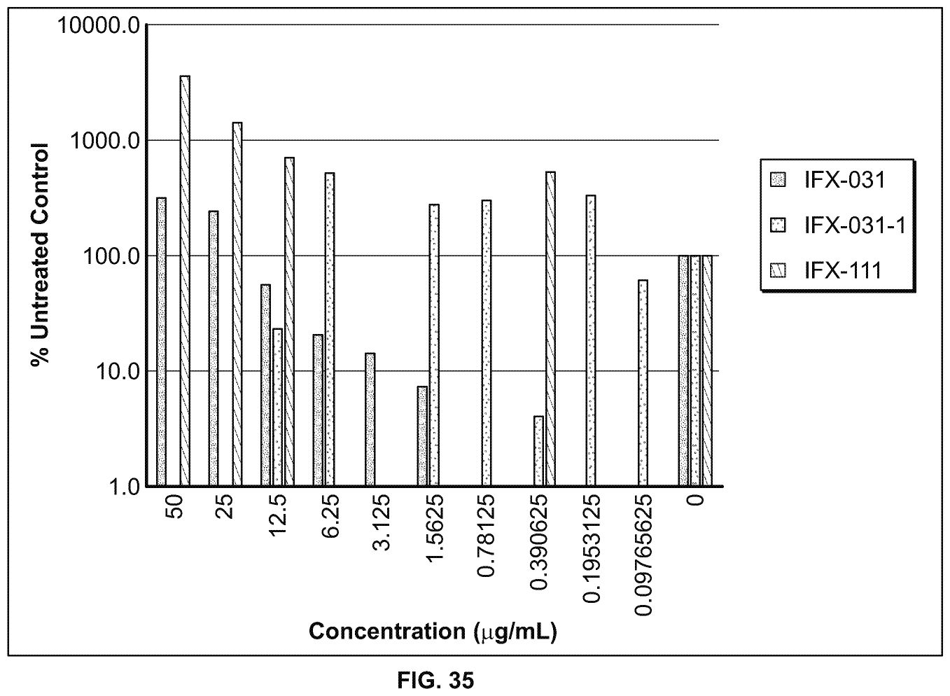

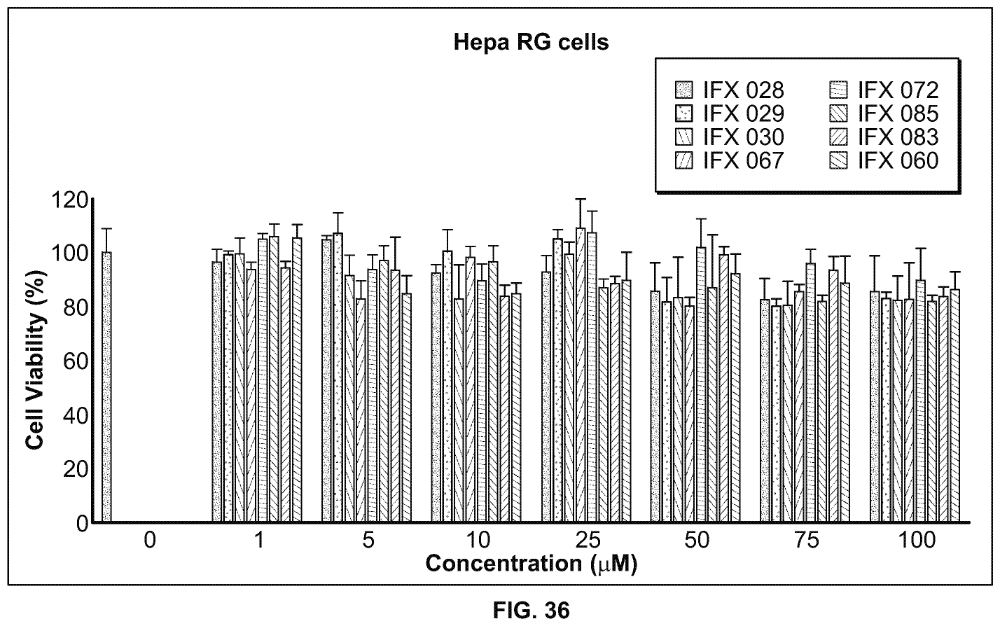

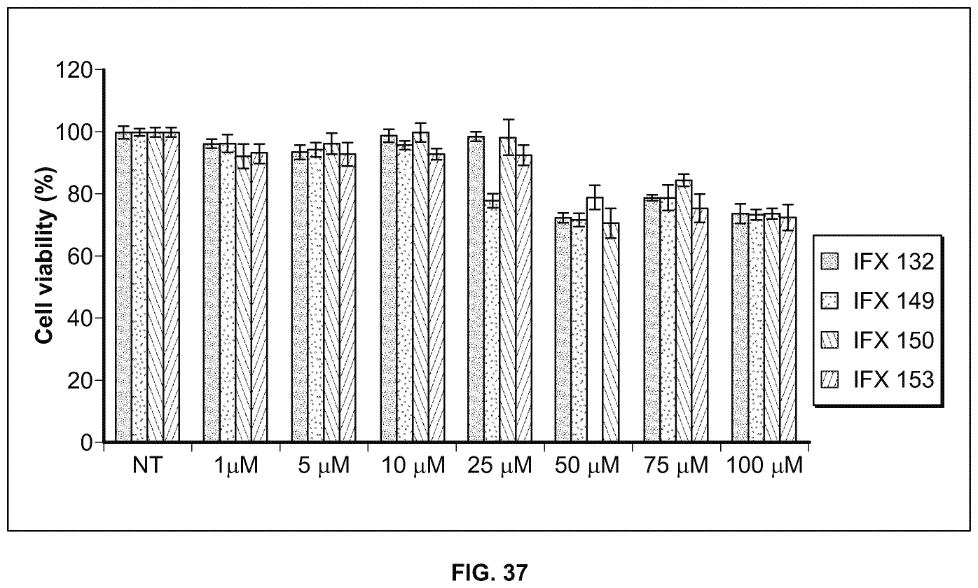

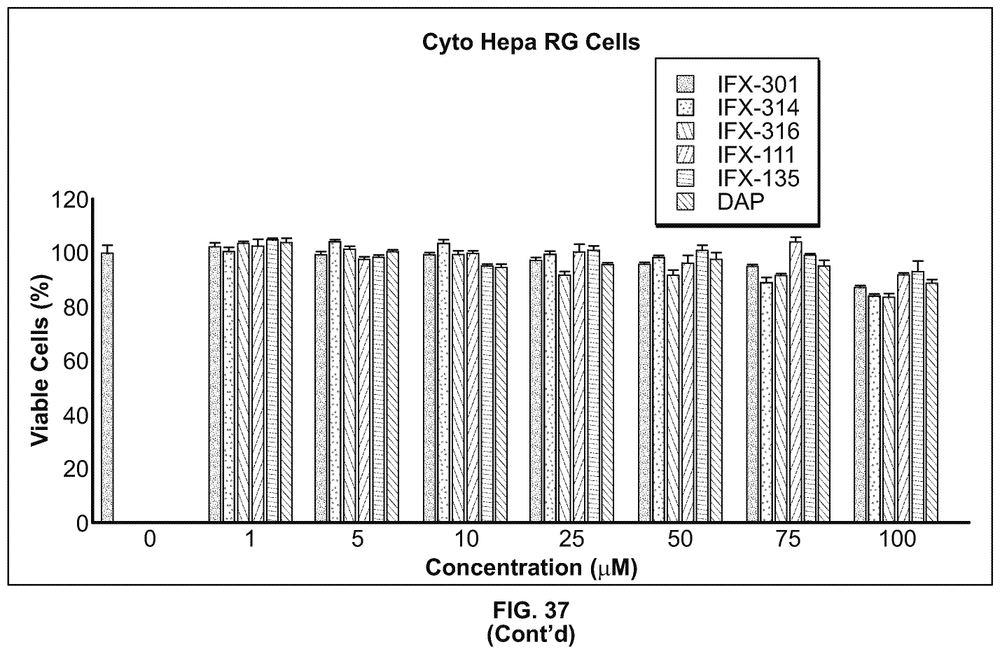

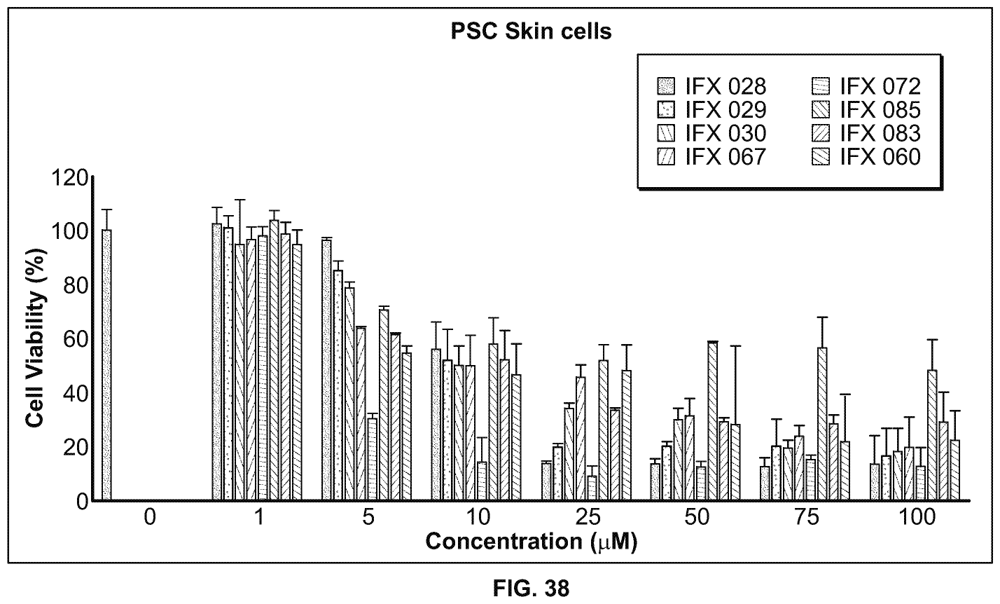

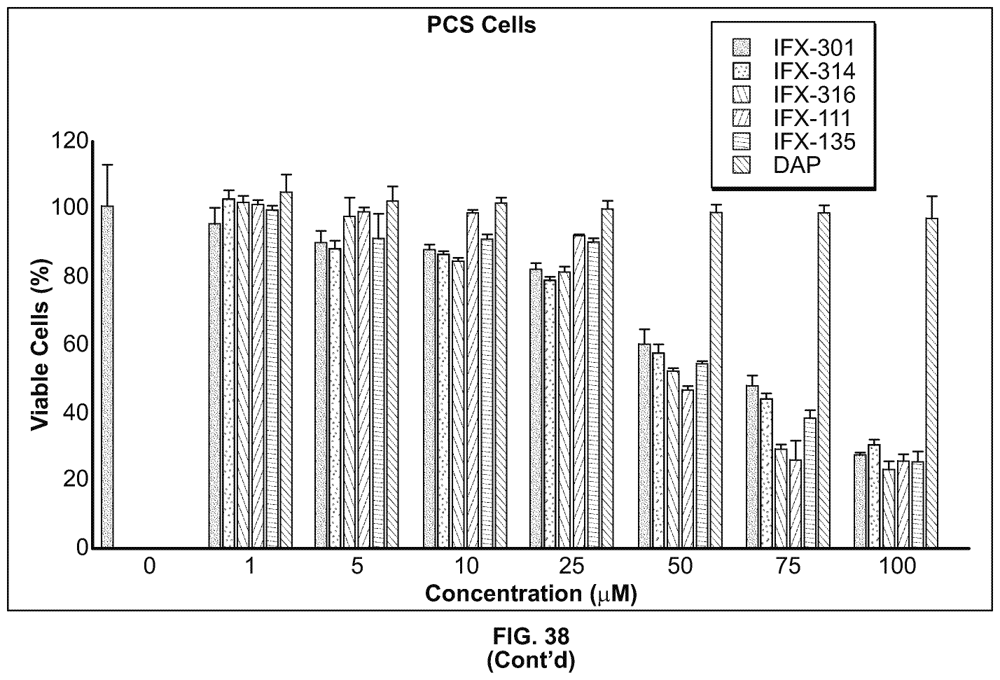

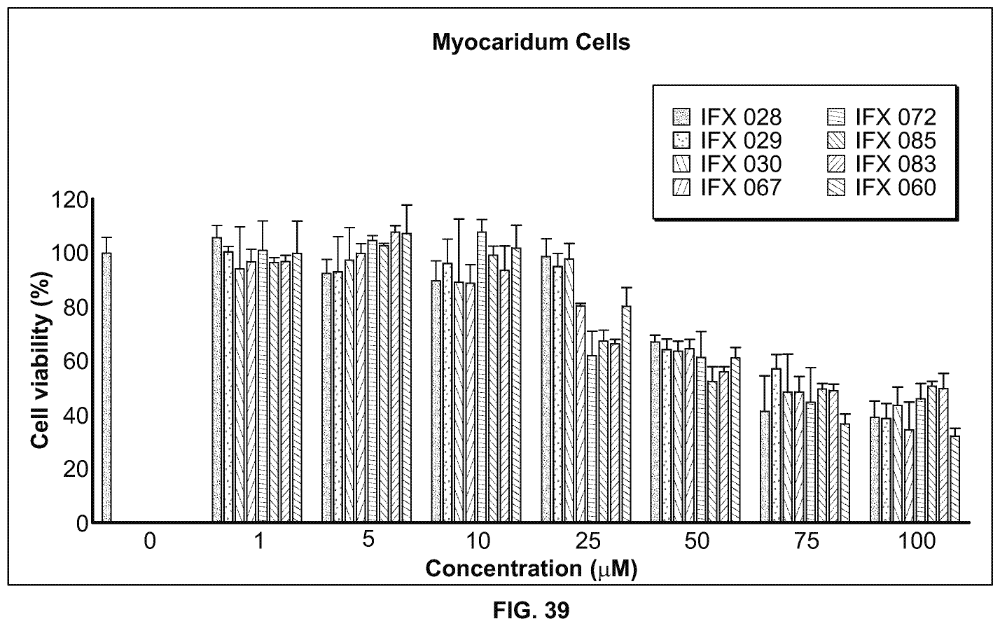

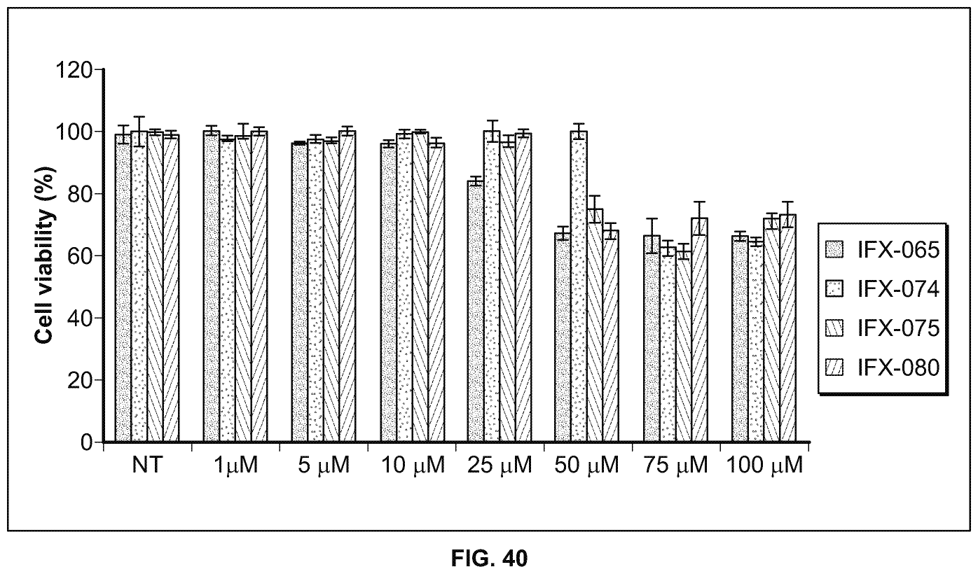

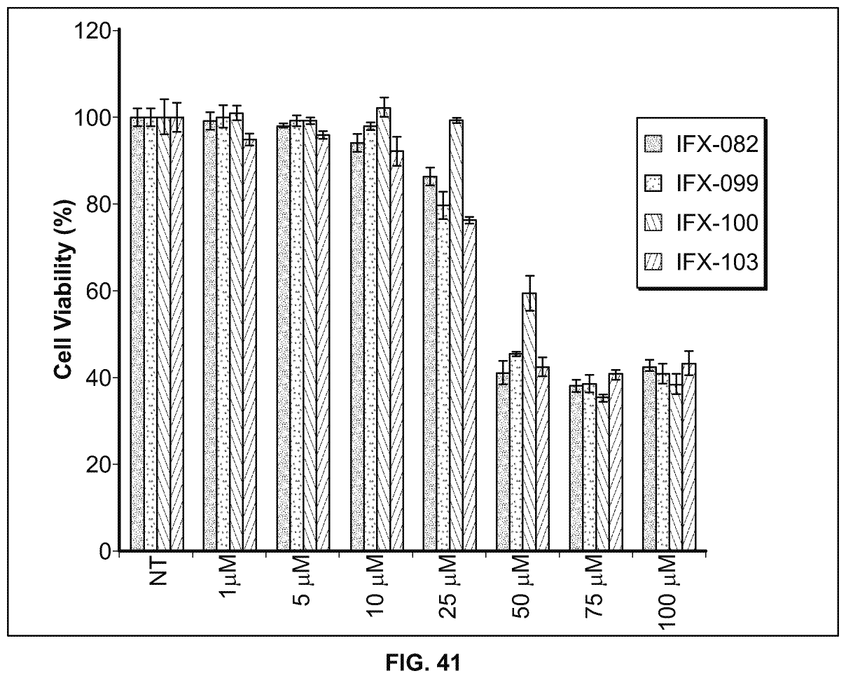

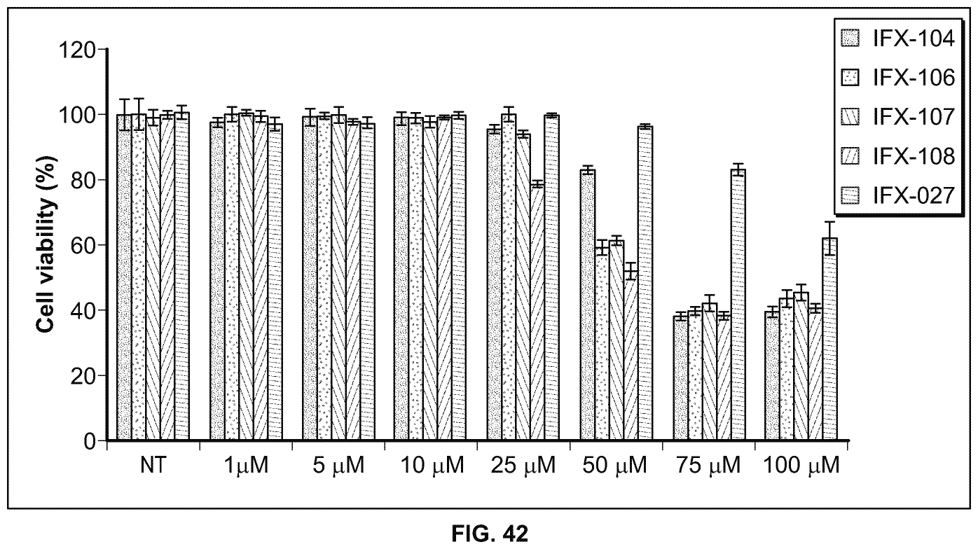

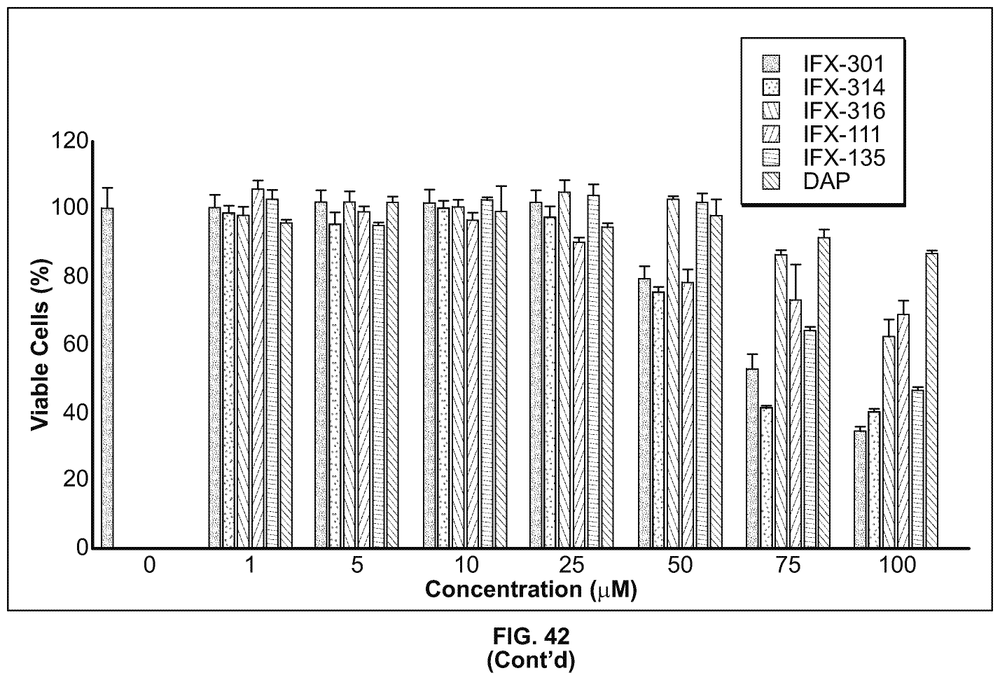

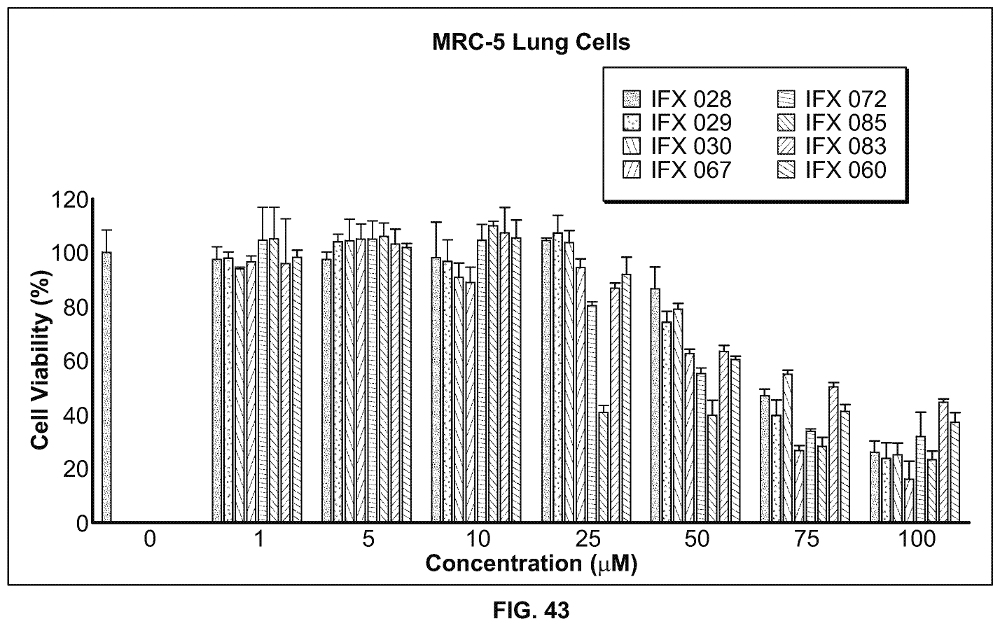

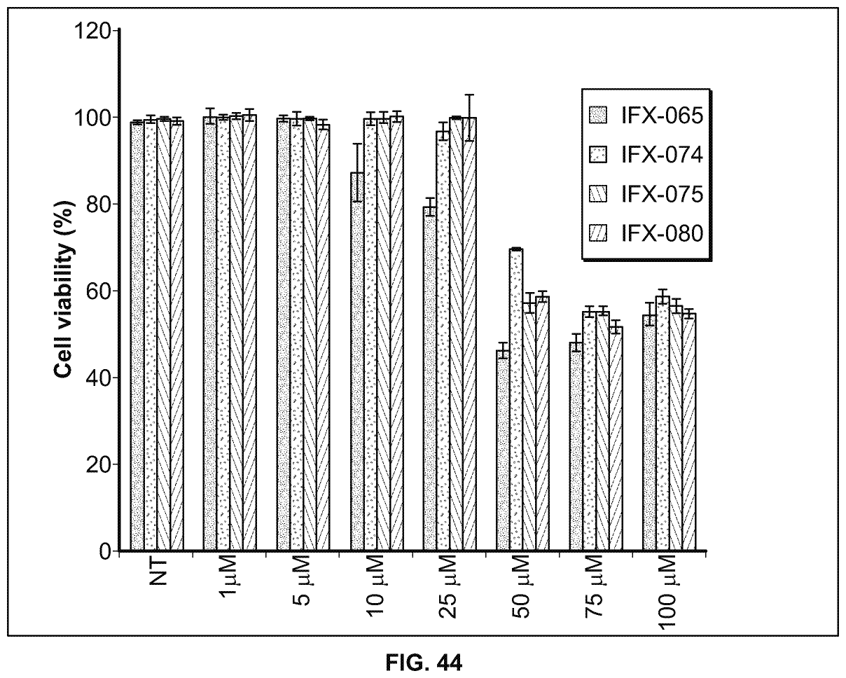

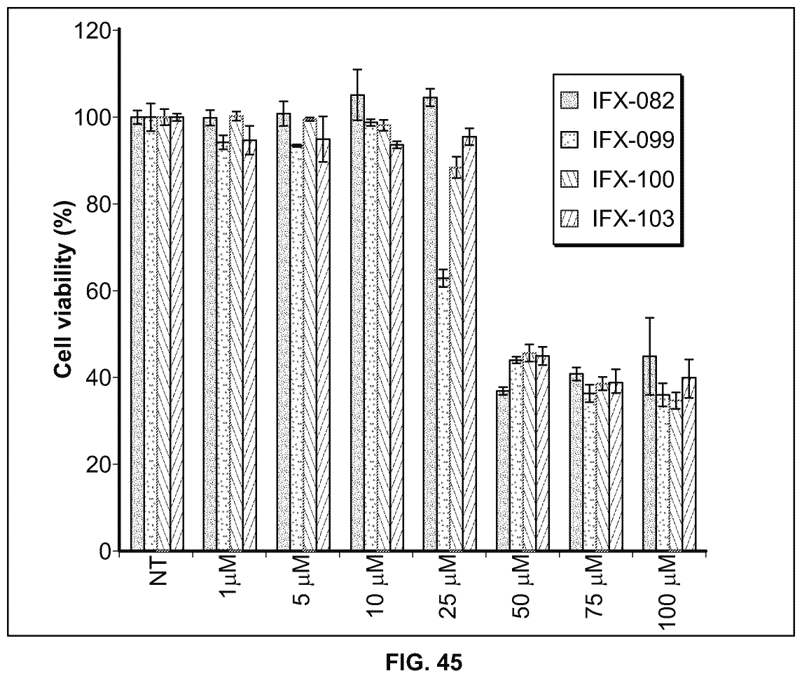

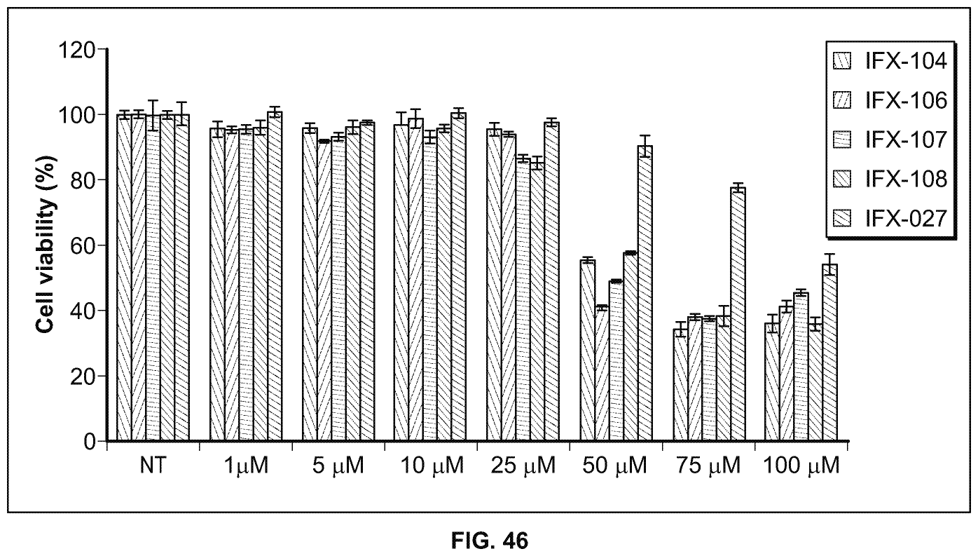

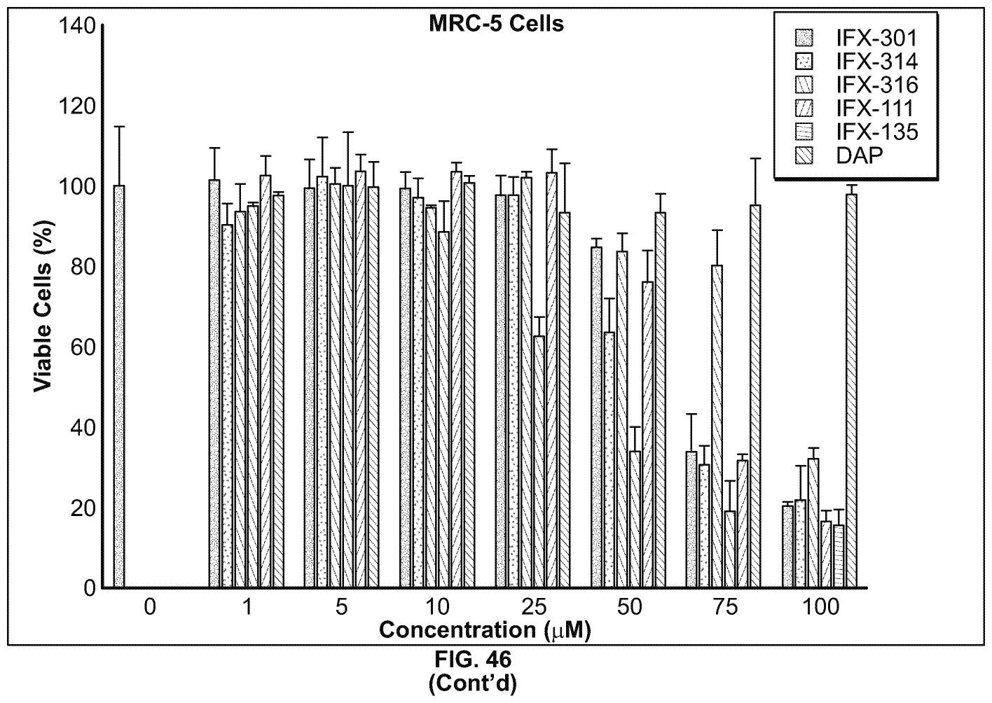

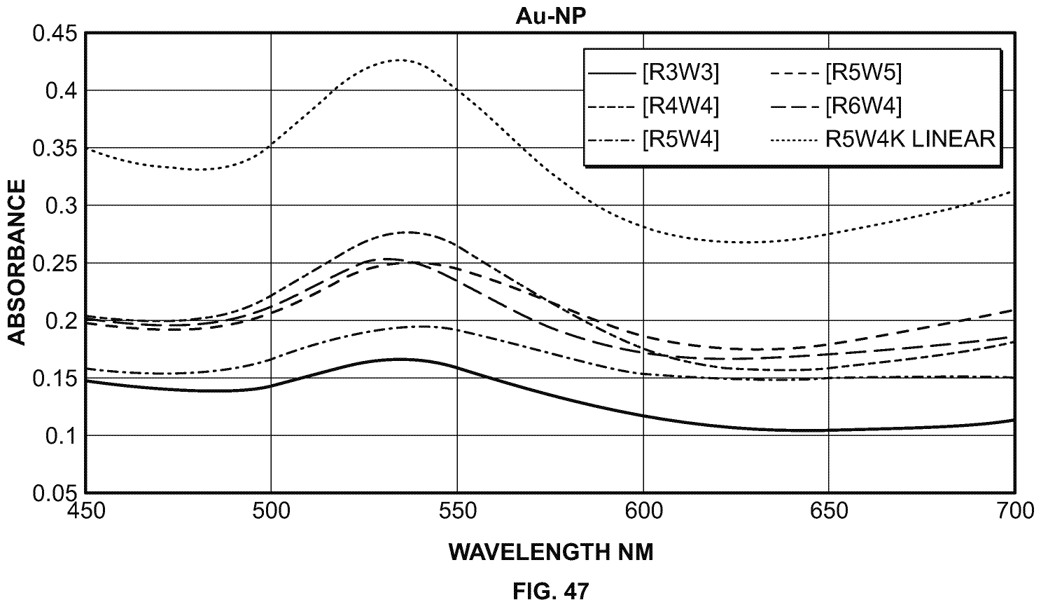

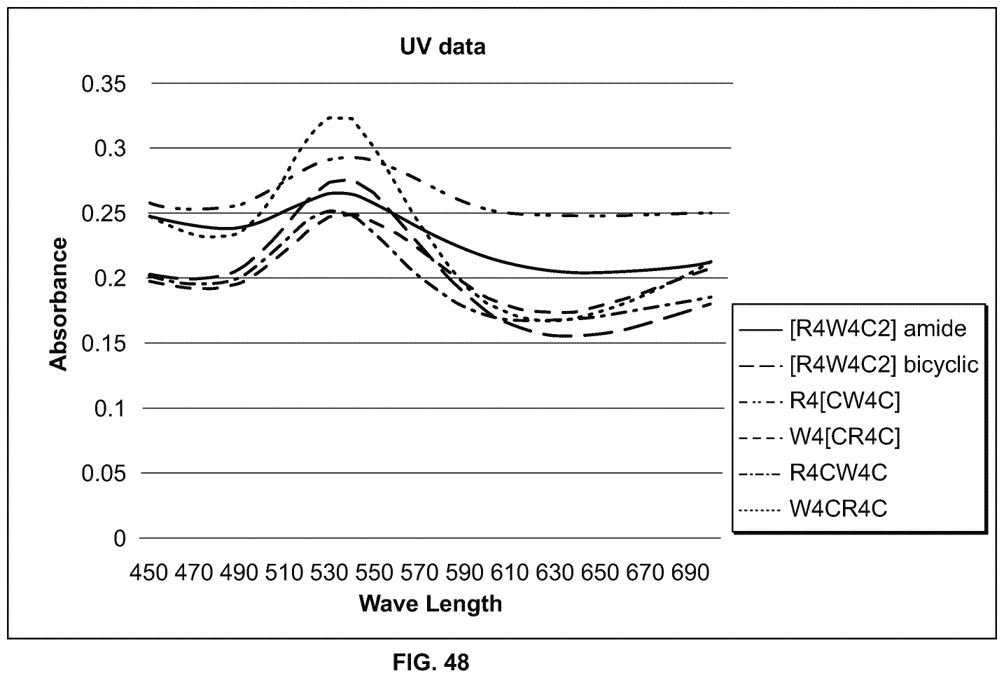

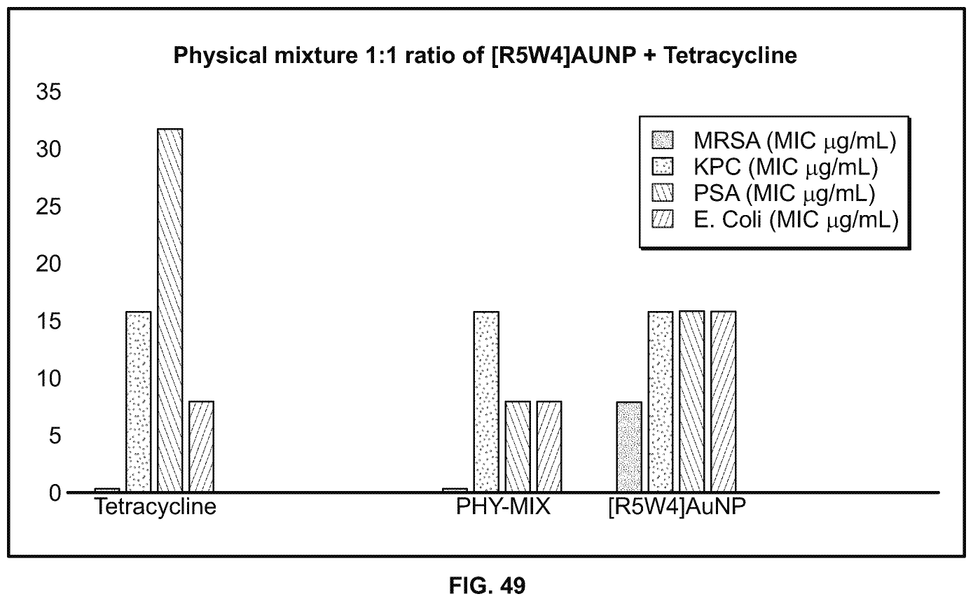

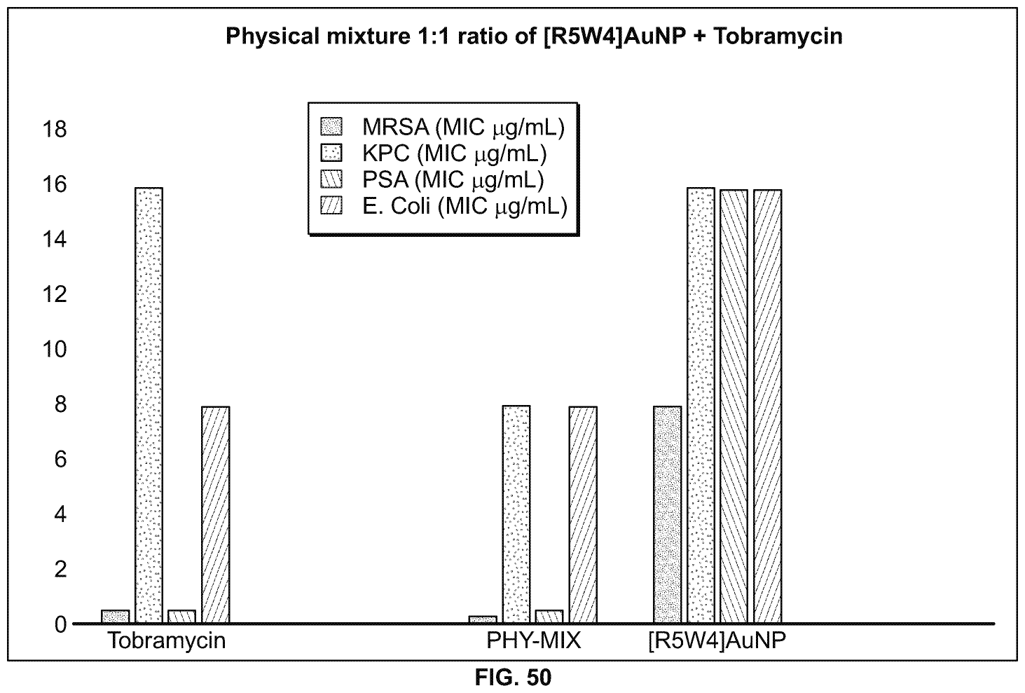

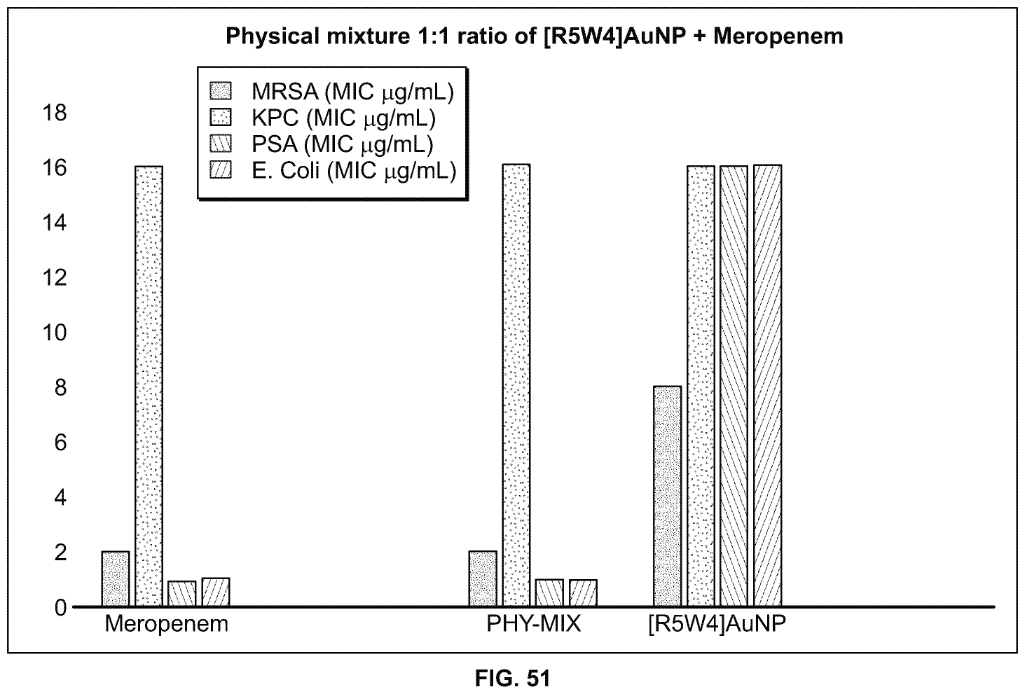

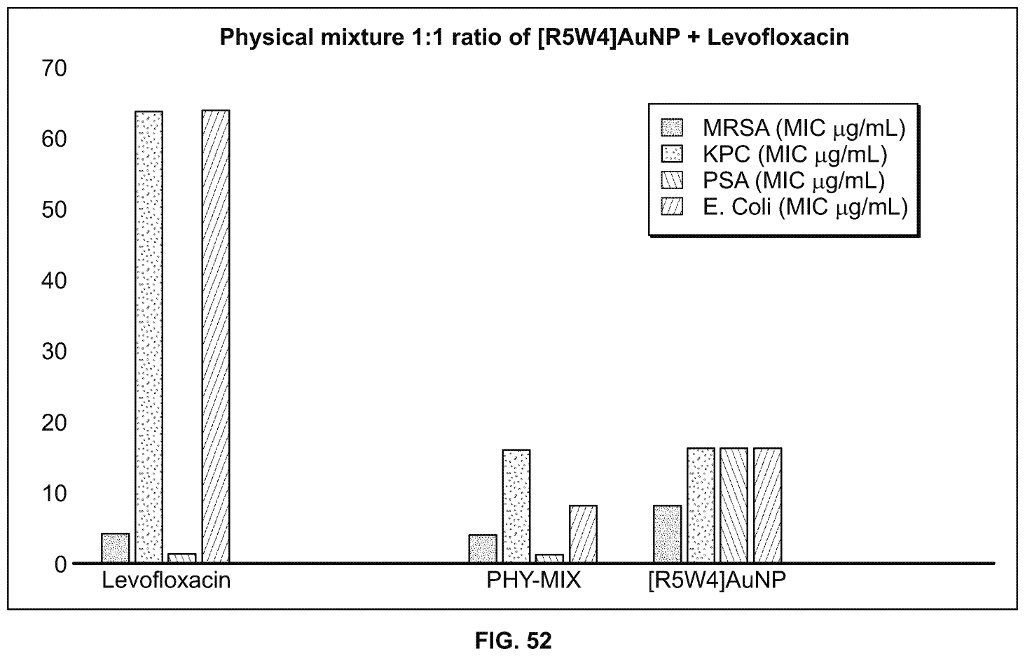

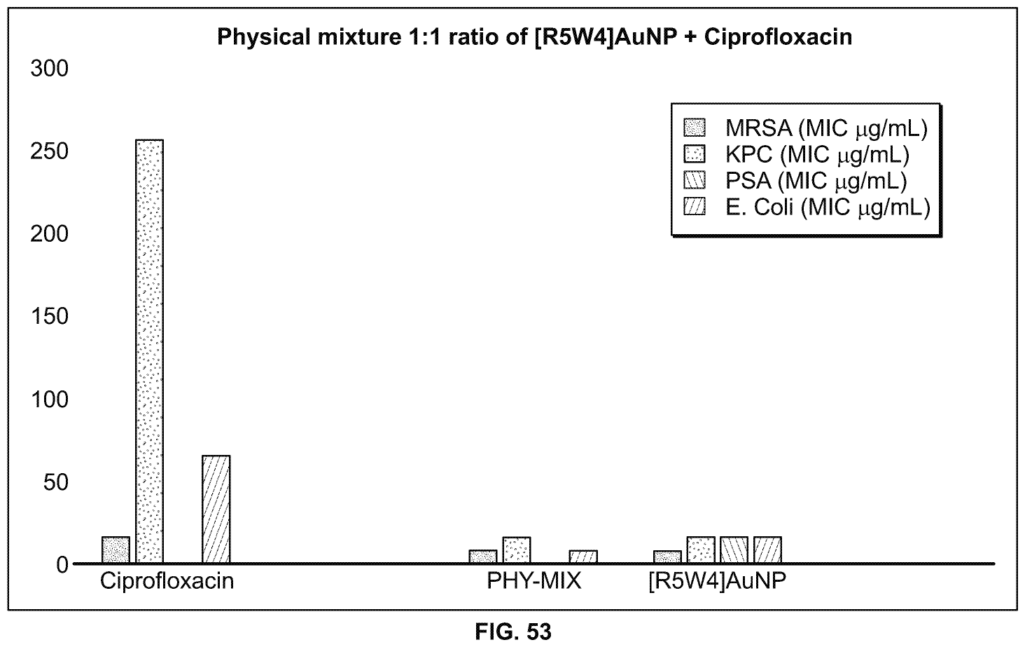

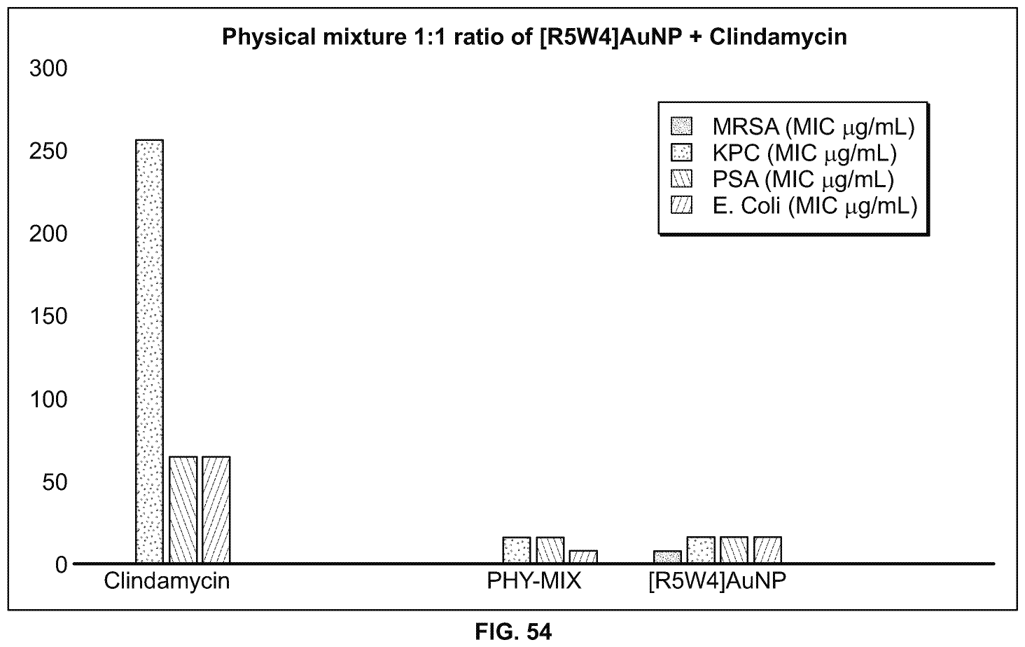

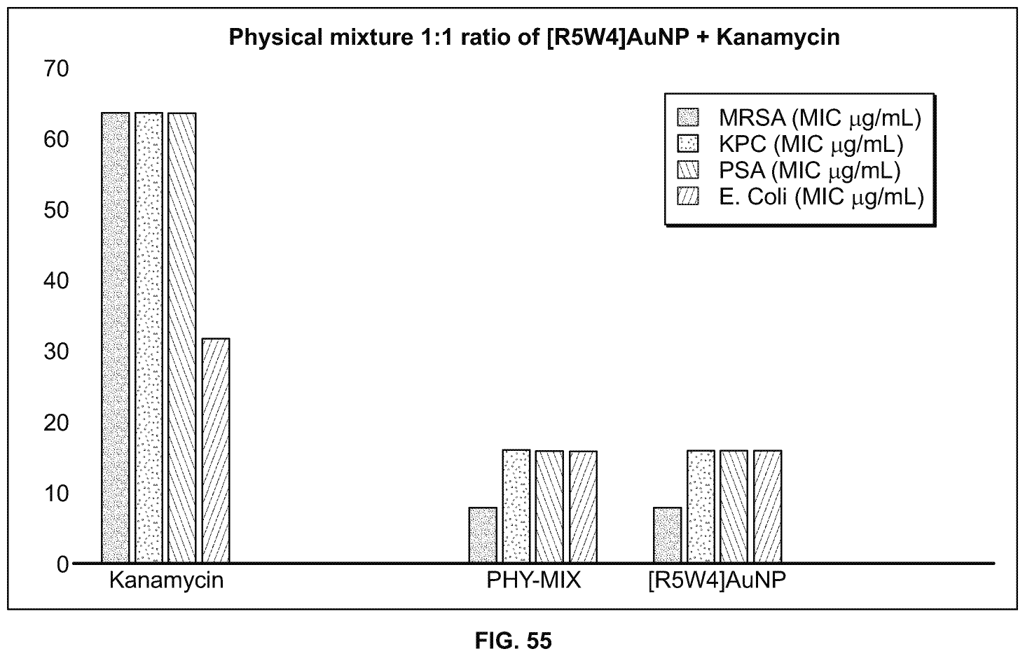

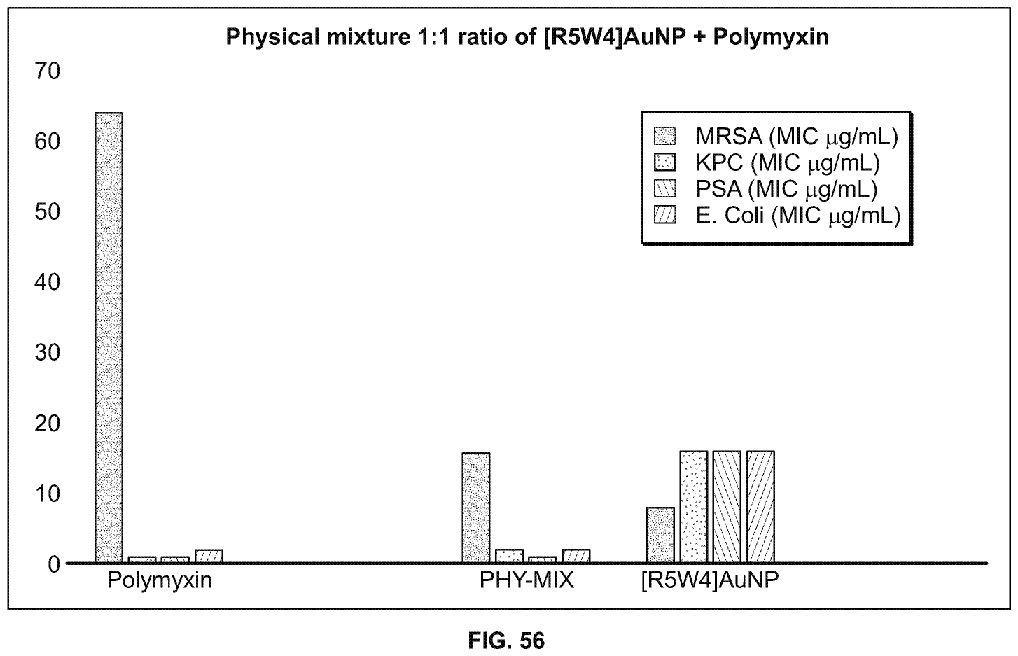

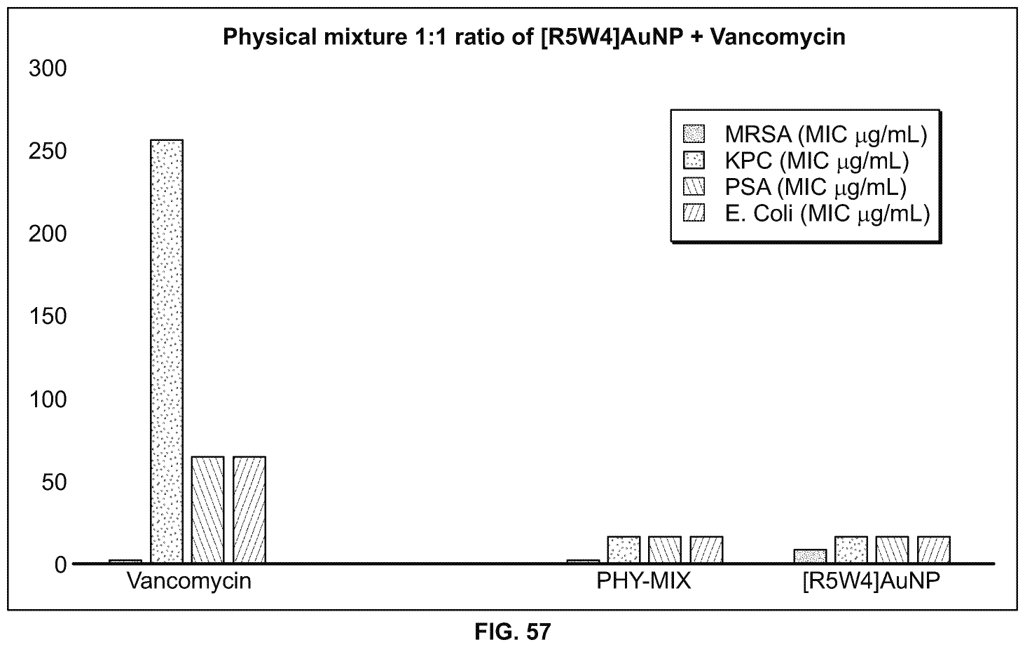

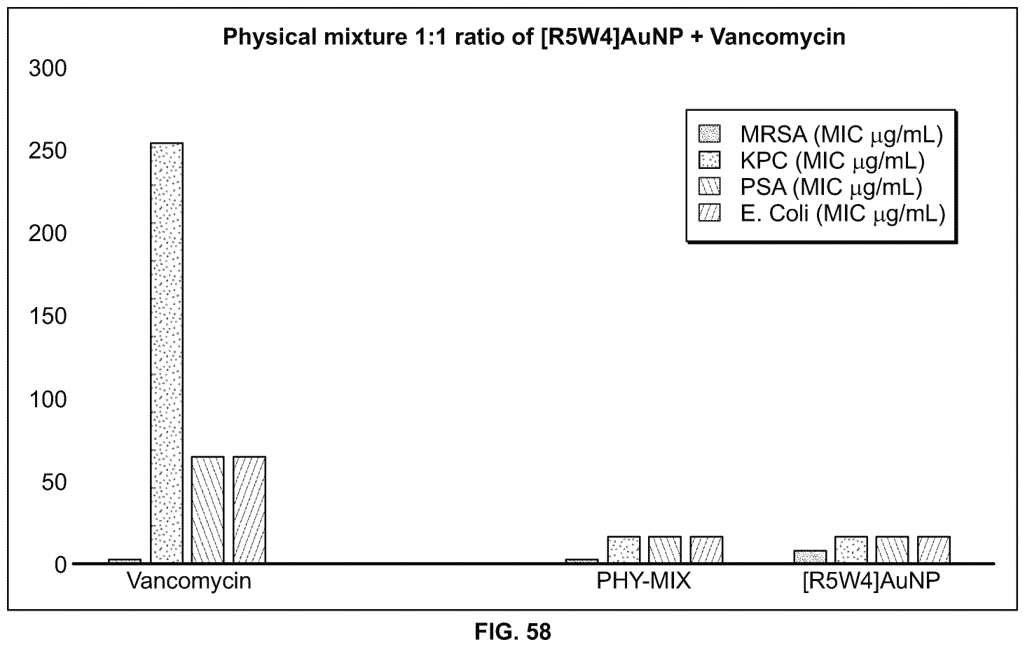

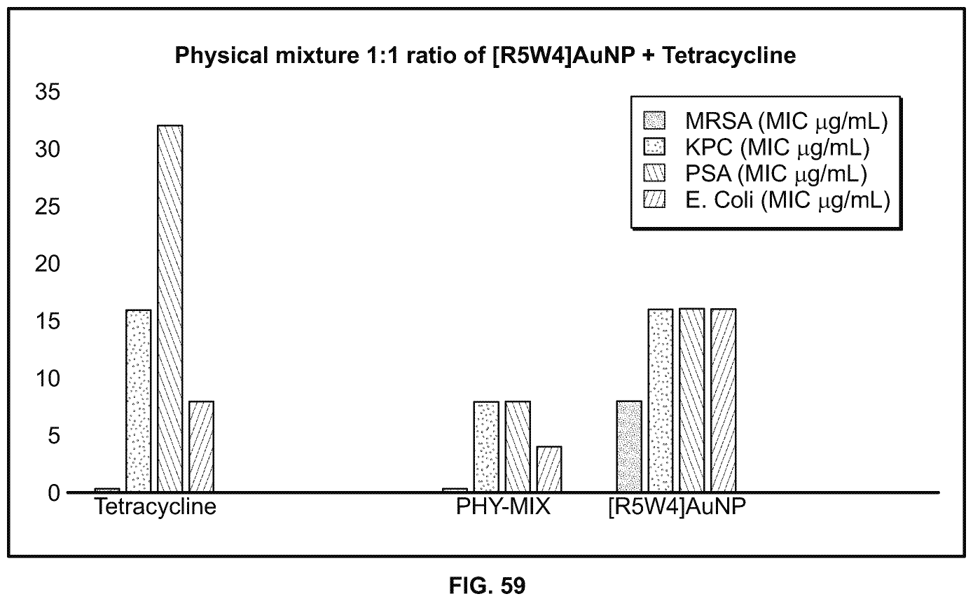

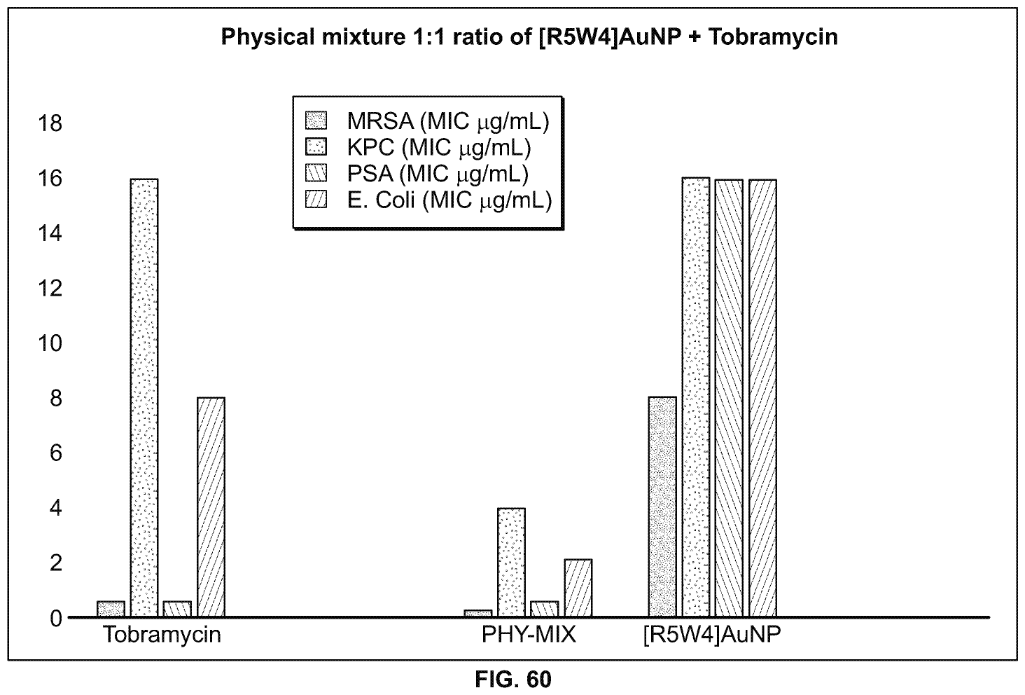

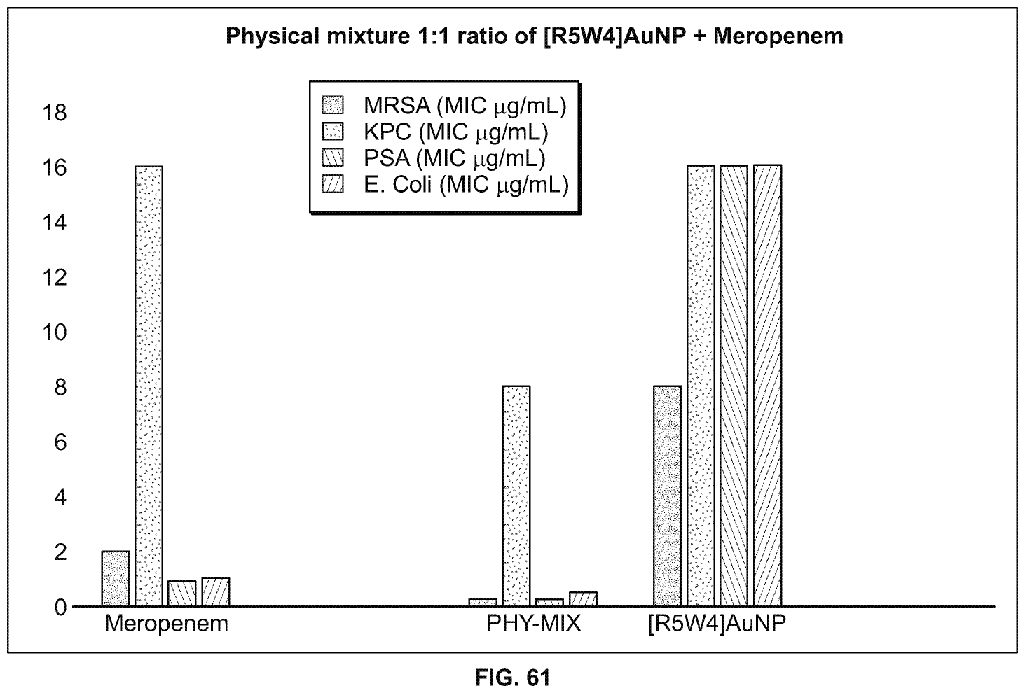

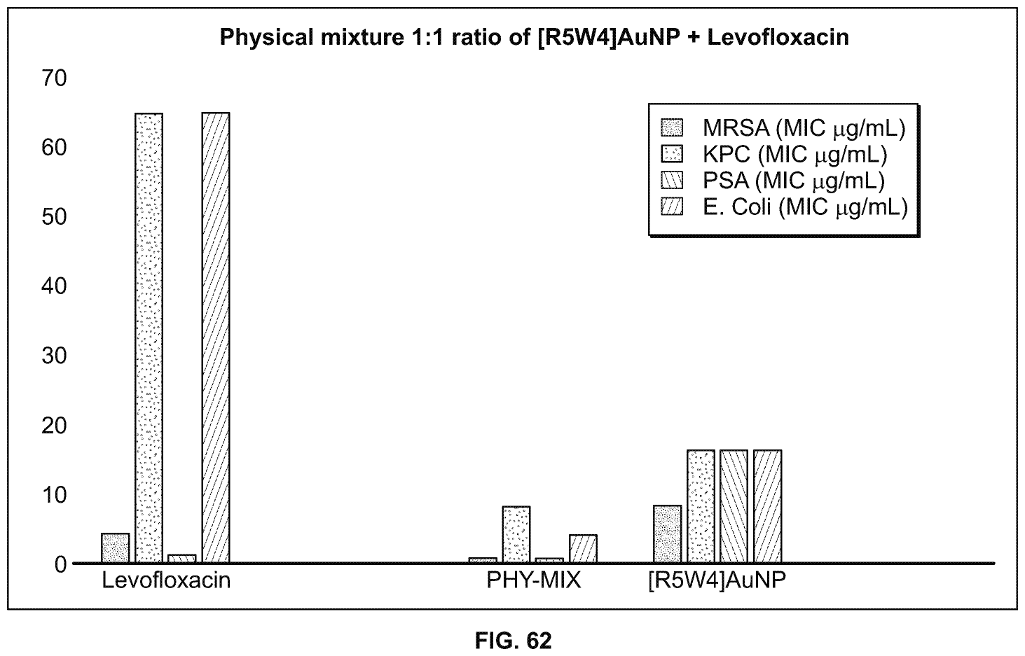

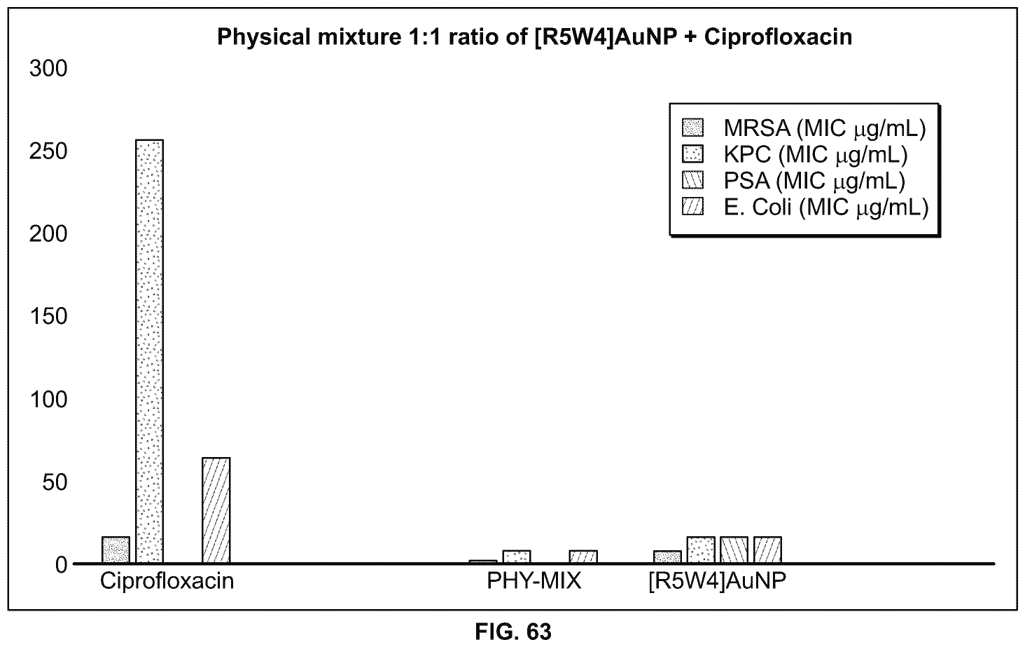

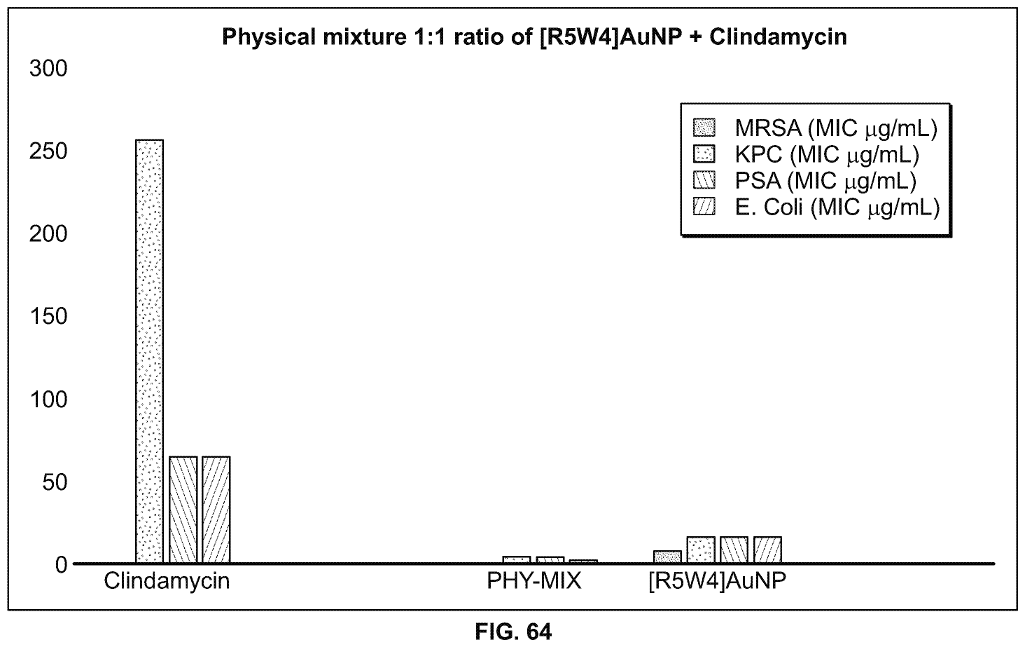

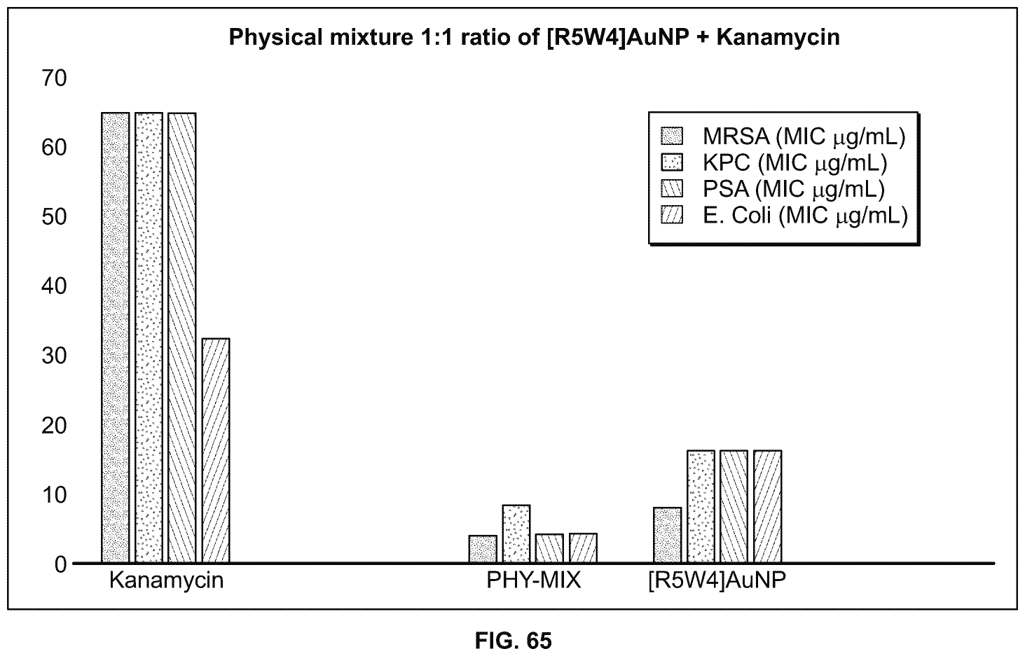

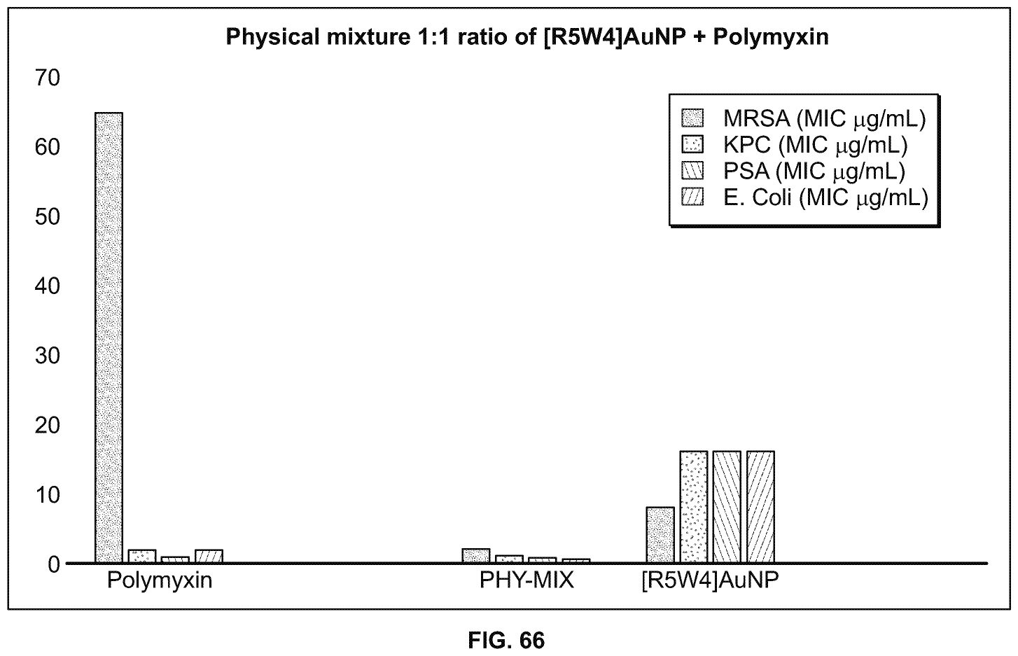

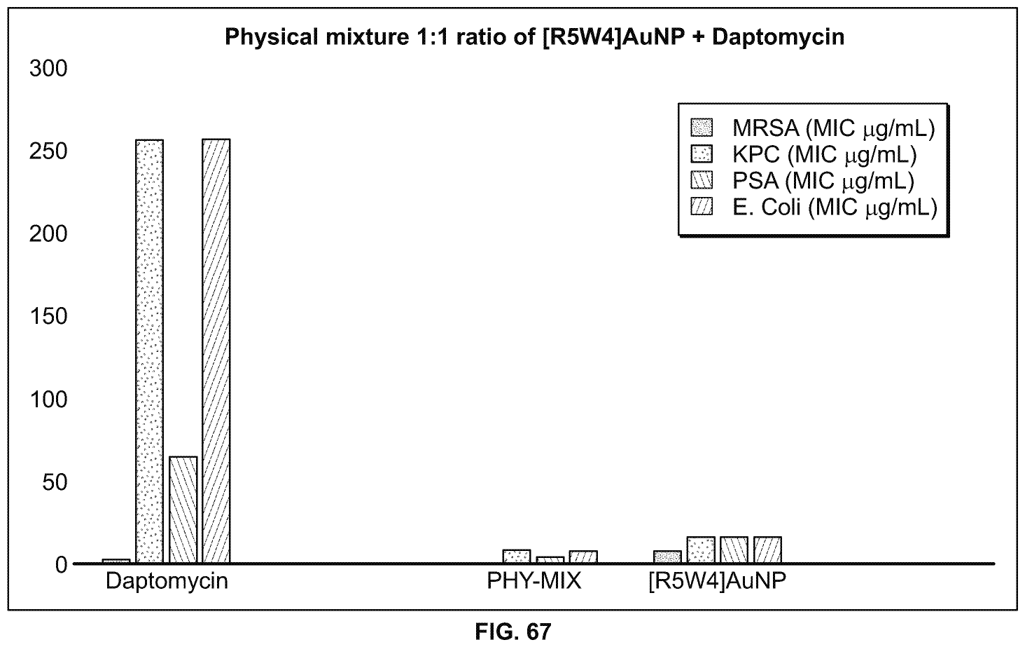

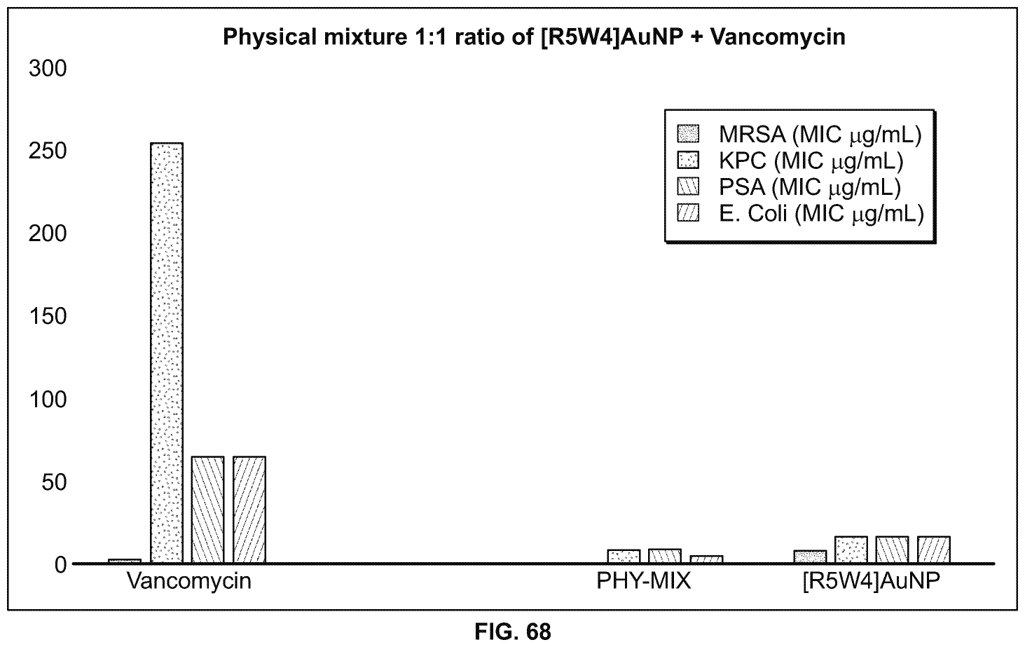

Micro-broth dilution method was employed to determine the minimum inhibitory concentration of each synthesized peptide using vancomycin and meropenem as positive controls against Gram-positive and Gram-negative strains, respectively. All bacteria pathogens tested clinically reported multi-drug resistant strains. The antibacterial activity was tested against Gram-negative strains namely; Pseudomonas aeruginosa (PSA), Klebsiella pneumoniae (KPC), Escherichia coli (E. coli) and Gram-positive Methicillin-resistant Staphylococcus aureus (MRSA). The minimum inhibitory concentration (MIC) is the lowest concentration of the antibiotic that inhibits microbial growths. MIC is determined by visual inspection or use of spectrophotometer plate reader to determine media turbidity. On the other hand, the minimum bactericidal concentration (MBC) is the lowest concentration that kills 99% of bacterial growth. The MIC and MBC values for a number of compounds are shown in Tables 4-23 below. The antibacterial activities in combination with antibiotics are shown in Tables 24-31 and FIGS. 8-27. The antibacterial activity of a conjugate of antibiotic with a peptide is shown in Table 32 and FIG. 28. The effects of peptides on biofilm formation are shown in FIG. 29A-35. Cytotoxicity of peptides in the hepatic cell line, human skin fibroblast cell line, heart/myocardium cells, and human lung fibroblast cells is shown in FIGS. 36-46). The generation of gold nanoparticles by peptides determined by UV is shown in FIGS. 47 and 48. MIC of peptides and Peptide-capped Au-NPs against Gram-positive bacteria is shown in Tables 33 and 34. MIC of peptides and Peptide-capped Au-NPs against Gram-negative bacteria is shown in Tables 35 and 36. Antibacterial activity of peptides and peptide-capped gold nanoparticles is shown in Table 37. Physical mixture MIC determination of combination between [R₅W₄] (IFX-315)-Au-NP with antibiotics are shown in FIGS. 49-58 when the gold nanoparticles are formed by the peptide followed by addition of the antibiotic. The effect of mixing of the peptide [R₅W₄] (IFX-301) with antibiotics (1:1 ratio) first and then used in the synthesis of Au-NP in antibacterial activities is shown in FIGS. 59-68.

Data revealed that many of the linear and cyclic peptides had a broad-spectrum antibacterial activity against Gram-positive and Gram-negative strains.

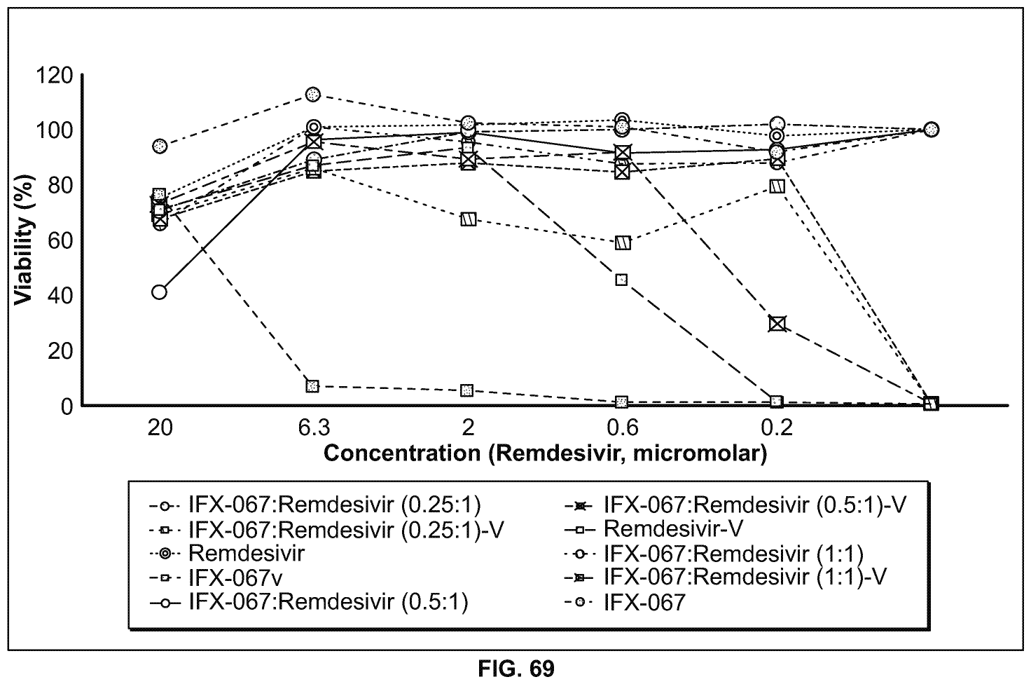

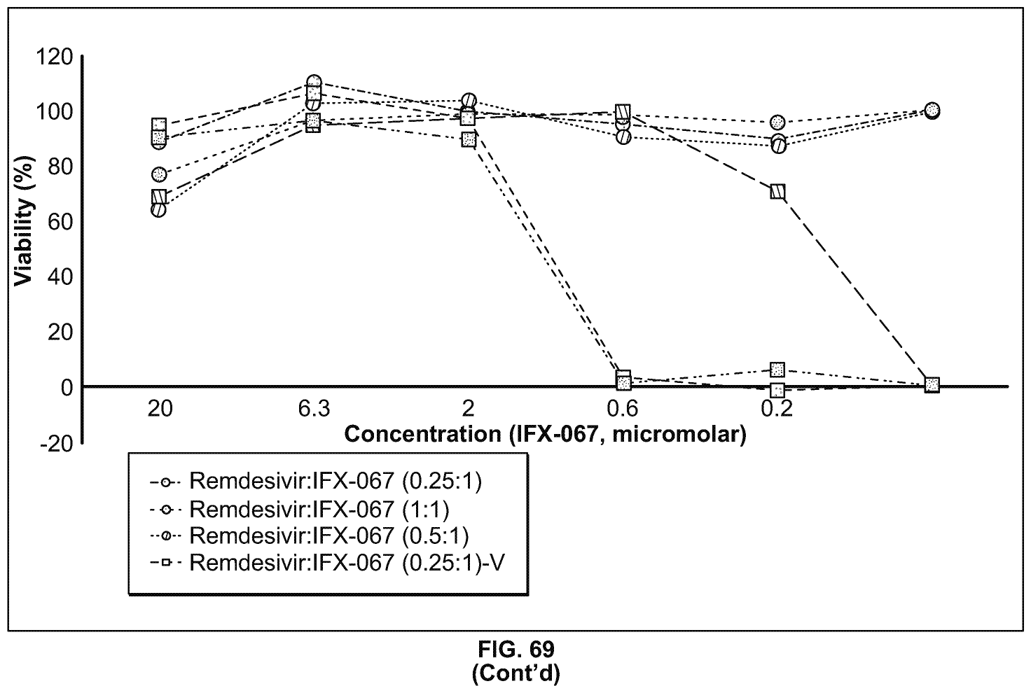

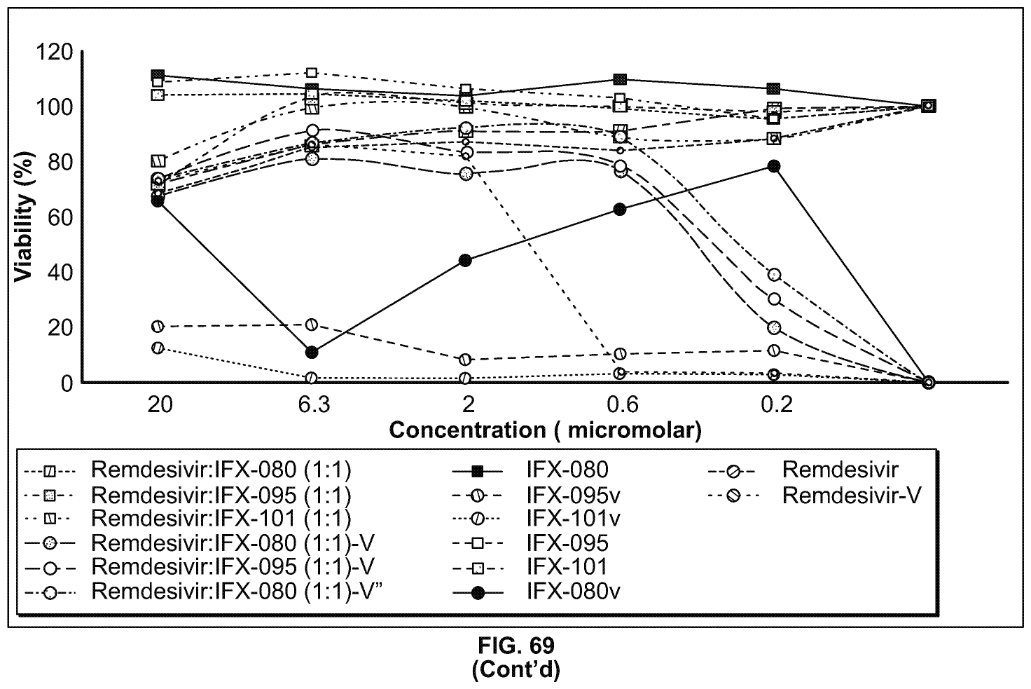

Data revealed combination of peptide with Remdesivir generated significant synergistic activity against human coronavirus 229E (HCoV-229E) (FIG. 69).

### EXAMPLES

Materials. All amino acids building blocks and preloaded amino acid on the resin used in this study were purchased from AAPPTEC. Other reagents, chemicals, and solvents were procured from Sigma-Aldrich. The chemical structure of linear and cyclic peptides, intermediates, and final products were characterized by high-resolution MALDI-TOF (GT-204) from Bruker Inc. The final compounds used in further studies were purified by employing a reversed-phase High-performance liquid chromatography from Shimadzu (LC-20AP) with a binary gradient system of acetonitrile 0.1% TFA and water 0.1% TFA and a reversed-phase preparative column (X Bridge BEH130 Prep C18, 10 μm 18×250 μm Waters, Inc). Mueller Hinton II agar (MH), Methicillin-resistant Staphylococcus aureus MRSA (ATCC BAA-1556), Pseudomonas aeruginosa (ATCC 27883), Klebsiella pneumoniae (ATCC BAA-1705), and Escherichia coli (ATCC 25922) were purchased from ATCC. Human red blood (hRBC) was purchased from BioIVT for hemolytic assay.

Peptide Synthesis; General. The synthesis of linear and cyclic peptides was performed by Fmoc/tBu solid-phase peptide synthesis method using appropriate resin and Fmoc-protected amino acids. For example, the protected amino acid-2-chorotrityl resin was used as building blocks and swelled in the peptide synthesis glass vessel for 1 h in N,N-dimethylformamide (DMF). The amino acids in the sequence were conjugated using Fmoc-amino acid building blocks in the presence of HCTU or 2-(1H-benzotriazole-1-yl)-1,1,3,3-tetramethyluronium hexafluorophosphate (HBTU), hydroxybenzotriazole (HOBt), and diisopropylethylamine DIPEA in DMF. After each coupling, the Fmoc protecting group was cleaved with 20% (v/v) piperidine in DMF. The resin was washed 3 times before the next amino acid in the sequence was added in the sequence. The progress of the reaction was monitored by analyzing few resin beads in the presence of freshly prepared cleavage cocktail reagents Trifluoroacetic acid/Triisopropyl silane/water (92.5%:2.5%:5.0%, v/v/v, 9.25 μL, 2.5 μL and 5 μL), respectively, and was shaken for 1 h. The peptide was precipitated using diethyl ether and characterized using MALDI-TOF mass spectroscopy with α-cyano hydroxycinnamic acid (CHCA) as a matrix. Once the linear peptide was assembled on the resin, the resin was removed by agitation with the cleavage cocktail, dichloromethane/Trifluoroethanol/Acetic acid 7:2:1 (v/v/v) for 3 h. The solvent was evaporated under reduced pressure using rotavapor with the addition of a mixture of hexane and DCM, which resulted in the solid white precipitate of a protected linear peptide. The synthesized linear peptide was cyclized for 24 h with stirring using 1-hydroxy-7-azabenzotriazole (HOAT) and N,N′-diisopropylcarbodiimides (DIC) in an anhydrous DMF/DCM (4:1 v/v, 200 mL:40 mL) mixture. The cyclized peptide was fully deprotected by using cleavage cocktail reagents trifluoroacetic acid/triisopropyl silane/water (92:3:5, v/v/v) for 3 h. The cyclized peptide was precipitated using cold diethyl ether and centrifuged to obtain crude solid peptide. The crude cyclic peptide was purified by a reversed-phase high-performance liquid chromatography (RP-HPLC) with a binary gradient using solvent A containing 0.1% TFA (v/v) in water and solvent B 0.1% TFA (v/v) in acetonitrile for 1 h at a flow rate of 8 mL/min monitored at a wavelength of 214 nm. The fractions showing desired compounds were pools after multiple purification run. The solvents were removed using a rotatory evaporator and lyophilized to obtain powdered peptides with TFA salts.

Synthesis of Linear Peptides. The linear peptide analogs were synthesized by using solid-phase synthesis strategies. Amino acid-loaded 2Cl-Trt resin and Fmoc-amino acid building block was used for synthesis on a scale of 0.3 mmol. HBTU/DIPEA was used as coupling and activating reagent, respectively. Piperidine in DMF (20% v/v) was used for Fmoc deprotection. The peptide was cleaved using cleavage cocktail of TFA/anisole/thioanisole (90:2:5 v/v/v) for 3 h. The crude product was precipitated by the addition of cold diethyl ether purified using reverse-phase HPLC using a gradient of 0-90% acetonitrile (0.1% TFA) and water (0.1% TFA) over 60 min with C-18 column. The purified peptide was lyophilized to yield a white powder (100 mg). The chemical structure of all synthesized peptide was elucidated using mass-to-charge (m/z) mass spectrometry, the ion source is matrix-assisted laser desorption/ionization (MALDI), and the mass analyzer is time-of-flight (TOF) analyzer.

Synthesis of cyclic peptides via head to tail amide cyclization. The cyclic peptides were synthesized from side-chain-protected linear peptides using appropriate cyclization methods. Amino acid-loaded Trt resin and Fmoc-amino acid building block was used for synthesis on a scale of 0.3 mmol. HBTU and DIPEA were used as coupling and activating reagents, respectively. Piperidine in DMF (20% v/v) was used for Fmoc deprotection. The side-chain-protected peptide was detached from the resin by TFE/acetic acid/DCM [2:1:7 (v/v/v)] then subjected to cyclization using HOAT and DIC in an anhydrous DMF/DCM mixture overnight. All protecting groups were removed with cleavage cocktail of TFA/anisole/thioanisole (90:2:5 v/v/v) for 3 h. the crude product was precipitated by the addition of cold diethyl ether and purified using reverse-phase HPLC using a gradient of 0-90% acetonitrile (0.1% TFA) and water (0.1% TFA) over 60 min with C-18 column. The purified peptide was lyophilized to yield a white powder (100 mg). The chemical structures of all synthesized peptides were elucidated using mass-to-charge (m/z) mass spectrometry, the ion source is matrix-assisted laser desorption/ionization (MALDI), and the mass analyzer is time-of-flight (TOF) analyzer.

Synthesis of disulfide cyclized peptides. About 30 mg of linear peptide containing free (SH) group was dissolved in 10% DMSO-H₂O solution (150 ml). The reaction mixture was stirred for 24 h at room temperature in open round-bottomed flask. The reaction mixture was injected directly in reverse phase HPLC using a gradient of 0-90% acetonitrile (0.1% TFA) and water (0.1% TFA) over 60 min with C-18 column. The purified peptide was lyophilized to yield a white powder (20 mg). The chemical structures of all synthesized peptides were elucidated using mass-to-charge (m/z) mass spectrometry, the ion source is matrix-assisted laser desorption/ionization (MALDI), and the mass analyzer is time-of-flight (TOF) analyzer.

Antibacterial Assay. The antibacterial activities of synthesized linear and cyclized peptides were evaluated against these following clinically reported strains; Methicillin-resistant Staphylococcus aureus MRSA (ATCC BAA-1556), Pseudomonas aeruginosa (ATCC 27883), Klebsiella pneumoniae (ATCC BAA-1705), and Escherichia coli (ATCC 25922) using meropenem and vancomycin HCl as positive controls. The minimum inhibitory concentration (MIC) was determined by micro-broth dilution, where the minimal concentrations were determined to be at concentrations in wells in which no visible bacterial growth was present. An aliquot of an overnight culture of bacteria was grown in Tryptic Soya Broth (TSB) or Luria Broth (LB) diluted in 1 mL normal saline to achieve 0.5 McFarland turbidity (1.5×108 bacterial cell CFU/mL). 60 μL of the 0.5 McFarland solution was added to 8940 μL of MH media (this was a 1/150 dilution). Also, 512 μg/ml of the compound was prepared from a stock solution of the samples for testing in LB media. An amount of 100 μL MH media was pipetted into the sterile plate wells except for the first well. An amount of 200 μL of 512 μg/mL compound samples was added by pipette into the first well and serially diluted with the MH media sterile 96 wells using a multi-tip pipette except the last well. An amount of 100 μL aliquot of bacteria solution was added to each well, and the plate was incubated at 37° C. for 18-24 h. All experiments were conducted in triplicate.

The Minimum Bactericidal Concentration (MBC) is the lowest concentration of an antibacterial agent required to kill a bacterium over a fixed period, such as 24 hours, under a specific set of conditions. We determined MBC of the promising peptides from the broth dilution of MIC tests by sub-culturing to agar plates and applying for 24 h incubation at 37° C. The MBC is identified by determining the lowest concentration of antibacterial agent that reduces the viability (concentration of peptides necessary to achieve a bactericidal effect) of the initial bacterial inoculum by ≥99.9%.

The methodology of determination of the minimum inhibitory concentration (MIC) of peptides with antibiotics. The physical mixture (1:1 w/w ratio) of all synthesized peptides were evaluated against four clinically reported strains; Methicillin-resistant Staphylococcus aureus MRSA (ATCC BAA-1556), Pseudomonas aeruginosa (PSA, ATCC 27883), Klebsiella pneumoniae (KPC, ATCC BAA-1705), and Escherichia coli (E. Coli, ATCC 25922) using 11 commercially available antibiotics. The MIC was determined by micro-broth dilution, where the minimal concentrations were determined to be at concentrations in wells in which no visible bacterial growth was present. An aliquot of an overnight culture of bacteria was grown in Luria Broth (LB) diluted in 1 mL normal saline to achieve 0.5 McFarland turbidity (1.5×10⁸ bacterial cell CFU/mL). 60 μL of the 0.5 McFarland solution was added to 8940 μL of MH media (this was a 1/150 dilution). 128 μg/ml (1:1 ratio) of the tested peptides and antibiotics were prepared from a stock solution of the samples for testing in Mueller Hinton Broth MH media. An amount of 100 μL MH media was pipetted into the sterile 96 wells plate except for the first well. An amount of 200 of 128 μg/mL compound samples was added by pipette into the first well and serially diluted with the MH media along sterile 96 wells using a multi-tip pipette except the last well (non-treated well). An amount of 100 μL aliquot of bacteria solution was added to each well, and the plate was incubated at 37° C. for 24 h. All experiments were conducted in triplicate.

MIC determination for the physical mixture of peptides IFX-301, IFX-315, IFX-318, IFX-031, and IFX-067 with 11 commercially available antibiotics to evaluate synergistic activity. Combination therapy offers a perspective on an effective strategy to fight antibiotic resistance and maximize the activity of commercially available antibiotics. The in vitro synergistic results suggest the best appropriate combination therapy that effectively inhibits the bacterial growth in different clinically isolated resistant strains. We selected several peptides for synergistic assay in combination with 11 commercially available antibiotics (Tetracycline, Tobramycin, Levofloxacin, Ciprofloxacin, Meropenem, Vancomycin, Kanamycin, Polymyxin, Daptomycin, Clindamycin, and Metronidazole) were evaluated against four clinically reported strains; (MRSA, KPC, PSA, and E. coli). The MIC was determined by micro-broth dilution, where the minimal concentrations were determined to be at concentrations in wells in which no visible bacterial growth was present. An aliquot of an overnight culture of bacteria was grown in Luria Broth (LB) diluted in 1 mL normal saline to achieve 0.5 McFarland turbidity (1.5×10⁸ bacterial cell CFU/mL). 60 μL of the 0.5 McFarland solution was added to 8940 μL of MH media (this was a 1/150 dilution). 512 μg/ml of the tested compounds were prepared from a stock solution of the samples for testing in Mueller Hinton Broth MH media. An amount of 100 μL MH media was pipetted into the sterile 96 wells plate except for the first well. An amount of 200 μL of 512 μg/mL compound samples was added by pipette into the first well and serially diluted with the MH media along sterile 96 wells using a multi-tip pipette except the last well. An amount of 100 aliquot of bacteria solution was added to each well, and the plate was incubated at 37° C. for 24 h. All experiments were conducted in triplicate.

Synergy Checkerboard Assay. The first step, MIC tested against the strain selected to determine an appropriate range of test concentrations for the synergy test. An aliquot of an overnight culture of bacteria was grown in Luria Broth (LB) diluted in 1 mL normal saline to achieve 0.5 McFarland turbidity (1.5×10⁸ bacterial cell CFU/mL). 60 μL of the 0.5 McFarland solution was added to 8940 μL of MH media (this was a 1/150 dilution). Antibiotics are tested as eleven (11) point, two-fold serial dilutions across the assay plate (from 1-11) in combination with a seven (7) point, a two-fold serial dilution of the peptides down the assay plate. To determine the MIC value for each test compound, two-fold serial dilution in the row H (from 1-11) for antibiotic alone were performed. In column 12 (A-G) down the assay plate, two-fold serial dilution of the peptide alone was performed. Assay plates are inoculated with 100 micro-liters of bacterial suspensions, incubated at 37° C. for 24 hours.

Data analysis of the checkerboard assay. Checkerboard assay was used to determine the impact on antimicrobial potency of the combination of antimicrobial agents in comparison to their individual activities. This comparison is represented as the Fractional Inhibitory Concentration (FIC) index value. The FIC index value takes into account the combination of antimicrobial agents that produces the greatest change from the individual's MIC. To quantify the interactions between the antimicrobial agents being tested (the FIC index), the following equation is used:

A/MICA+B/MICB=FICA+FICB=FIC Index

where A and B are the MIC of each antimicrobial agents in combination (in a single well), and MICA and MICB are the MIC of each drug individually.

The FIC Index value is then used to categorize the interaction of the two antibiotics tested.

Synergy. When the combination of compounds results in a FIC value of ≤0.5, then the combination of the compounds increases the inhibitory activity (decrease in MIC) of one or both compounds than the compounds alone.

Additive or indifference. When the combination of compounds results in an FIC value of <0.5-4, the combination has no increase in inhibitory activity or a slight increase in inhibitory activity from the additive effect of both compounds combined.

Antagonism. When the combination of compounds results in an FIC value of >4, the combination of compounds increases the MIC, or lowers the activity of the compounds.

Minimal Biofilm Inhibitory Concentration (MBIC) Determination of IFX-031, IFX-031-1 (, and IFX-111 Against Representative ESKAPE Pathogens. Each compound was solubilized at 40 mg/mL in DMSO and stored at 4° C. The positive control antibiotics evaluated in parallel and their solubilization information is summarized below.

| | | Stock | |
|---|---|---|---|
| Compound | Source | Concentration | Solvent |
| Vancomycin | Sigma | 10 mg/mL | dH₂O |
| Ciprofloxacin | Sigma | 10 mg/mL | dH₂O |
| Tigecycline | Sigma | 10 mg/mL | DMSO |

Bacteria. The bacterial strains employed in these assays were obtained from the American Type Culture Collection (ATCC). Each strain was propagated as recommended by the ATCC and each strain was stored as a frozen glycerol stock at −80° C. The strains with their classification and properties are listed below.

Bacterial Strains and Characteristics

| | | | | Growth | | Positive |
|---|---|---|---|---|---|---|
| Bacteria Strain | ATCC # | Classification | Properties | Media/Agar | Incubation | Control |
| Staphylococcus | 33592 | Gram Positive | Methicillin | Trypticase | +37° C. | Vancomycin |
| aureus | | Cocci | and | Soy Broth | Aerobic | |
| | | | Gentamicin | (TSB) | | |
| | | | Resistant | | | |
| Klebsiella | BAA- | Gram | NDM-1 and | NB + 25 | +37° C. | Tigecycline |
| pneumoniae | 2470 | Negative | Carbapenem | μg/mL | Aerobic | |
| | | Rod | resistant | Imipenem | | |
| Pseudomonas | 47085 | Gram | QC strain | LB + 10 | +30° C. | Ciprofloxacin |
| aeruginosa | | Negative Rod | | μg/mL | Aerobic | |
| | | | | Tetracycline | | |
| Escherichia | BAA- | Gram | NDM-1 and | NB + 25 | +37° C. | Tigecycline |
| coli | 2471 | Negative | Carbapenem | ug/mL | Aerobic | |
| | | Rod | resistant | Imipenem | | |

Bacterial Propagation. A bacterial colony grown on the appropriate agar as indicated in Table 1 was used to inoculate the appropriate broth and the culture was incubated at the appropriate conditions as in Table 1. Following the incubation, the culture was diluted to an optical density 625 nm (OD₆₂₅) of 0.1 in cation adjusted Mueller Hinton Broth (CAMHB), which is equivalent to 1×10⁸ CFU/mL. The culture was further diluted to 1×10⁶ CFU/mL which was used for the assay.

Determination of the Temporal Effects of SPL7013 Addition to Inhibit Biofilm Formation. Each strain of bacteria was adjusted to a concentration of 1×10⁶ CFU/mL and added to a 96-well flat-bottomed plate in a volume of 100 mL. One-hundred microliters (100 mL) of each compound at 10 concentrations was added in triplicate wells. The cultures were incubated for 24 hours at 37° C. under the appropriate growth conditions for each organism. Following the incubation, the media was removed, and the formed biofilms were fixed for 1 hour at 60° C. Two-hundred microliters (200 mL) of 0.06% crystal violet was added to the wells for 5 to 10 minutes and the wells were then gently washed three times with deionized H2O to remove the crystal violet. Following crystal violet staining 200 mL of 70% ethanol was added to the wells. The same volume was transferred to a 96-well round-bottomed plate and the OD₆₀₀ was measured on a Molecular Devices SpectraMax Plus 384 plate reader.

IFX-031, IFX-031-1, and IFX-111 were evaluated for their ability to prevent biofilm formation by MRSA, K. pneumoniae, P. aeruginosa and E. coli. All three compounds were able to inhibit biofilm formation by MRSA and P. aeruginosa but not K. pneumoniae. The inhibitory effect on E. coli could not be evaluated due to the strain of E. coli used being a poor biofilm producer. It should also be noted that there was an increase in biofilm formation at lower concentrations when MRSA was exposed to IFX-111. An increase was also observed at higher concentrations when K. pneumoniae was exposed to IFX-031 and IFX-111 and E. coli. These results are not uncommon and have been reported in the scientific literature.

Methicillin Resistant Staphylococcus aureus. IFX-031, IFX-031-1, and IFX-111 were evaluated for their ability to inhibit biofilm formation by methicillin resistant S. aureus strain ATCC 333592. Fifty percent (50%) inhibition was observed for IFX-031 and IFX-031-1 at concentrations ranging from 50 μg/mL to 0.78 μg/mL and from 25 μg/mL to 1.56 μg/mL for IFX-111. Increased biofilm formation was observed at lower concentrations of IFX-111. Vancomycin was evaluated in parallel and had approximately 97% inhibition at 5 mg/mL and maintained approximately 50% inhibition at all other concentrations (2.5 μg/mL to 0.001 μg/mL). Data are presented in FIGS. 29A and 29B.

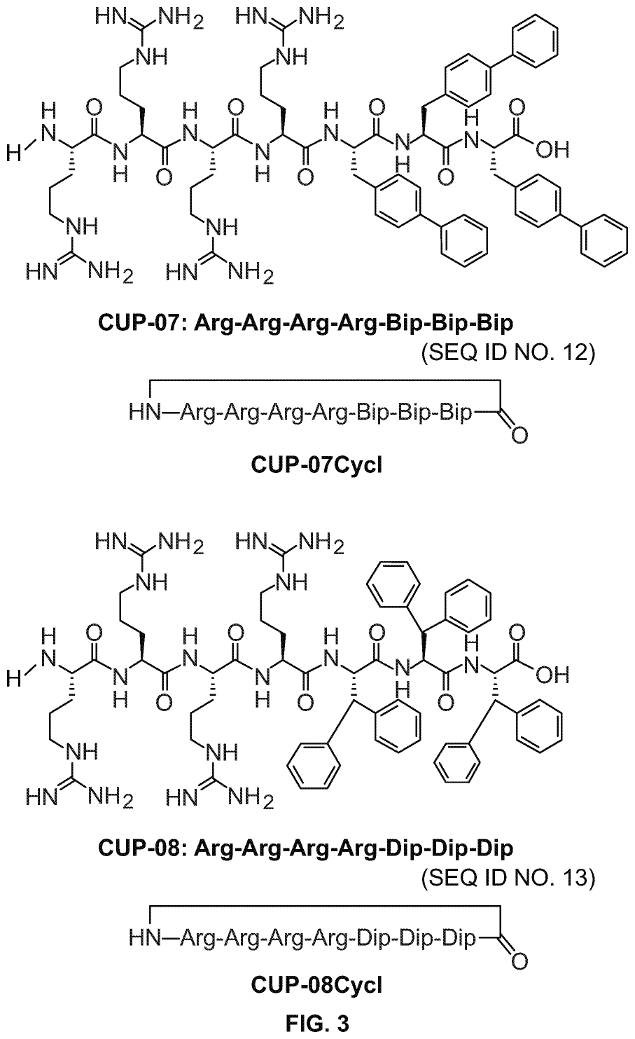

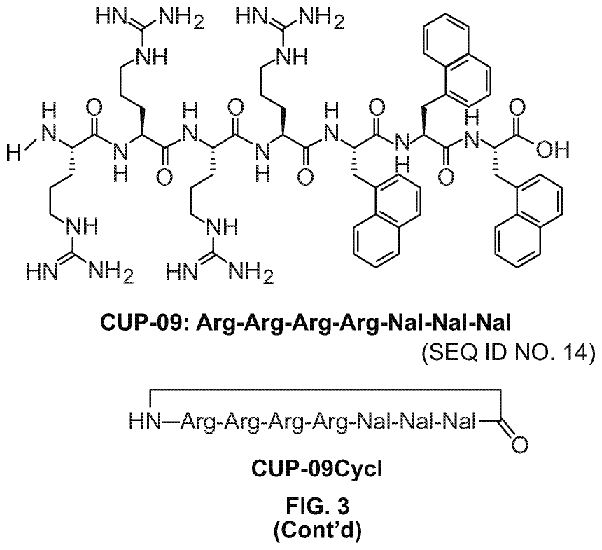

Klebsiella pneumoniae. IFX-031, IFX-031-1, and IFX-111 were evaluated for their ability to inhibit biofilm formation by K. pneumoniae strain ATCC BAA-2470. Less than or equal to fifty percent (≤50%) inhibition was observed for IFX-031 at 12.5 μg/mL and at 3.13 μg/mL and 1.56 μg/mL for IFX-031-1. IFX-111 did not have greater than 23% inhibition of biofilm formation at any concentration evaluated. Increased biofilm formation was observed at 50 μg/mL and 25 μg/mL for IFX-031 and IFX-111. Tigecycline was evaluated in parallel and had ≤50% inhibition at concentrations ranging from 50 mg/mL to 0.78 mg/mL and an increase in biofilm formation at two of the lowest concentrations, 0.2 mg/mL and 0.1 mg/mL. Data are presented in FIG. 3 and FIGS. 30 and 31.

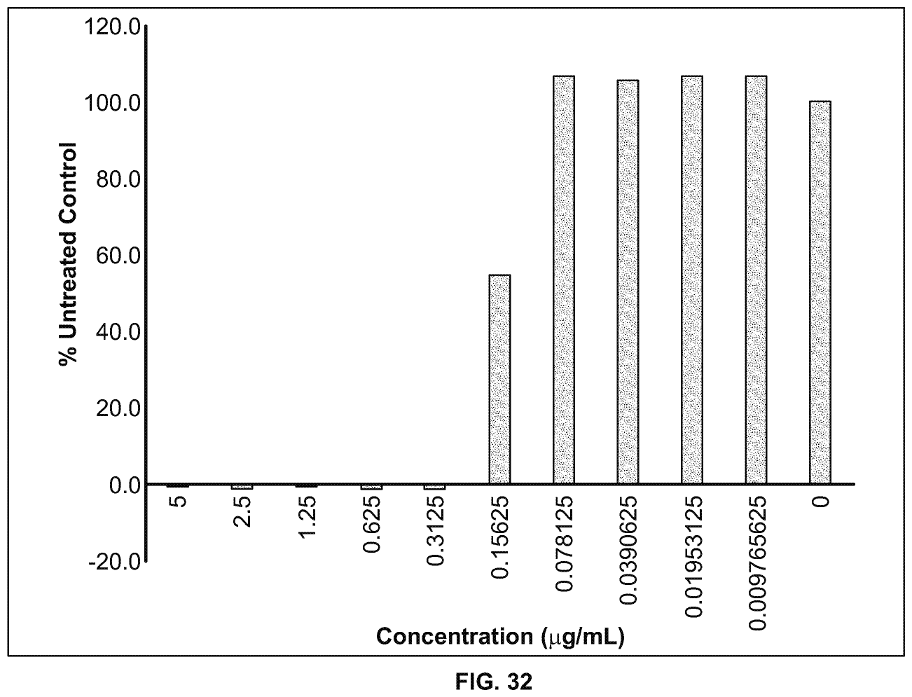

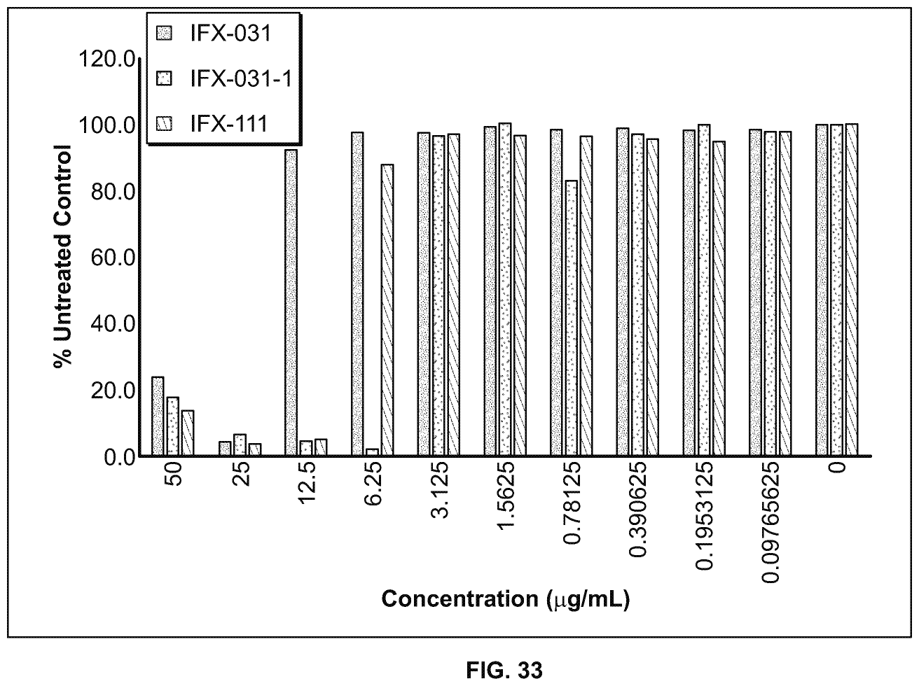

Pseudomonas aeruginosa. IFX-031, IFX-031-1, and IFX-111 were evaluated for their ability to inhibit biofilm formation by P. aeruginosa strain ATCC 47085. Less than or equal to fifty percent (≤50%) inhibition was observed for IFX-031 at 50 μg/mL and 25 mg/mL. IFX-031-1 showed ≤50% inhibition at concentrations ranging from 50 μg/mL to 6.25 mg/mL IFX-111 showed ≤50% inhibition at concentrations ranging from 50 μg/mL to 12.5 μg/mL. Ciprofloxacin was evaluated in parallel and had ≤50% inhibition at concentrations ranging from 50 μg/mL to 0.31 μg/mL. Data are presented in FIGS. 32 and 33.

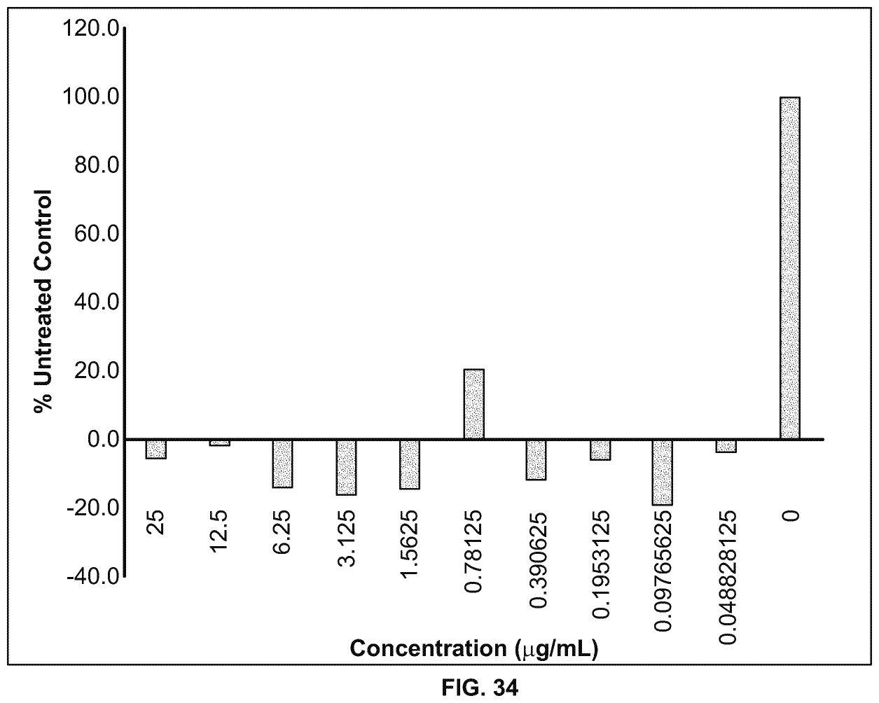

Escherichia coli. IFX-031, IFX-031-1, and IFX-111 were evaluated for their ability to inhibit biofilm formation by E. coli strain ATCC BAA-2471. This strain of E. coli did not produce a biofilm that could be used for an accurate assessment of compound inhibition. From these data it could be determined that there was an increase in biofilm formation at higher concentrations of the compounds when the bacteria were exposed to IFX-031 and IFX-111. Evaluations with an E. coli strain that is a better producer of biofilm formation would need to be performed in order to make an accurate assessment of compound inhibition. Data are presented in FIG. 33 and FIG. 34.

Evaluation of Broad-Spectrum activity. Each compound was solubilized at 40 mg/mL in DMSO and stored at 4° C. The positive control antibiotics evaluated in parallel and their solubilization information is summarized below.

Control Antibiotics

| | | Stock | | |
|---|---|---|---|---|
| Compound | Source | Concentration | | Solvent |
| Penicillin G | Sigma | 10 | mg/mL | dH₂O |
| Linezolid | Pfizer | 1 | mg/mL | dH₂O |
| Vancomycin | Sigma | 10 | mg/mL | dH₂O |
| Ciprofloxacin | Sigma | 10 | mg/mL | dH₂O |
| Tigecycline | Sigma | 10 | mg/mL | DMSO |
| Teicoplanin | Sigma | 10 | mg/mL | dH₂O |
| Ceftriaxone | Sigma | 10 | mg/mL | dH₂O |
| Gentamicin | Sigma | 10 | mg/mL | dH₂O |
| Imipenem | Sigma | 5 | mg/mL | dH₂O |
| Methicillin | Sigma | 10 | mg/mL | dH₂O |

Bacteria. The bacterial strains employed in these assays were obtained from the American Type Culture Collection (ATCC). Each strain was propagated as recommended by the ATCC and each strain was stored as a frozen glycerol stock at −80° C. The strains with their classification and properties are listed below.

Bacterial Strains and Characteristics

| | | | | Growth | | Positive |
|---|---|---|---|---|---|---|
| Bacteria Strain | ATCC # | Classification | Properties | Media/Agar | Incubation | Control |
| Enterococcus | 27270 | Gram | QC Strain | Brain Heart | +37° C. | Penicillin |
| faecium | 700221 | Positive | VRE and | Infusion | Aerobic | Linezolid |
| | | Cocci | teicoplanin | Broth | | |
| | | | resistance | (BHIB) | | |
| Staphylococcus | 29213 | Gram | QC strain | Trypticase | +37° C. | Penicillin |
| aureus | 33592 | Positive | Methicillin | Soy Broth | Aerobic | Vancomycin |
| | | Cocci | and | (TSB | | |
| | | | Gentamicin | | | |
| | | | Resistant | | | |
| Klebsiella | 13883 | Gram | QC strain | Nutrient | +37° C. | Ciprofloxacin |
| pneumoniae | | Negative | | Broth | Aerobic | |
| | | Rod | | (NB) | | |
| | BAA- | | NDM-1 and | NB + 25 | | Tigecycline |
| | 2470 | | Carbapenem | μg/mL | | |
| | | | resistant | Imipenem | | |
| Acinetobacter | 19606 | Gram | MDR | NB | +37° C. | Tigecycline |
| baumannii | | Negative | | | Aerobic | |
| | FDA | Rod | MDR | | +37° C. | Tigecycline |
| | strain | | | | Aerobic | |
| | 0267 | | | | | |
| Pseudomonas | 47085 | Gram | QC strain | LB + 10 | +30° C. | Ciprofloxacin |
| aeruginosa | | Negative | | μg/mL | Aerobic | |
| | | Rod | | Tetracycline | | |
| Enterococcus | 29212 | Gram | QC strain | BHIB/TSA | +37° C. | Vancomycin |
| faecalis | 51575 | Positive | VRE | | Aerobic | Teicoplanin |
| | | Rod | | | | |
| Streptococcus | 49619 | Gram | QC strain | TSB/TSA | +37° C., | Ciprofloxacin |
| pneumoniae | | Positive | | | 5% CO₂ | |
| | | Diplococci | | | Aerobic | |
| | 51938 | | MDR | | +37° C. | Ceftriaxone |
| | | | | | Aerobic | |
| Escherichia | 25922 | Gram | QC Strain | TSB/TSA | +37° C. | Gentamicin |
| coli | BAA- | Negative | NDM-1 and | NB + 25 | Aerobic | Tigecycline |
| | 2471 | Rod | Carbapenem | ug/mL | | |
| | | | resistant | Imipenem | | |
| Clostridium | 700057 | Gram | Ribotype | BHIB | +37° C. | Vancomycin |
| difficile | | Positive | 038/Non- | Oxyrase | Anaerobic | |
| | | | toxicgenic | Brucella | | |
| | NAP1027 | | Reduced | Agar | +37° C. | Vancomycin |
| | | | susceptibility | | Anaerobic | |
| | | | to antibiotics | | | |

Bacterial Propagation. A bacterial colony grown on the appropriate agar as indicated in Table 1 was used to inoculate the appropriate broth and the culture was incubated at the appropriate conditions as in Table 1. Following the incubation, the culture was diluted to an optical density 625 nm (OD₆₂₅) of 0.1 in cation adjusted Mueller Hinton Broth (CAMHB), which is equivalent to 1×10⁸ CFU/mL. The culture was further diluted to 1×10⁶ CFU/mL which was used for the assay. For the S. pneumoniae strains CAMHB+2.5% lysed horse blood was required for the assay and C. difficile used BHIB.

Minimal Inhibitory Concentration (MIC) Determination—Bacteria. The susceptibility of the bacterial organisms to the test compound was evaluated by determining the MIC of each compound using a broth microdilution analysis according to the methods recommended by the Clinical and Laboratory Standards Institute (CLSI). Evaluation of the susceptibility of each organism against the test sample included a positive control antibiotic and for the resistant organisms included a negative control antibiotic. For each organism, a standardized inoculum was prepared by diluting a broth culture that was prepared with freshly plated colonies 18 to 20 hours prior to assay initiation in the appropriate media as indicated in Table 1 to an OD₆₂₅ of 0.1 (equivalent to a 0.5 McFarland standard or 1×10⁸ CFU/mL). The bacteria were centrifuged at 4000 rpm, resuspended in the appropriate media and the suspended inoculum was diluted to a concentration of approximately 1×10⁶ CFU/mL. One-hundred microliters (100 μL) of this suspension was added to triplicate wells of a 96-well plate containing 100 μL of test and control compounds serially diluted 2-fold in the appropriate media. One hundred microliters (100 L) of the inoculum was also added to triplicate wells containing 100 μL of two-fold serial dilutions of a positive control antibiotic and to wells containing 100 μL of media only. This dilution scheme yielded final concentrations for each microbial organism estimated to be 5×10⁵ CFU/mL. The plates were incubated for 24 hours at the appropriate growth conditions for each organism and the microbial growth at each concentration of compound was determined by measuring the OD₆₂₅ on a Molecular Devices SpectraMax Plus-384 plate reader and visually scoring the wells+/−for bacterial growth. The MIC for each compound was determined as the lowest compound dilution that completely inhibited microbial growth.

Hemolytic Assay. We investigated the hemolytic effect (hemolytic assay) of the compounds on fresh human red blood cells to determine the cytotoxicity of the compounds. The result is as shown below. The hemolytic assay was conducted by serial dilution using 1% Triton X, 0.2% Triton X and PBS buffer pH 7.4 as controls. TritonX is a non-ionic surfactant that is capable of lysing cells by the interaction of its polar head with hydrogen bonding present within the cell's lipid bilayer. An aliquot of 2.5 μL from the peptide stock (5 mg/mL) solution was added to 17.5 μL PBS buffer pH 7.4 to achieve a concentration of 640 μg/mL in the solution. PBS buffer solution (20 μL of the 640 μg/mL) was serially diluted in a plate to achieve 320 μg/mL, 160 μg/mL, 80 μg/mL, 40 μg/mL, and 20 μg/mL. 3 mL of the fresh blood sample was washed severally by adding about 10 mL PBS buffer pH 7.4 and centrifuge at 4000 G until the supernatant was cleared. The washed blood sample was diluted to 20 mL volume to be used in the study. An aliquot of 190 μL blood sample was added to 10 μL compound sample in an Eppendorf tube and incubated for 30 min. After incubation, it was centrifuged at 4000 G for 5 min. 100 μL supernatant aliquot was diluted with 1 mL PBS buffer, and the absorbance was measured at 567 nm for the sample. % hemolysis is calculated as follows:

$$\% Hemolysis = \frac{{A}_{x} - {A}_{0} \times 100}{{A}_{100} - {A}_{0}}$$

Where A_{X} is the absorbance at various serial concentrations
A₀ absorbance of PBS buffer pH 7.4
A_{X} absorbance of 0.2% Triton X control equivalent to 100%

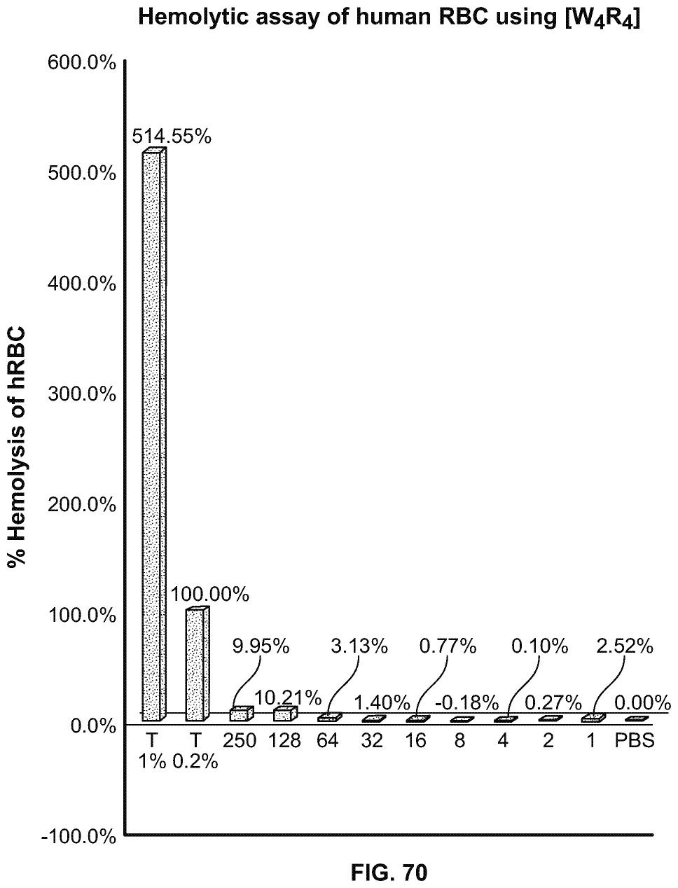

The compounds showed that they cause low hemolysis to red blood cells at the concentration range of the assay. As example of hemolytic assay result is shown in FIG. 70. All the results are shown in Tables 4-14.

Cytotoxicity. The in vitro cytotoxicity of the peptides was evaluated using human lung fibroblast cell (MRC-5, ATCC No. CCL-171), hepatic cell line (HepaRG, ThermoFisher HPRGC10), heart/mycocardium cells (H9C2, ATCC No. CRL 1446), and human skin fibroblast cell line (HeKa, ATCC PCS-200-011) to determine the toxicity of the peptides. All cells were seeded at 5,000 per well in 0.1 mL media in 96 well plates 24 h prior to the experiment. HepaRG cells were seeded in William's E medium with GlutaMAX supplement. Lung cells and heart cells were seeded in DMEM medium containing FBS (10%). The peptides were added to each well in triplicates at a variable concentration of 1-100 μM and incubated for 72 h at 37° C. in a humidified atmosphere of 5% CO₂. After incubation period, MTS solution (20 μL) was added to each well. Then the cells were incubated for 2 h at 37° C. and cell viability was determined by measuring the absorbance at 490 nm using a SpectraMaxM2 microplate spectrophotometer. The percentage of cell survival was calculated as [(OD value of cells treated with the test mixture of compounds)−(OD value of culture medium)]/[(OD value of control cells)−(OD value of culture medium)]×100%.

Synthesis of Meropenem-[R₅W₄K] conjugate, determine of MIC of the conjugate against 4 bacteria strain (MRSA, KPC, PSA, and E. coli).

Synthesis of [R₅W₄K] (IFX-315). The preloaded amino acid on resin, H-Arg(Pbf)-2-chlorotrityl resin and Fmoc-amino acid building block was used for synthesis on a scale of 0.3 mmol. HBTU/DIPEA was used as coupling and activating reagent, respectively. Piperidine in DMF (20% v/v) was used for Fmoc deprotection. The side-chain protected peptide were detached from the resin by TFE/acetic acid/DCM [2:1:7 (v/v/v)] then subjected to cyclization using HOAT and DIC in an anhydrous DMF/DCM mixture over night. All protecting group were removed with cleavage cocktail of TFA/anisole/thioanisole (90:2:5 v/v/v) for 3 h. the crude product was precipitated by the addition of cold diethyl ether purified using reverse-phase HPLC using a gradient of 0-90% acetonitrile (0.1% TFA) and water (0.1% TFA) over 60 min with C-18 column. The purified peptide was lyophilized to yield a white powder (100 mg). The chemical structure of the synthesized peptide was elucidated using mass-to-charge (m/z) mass spectrometry, the ion source is matrix-assisted laser desorption/ionization (MALDI), and the mass analyzer is time-of-flight (TOF) analyzer.

MIC determination. The antibacterial assay of the synthesized conjugate was evaluated against four clinically reported strains; Methicillin-resistant Staphylococcus aureus, Escherichia coli, Pseudomonas aeruginosa, and Klebsiella pneumoniae using meropenem and [R₄W₄] as positive controls. The MIC was determined by micro-broth dilution, where the minimal concentrations were determined to be at concentrations in wells in which no visible bacterial growth was present. An aliquot of an overnight culture of bacteria was grown in Luria Broth (LB) diluted in 1 mL normal saline to achieve 0.5 McFarland turbidity (1.5×10⁸ bacterial cell CFU/mL). 60 μL of the 0.5 McFarland solution was added to 8940 μL of MH media (this was a 1/150 dilution). 512 μg/ml of the tested peptides were prepared from a stock solution of the samples for testing in Mueller Hinton Broth MH media. An amount of 100 μL MH media was pipetted into the sterile 96 wells plate except for the first well. An amount of 200 μL of 512 μg/mL compound samples was added by pipette into the first well and serially diluted with the MH media along sterile 96 wells using a multi-tip pipette except the last well. An amount of 100 μL aliquot of bacteria solution was added to each well, and the plate was incubated at 37° C. for 24 h. All experiments were conducted in triplicate.

In-vivo toxicity study of IFX301. 1. Dose formulations were prepared freshly on dosing day. Doses started from the low to high levels and staggered with 4 h up to 24 h between doses with animals. A dose was escalated or decreased if the test compound appears to be generally tolerated well or not. The cage side observation was done twice daily. The clinical observation was conducted prior to randomization and dosing daily thereafter (at least hourly for the first 4 hours after dosing) and prior to termination for mortality, morbidity check, and clinical signs. Bodyweight was monitored prior to randomization and dosing, daily thereafter, and prior to termination. Gross necropsy was done for all scheduled animals on day 8. Full gross necropsy was conducted, this includes a macroscopic examination of the external surface of the body, all orifices, cranial cavity, the external surface of the brain and cut surfaces of the spinal cord, and the cranial, thoracic, abdominal and pelvic cavities and their viscera, cervical areas, carcass, and genitalia; The animal identification and selected tissues (sex appropriate) identified in Table 38 were sampled and preserved in 10% neutral buffered formalin (NBF). For unscheduled animals (found dead and moribund sacrificed animals): gross necropsy was conducted, and tissues were preserved for possible histology to determine the cause of death. Histopathology was done for adrenal glands, brain, cecum, colon, duodenum, heart, ileum, jejunum, kidneys, liver, rectum, spleen, stomach, thymus lung, pancreas and the testis (ovaries); total 16 tissues/mouse. For all animals, the clinical observation did not show any abnormalities (Table 38).

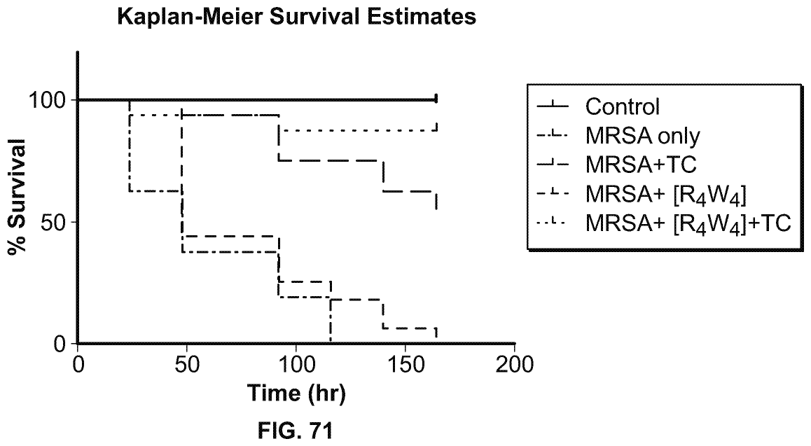

In vivo Galleria Mellonella Assay. Among the peptides showing antimicrobial activity, [R₄W₄] (1) was selected for in vivo study because of its low MIC. The Galleria mellonella is a larva of the greater wax moth, and it has been used as an invertebrate model for antifungal and antibacterial studies. The larvae were contaminated by injecting MRSA inoculums into the pro-leg of larvae. Thereafter peptide 1 was injected with tetracycline to the infected larvae, and the survival rate of them was monitored for one week. We evaluated the antibacterial activity of peptide 1 and the synergistic effect of it with tetracycline against MRSA (FIG. 71). After one week of the assay, 87.5 percent of larvae treated with peptide 1 and tetracycline were survived. However, 56.3 percent and all larvae were killed after one week, which was treated with tetracycline and peptide alone, respectively.

TABLE 1
Examples of peptides containing mixed D-arginine (arg) or L-arginine (Arg) as positively-
charged residues along with hydrophobic 3,3-diphenyl-L-alanine (Dip), 3(2-naphthyl)-
L-alanine (NaI), 3,3-diphenyl-D-alanine (dip), 3(2-naphthyl)-D-alanine (naI).

| | | Mol. Wt. | |
|---|---|---|---|
| Code | Peptides Sequence | Calculated | Found |
| IFX-001 | c[Arg-Arg-Arg-Bip-Bip-Bip] | 1138.39 | 1139.10 |
| IFX-002 | c[Arg-Arg-Arg-Dip-Dip-Dip] | 1138.39 | 1139.00 |
| IFX-003 | c[Arg-Arg-Arg-NaI-NaI-NaI] | 1060.28 | 1060.50 |
| IFX-004 | c[Arg-Arg-ArgArg-Bip-Bip-Bip-Bip] | 1517.86 | 1517.20 |
| IFX-005 | c[Arg-Arg-ArgArg-Dip-Dip-Dip-Dip] | 1517.86 | 1517.20 |
| IFX-006 | c[Arg-Arg-ArgArg-NaI-NaI-NaI-NaI] | 1413.70 | 1413.10 |
| IFX-007 | c[Arg-Arg-ArgArg-Bip-Bip-Bip] | 1294.58 | 1294.10 |
| IFX-008 | c[Arg-Arg-ArgArg-Dip-Dip-Dip] | 1294.58 | 1294.10 |
| IFX-009 | c[Arg-Arg-ArgArg-NaI-NaI-NaI] | 1216.47 | 1216.05 |
| IFX-010 | c[Arg-Arg-Arg-Arg-Dip-Dip] | 1071.31 | 1070.90 |
| IFX-011 | c[Arg-Arg-Arg-Arg-NaI-NaI] | 1019.23 | 1018.70 |
| IFX-012 | c[Arg-Arg-Arg-Arg-Arg-Dip-Dip-Dip] | 1450.77 | 1450.10 |
| IFX-013 | c[Arg-Arg-Arg-Arg-Arg-NaI-NaI-NaI] | 1372.66 | 1372.10 |
| IFX-014 | c[Lys-Lys-Lys-Lys-Dip-Dip-Dip] | 1182.53 | 1182.10 |
| IFX-015 | c[Lys-Lys-Lys-Lys-Lys-Dip-Dip-Dip] | 1310.70 | 1310.20 |
| IFX-016 | c[Arg-Dip-Arg-Arg-Dip-Dip-Arg] | 1294.58 | 1294.10 |
| IFX-017 | c[Arg-Arg-Dip-Dip-Arg-Arg-Dip] | 1294.58 | 1294.20 |
| IFX-018 | c[Arg-NaI-Arg-Arg-NaI-NaI-Arg] | 1216.47 | 1294.10 |
| IFX-019 | c[Arg-Arg-NaI-NaI-Arg-Arg-NaI] | 1216.47 | 1294.10 |
| IFX-020 | c[Arg-Arg-Arg-Trp-Dip-Dip] | 1101.33 | 1101.10 |
| IFX-021 | c[Arg-Arg-Arg-Dip-Trp-Dip] | 1101.33 | 1101.10 |
| IFX-022 | c[Arg-Arg-Arg-Dip-Dip-Trp] | 1101.33 | 110110 |
| IFX-023 | c[Arg-Arg-Arg-Trp-NaI-NaI] | 1049.26 | 1049.10 |
| IFX-024 | c[Arg-Arg-Arg-NaI-Trp-NaI] | 1049.26 | 1049.05 |
| IFX-025 | c[Arg-Arg-Arg-NaI-NaI-Trp] | 1049.26 | 1049.10 |
| IFX-026 | c[Arg-Arg-Arg-Arg-Trp-Dip-Dip] | 1257.54 | 1257.10 |
| IFX-027 | c[Arg-Arg-Arg-Arg-Dip-Trp-Dip] | 1257.54 | 1257.10 |
| IFX-028 | c[Arg-Arg-Arg-Arg-Dip-Dip-Trp] | 1257.52 | 1257.10 |
| IFX-029 | c[Arg-Arg-Arg-Arg-Trp-NaI-NaI] | 1205.44 | 1205.10 |
| IFX-030 | c[Arg-Arg-Arg-Arg-NaI-Trp-NaI] | 1205.44 | 1205.10 |
| IFX-031 | c[Arg-Arg-Arg-Arg-NaI-NaI-Trp] | 1205.44 | 120510 |
| IFX-032 | c[arg-arg-arg-arg-trp-trp-trp-trp] | 1369.61 | 1369.10 |
| IFX-033 | c[Arg-arg-Arg-arg-Trp-trp-Trp-trp] | 1369.61 | 1369.20 |
| IFX-034 | c[arg-Arg-arg-Arg-trp-Trp-trp-Trp] | 1369.61 | 1369.10 |
| IFX-035 | c[Arg-Arg-Arg-Arg-trp-trp-trp-trp] | 1369.61 | 1369.10 |
| IFX-036 | c[arg-arg-arg-arg-Trp-Trp-Trp-Trp] | 1369.61 | 1369.05 |
| IFX-037 | c[arg-arg-arg-arg-arg-trp-trp-trp-trp] | 1525.80 | 1525.00 |
| IFX-038 | c[Arg-Arg-Arg-Arg-5fW-5fW-5fW-5fW] | 1441.57 | 1441.10 |
| IFX-039 | c[Arg-Arg-Arg-Arg-5brW-5brW-5brW-5brW] | 1685.20 | 1684.90 |
| IFX-040 | c[Arg-Arg-Arg-Arg-6clW-6clW-6clW-6clW] | 1507.38 | 1507.10 |
| IFX-041 | c[Arg-Arg-Arg-Arg-1meW-1meW-1meW-1meW] | 1425.72 | 1425.10 |
| IFX-042 | c[Arg-Arg-Arg-Arg-7metW-7metW-7metW-7metW] | 1425.72 | 1425.10 |
| IFX-043 | c[Gly-Arg-Arg-Arg-Arg-Gly-Trp-Trp-Trp-Trp] | 1483.72 | 1483.10 |
| IFX-044 | c[Gly-Gly-Arg-Arg-Arg-Arg-Gly-Gly-Trp-Trp-Trp-Trp] | 1597.82 | 1597.20 |
| IFX-045 | c[Ala-Ala-Arg-Arg-Arg-Arg-Ala-Ala-Trp-Trp-Trp-Trp] | 1653.93 | 1653.20 |
| IFX-046 | c[PEG1-Arg-Arg-Arg-Arg-PEG1-Trp-Trp-Trp-Trp] | 1571.82 | 1571.20 |
| IFX-047 | c[PEG2-Arg-Arg-Arg-Arg-PEG2-Trp-Trp-Trp-Trp] | 1659.93 | 1659.20 |
| IFX-048 | c[PEG1-Arg-Arg-Arg-Arg-PEG1-Dip-Dip-Dip] | 1496.79 | 1496.20 |
| IFX-049 | c[PEG2-Arg-Arg-Arg-Arg-PEG2-Dip-Dip-Dip] | 1584.90 | 1584.10 |
| IFX-050 | c[PEG1-Arg-Arg-Arg-Arg-Arg-PEG1-Dip-Dip-Dip] | 1652.98 | 1652.30 |
| IFX-051 | c[Arg-arg-Arg-arg-PEG4-Trp-naI-naI] | 1452.74 | 1452.10 |
| IFX-052 | c[Arg-arg-PEG4-Arg-arg-Trp-naI-naI] | 1452.74 | 1452.10 |
| IFX-053 | c[PEG4-Arg-arg-Arg-arg-Trp-naI-naI] | 1452.74 | 1452.20 |
| IFX-054 | c[Arg-arg-Arg-arg-Dab-Trp-naI-naI] | 1305.57 | 1305.10 |
| IFX-055 | c[Arg-arg-Dab-Arg-arg-Trp-naI-naI] | 1305.57 | 1305.10 |
| IFX-056 | c[Dab-Arg-arg-Arg-arg-Trp-naI-naI] | 1305.57 | 1305.10 |
| IFX-057 | c[Dab-Arg-arg-Arg-arg-Dab-Trp-naI-naI] | 1405.69 | 1405.20 |
| IFX-058 | c[Dab-Arg-arg-Dab-Arg-arg-Trp-naI-naI] | 1405.69 | 1405.15 |
| IFX-059 | c[Arg-arg-Dab-Arg-arg-Dab-Trp-naI-naI] | 1405.69 | 1405.10 |
| IFX-060 | c[Arg-Arg-Arg-Arg-Dip-Dip-dip] | 1294.58 | 1294.10 |
| IFX-061 | c[Arg-Arg-Arg-Arg-Dip-dip-Dip] | 1294.58 | 1294.10 |
| IFX-062 | c[Arg-Arg-Arg-Arg-dip-Dip-Dip] | 1294.58 | 1294.20 |
| IFX-063 | c[Arg-Arg-Arg-arg-Dip-Dip-Dip] | 1294.58 | 1294.10 |
| IFX-064 | c[Arg-Arg-arg-Arg-Dip-Dip-Dip] | 1294.58 | 1294.10 |
| IFX-065 | c[Arg-arg-Arg-Arg-Dip-Dip-Dip] | 1294.58 | 1294.10 |
| IFX-066 | c[arg-Arg-Arg-Arg-Dip-Dip-Dip] | 1294.58 | 1294.20 |
| IFX-067 | c[arg-arg-arg-arg-dip-dip-dip] | 1294.58 | 1294.10 |
| IFX-068 | c[arg-Arg-arg-Arg-dip-dip-dip] | 1294.58 | 1294.10 |
| IFX-069 | c[arg-Arg-arg-Arg-dip-Dip-dip] | 1294.58 | 1294.20 |
| IFX-070 | c[Arg-arg-Arg-arg-dip-dip-dip] | 1294.58 | 1294.10 |
| IFX-071 | c[Arg-arg-Arg-arg-Dip-dip-Dip] | 1294.58 | 1294.10 |
| IFX-072 | c[Arg-Arg-Arg-Arg-dip-dip-dip] | 1294.58 | 1294.20 |
| IFX-073 | c[arg-arg-arg-arg-Dip-Dip-Dip] | 1294.58 | 1294.10 |
| IFX-074 | c[Arg-Arg-Arg-Arg-NaI-NaI-naI] | 1216.47 | 1216.00 |
| IFX-075 | c[Arg-Arg-Arg-Arg-NaI-naI-NaI] | 1216.47 | 1216.10 |
| IFX-076 | c[Arg-Arg-Arg-Arg-naI-NaI-NaI] | 1216.47 | 1215.90 |
| IFX-077 | c[Arg-Arg-Arg-arg-NaI-NaI-NaI] | 1216.47 | 1216.10 |
| IFX-078 | c[Arg-Arg-arg-Arg-NaI-NaI-NaI] | 1216.47 | 1216.10 |
| IFX-079 | c[Arg-arg-Arg-Arg-NaI-NaI-NaI] | 1216.47 | 1216.05 |
| IFX-080 | c[arg-Arg-Arg-Arg-NaI-NaI-NaI] | 1216.47 | 1216.10 |
| IFX-081 | c[arg-arg-arg-arg-naI-naI-naI] | 1216.47 | 1216.10 |
| IFX-082 | c[arg-Arg-arg-Arg-naI-naI-naI] | 1216.47 | 1216.05 |
| IFX-083 | c[arg-Arg-arg-Arg-naI-NaI-naI] | 1216.47 | 1216.10 |
| IFX-084 | c[Arg-arg-Arg-arg-naI-naI-naI] | 1216.47 | 1216.05 |
| IFX-085 | c[Arg-arg-Arg-arg-NaI-naI-NaI] | 1216.47 | 1216.05 |
| IFX-086 | c[Arg-Arg-Arg-Arg-naI-naI-naI] | 1216.47 | 1216.10 |
| IFX-087 | c[arg-arg-arg-arg-NaI-NaI-NaI] | 1216.47 | 1216.10 |
| IFX-088 | c[Arg-Arg-Arg-Arg-Dip-Trp-dip] | 1257.54 | 1257.05 |
| IFX-089 | c[Arg-Arg-Arg-Arg-Dip-trp-Dip] | 1257.54 | 1257.10 |
| IFX-090 | c[Arg-Arg-Arg-Arg-dip-Trp-Dip] | 1257.54 | 1257.10 |
| IFX-091 | c[Arg-Arg-Arg-arg-Dip-Trp-Dip] | 1257.54 | 1257.10 |
| IFX-092 | c[Arg-Arg-arg-Arg-Dip-Trp-Dip] | 1257.54 | 1257.10 |
| IFX-093 | c[Arg-arg-Arg-Arg-Dip-Trp-Dip] | 1257.54 | 1257.20 |
| IFX-094 | c[arg-Arg-Arg-Arg-Dip-Trp-Dip] | 1257.54 | 1257.20 |
| IFX-095 | c[arg-arg-arg-arg-dip-Trp-dip] | 1257.54 | 1257.05 |
| IFX-096 | c[arg-Arg-arg-Arg-dip-Trp-dip] | 1257.54 | 1257.05 |
| IFX-097 | c[Arg-arg-Arg-arg-dip-Trp-dip] | 1257.54 | 1257.20 |
| IFX-098 | c[Arg-Arg-Arg-Arg-dip-Trp-dip] | 1257.54 | 1257.10 |
| IFX-099 | c[arg-arg-arg-arg-Dip-Trp-Dip] | 1257.54 | 1257.05 |
| IFX-100 | c[Arg-Arg-Arg-Arg-dip-trp-dip] | 1257.54 | 1257.05 |
| IFX-101 | c[arg-arg-arg-arg-dip-trp-dip] | 1257.54 | 1257.10 |
| IFX-102 | c[Arg-Arg-Arg-Arg-Trp-NaI-naI] | 1205.44 | 1205.00 |
| IFX-103 | c[Arg-Arg-Arg-Arg-Trp-naI-NaI] | 1205.44 | 1205.10 |
| IFX-104 | c[Arg-Arg-Arg-Arg-trp-NaI-NaI] | 1205.44 | 1205.10 |
| IFX-105 | c[Arg-Arg-Arg-arg-Trp-NaI-NaI] | 1205.44 | 1205.05 |
| IFX-106 | c[Arg-Arg-arg-Arg-Trp-NaI-NaI] | 1205.44 | 1205.00 |
| IFX-107 | c[Arg-arg-Arg-Arg-Trp-NaI-NaI] | 1205.44 | 1205.10 |
| IFX-108 | c[arg-Arg-Arg-Arg-Trp-NaI-NaI] | 1205.44 | 1205.10 |
| IFX-109 | c[arg-arg-arg-arg-Trp-naI-naI] | 1205.44 | 1205.05 |
| IFX-110 | c[arg-Arg-arg-Arg-Trp-naI-naI] | 1205.44 | 1205.10 |
| IFX-111 | c[Arg-arg-Arg-arg-Trp-naI-naI] | 1205.44 | 1205.10 |
| IFX-112 | c[Arg-Arg-Arg-Arg-Trp-naI-naI] | 1205.44 | 1205.10 |
| IFX-113 | c[arg-arg-arg-arg-Trp-NaI-NaI] | 1205.44 | 1205.00 |
| IFX-114 | c[Arg-Arg-Arg-Arg-trp-naI-naI] | 1205.44 | 1205.05 |
| IFX-115 | c[arg-arg-arg-arg-trp-naI-naI] | 1205.44 | 1205.10 |
| IFX-116 | c[Arg-Arg-arg-arg-Trp-naI-naI] | 1205.44 | 1204.80 |
| IFX-117 | c[arg-arg-Arg-Arg-Trp-naI-naI] | 1205.44 | 1205.00 |
| IFX-118 | c[Arg-Arg-arg-arg-trp-naI-naI] | 1205.44 | 1204.90 |
| IFX-119 | c[arg-arg-Arg-Arg-trp-naI-naI] | 1205.44 | 1204.80 |
| IFX-120 | c[Arg-Arg-arg-arg-trp-NaI-NaI] | 1205.44 | 1204.00 |
| IFX-121 | c[arg-arg-Arg-Arg-trp-NaI-NaI] | 1205.44 | 1204.90 |
| IFX-122 | c[arg-arg-arg-arg-trp-NaI-NaI] | 1205.44 | 1204.08 |
| IFX-123 | c[arg-arg-arg-arg-arg-trp-NaI-NaI] | 1361.63 | 1361.20 |
| IFX-124 | c[arg-arg-arg-arg-arg-trp-naI-naI] | 1361.63 | 1361.10 |
| IFX-125 | c[Arg-Arg-Arg-arg-arg-Trp-naI-naI] | 1361.63 | 1361.10 |
| IFX-126 | c[Lys-Lys-Lys-Lys-Trp-NaI-NaI] | 1093.39 | 1093.10 |
| IFX-127 | c[Lys-Lys-Lys-Lys-Trp-naI-naI] | 1093.39 | 1093.20 |
| IFX-128 | c[lys-Lys-lys-Lys-Trp-naI-naI] | 1093.39 | 1093.05 |
| IFX-129 | c[Lys-lys-Lys-lys-Trp-naI-naI] | 1093.39 | 1093.05 |
| IFX-130 | c[lys-lys-lys-lys-trp-naI-naI] | 1093.39 | 1093.10 |
| IFX-131 | c[Lys-Lys-Lys-Lys-Lys-Trp-NaI-NaI] | 1221.56 | 1221.40 |
| IFX-132 | c[Lys-Lys-Lys-Lys-Lys-trp-naI-naI] | 1221.56 | 1221.20 |
| IFX-133 | c[lys-lys-lys-lys-lys-trp-naI-naI] | 1221.56 | 1221.40 |
| IFX-134 | c[lys-lys-lys-lys-lys-Trp-NaI-NaI] | 1221.56 | 1221.40 |
| IFX-135 | c[Arg-Arg-arg-arg-naI-Trp-naI] | 1205.44 | 1205.10 |
| IFX-136 | c[arg-arg-Arg-Arg-naI-Trp-naI] | 1205.44 | 1205.10 |
| IFX-137 | c[Arg-Arg-arg-arg-naI-trp-naI] | 1205.44 | 1205.05 |
| IFX-138 | c[arg-arg-Arg-Arg-naI-trp-naI] | 1205.44 | 1205.05 |
| IFX-139 | c[Arg-Arg-arg-arg-NaI-trp-NaI] | 1205.44 | 1205.00 |
| IFX-140 | c[arg-arg-Arg-Arg-NaI-trp-NaI] | 1205.44 | 1205.10 |
| IFX-141 | c[arg-arg-arg-arg-NaI-trp-NaI] | 1205.44 | 1205.10 |
| IFX-142 | c[arg-arg-arg-arg-naI-trp-naI] | 1205.44 | 1205.10 |
| IFX-143 | c[Arg-Arg-Arg-Arg-naI-trp-naI] | 1205.44 | 1205.10 |

TABLE 2
Examples of peptides covered by this disclosure.

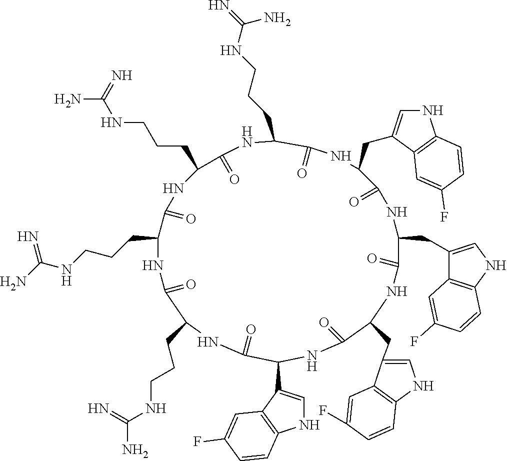 [SMILES: N=C(N)NCCC[C@@H]1NC(=O)[C@H](CCCNC(=N)N)NC(=O)[C@H](CCCNC(=N)N)NC(=O)[C@H](CCCNC(=N)N)NC(=O)[C@H](c2c[nH]c3ccc(F)cc23)NC(=O)[C@H](Cc2c[nH]c3ccc(F)cc23)NC(=O)[C@H](Cc2c[nH]c3ccc(F)cc23)NC(=O)[C@H](Cc2c[nH]c3ccc(F)cc23)NC1=O]

IFX-038
Exact Mass: 1440.68
Molecular Weight: 1441.57

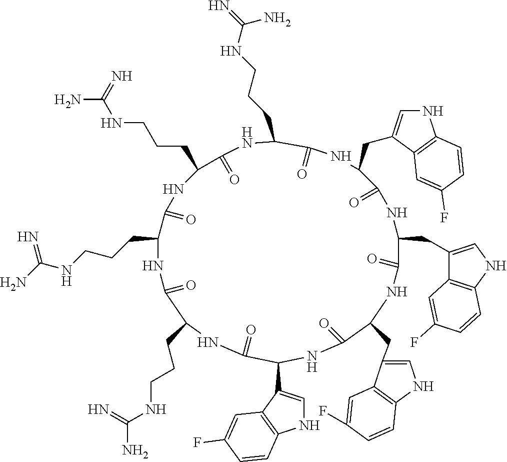 [SMILES: N=C(N)NCCC[C@@H]1NC(=O)[C@H](CCCNC(=N)N)NC(=O)[C@H](CCCNC(=N)N)NC(=O)[C@H](CCCNC(=N)N)NC(=O)[C@H](c2c[nH]c3ccc(F)cc23)NC(=O)[C@H](Cc2c[nH]c3ccc(F)cc23)NC(=O)[C@H](Cc2c[nH]c3ccc(F)cc23)NC(=O)[C@H](Cc2c[nH]c3ccc(F)cc23)NC1=O]

IFX-038
Exact Mass: 1440.68
Molecular Weight: 1441.57

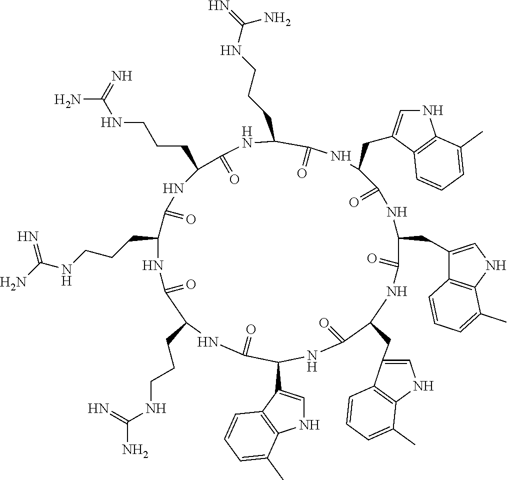 [SMILES: Cc1cccc2c(C[C@@H]3NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)[C@H](c4c[nH]c5c(C)cccc45)NC(=O)[C@H](Cc4c[nH]c5c(C)cccc45)NC(=O)[C@H](Cc4c[nH]c5c(C)cccc45)NC3=O)c[nH]c12]

IFX-042
Exact Mass: 1424.78
Molecular Weight: 1425.72

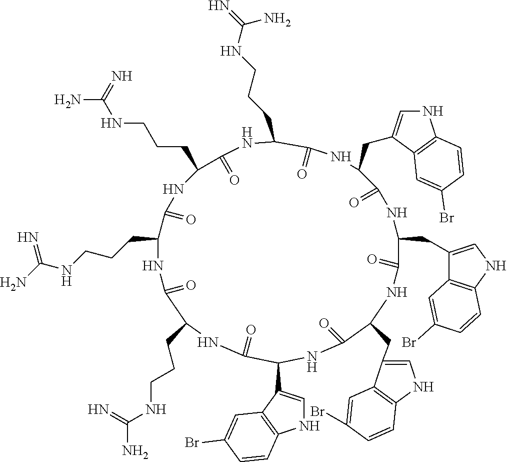 [SMILES: N=C(N)NCCC[C@@H]1NC(=O)[C@H](CCCNC(=N)N)NC(=O)[C@H](CCCNC(=N)N)NC(=O)[C@H](CCCNC(=N)N)NC(=O)[C@H](c2c[nH]c3ccc(Br)cc23)NC(=O)[C@H](Cc2c[nH]c3ccc(Br)cc23)NC(=O)[C@H](Cc2c[nH]c3ccc(Br)cc23)NC(=O)[C@H](Cc2c[nH]c3ccc(Br)cc23)NC1=O]

IFX-039
Exact Mass: 1680.36
Molecular Weight: 1685.20

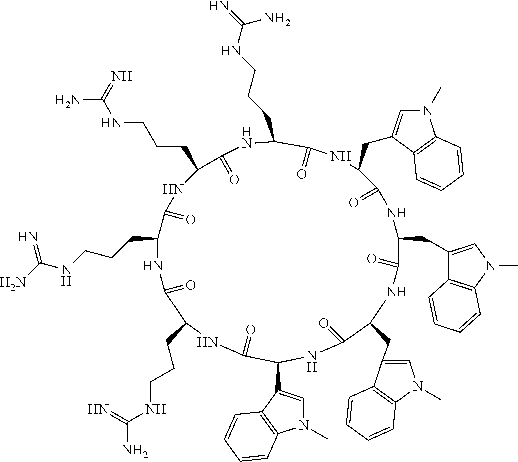 [SMILES: Cn1cc(C[C@@H]2NC(=O)[C@H](CCCNC(=N)N)NC(=O)[C@H](CCCNC(=N)N)NC(=O)[C@H](CCCNC(=N)N)NC(=O)[C@H](CCCNC(=N)N)NC(=O)[C@H](c3cn(C)c4ccccc34)NC(=O)[C@H](Cc3cn(C)c4ccccc34)NC(=O)[C@H](Cc3cn(C)c4ccccc34)NC2=O)c2ccccc21]

IFX-041
Exact Mass: 1424.78
Molecular Weight: 1425.72

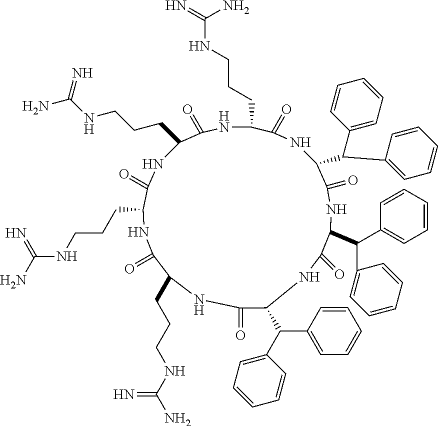 [SMILES: N=C(N)NCCC[C@@H]1NC(=O)[C@@H](CCCNC(=N)N)NC(=O)[C@H](CCCNC(=N)N)NC(=O)[C@@H](C(c2ccccc2)c2ccccc2)NC(=O)[C@H](C(c2ccccc2)c2ccccc2)NC(=O)[C@@H](C(c2ccccc2)c2ccccc2)NC(=O)[C@@H](CCCNC(=N)N)NC1=O]

IFX-060
Exact Mass: 1293.70
Molecular Weight: 1294.58

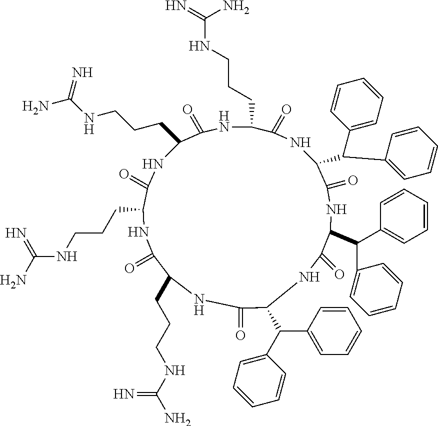 [SMILES: N=C(N)NCCC[C@@H]1NC(=O)[C@@H](CCCNC(=N)N)NC(=O)[C@H](CCCNC(=N)N)NC(=O)[C@@H](C(c2ccccc2)c2ccccc2)NC(=O)[C@H](C(c2ccccc2)c2ccccc2)NC(=O)[C@@H](C(c2ccccc2)c2ccccc2)NC(=O)[C@@H](CCCNC(=N)N)NC1=O]

IFX-061
Exact Mass: 1293.70
Molecular Weight: 1294.58

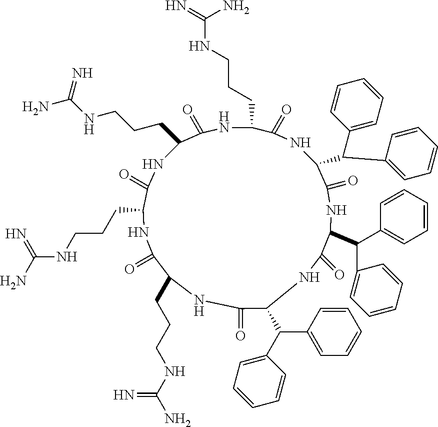 [SMILES: N=C(N)NCCC[C@@H]1NC(=O)[C@@H](CCCNC(=N)N)NC(=O)[C@H](CCCNC(=N)N)NC(=O)[C@@H](C(c2ccccc2)c2ccccc2)NC(=O)[C@H](C(c2ccccc2)c2ccccc2)NC(=O)[C@@H](C(c2ccccc2)c2ccccc2)NC(=O)[C@@H](CCCNC(=N)N)NC1=O]

IFX-061
Exact Mass: 1293.70
Molecular Weight: 1294.58

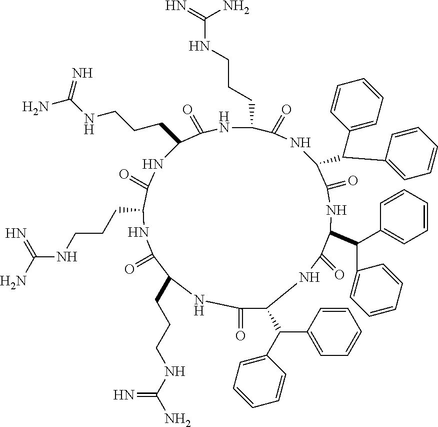 [SMILES: N=C(N)NCCC[C@@H]1NC(=O)[C@@H](CCCNC(=N)N)NC(=O)[C@H](CCCNC(=N)N)NC(=O)[C@@H](C(c2ccccc2)c2ccccc2)NC(=O)[C@H](C(c2ccccc2)c2ccccc2)NC(=O)[C@@H](C(c2ccccc2)c2ccccc2)NC(=O)[C@@H](CCCNC(=N)N)NC1=O]

IFX-062
Exact Mass: 1293.70
Molecular Weight: 1294.58

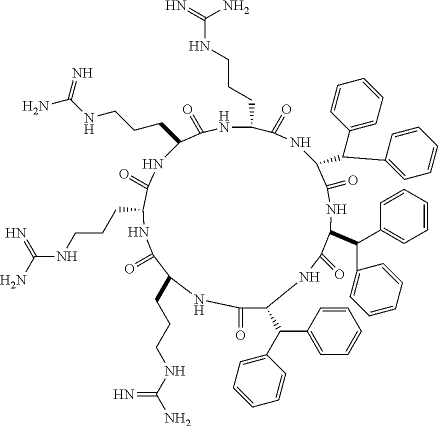 [SMILES: N=C(N)NCCC[C@@H]1NC(=O)[C@@H](CCCNC(=N)N)NC(=O)[C@H](CCCNC(=N)N)NC(=O)[C@@H](C(c2ccccc2)c2ccccc2)NC(=O)[C@H](C(c2ccccc2)c2ccccc2)NC(=O)[C@@H](C(c2ccccc2)c2ccccc2)NC(=O)[C@@H](CCCNC(=N)N)NC1=O]

IFX-063
Exact Mass: 1293.70
Molecular Weight: 1294.58

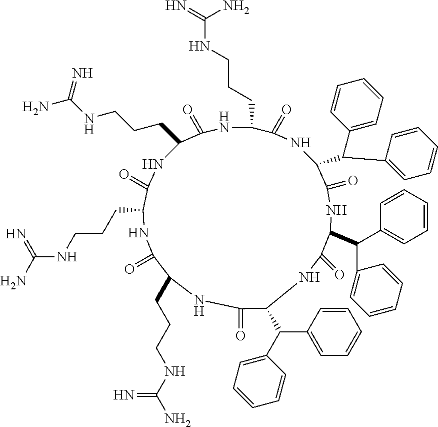 [SMILES: N=C(N)NCCC[C@@H]1NC(=O)[C@@H](CCCNC(=N)N)NC(=O)[C@H](CCCNC(=N)N)NC(=O)[C@@H](C(c2ccccc2)c2ccccc2)NC(=O)[C@H](C(c2ccccc2)c2ccccc2)NC(=O)[C@@H](C(c2ccccc2)c2ccccc2)NC(=O)[C@@H](CCCNC(=N)N)NC1=O]

IFX-064
Exact Mass: 1293.70
Molecular Weight: 1294.58

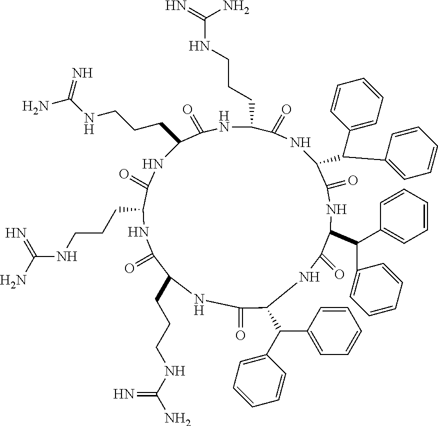 [SMILES: N=C(N)NCCC[C@@H]1NC(=O)[C@@H](CCCNC(=N)N)NC(=O)[C@H](CCCNC(=N)N)NC(=O)[C@@H](C(c2ccccc2)c2ccccc2)NC(=O)[C@H](C(c2ccccc2)c2ccccc2)NC(=O)[C@@H](C(c2ccccc2)c2ccccc2)NC(=O)[C@@H](CCCNC(=N)N)NC1=O]

IFX-065
Exact Mass: 1293.70
Molecular Weight: 1294.58

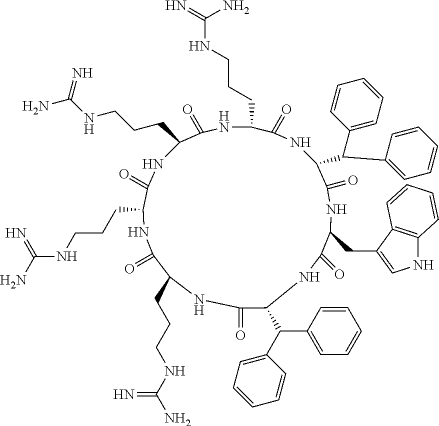 [SMILES: N=C(N)NCCC[C@@H]1NC(=O)[C@@H](CCCNC(=N)N)NC(=O)[C@H](CCCNC(=N)N)NC(=O)[C@@H](C(c2ccccc2)c2ccccc2)NC(=O)[C@H](Cc2c[nH]c3ccccc23)NC(=O)[C@@H](C(c2ccccc2)c2ccccc2)NC(=O)[C@@H](CCCNC(=N)N)NC1=O]

IFX-088
Exact Mass: 1256.68
Molecular Weight: 1257.52

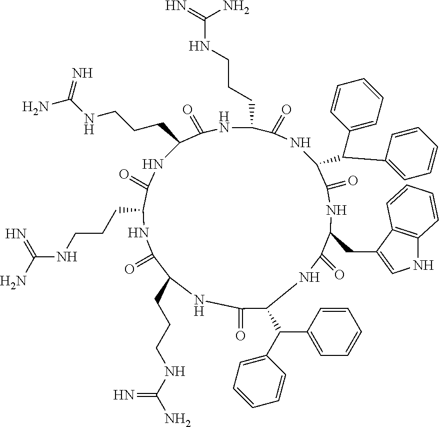 [SMILES: N=C(N)NCCC[C@@H]1NC(=O)[C@@H](CCCNC(=N)N)NC(=O)[C@H](CCCNC(=N)N)NC(=O)[C@@H](C(c2ccccc2)c2ccccc2)NC(=O)[C@H](Cc2c[nH]c3ccccc23)NC(=O)[C@@H](C(c2ccccc2)c2ccccc2)NC(=O)[C@@H](CCCNC(=N)N)NC1=O]

IFX-089
Exact Mass: 1256.68
Molecular Weight: 1257.52

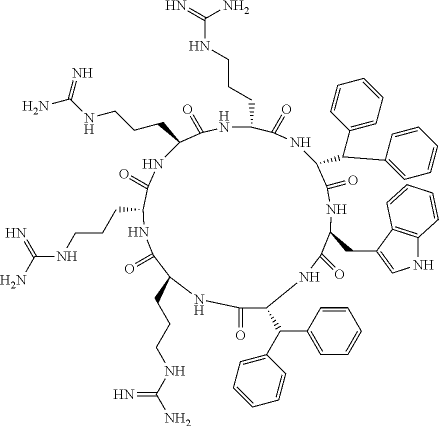 [SMILES: N=C(N)NCCC[C@@H]1NC(=O)[C@@H](CCCNC(=N)N)NC(=O)[C@H](CCCNC(=N)N)NC(=O)[C@@H](C(c2ccccc2)c2ccccc2)NC(=O)[C@H](Cc2c[nH]c3ccccc23)NC(=O)[C@@H](C(c2ccccc2)c2ccccc2)NC(=O)[C@@H](CCCNC(=N)N)NC1=O]

IFX-090
Exact Mass: 1256.68
Molecular Weight: 1257.52

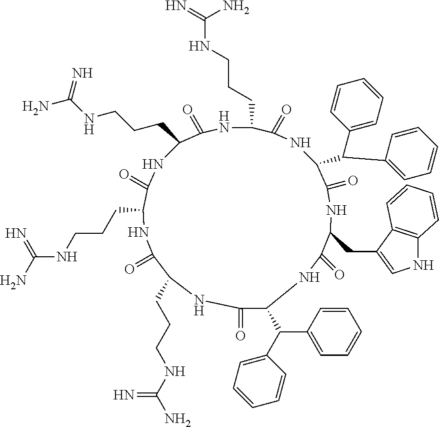 [SMILES: N=C(N)NCCC[C@@H]1NC(=O)[C@@H](CCCNC(=N)N)NC(=O)[C@@H](CCCNC(=N)N)NC(=O)[C@@H](C(c2ccccc2)c2ccccc2)NC(=O)[C@H](Cc2c[nH]c3ccccc23)NC(=O)[C@@H](C(c2ccccc2)c2ccccc2)NC(=O)[C@@H](CCCNC(=N)N)NC1=O]

IFX-091
Exact Mass: 1256.68
Molecular Weight: 1257.52

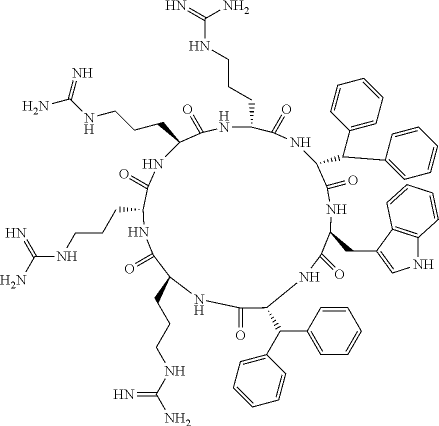 [SMILES: N=C(N)NCCC[C@@H]1NC(=O)[C@@H](CCCNC(=N)N)NC(=O)[C@H](CCCNC(=N)N)NC(=O)[C@@H](C(c2ccccc2)c2ccccc2)NC(=O)[C@H](Cc2c[nH]c3ccccc23)NC(=O)[C@@H](C(c2ccccc2)c2ccccc2)NC(=O)[C@@H](CCCNC(=N)N)NC1=O]

IFX-092
Exact Mass: 1256.68
Molecular Weight: 1257.52

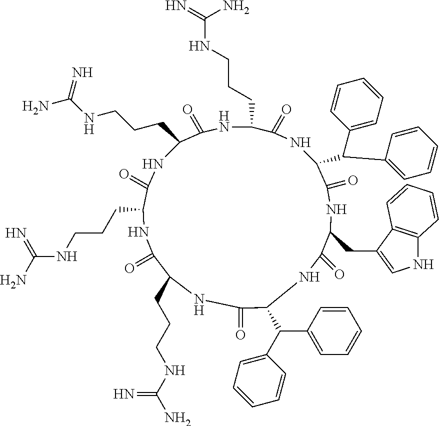 [SMILES: N=C(N)NCCC[C@@H]1NC(=O)[C@@H](CCCNC(=N)N)NC(=O)[C@H](CCCNC(=N)N)NC(=O)[C@@H](C(c2ccccc2)c2ccccc2)NC(=O)[C@H](Cc2c[nH]c3ccccc23)NC(=O)[C@@H](C(c2ccccc2)c2ccccc2)NC(=O)[C@@H](CCCNC(=N)N)NC1=O]

IFX-093
Exact Mass: 1256.68
Molecular Weight: 1257.52

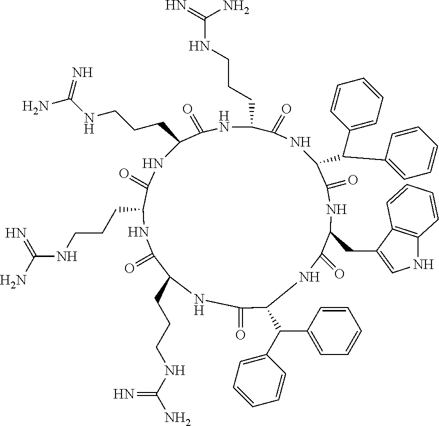 [SMILES: N=C(N)NCCC[C@@H]1NC(=O)[C@@H](CCCNC(=N)N)NC(=O)[C@H](CCCNC(=N)N)NC(=O)[C@@H](C(c2ccccc2)c2ccccc2)NC(=O)[C@H](Cc2c[nH]c3ccccc23)NC(=O)[C@@H](C(c2ccccc2)c2ccccc2)NC(=O)[C@@H](CCCNC(=N)N)NC1=O]

IFX-094
Exact Mass: 1256.68
Molecular Weight: 1257.52

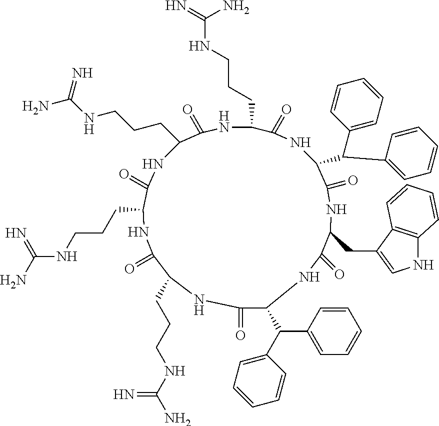 [SMILES: N=C(N)NCCCC1NC(=O)[C@@H](CCCNC(=N)N)NC(=O)[C@@H](CCCNC(=N)N)NC(=O)[C@@H](C(c2ccccc2)c2ccccc2)NC(=O)[C@H](Cc2c[nH]c3ccccc23)NC(=O)[C@@H](C(c2ccccc2)c2ccccc2)NC(=O)[C@@H](CCCNC(=N)N)NC1=O]

IFX-095
Exact Mass: 1256.68
Molecular Weight: 1257.52

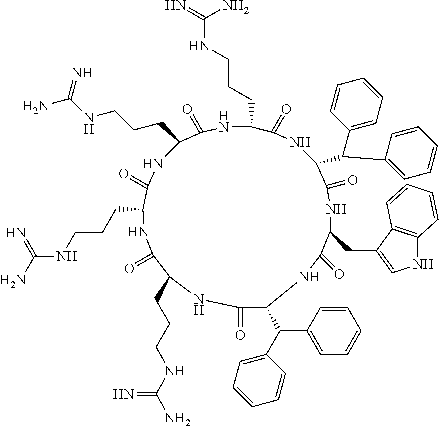 [SMILES: N=C(N)NCCC[C@@H]1NC(=O)[C@@H](CCCNC(=N)N)NC(=O)[C@H](CCCNC(=N)N)NC(=O)[C@@H](C(c2ccccc2)c2ccccc2)NC(=O)[C@H](Cc2c[nH]c3ccccc23)NC(=O)[C@@H](C(c2ccccc2)c2ccccc2)NC(=O)[C@@H](CCCNC(=N)N)NC1=O]

IFX-096
Exact Mass: 1256.68
Molecular Weight: 1257.52

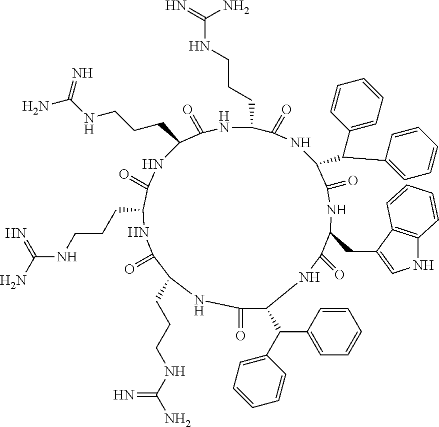 [SMILES: N=C(N)NCCC[C@@H]1NC(=O)[C@@H](CCCNC(=N)N)NC(=O)[C@@H](CCCNC(=N)N)NC(=O)[C@@H](C(c2ccccc2)c2ccccc2)NC(=O)[C@H](Cc2c[nH]c3ccccc23)NC(=O)[C@@H](C(c2ccccc2)c2ccccc2)NC(=O)[C@@H](CCCNC(=N)N)NC1=O]

IFX-097
Exact Mass: 1256.68
Molecular Weight: 1257.52

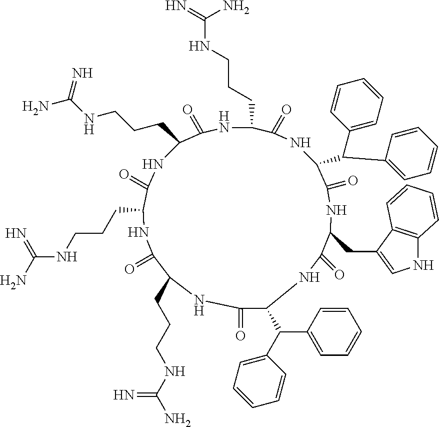 [SMILES: N=C(N)NCCC[C@@H]1NC(=O)[C@@H](CCCNC(=N)N)NC(=O)[C@H](CCCNC(=N)N)NC(=O)[C@@H](C(c2ccccc2)c2ccccc2)NC(=O)[C@H](Cc2c[nH]c3ccccc23)NC(=O)[C@@H](C(c2ccccc2)c2ccccc2)NC(=O)[C@@H](CCCNC(=N)N)NC1=O]

IFX-098
Exact Mass: 1256.68
Molecular Weight: 1257.52

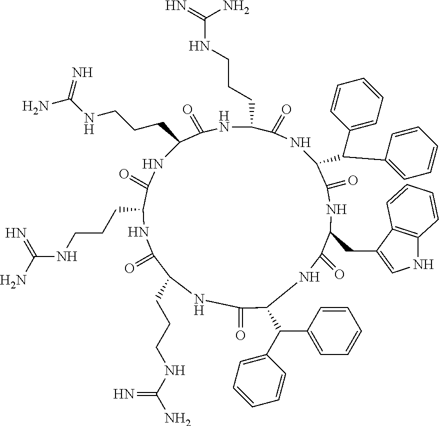 [SMILES: N=C(N)NCCC[C@@H]1NC(=O)[C@@H](CCCNC(=N)N)NC(=O)[C@@H](CCCNC(=N)N)NC(=O)[C@@H](C(c2ccccc2)c2ccccc2)NC(=O)[C@H](Cc2c[nH]c3ccccc23)NC(=O)[C@@H](C(c2ccccc2)c2ccccc2)NC(=O)[C@@H](CCCNC(=N)N)NC1=O]

IFX-099
Exact Mass: 1256.68
Molecular Weight: 1257.52

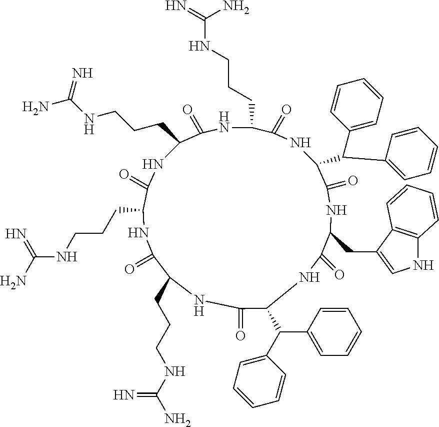 [SMILES: N=C(N)NCCC[C@@H]1NC(=O)[C@@H](CCCNC(=N)N)NC(=O)[C@H](CCCNC(=N)N)NC(=O)[C@@H](C(c2ccccc2)c2ccccc2)NC(=O)[C@H](Cc2c[nH]c3ccccc23)NC(=O)[C@@H](C(c2ccccc2)c2ccccc2)NC(=O)[C@@H](CCCNC(=N)N)NC1=O]

IFX-100
Exact Mass: 1256.68
Molecular Weight: 1257.52

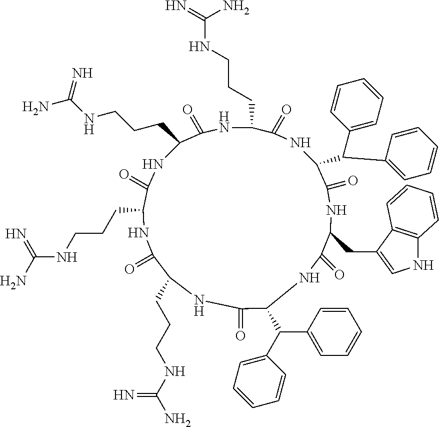 [SMILES: N=C(N)NCCC[C@@H]1NC(=O)[C@@H](CCCNC(=N)N)NC(=O)[C@@H](CCCNC(=N)N)NC(=O)[C@@H](C(c2ccccc2)c2ccccc2)NC(=O)[C@H](Cc2c[nH]c3ccccc23)NC(=O)[C@@H](C(c2ccccc2)c2ccccc2)NC(=O)[C@@H](CCCNC(=N)N)NC1=O]

IFX-101
Exact Mass: 1256.68
Molecular Weight: 1257.52

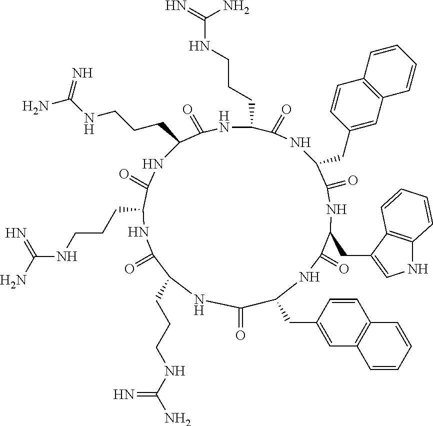 [SMILES: N=C(N)NCCC[C@@H]1NC(=O)[C@@H](CCCNC(=N)N)NC(=O)[C@@H](CCCNC(=N)N)NC(=O)[C@@H](Cc2ccc3ccccc3c2)NC(=O)[C@H](Cc2c[nH]c3ccccc23)NC(=O)[C@@H](Cc2ccc3ccccc3c2)NC(=O)[C@@H](CCCNC(=N)N)NC1=O]

IFX-135
Molecular Weight: 1205.44

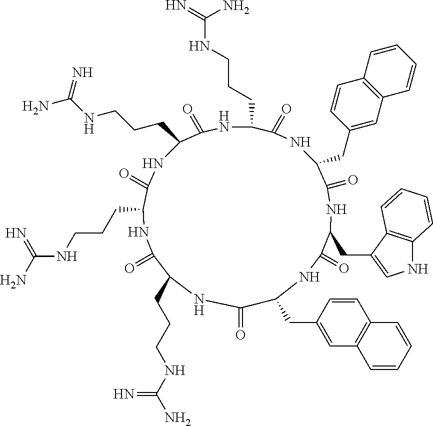 [SMILES: N=C(N)NCCC[C@@H]1NC(=O)[C@@H](Cc2ccc3ccccc3c2)NC(=O)[C@H](Cc2c[nH]c3ccccc23)NC(=O)[C@@H](Cc2ccc3ccccc3c2)NC(=O)[C@@H](CCCNC(=N)N)NC(=O)[C@H](CCCNC(=N)N)NC(=O)[C@@H](CCCNC(=N)N)NC1=O]

IFX-136
Molecular Weight: 1205.44

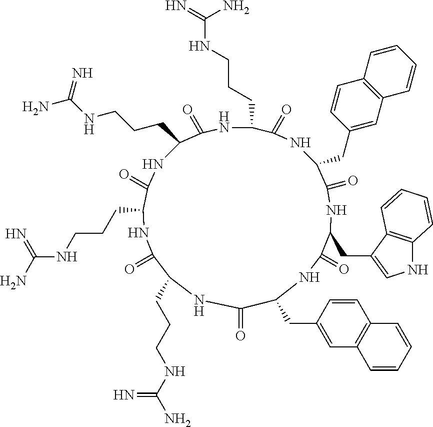 [SMILES: N=C(N)NCCC[C@@H]1NC(=O)[C@@H](CCCNC(=N)N)NC(=O)[C@@H](CCCNC(=N)N)NC(=O)[C@@H](Cc2ccc3ccccc3c2)NC(=O)[C@H](Cc2c[nH]c3ccccc23)NC(=O)[C@@H](Cc2ccc3ccccc3c2)NC(=O)[C@@H](CCCNC(=N)N)NC1=O]

IFX-137
Molecular Weight: 1205.44

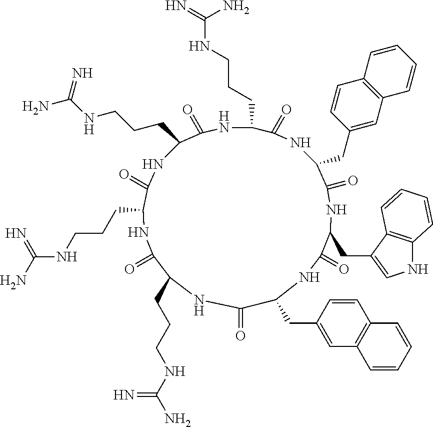 [SMILES: N=C(N)NCCC[C@@H]1NC(=O)[C@@H](Cc2ccc3ccccc3c2)NC(=O)[C@H](Cc2c[nH]c3ccccc23)NC(=O)[C@@H](Cc2ccc3ccccc3c2)NC(=O)[C@@H](CCCNC(=N)N)NC(=O)[C@H](CCCNC(=N)N)NC(=O)[C@@H](CCCNC(=N)N)NC1=O]

IFX-138
Molecular Weight: 1205.44

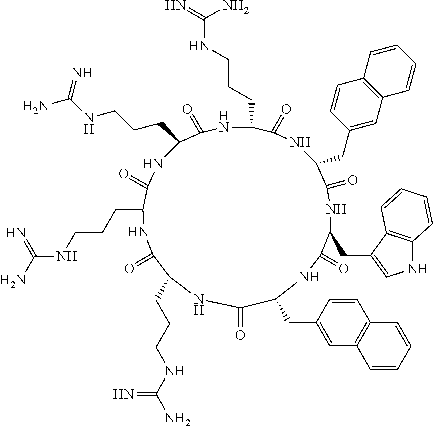 [SMILES: N=C(N)NCCCC1NC(=O)[C@@H](CCCNC(=N)N)NC(=O)[C@@H](Cc2ccc3ccccc3c2)NC(=O)[C@H](Cc2c[nH]c3ccccc23)NC(=O)[C@@H](Cc2ccc3ccccc3c2)NC(=O)[C@@H](CCCNC(=N)N)NC(=O)[C@H](CCCNC(=N)N)NC1=O]

IFX-139
Molecular Weight: 1205.44

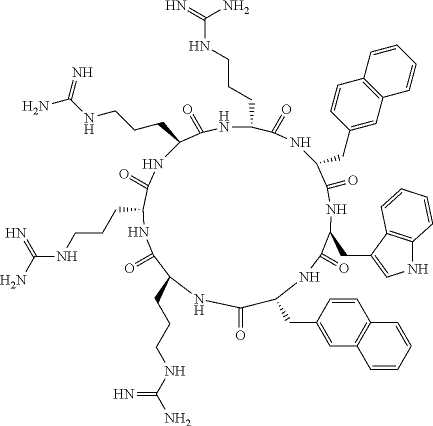 [SMILES: N=C(N)NCCC[C@@H]1NC(=O)[C@@H](Cc2ccc3ccccc3c2)NC(=O)[C@H](Cc2c[nH]c3ccccc23)NC(=O)[C@@H](Cc2ccc3ccccc3c2)NC(=O)[C@@H](CCCNC(=N)N)NC(=O)[C@H](CCCNC(=N)N)NC(=O)[C@@H](CCCNC(=N)N)NC1=O]

IFX-140
Molecular Weight: 1205.44

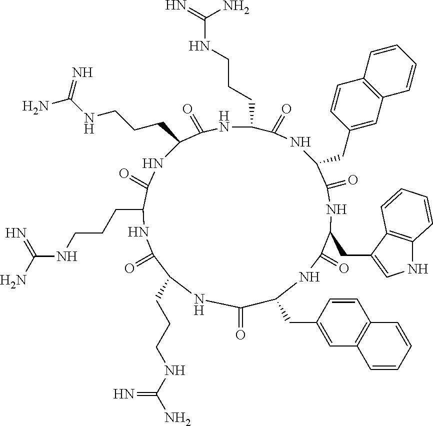 [SMILES: N=C(N)NCCCC1NC(=O)[C@@H](CCCNC(=N)N)NC(=O)[C@@H](Cc2ccc3ccccc3c2)NC(=O)[C@H](Cc2c[nH]c3ccccc23)NC(=O)[C@@H](Cc2ccc3ccccc3c2)NC(=O)[C@@H](CCCNC(=N)N)NC(=O)[C@H](CCCNC(=N)N)NC1=O]

IFX-141
Molecular Weight: 1205.44

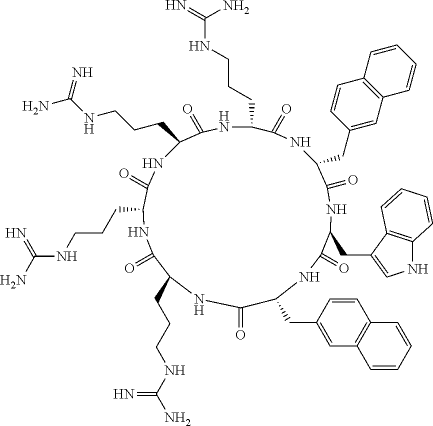 [SMILES: N=C(N)NCCC[C@@H]1NC(=O)[C@@H](Cc2ccc3ccccc3c2)NC(=O)[C@H](Cc2c[nH]c3ccccc23)NC(=O)[C@@H](Cc2ccc3ccccc3c2)NC(=O)[C@@H](CCCNC(=N)N)NC(=O)[C@H](CCCNC(=N)N)NC(=O)[C@@H](CCCNC(=N)N)NC1=O]

IFX-143
Molecular Weight: 1205.44

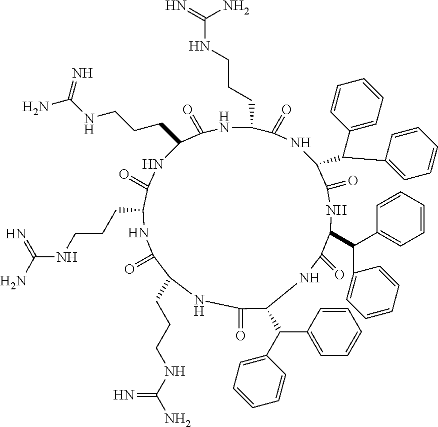 [SMILES: N=C(N)NCCC[C@@H]1NC(=O)[C@@H](CCCNC(=N)N)NC(=O)[C@@H](CCCNC(=N)N)NC(=O)[C@@H](C(c2ccccc2)c2ccccc2)NC(=O)[C@H](C(c2ccccc2)c2ccccc2)NC(=O)[C@@H](C(c2ccccc2)c2ccccc2)NC(=O)[C@@H](CCCNC(=N)N)NC1=O]

IFX-066
Exact Mass: 1293.70
Molecular Weight: 1294.58

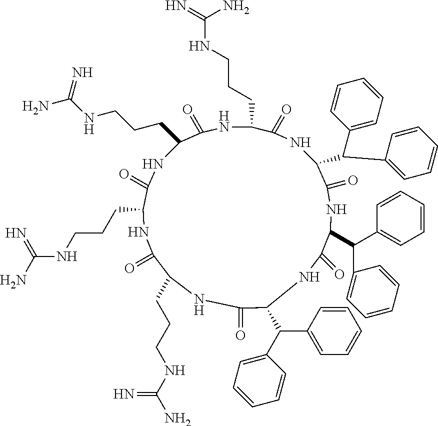 [SMILES: N=C(N)NCCC[C@@H]1NC(=O)[C@@H](CCCNC(=N)N)NC(=O)[C@@H](CCCNC(=N)N)NC(=O)[C@@H](C(c2ccccc2)c2ccccc2)NC(=O)[C@H](C(c2ccccc2)c2ccccc2)NC(=O)[C@@H](C(c2ccccc2)c2ccccc2)NC(=O)[C@@H](CCCNC(=N)N)NC1=O]

IFX-067
Exact Mass: 1293.70
Molecular Weight: 1294.58

TABLE 3
Examples of bacteria that peptides can have antibacterial activity.
Acinetobacter baumannii

| | | |
|---|---|---|
| Acinetobacter baumannii | 19606 | QC |
| Acinetobacter baumannii | BAA-747 | QC |
| Acinetobacter baumannii | FDA strain 0267 | |

Clostridium difficile

| | | |
|---|---|---|
| Clostridium difficile | 9689 | tcdA, tcdB |
| Clostridium difficile | 43255 | tcdA, tcdB |
| Clostridium difficile | BAA-1382, 630 | tcdA, tcdB |
| Clostridium difficile | BAA-1870 | tcdA, tcdB |
| Clostridium difficile | 43593 | tcdA-, tcdB- |
| Clostridium difficile | 700057 | tcdA-, tcdB- |
| Clostridium difficile | St. M3 | |
| Clostridium difficile | CDC 2009217 | |
| Clostridium difficile | CDC 2007019 | |
| Clostridium difficile | CDC 2007054 | |
| Clostridium difficile | NAP1 027 | |
| Clostridium difficile | NR-13427 | |
| Clostridium difficile | NR-13428 | |
| Clostridium difficile | NR-13429 | |
| Clostridium difficile | NR-13430 | |
| Clostridium difficile | NR-13432 | |
| Clostridium difficile | NR-13433 | |
| Clostridium difficile | NR-13434 | |
| Clostridium difficile | NR-13435 | |
| Clostridium difficile | NR-13436 | |
| Clostridium difficile | NR-13437 | |
| Clostridium difficile | NR-13553 | |

Enteric Bacteria

| | | |
|---|---|---|
| Enteric GRP 137 | BAA-72 | ESBL |
| Enterobacter cloacae | 1000654, BAA-2468 | NDM-1 |
| Enterococcus faecalis | 29212 | QC |
| Enterococcus faecalis | 51299 | QC, VRE |
| Enterococcus faecalis | 51575 | VRE |
| Enterococcus faecium | 19434 | QC |
| Enterococcus faecium | 51559 | VRE |
| Enterococcus faecium | | |

Escherichia coli

| | | |
|---|---|---|
| Escherichia coli | 8739 | QC |
| Escherichia coli | 25922 | QC |
| Escherichia coli | 35218 | QC |
| Escherichia coli | 10798 | |
| Escherichia coli | 81371 | |
| Escherichia coli | 1001728, BAA-2469 | NDM-1 |
| Escherichia coli | BAA-196 | ESBL |
| Escherichia coli | CDC 081371 | |
| Escherichia coli | CDC 1001720 | |

Klebsiella pneumoniae

| | | |
|---|---|---|
| Klebsiella pneumoniae | 700603 | QC, ESBL |
| Klebsiella pneumoniae | BAA-2146 | NDM-1 |
| Klebsiella pneumoniae | BAA-1705 | |

Pseudomonas aeruginosa

| | | |
|---|---|---|
| Pseudomonas aeruginosa | 9027 | QC |
| Pseudomonas aeruginosa | 27853 | QC |
| Pseudomonas aeruginosa | 6077 | |
| Pseudomonas aeruginosa | BAA-47, PA01 | |
| Pseudomonas aeruginosa | 29260, PA103 | |
| Pseudomonas aeruginosa | Clinical, PA6077 | |

Staphylococcus sp.

| | | |
|---|---|---|
| Staphylococcus aureus | 6538 | QC |
| Staphylococcus aureus | 29213 | QC |
| Staphylococcus aureus | 14154 | MDR |
| Staphlyococcus aureus | NRS119, BAA-1763 | MRSA, Linezolid R |
| Staphlyococcus aureus | NRS123 | MRSA |
| Staphlyococcus aureus | NRS192, BAA-1707 | MRSA |
| Staphlyococcus aureus | NRS382, BAA-1767 | MRSA |
| Staphlyococcus aureus | NRS383, BAA-1720 | MRSA |
| Staphlyococcus aureus | NRS384 | MRSA |
| Staphylococcus aureus | NRS71 | MRSA |
| Staphylococcus aureus | 33591 | MRSA |
| Staphylococcus aureus | 33592 | MRSA |
| Staphylococcus aureus | 700699 | MRSA |
| Staphylococcus aureus | 700787 | MRSA |
| Staphylococcus aureus | NRS71 | MRSA |
| Staphlyococcus aureus | NRS4 | VISA, GISA |
| Staphlyococcus aureus | VRS1 | VRSA |
| Staphylococcus aureus | 43300 | |
| Staphylococcus aureus | CDC 1000361 | Linezolid R |
| Staphylococcus aureus | 35556 | |
| Staphylococcus epidermidis | 12228 | QC |
| Staphylococcus epidermidis | 35984 | |

Streptococcus sp.

| | | |
|---|---|---|
| Streptococcus pneumoniae | BAA-475 | CDC uses as QC |
| Streptococcus pneumoniae | 49619 | QC |
| Streptococcus pneumoniae | 700677 | |
| Streptococcus pneumoniae | D39 wt | Client Specific |
| Streptococcus pneumoniae | D39 ΔNanA | Client Specific |
| Streptococcus mutans | 35668 | QC |
| Streptococcus pyogenes | 10389 | |

TABLE 4
Antibacterial and hemolytic activities of cyclic peptides
containing various cationic and hydrophobic residues.

| | | MIC (μg/mL) | | | | |
|---|---|---|---|---|---|---|
| | | MRSA | S. aureus | P. aeruginosa | E. coli | |
| | | ATCC | ATCC | ATCC | ATCC | |
| Code | Peptides Sequence | BAA-1556 | 29213 | 27883 | 25922 | HC₅₀ |
| IFX-001 | c[Arg-Arg-Arg-Bip-Bip-Bip] | 50 | 50 | >100 | >100 | NA |
| IFX-002 | c[Arg-Arg-Arg-Dip-Dip-Dip] | 3.1 | 3.1 | 12.5 | 12.5 | 205 |
| IFX-003 | c[Arg-Arg-Arg-NaI-NaI-NaI] | 6.2 | 3.1 | 50 | 25 | 240 |
| IFX-004 | c[Arg-Arg-Arg-Arg-Bip-Bip-Bip-Bip] | >100 | 50 | >100 | >100 | NA |
| IFX-005 | c[Arg-Arg-Arg-Arg-Dip-Dip-Dip-Dip] | 6.2 | 3.1 | 25 | 25 | 95 |
| IFX-006 | c[Arg-Arg-Arg-Arg-NaI-NaI-NaI-NaI] | 6.2 | 3.1 | 50 | 25 | 120 |
| IFX-007 | c[Arg-Arg-Arg-Arg-Bip-Bip-Bip] | 6.2 | 3.1 | 25 | >100 | NA |
| IFX-008 | c[Arg-Arg-Arg-Arg-Dip-Dip-Dip] | 1.5 | 1.5 | 12.5 | 12.5 | 190 |
| IFX-009 | c[Arg-Arg-Arg-Arg-NaI-NaI-NaI] | 1.5 | 1.5 | 12.5 | 25 | 205 |
| IFX-010 | c[Arg-Arg-Arg-Arg-Dip-Dip] | 6.2 | 6.2 | 25 | 50 | 270 |
| IFX-011 | c[Arg-Arg-Arg-Arg-NaI-NaI] | 6.2 | 6.2 | 50 | 50 | 295 |
| IFX-012 | c[Arg-Arg-Arg-Arg-Arg-Dip-Dip-Dip] | 6.2 | 6.2 | 25 | 25 | 195 |
| IFX-013 | c[Arg-Arg-Arg-Arg-Arg-NaI-NaI-NaI] | 6.2 | 6.2 | 25 | 50 | 230 |
| IFX-014 | c[Lys-Lys-Lys-Lys-Dip-Dip-Dip] | 12.5 | 12.5 | >100 | >100 | 415 |
| IFX-015 | c[Lys-Lys-Lys-Lys-Lys-Dip-Dip-Dip] | 12.5 | 6.2 | >100 | 50 | 480 |
| IFX-016 | c[Arg-Dip-Arg-Arg-Dip-Dip-Arg] | 6.2 | 6.2 | 50 | >100 | 310 |
| IFX-017 | c[Arg-Arg-Dip-Dip-Arg-Arg-Dip] | 12.5 | 6.2 | >100 | >100 | 290 |
| IFX-018 | c[Arg-NaI-Arg-Arg-NaI-NaI-Arg] | 6.2 | 6.2 | >100 | 25 | 300 |
| IFX-019 | c[Arg-Arg-NaI-NaI-Arg-Arg-NaI] | 6.2 | 6.2 | 50 | 50 | 325 |
| IFX-020 | c[Arg-Arg-Arg-Trp-Dip-Dip] | 6.2 | 6.2 | 25 | 25 | 180 |
| IFX-021 | c[Arg-Arg-Arg-Dip-Trp-Dip] | 6.2 | 3.1 | 25 | 12.5 | 260 |
| IFX-022 | c[Arg-Arg-Arg-Dip-Dip-Trp] | 12.5 | 6.2 | 25 | 25 | 215 |
| IFX-023 | c[Arg-Arg-Arg-Trp-NaI-NaI] | 6.2 | 3.1 | 25 | 25 | 175 |
| IFX-024 | c[Arg-Arg-Arg-NaI-Trp-NaI] | 6.2 | 3.1 | 25 | 12.5 | 250 |
| IFX-025 | c[Arg-Arg-Arg-NaI-NaI-Trp] | 6.2 | 6.2 | 25 | 25 | 190 |
| IFX-026 | c[Arg-Arg-Arg-Arg-Trp-Dip-Dip] | 1.5 | 1.5 | 6.2 | 12.5 | 240 |
| IFX-027 | c[Arg-Arg-Arg-Arg-Dip-Trp-Dip] | 1.5 | 1.5 | 6.2 | 12.5 | 860 |
| IFX-028 | c[Arg-Arg-Arg-Arg-Dip-Dip-Trp] | 3.1 | 3.1 | 6.2 | 25 | 370 |
| IFX-029 | c[Arg-Arg-Arg-Arg-Trp-NaI-NaI] | 1.5 | 1.5 | 6.2 | 12.5 | 200 |
| IFX-030 | c[Arg-Arg-Arg-Arg-NaI-Trp-NaI] | 3.1 | 1.5 | 6.2 | 12.5 | 430 |
| IFX-031 | c[Arg-Arg-Arg-Arg-NaI-NaI-Trp] | 1.5 | 1.5 | 6.2 | 12.5 | 350 |

TABLE 5
Antibacterial and hemolytic activities of linear peptides containing various cationic and hydrophobic residues.

| | | MIC (μg/mL) | | | | |
|---|---|---|---|---|---|---|
| | | MRSA | S. aureus | P. aeruginosa | E. coli | |
| | | ATCC | ATCC | ATCC | ATCC | |
| Code | Peptides Sequence | BAA-1556 | 29213 | 27883 | 25922 | HC₅₀ |
| IFX-001-1 | NH₂-Arg-Arg-Arg-Bip-Bip-Bip-OH | 50 | 50 | >100 | >100 | NA |
| | (SEQ ID NO. 6) | | | | | |
| IFX-002-1 | NH₂-Arg-Arg-Arg-Dip-Dip-Dip-OH | 3.1 | 3.1 | 25 | 6.2 | 265 |
| | (SEQ ID NO. 7) | | | | | |
| IFX-003-1 | NH₂-Arg-Arg-Arg-NaI -NaI-NaI-OH | 3.1 | 3.1 | 25 | 16 | 310 |
| | (SEQ ID NO. 8 ) | | | | | |
| IFX-004-1 | NH₂-Arg-Arg-Arg-Arg-Bip-Bip-Bip-Bip-OH | >100 | >100 | >100 | >100 | NA |
| | (SEQ ID NO. 9) | | | | | |
| IFX-005-1 | NH₂-Arg-Arg-Arg-Arg-Dip-Dip-Dip-Dip-OH | 6.2 | 6.2 | 50 | 50 | 135 |
| | (SEQ ID NO. 10) | | | | | |
| IFX-006-1 | NH₂-Arg-Arg-Arg-Arg-NaI-NaI-NaI-NaI-OH | 6.2 | 3.1 | >100 | 50 | 140 |
| | (SEQ ID NO. 11) | | | | | |
| IFX-007-1 | NH₂-Arg-Arg-Arg-Arg-Bip-Bip-Bip-OH | 6.2 | 6.2 | 25 | >100 | NA |
| | (SEQ ID NO. 12) | | | | | |
| IFX-008-1 | NH₂-Arg-Arg-Arg-Arg-Dip-Dip-Dip-OH | 3.1 | 3.1 | 6.2 | 6.2 | 210 |
| | (SEQ ID NO. 13) | | | | | |
| IFX-009-1 | NH₂-Arg-Arg-Arg-Arg-NaI-NaI-NaI-OH | 3.1 | 3.1 | 12.5 | 12.5 | 260 |
| | (SEQ ID NO. 14) | | | | | |
| IFX-010-1 | NH₂-Arg-Arg-Arg-Arg-Dip-Dip-OH | 12.5 | 12.5 | 50 | 50 | 360 |
| | (SEQ ID NO. 15) | | | | | |
| IFX-011-1 | NH₂-Arg-Arg-Arg-Arg-NaI-NaI-OH | 6.2 | 6.2 | >100 | 50 | 310 |
| | (SEQ ID NO. 16) | | | | | |
| IFX-012-1 | NH₂-Arg-Arg-Arg-Arg-Arg-Dip-Dip-Dip-OH | 3.1 | 3.1 | 50 | 25 | 225 |
| | (SEQ ID NO. 17) | | | | | |
| IFX-013-1 | NH₂-Arg-Arg-Arg-Arg-Arg-NaI-NaI-NaI-OH | 3.1 | 3.1 | 25 | 25 | 200 |
| | (SEQ ID NO. 18) | | | | | |
| IFX-016-1 | NH₂-Arg-Dip-Arg-Arg-Dip-Dip-Arg-OH | 25 | 12.5 | >100 | 25 | 320 |
| | (SEQ ID NO. 19) | | | | | |
| IFX-017-1 | NH₂-Arg-Arg-Dip-Dip-Arg-Arg-Dip-OH | 12.5 | 12.5 | 25 | 25 | 370 |
| | (SEQ ID NO. 20) | | | | | |
| IFX-018-1 | NH₂-Arg-NaI-Arg-Arg-NaI-NaI-Arg-OH | 25 | 12.5 | 50 | 25 | 405 |
| | (SEQ ID NO. 21) | | | | | |
| IFX-019-1 | NH₂-Arg-Arg-NaI-NaI-Arg-Arg-NaI-OH | 25 | 12.5 | 25 | 25 | 395 |
| | (SEQ ID NO. 22) | | | | | |
| IFX-020-1 | NH₂-R-R-R-Trp-Dip-Dip-OH | 6.2 | 6.2 | 25 | 6.2 | 310 |
| | (SEQ ID NO. 23) | | | | | |
| IFX-021-1 | NH₂-R-R-R-Dip-Trp-Dip-OH | 6.2 | 6.2 | 50 | 12.5 | 325 |
| | (SEQ ID NO. 24) | | | | | |
| IFX-022-1 | NH₂-R-R-R-Dip-Dip-Trp-OH | 12.5 | 12.5 | 50 | 25 | 380 |
| | (SEQ ID NO. 25) | | | | | |
| IFX-023-1 | NH₂-R-R-R-Trp-NaI-NaI-OH | 6.2 | 6.2 | 25 | 12.5 | 495 |
| | (SEQ ID NO. 26) | | | | | |
| IFX-024-1 | NH₂-R-R-R-NaI-Trp-NaI-OH | 6.2 | 6.2 | 25 | 12.5 | 410 |
| | (SEQ ID NO. 27) | | | | | |
| IFX-025-1 | NH₂-R-R-R-NaI-NaI-Trp-OH | 12.5 | 12.5 | 50 | 25 | 375 |
| | (SEQ ID NO. 28) | | | | | |
| IFX-026-1 | NH₂-R-R-R-R-Trp-Dip-Dip-OH | 6.2 | 6.2 | 25 | 12.5 | 430 |
| | (SEQ ID NO. 29) | | | | | |
| IFX-027-1 | NH₂-R-R-R-R-Dip-Trp-Dip-OH | 3.1 | 3.1 | 25 | 12.5 | 910 |
| | (SEQ ID NO. 30) | | | | | |
| IFX-028-1 | NH₂-R-R-R-R-Dip-Dip-Trp-OH | 6.2 | 6.2 | 50 | 25 | 570 |
| | (SEQ ID NO. 31) | | | | | |
| IFX-029-1 | NH₂-R-R-R-R-Trp-NaI-NaI-OH | 3.1 | 3.1 | 50 | 12.5 | 400 |
| | (SEQ ID NO. 32) | | | | | |
| IFX-030-1 | NH₂-R-R-R-R-NaI-Trp-NaI-OH | 6.2 | 6.2 | 25 | 12.5 | 430 |
| | (SEQ ID NO. 33) | | | | | |
| IFX-031-1 | NH₂-R-R-R-R-NaI-NaI-Trp-OH | 6.2 | 6.2 | 25 | 25 | 450 |
| | (SEQ ID NO. 34) | | | | | |

Table 6. Antibacterial and hemolytic activities of cyclic peptides
containing L- or D-arginine and L- or D-tryptophan.

| | | MIC (μg/mL) | | | | |
|---|---|---|---|---|---|---|
| | | MRSA | S. aureus | P. aeruginosa | E. coli | |
| | | ATCC | ATCC | ATCC | ATCC | |
| Code | Peptides Sequence | BAA-1556 | 29213 | 27883 | 25922 | HC₅₀ |
| IFX-032 | c[arg-arg-arg-arg-trp-trp-trp-trp] | 6.2 | 6.2 | 25 | 25 | 230 |
| IFX-033 | c[Arg-arg-Arg-arg-Trp-trp-Trp-trp] | 6.2 | 6.2 | 25 | 12.5 | 95 |
| IFX-034 | c[arg-Arg-arg-Arg-trp-Trp-trp-Trp] | 6.2 | 6.2 | 25 | 12.5 | 320 |
| IFX-035 | c[Arg-Arg-Arg-Arg-trp-trp-trp-trp] | 6.2 | 6.2 | 25 | 12.5 | 670 |
| IFX-036 | c[arg-arg-arg-arg-Trp-Trp-Trp-Trp] | 6.2 | 6.2 | 25 | 12.5 | 600 |
| IFX-037 | c[arg-arg-arg-arg-arg-trp-trp-trp-trp] | 6.2 | 3.1 | 25 | 12.5 | 670 |

TABLE 7
Antibacterial and hemolytic activities of cyclic peptides containing L-arginine and substituted tryptophan residues

| | | MIC (μg/mL) | | | | |
|---|---|---|---|---|---|---|
| | | MRSA | S. aureus | P. aeruginosa | E. coli | |
| | | ATCC | ATCC | ATCC | ATCC | |
| Code | Peptides Sequence | BAA-1556 | 29213 | 27883 | 25922 | HC₅₀ |
| IFX-038 | c[Arg-Arg-Arg-Arg-5fW-5fW-5fW-5fW] | 25 | 12.5 | 100 | 50 | 585 |
| IFX-039 | c[Arg-Arg-Arg-Arg-5brW-5brW-5brW-5brW] | 25 | 25 | >100 | >100 | 615 |
| IFX-040 | c[Arg-Arg-Arg-Arg-6clW-6clW-6clW-6clW] | 3.1 | 3.1 | 100 | 100 | 490 |
| IFX-041 | c[Arg-Arg-Arg-Arg-1meW-1meW-1meW-1meW] | 6.2 | 3.1 | 25 | 25 | 510 |
| IFX-042 | c[Arg-Arg-Arg-Arg-7metW-7metW-7metW-7metW] | 6.2 | 6.1 | 50 | 25 | 545 |

TABLE 8
Antibacterial and hemolytic activities of cyclic peptides containing various cationic
and hydrophobic residues connected directly or through a spacer, such as Dab or PEG.

| | | MIC (μg/mL) | | | | |
|---|---|---|---|---|---|---|
| | | MRSA | S. aureus | P. aeruginosa | E. coli | |
| | | ATCC | ATCC | ATCC | ATCC | |
| Code | Peptides Sequence | BAA-1556 | 29213 | 27883 | 25922 | HC₅₀ |
| IFX-043 | c[Gly-Arg-Arg-Arg-Arg-Gly-Trp-Trp-Trp-Trp] | 6.2 | 6.2 | 50 | 25 | 580 |
| IFX-044 | c[Gly-Gly-Arg-Arg-Arg-Arg-Gly-Gly-Trp-Trp-Trp-Trp] | 25 | 25 | 50 | 50 | 355 |
| IFX-045 | c[Ala-Ala-Arg-Arg-Arg-Arg-Ala-Ala-Trp-Trp-Trp-Trp] | 25 | 25 | 50 | 50 | 390 |
| IFX-046 | c[PEG1-Arg-Arg-Arg-Arg-PEG1-Trp-Trp-Trp-Trp] | 25 | 25 | 50 | 50 | >1000 |
| IFX-047 | c[PEG2-Arg-Arg-Arg-Arg-PEG2-Trp-Trp-Trp-Trp] | 50 | 50 | 50 | 100 | >1000 |
| IFX-048 | c[PEG1-Arg-Arg-Arg-Arg-PEG1-Dip-Dip-Dip] | 16 | 16 | 64 | 64 | 425 |
| IFX-049 | c[PEG2-Arg-Arg-Arg-Arg-PEG2-Dip-Dip-Dip] | 8 | 8 | 64 | 128 | 765 |
| IFX-050-1 | c[PEG1-Arg-Arg-Arg-Arg-Arg-PEG1-Dip-Dip-Dip] | 32 | 16 | 128 | 32 | >1000 |
| IFX-050-2 | c[PEG2-Arg-Arg-Arg-Arg-Arg-PEG2-Dip-Dip-Dip] | 16 | 16 | 128 | 64 | >1000 |
| IFX-051 | c[Arg-arg-Arg-arg-PEG4-Trp-naI-naI] | 32 | 32 | 128 | 128 | 910 |
| IFX-052 | c[Arg-arg-PEG4-Arg-arg-Trp-naI-naI] | 32 | 32 | 128 | 64 | >1000 |
| IFX-053 | c[PEG4-Arg-arg-Arg-arg-Trp-naI-naI] | 16 | 16 | 128 | 64 | 790 |
| IFX-054 | c[Arg-arg-Arg-arg-Dab-Trp-naI-naI] | 64 | 32 | 64 | 32 | 620 |
| IFX-055 | c[Arg-arg-Dab-Arg-arg-Trp-naI-naI] | 32 | 32 | 32 | 16 | 575 |
| IFX-056 | c[Dab-Arg-arg-Arg-arg-Trp-naI-naI] | 64 | 32 | 32 | 32 | 415 |
| IFX-057 | c[Dab-Arg-arg-Arg-arg-Dab-Trp-naI-naI] | 32 | 16 | 32 | 32 | 520 |
| IFX-058 | c[Dab-Arg-arg-Dab-Arg-arg-Trp-naI-naI] | 64 | 32 | 32 | 32 | 655 |
| IFX-059 | c[Arg-arg-Dab-Arg-arg-Dab-Trp-naI-naI] | 128 | 64 | 32 | 32 | 780 |

TABLE 9
Antibacterial and hemolytic activities of cyclic peptides containing D- or L-
arginine and 3,3-diphenyl-L-alanine (Dip) or 3,3-diphenyl-D-alanine (dip).

| | | MIC (μg/mL) | | | | |
|---|---|---|---|---|---|---|
| | | MRSA | S. aureus | P. aeruginosa | E. coli | |
| | | ATCC | ATCC | ATCC | ATCC | |
| Code | Peptides Sequence | BAA-1556 | 29213 | 27883 | 25922 | HC₅₀ |
| IFX-060 | c[Arg-Arg-Arg-Arg-Dip-Dip-dip] | 1.5 | 1.5 | 12.5 | 6.2 | 230 |
| IFX-061 | c[Arg-Arg-Arg-Arg-Dip-dip-Dip] | 1.5 | 3.1 | 12.5 | 12.5 | 200 |
| IFX-062 | c[Arg-Arg-Arg-Arg-dip-Dip-Dip] | 3.1 | 3.1 | 6.2 | 12.5 | 225 |
| IFX-063 | c[Arg-Arg-Arg-arg-Dip-Dip-Dip] | 3.1 | 3.1 | 12.5 | 12.5 | 200 |
| IFX-064 | c[Arg-Arg-arg-Arg-Dip-Dip-Dip] | 3.1 | 3.1 | 12.5 | 12.5 | 140 |
| IFX-065 | c[Arg-arg-Arg-Arg-Dip-Dip-Dip] | 1.5 | 3.1 | 12.5 | 12.5 | 180 |
| IFX-066 | c[arg-Arg-Arg-Arg-Dip-Dip-Dip] | 1.5 | 3.1 | 12.5 | 12.5 | 240 |
| IFX-067 | c[arg-arg-arg-arg-dip-dip-dip] | 1.5 | 3.1 | 12.5 | 6.2 | 160 |
| IFX-068 | c[arg-Arg-arg-Arg-dip-dip-dip] | 1.5 | 3.1 | 12.5 | 6.2 | 280 |
| IFX-069 | c[arg-Arg-arg-Arg-dip-Dip-dip] | 3.1 | 3.1 | 6.2 | 6.2 | 200 |
| IFX-070 | c[Arg-arg-Arg-arg-dip-dip-dip] | 1.5 | 3.1 | 12.5 | 12.5 | 340 |
| IFX-071 | c[Arg-arg-Arg-arg-Dip-dip-Dip] | 3.1 | 3.1 | 12.5 | 12.5 | 150 |
| IFX-072 | c[Arg-Arg-Arg-Arg-dip-dip-dip] | 1.5 | 1.5 | 12.5 | 6.2 | 265 |
| IFX-073 | c[arg-arg-arg-arg-Dip-Dip-Dip] | 1.5 | 3.1 | 6.2 | 6.2 | 245 |

TABLE 10
Antibacterial and hemolytic activities of cyclic peptides containing D- or
L-arginine and naphthyl-L-alanine (NaI) or 3(2-naphthyl-D-alanine (naI).

| | | MIC (μg/mL) | | | | |
|---|---|---|---|---|---|---|
| | | MRSA | S. aureus | P. aeruginosa | E. coli | |
| | | ATCC | ATCC | ATCC | ATCC | |
| Code | Peptides Sequence | BAA-1556 | 29213 | 27883 | 25922 | HC₅₀ |
| IFX-074 | c[Arg-Arg-Arg-Arg-NaI-NaI-naI] | 3.1 | 3.1 | 12.5 | 12.5 | 200 |
| IFX-075 | c[Arg-Arg-Arg-Arg-NaI-naI-NaI] | 3.1 | 3.1 | 12.5 | 25 | 100 |
| IFX-076 | c[Arg-Arg-Arg-Arg-naI-NaI-NaI] | 3.1 | 6.2 | 12.5 | 12.5 | 100 |
| IFX-077 | c[Arg-Arg-Arg-arg-NaI-NaI-NaI] | 3.1 | 6.2 | 12.5 | 12.5 | 100 |
| IFX-078 | c[Arg-Arg-arg-Arg-NaI-NaI-NaI] | 3.1 | 6.2 | 12.5 | 12.5 | 95 |
| IFX-079 | c[Arg-arg-Arg-Arg-NaI-NaI-NaI] | 3.1 | 3.1 | 12.5 | 12.5 | 100 |
| IFX-080 | c[arg-Arg-Arg-Arg-NaI-NaI-NaI] | 3.1 | 3.1 | 12.5 | 12.5 | 200 |
| IFX-081 | c[arg-arg-arg-arg-naI-naI-naI] | 6.2 | 6.2 | 50 | 25 | 200 |
| IFX-082 | c[arg-Arg-arg-Arg-naI-naI-naI] | 3.1 | 3.1 | 12.5 | 25 | 150 |
| IFX-083 | c[arg-Arg-arg-Arg-naI-NaI-naI] | 1.5 | 3.1 | 12.5 | 25 | 170 |
| IFX-084 | c[Arg-arg-Arg-arg-naI-naI-naI] | 1.5 | 3.1 | 12.5 | 25 | 400 |
| IFX-085 | c[Arg-arg-Arg-arg-NaI-naI-NaI] | 1.5 | 3.1 | 12.5 | 25 | 145 |
| IFX-086 | c[Arg-Arg-Arg-Arg-naI-naI-naI] | 3.1 | 3.1 | 12.5 | 12.5 | 155 |
| IFX-087 | c[arg-arg-arg-arg-NaI-NaI-NaI] | 1.5 | 3.1 | 12.5 | 25 | 145 |

TABLE 11
Antibacterial and hemolytic activities of cyclic peptides containing
D- or L-arginine and D- or L-3,3-diphenylalanine and tryptophan.

| | | MIC (μg/mL) | | | | |
|---|---|---|---|---|---|---|
| | | MRSA | S. aureus | P. aeruginosa | E. coli | |
| | | ATCC | ATCC | ATCC | ATCC | |
| Code | Peptides Sequence | BAA-1556 | 29213 | 27883 | 25922 | HC₅₀ |
| IFX-088 | c[Arg-Arg-Arg-Arg-Dip-Trp-dip] | 3.1 | 3.1 | 12.5 | 12.5 | 540 |
| IFX-089 | c[Arg-Arg-Arg-Arg-Dip-trp-Dip] | 3.1 | 3.1 | 12.5 | 12.5 | 470 |
| IFX-090 | c[Arg-Arg-Arg-Arg-dip-Trp-Dip] | 3.1 | 3.1 | 12.5 | 12.5 | 530 |
| IFX-091 | c[Arg-Arg-Arg-arg-Dip-Trp-Dip] | 3.1 | 3.1 | 12.5 | 12.5 | 340 |
| IFX-092 | c[Arg-Arg-arg-Arg-Dip-Trp-Dip] | 3.1 | 3.1 | 12.5 | 25 | 450 |
| IFX-093 | c[Arg-arg-Arg-Arg-Dip-Trp-Dip] | 3.1 | 3.1 | 12.5 | 12.5 | 530 |
| IFX-094 | c[arg-Arg-Arg-Arg-Dip-Trp-Dip] | 3.1 | 3.1 | 6.2 | 12.5 | 285 |
| IFX-095 | c[arg-arg-arg-arg-dip-Trp-dip] | 3.1 | 3.1 | 12.5 | 12.5 | 450 |
| IFX-096 | c[arg-Arg-arg-Arg-dip-Trp-dip] | 3.1 | 3.1 | 6.2 | 6.2 | 260 |
| IFX-097 | c[Arg-arg-Arg-arg-dip-Trp-dip] | 3.1 | 3.1 | 12.5 | 12.5 | 600 |
| IFX-098 | c[Arg-Arg-Arg-Arg-dip-Trp-dip] | 1.5 | 3.1 | 12.5 | 12.5 | 355 |
| IFX-099 | c[arg-arg-arg-arg-Dip-Trp-Dip] | 1.5 | 1.5 | 12.5 | 6.2 | 230 |
| IFX-100 | c[Arg-Arg-Arg-Arg-dip-trp-dip] | 1.5 | 1.5 | 12.5 | 12.5 | 320 |
| IFX-101 | c[arg-arg-arg-arg-dip-trp-dip] | 3.1 | 3.1 | 25 | 12.5 | 350 |

TABLE 12
Antibacterial and hemolytic activities of cyclic peptides containing
D- or L-arginine, D- or L-3(2-naphthyl)-alanine, and tryptophan.

| | | MIC (μg/mL) | | | | |
|---|---|---|---|---|---|---|
| | | MRSA | S. aureus | P. aeruginosa | E. coli | |
| | | ATCC | ATCC | ATCC | ATCC | |
| Code | Peptides Sequence | BAA-1556 | 29213 | 27883 | 25922 | HC₅₀ |
| IFX-102 | c[Arg-Arg-Arg-Arg-Trp-NaI-naI] | 3.1 | 3.1 | 25 | 12.5 | 200 |
| IFX-103 | c[Arg-Arg-Arg-Arg-Trp-naI-NaI] | 3.1 | 3.1 | 12.5 | 12.5 | 170 |
| IFX-104 | c[Arg-Arg-Arg-Arg-trp-NaI-NaI] | 3.1 | 3.1 | 12.5 | 12.5 | 155 |
| IFX-105 | c[Arg-Arg-Arg-arg-Trp-NaI-NaI] | 3.1 | 3.1 | 12.5 | 12.5 | 130 |
| IFX-106 | c[Arg-Arg-arg-Arg-Trp-NaI-NaI] | 1.5 | 1.5 | 12.5 | 25 | 175 |
| IFX-107 | c[Arg-arg-Arg-Arg-Trp-NaI-NaI] | 1.5 | 1.5 | 6.2 | 12.5 | 175 |
| IFX-108 | c[arg-Arg-Arg-Arg-Trp-NaI-NaI] | 1.5 | 1.5 | 6.2 | 12.5 | 350 |
| IFX-109 | c[arg-arg-arg-arg-Trp-naI-naI] | 1.5 | 3.1 | 12.5 | 12.5 | 190 |
| IFX-110 | c[arg-Arg-arg-Arg-Trp-naI-naI] | 1.5 | 3.1 | 12.5 | 12.5 | 225 |
| IFX-111 | c[Arg-arg-Arg-arg-Trp-naI-naI] | 1.5 | 1.5 | 12.5 | 6.2 | 205 |
| IFX-112 | c[Arg-Arg-Arg-Arg-Trp-naI-naI] | 1.5 | 3.1 | 12.5 | 12.5 | 295 |
| IFX-113 | c[arg-arg-arg-arg-Trp-NaI-NaI] | 1.5 | 3.1 | 12.5 | 12.5 | 365 |
| IFX-114 | c[Arg-Arg-Arg-Arg-trp-naI-naI] | 3.1 | 3.1 | 25 | 12.5 | 380 |
| IFX-115 | c[arg-arg-arg-arg-trp-naI-naI] | 3.1 | 3.1 | 25 | 12.5 | 360 |

TABLE 13
Antibacterial and hemolytic activities of linear peptides containing D- or L-arginine with D-
or L-3,3-diphenylalanine and tryptophan or with D- or L-3(2-naphthyl)alanine and tryptophan.

| | | MIC (μg/mL) | | | | |
|---|---|---|---|---|---|---|
| | | MRSA | | P. | | |
| | | ATCC | S. aureus | aeruginosa | E. coli | |
| | | BAA- | ATCC | ATCC | ATCC | |
| Code | Peptides Sequence | 1556 | 29213 | 27883 | 25922 | HC₅₀ |
| IFX-067-1 | NH₂-arg-arg-arg-arg-dip-dip-dip-OH | 3.1 | 3.1 | 12.5 | 6.2 | 390 |
| IFX-068-1 | NH₂-arg-Arg-arg-Arg-dip-dip-dip-OH | 6.2 | 3.1 | 25 | 12.5 | 355 |
| IFX-070-1 | NH₂-Arg-arg-Arg-arg-dip-dip-dip-OH | 3.1 | 3.1 | 12.5 | 6.2 | 380 |
| IFX-072-1 | NH₂-Arg-Arg-Arg-Arg-dip-dip-dip-OH | 6.2 | 3.1 | 12.5 | 6.2 | 470 |
| IFX-073-1 | NH₂-arg-arg-arg-arg-Dip-Dip-Dip-OH | 6.2 | 3.1 | 12.5 | 6.2 | 410 |
| IFX-109-1 | NH₂-arg-arg-arg-arg-Trp-naI-naI-OH | 3.1 | 3.1 | 12.5 | 12.5 | 375 |
| IFX-110-1 | NH₂-arg-Arg-arg-Arg-Trp-naI-naI-OH | 3.1 | 3.1 | 25 | 25 | 330 |
| IFX-111-1 | NH₂-Arg-arg-Arg-arg-Trp-naI-naI-OH | 3.1 | 3.1 | 25 | 25 | 310 |
| IFX-112-1 | NH₂-Arg-Arg-Arg-Arg-Trp-naI-naI-OH | 3.1 | 6.2 | 25 | 12.5 | 370 |
| IFX-113-1 | NH₂-arg-arg-arg-arg-Trp-NaI-NaI-OH | 6.2 | 6.2 | 12.5 | 12.5 | 300 |

TABLE 14
Antibacterial and hemolytic activities of cyclic peptides containing D-
or L-arginine or lysine with D- or L-3(2-naphthyl)alanine and tryptophan.

| | | MIC (μg/mL) | | | | |
|---|---|---|---|---|---|---|
| | | MRSA | | P. | | |
| | | ATCC | S. aureus | aeruginosa | E. coli | |
| | | BAA- | ATCC | ATCC | ATCC | |
| Code | Peptides Sequence | 1556 | 29213 | 27883 | 25922 | HC₅₀ |
| IFX-116 | c[Arg-Arg-arg-arg-Trp-naI-naI] | 3.1 | 3.1 | 32 | 32 | 480 |
| IFX-117 | c[arg-arg-Arg-Arg-Trp-naI-naI] | 3.1 | 3.1 | 32 | 32 | 190 |
| IFX-118 | c[Arg-Arg-arg-arg-trp-naI-naI] | 3.1 | 3.1 | 16 | 16 | 210 |
| IFX-119 | c[arg-arg-Arg-Arg-trp-naI-naI] | 3.1 | 3.1 | 32 | 16 | 200 |
| IFX-120 | c[Arg-Arg-arg-arg-trp-NaI-NaI] | 3.1 | 3.1 | 32 | 32 | 195 |
| IFX-121 | c[arg-arg-Arg-Arg-trp-NaI-NaI] | 6.2 | 3.1 | 32 | 32 | 425 |
| IFX-122 | c[arg-arg-arg-arg-trp-NaI-NaI] | 3.1 | 3.1 | 32 | 32 | 220 |
| IFX-123 | c[arg-arg-arg-arg-arg-trp-NaI-NaI] | 3.1 | 3.1 | 16 | 16 | 190 |
| IFX-124 | c[arg-arg-arg-arg-arg-trp-naI-naI] | 3.1 | 3.1 | 16 | 16 | 410 |
| IFX-125 | c[Arg-Arg-Arg-arg-arg-Trp-naI-naI] | 3.1 | 3.1 | 32 | 16 | 485 |
| IFX-126 | c[Lys-Lys-Lys-Lys-Trp-NaI-NaI] | 12.5 | 12.5 | 128 | 64 | 610 |
| IFX-127 | c[Lys-Lys-Lys-Lys-Trp-naI-naI] | 6.2 | 6.2 | 128 | 64 | 540 |
| IFX-128 | c[lys-Lys-lys-Lys-Trp-naI-naI] | 6.2 | 6.2 | 64 | 64 | 590 |
| IFX-129 | c[Lys-lys-Lys-lys-Trp-naI-naI] | 3.1 | 3.1 | 64 | 64 | 420 |
| IFX-130 | c[lys-lys-lys-lys-trp-naI-naI] | 12.5 | 6.2 | 128 | 128 | 880 |
| IFX-131 | c[Lys-Lys-Lys-Lys-Lys-Trp-NaI-NaI] | 12.5 | 6.2 | 64 | 128 | 690 |
| IFX-132 | c[Lys-Lys-Lys-Lys-Lys-trp-naI-naI] | 12.5 | 12.5 | 128 | 128 | 930 |
| IFX-133 | c[lys-lys-lys-lys-lys-trp-naI-naI] | 6.2 | 6.2 | 128 | 128 | 580 |
| IFX-134 | c[lys-lys-lys-lys-lys-Trp-NaI-NaI] | 12.5 | 12.5 | 128 | 128 | 890 |
| IFX-135 | c[Arg-Arg-arg-arg-naI-Trp-naI] | 1.5 | 1.5 | 32 | 8 | 680 |
| IFX-136 | c[arg-arg-Arg-Arg-naI-Trp-naI] | 3.1 | 1.5 | 32 | 16 | 390 |
| IFX-137 | c[Arg-Arg-arg-arg-naI-trp-naI] | 3.1 | 3.1 | 32 | 16 | 720 |
| IFX-138 | c[arg-arg-Arg-Arg-naI-trp-naI] | 3.1 | 1.5 | 32 | 16 | 480 |
| IFX-139 | c[Arg-Arg-arg-arg-NaI-trp-NaI] | 3.1 | 3.1 | 32 | 32 | 425 |
| IFX-140 | c[arg-arg-Arg-Arg-NaI-trp-NaI] | 3.1 | 3.1 | 32 | 32 | 390 |
| IFX-141 | c[arg-arg-arg-arg-NaI-trp-NaI] | 6.2 | 3.1 | 32 | 32 | 370 |
| IFX-142 | c[arg-arg-arg-arg-naI-trp-naI] | 3.1 | 3.1 | 32 | 16 | 285 |
| IFX-143 | c[Arg-Arg-Arg-Arg-naI-trp-naI] | 3.1 | 1.5 | 64 | 16 | 690 |

TABLE 15
Broad-spectrum activity of peptides in this invention against Gram-Positive bacteria.

| | | E. | E. | E. | E. | S. | S. | B. | B. |
|---|---|---|---|---|---|---|---|---|---|
| | | faecium | faecium | faecalis | faecalis | pneumoniae | pneumoniae | subtilis | cereus |
| Peptide | Peptide | (ATCC | (ATCC | (ATCC | (ATCC | (ATCC | (ATCC | (ATCC- | (ATCC- |
| Code | Sequence | 27270) | 700221) | 29212) | 51575) | 49619) | 51938) | 6633) | 13061) |
| IFX-002 | c[Arg-Arg-Arg-Dip-Dip-Dip] | 6.2 | 6.2 | 12.5 | 12.5 | 50 | 25 | 6.2 | 12.5 |
| IFX-008 | c[Arg-Arg-ArgArg-Dip-Dip-Dip] | 3.1 | 1.5 | 6.2 | 12.5 | 25 | 12.5 | 1.5 | 6.2 |
| IFX-009 | c[Arg-Arg-Arg Arg-NaI-NaI-NaI] | 3.1 | 3.1 | 6.2 | 12.5 | 25 | 12.5 | 3.1 | 6.2 |
| IFX-016 | c[Arg-Arg-Arg-Arg-Trp-Dip-Dip] | 3.1 | 1.5 | 6.2 | 12.5 | 25 | 12.5 | 1.5 | 6.2 |
| IFX-017 | c[Arg-Arg-Arg-Arg-Dip-Trp-Dip] | 3.1 | 1.5 | 12.5 | 12.5 | 50 | 12.5 | 1.5 | 6.2 |
| IFX-018 | c[Arg-Arg-Arg-Arg-Dip-Dip-Trp] | 1.5 | 1.5 | 6.2 | 12.5 | 25 | 12.5 | 1.5 | 3.1 |
| IFX-019 | c[Arg-Arg-Arg-Arg-Trp-NaI-NaI] | 1.5 | 1.5 | 3.1 | 6.2 | 25 | 12.5 | 1.5 | 1.5 |
| IFX-020 | c[Arg-Arg-Arg-Arg-NaI-Trp-NaI] | 3.1 | 1.5 | 6.2 | 12.5 | 25 | 12.5 | 1.5 | 3.1 |
| IFX-021 | c[Arg-Arg-Arg-Arg-NaI-NaI-Trp] | 6.2 | 3.1 | 12.5 | 12.5 | 50 | 25 | 3.1 | 6.2 |
| IFX-060 | c[Arg-Arg-Arg-Arg-Dip-Dip-dip] | 3.1 | 1.5 | 3.1 | 12.5 | 25 | 12.5 | 1.5 | 3.1 |
| IFX-061 | c[Arg-Arg-Arg-Arg-Dip-dip-Dip] | 3.1 | 3.1 | 6.2 | 12.5 | 50 | 12.5 | 1.5 | 3.1 |
| IFX-062 | c[Arg-Arg-Arg-Arg-dip-Dip-Dip] | 3.1 | 3.1 | 6.2 | 12.5 | 50 | 12.5 | 1.5 | 3.1 |
| IFX-063 | c[Arg-Arg-Arg-arg-Dip-Dip-Dip] | 3.1 | 1.5 | 6.2 | 12.5 | 25 | 12.5 | 1.5 | 3.1 |
| IFX-064 | c[Arg-Arg-arg-Arg-Dip-Dip-Dip] | 3.1 | 1.5 | 6.2 | 12.5 | 50 | 25 | 1.5 | 3.1 |
| IFX-065 | c[Arg-arg-Arg-Arg-Dip-Dip-Dip] | 3.1 | 3.1 | 6.2 | 12.5 | 25 | 12.5 | 1.5 | 3.1 |
| IFX-066 | c[arg-Arg-Arg-Arg-Dip-Dip-Dip] | 3.1 | 3.1 | 6.2 | 12.5 | 25 | 12.5 | 1.5 | 6.2 |
| IFX-067 | c[arg-arg-arg-arg-dip-dip-dip] | 3.1 | 3.1 | 6.2 | 6.2 | 25 | 12.5 | 1.5 | 3.1 |
| IFX-068 | c[arg-Arg-arg-Arg-dip-dip-dip] | 3.1 | 3.1 | 6.2 | 12.5 | 50 | 25 | 1.5 | 3.1 |
| IFX-069 | c[arg-Arg-arg-Arg-dip-Dip-dip] | 3.1 | 3.1 | 6.2 | 12.5 | 25 | 12.5 | 1.5 | 3.1 |
| IFX-070 | c[Arg-arg-Arg-arg-dip-dip-dip] | 3.1 | 3.1 | 6.2 | 12.5 | 50 | 12.5 | 1.5 | 3.1 |
| IFX-071 | c[Arg-arg-Arg-arg-Dip-dip-Dip] | 6.2 | 3.1 | 12.5 | 12.5 | 50 | 12.5 | 1.5 | 6.2 |
| IFX-072 | c[Arg-Arg-Arg-Arg-dip-dip-dip] | 3.1 | 1.5 | 6.2 | 12.5 | 25 | 12.5 | 1.5 | 3.1 |
| IFX-073 | c[arg-arg-arg-arg-Dip-Dip-Dip] | 3.1 | 3.1 | 3.1 | 12.5 | 50 | 12.5 | 1.5 | 3.1 |
| IFX-074 | c[Arg-Arg-Arg-Arg-NaI-NaI-naI] | 3.1 | 3.1 | 6.2 | 12.5 | 25 | 12.5 | 1.5 | 3.1 |
| IFX-075 | c[Arg-Arg-Arg-Arg-NaI-naI-NaI] | 6.2 | 3.1 | 6.2 | 6.2 | 25 | 12.5 | 1.5 | 3.1 |
| IFX-076 | c[Arg-Arg-Arg-Arg-naI-NaI-NaI] | 6.2 | 3.1 | 6.2 | 6.2 | 25 | 12.5 | 1.5 | 3.1 |
| IFX-077 | c[Arg-Arg-Arg-arg-NaI-NaI-NaI] | 6.2 | 6.2 | 6.2 | 12.5 | 50 | 25 | 3.1 | 6.2 |
| IFX-078 | c[Arg-Arg-arg-Arg-NaI-NaI-NaI] | 6.2 | 6.2 | 6.2 | 6.2 | 25 | 12.5 | 1.5 | 3.1 |
| IFX-079 | c[Arg-arg-Arg-Arg-NaI-NaI-NaI] | 6.2 | 3.1 | 6.2 | 6.2 | 50 | 25 | 1.5 | 3.1 |
| IFX-080 | c[arg-Arg-Arg-Arg-NaI-NaI-NaI] | 6.2 | 6.2 | 6.2 | 12.5 | 50 | 25 | 1.5 | 6.2 |
| IFX-081 | c[arg-arg-arg-arg-naI-naI-naI] | 12.5 | 6.2 | 12.5 | 12.5 | 50 | 25 | 3.1 | 12.5 |
| IFX-082 | c[arg-Arg-arg-Arg-naI-naI-naI] | 6.2 | 6.2 | 6.2 | 12.5 | 50 | 25 | 1.5 | 6.2 |
| IFX-083 | c[arg-Arg-arg-Arg-naI-NaI-naI] | 3.1 | 3.1 | 3.1 | 6.2 | 25 | 12.5 | 1.5 | 3.1 |
| IFX-084 | c[Arg-arg-Arg-arg-naI-naI-naI] | 3.1 | 6.2 | 6.2 | 6.2 | 50 | 12.5 | 1.5 | 6.2 |
| IFX-085 | c[Arg-arg-Arg-arg-NaI-naI-NaI] | 3.1 | 3.1 | 3.1 | 6.2 | 25 | 12.5 | 1.5 | 3.1 |
| IFX-086 | c[Arg-Arg-Arg-Arg-naI-naI-naI] | 6.2 | 3.1 | 6.2 | 12.5 | 50 | 12.5 | 1.5 | 6.2 |
| IFX-087 | c[arg-arg-arg-arg-NaI-NaI-NaI] | 6.2 | 3.1 | 6.2 | 12.5 | 50 | 12.5 | 1.5 | 3.1 |
| IFX-088 | c[Arg-Arg-Arg-Arg-Dip-Trp-dip] | 3.1 | 12.5 | 12.5 | 12.5 | 25 | 12.5 | 1.5 | 6.2 |
| IFX-089 | c[Arg-Arg-Arg-Arg-Dip-trp-Dip] | 6.2 | 12.5 | 12.5 | 12.5 | 50 | 25 | 3.1 | 12.5 |
| IFX-090 | c[Arg-Arg-Arg-Arg-dip-Trp-Dip] | 3.1 | 6.2 | 6.2 | 12.5 | 25 | 12.5 | 1.5 | 6.2 |
| IFX-091 | c[Arg-Arg-Arg-arg-Dip-Trp-Dip] | 3.1 | 6.2 | 6.2 | 12.5 | 50 | 12.5 | 1.5 | 3.1 |
| IFX-092 | c[Arg-Arg-arg-Arg-Dip-Trp-Dip] | 6.2 | 12.5 | 12.5 | 12.5 | 50 | 25 | 1.5 | 6.2 |
| IFX-093 | c[Arg-arg-Arg-Arg-Dip-Trp-Dip] | 6.2 | 6.2 | 12.5 | 12.5 | 50 | 25 | 1.5 | 6.2 |
| IFX-094 | c[arg-Arg-Arg-Arg-Dip-Trp-Dip] | 6.2 | 12.5 | 12.5 | 12.5 | 25 | 25 | 3.1 | 6.2 |
| IFX-095 | c[arg-arg-arg-arg-dip-Trp-dip] | 3.1 | 6.2 | 6.2 | 12.5 | 50 | 12.5 | 1.5 | 3.1 |
| IFX-096 | c[arg-Arg-arg-Arg-dip-Trp-dip] | 3.1 | 6.2 | 12.5 | 12.5 | 25 | 25 | 3.1 | 6.2 |
| IFX-097 | c[Arg-arg-Arg-arg-dip-Trp-dip] | 3.1 | 6.2 | 6.2 | 12.5 | 50 | 25 | 1.5 | 6.2 |
| IFX-098 | c[Arg-Arg-Arg-Arg-dip-Trp-dip] | 3.1 | 6.2 | 6.2 | 12.5 | 50 | 25 | 1.5 | 3.1 |
| IFX-099 | c[arg-arg-arg-arg-Dip-Trp-Dip] | 3.1 | 6.2 | 6.2 | 12.5 | 25 | 12.5 | 1.5 | 3.1 |
| IFX-100 | c[Arg-Arg-Arg-Arg-dip-trp-dip] | 3.1 | 6.2 | 6.2 | 12.5 | 25 | 12.5 | 1.5 | 3.1 |
| IFX-101 | c[arg-arg-arg-arg-dip-trp-dip] | 3.1 | 12.5 | 12.5 | 12.5 | 50 | 12.5 | 1.5 | 6.2 |
| IFX-102 | c[Arg-Arg-Arg-Arg-Trp-NaI-naI] | 3.1 | 6.2 | 6.2 | 12.5 | 50 | 25 | 1.5 | 6.2 |
| IFX-103 | c[Arg-Arg-Arg-Arg-Trp-naI-NaI] | 3.1 | 3.1 | 6.2 | 6.2 | 25 | 12.5 | 1.5 | 3.1 |
| IFX-104 | c[Arg-Arg-Arg-Arg-trp-NaI-NaI] | 3.1 | 3.1 | 6.2 | 6.2 | 25 | 12.5 | 1.5 | 3.1 |
| IFX-105 | c[Arg-Arg-Arg-arg-Trp-NaI-NaI] | 3.1 | 6.2 | 6.2 | 6.2 | 50 | 25 | 3.1 | 6.2 |
| IFX-106 | c[Arg-Arg-arg-Arg-Trp-NaI-NaI] | 3.1 | 6.2 | 6.2 | 6.2 | 25 | 12.5 | 1.5 | 3.1 |
| IFX-107 | c[Arg-arg-Arg-Arg-Trp-NaI-NaI] | 3.1 | 6.2 | 6.2 | 6.2 | 25 | 12.5 | 1.5 | 3.1 |
| IFX-108 | c[arg-Arg-Arg-Arg-Trp-NaI-NaI] | 3.1 | 6.2 | 6.2 | 12.5 | 50 | 12.5 | 1.5 | 3.1 |
| IFX-109 | c[arg-arg-arg-arg-Trp-naI-naI] | 3.1 | 3.1 | 6.2 | 12.5 | 50 | 25 | 1.5 | 3.1 |
| IFX-110 | c[arg-Arg-arg-Arg-Trp-naI-naI] | 3.1 | 3.1 | 6.2 | 12.5 | 50 | 12.5 | 3.1 | 6.2 |
| IFX-111 | c[Arg-arg-Arg-arg-Trp-naI-naI] | 3.1 | 1.5 | 3.1 | 6.2 | 25 | 12.5 | 1.5 | 3.1 |
| IFX-112 | c[Arg-Arg-Arg-Arg-Trp-naI-naI] | 3.1 | 3.1 | 6.2 | 12.5 | 50 | 12.5 | 1.5 | 3.1 |
| IFX-113 | c[arg-arg-arg-arg-Trp-NaI-NaI] | 6.2 | 6.2 | 6.2 | 12.5 | 50 | 25 | 3.1 | 6.2 |
| IFX-114 | c[Arg-Arg-Arg-Arg-trp-naI-naI] | 6.2 | 3.1 | 6.2 | 12.5 | 50 | 12.5 | 1.5 | 6.2 |
| IFX-115 | c[arg-arg-arg-arg-trp-naI-naI] | 6.2 | 6.2 | 6.2 | 12.5 | 50 | 12.5 | 3.1 | 6.2 |
| IFX-116 | c[Arg-Arg-arg-arg-Trp-naI-naI] | 3.1 | 6.2 | 6.2 | 12.5 | 50 | 25 | 1.5 | 6.2 |
| IFX-117 | c[arg-arg-Arg-Arg-Trp-naI-naI] | 3.1 | 3.1 | 6.2 | 6.2 | 25 | 12.5 | 1.5 | 3.1 |
| IFX-118 | c[Arg-Arg-arg-arg-trp-naI-naI] | 3.1 | 3.1 | 6.2 | 6.2 | 25 | 12.5 | 1.5 | 3.1 |
| IFX-119 | c[arg-arg-Arg-Arg-trp-naI-naI] | 3.1 | 6.2 | 6.2 | 6.2 | 50 | 25 | 3.1 | 6.2 |
| IFX-120 | c[Arg-Arg-arg-arg-trp-NaI-NaI] | 3.1 | 6.2 | 6.2 | 6.2 | 25 | 12.5 | 1.5 | 3.1 |
| IFX-121 | c[arg-arg-Arg-Arg-trp-NaI-NaI] | 3.1 | 6.2 | 6.2 | 6.2 | 25 | 12.5 | 1.5 | 3.1 |
| IFX-122 | c[arg-arg-arg-arg-trp-NaI-NaI] | 3.1 | 6.2 | 6.2 | 12.5 | 50 | 12.5 | 1.5 | 3.1 |
| IFX-123 | c[arg-arg-arg-arg-arg-trp-NaI-NaI] | 3.1 | 3.1 | 6.2 | 12.5 | 50 | 25 | 1.5 | 3.1 |
| IFX-124 | c[arg-arg-arg-arg-arg-trp-naI-naI] | 3.1 | 3.1 | 6.2 | 12.5 | 50 | 12.5 | 3.1 | 6.2 |
| IFX-125 | c[Arg-Arg-Arg-arg-arg-Trp-naI-naI] | 3.1 | 1.5 | 3.1 | 6.2 | 25 | 12.5 | 1.5 | 3.1 |
| IFX-135 | c[Arg-Arg-arg-arg-naI-Trp-naI] | 3.1 | 3.1 | 6.2 | 12.5 | 12.5 | 12.5 | 1.5 | 6.2 |
| IFX-136 | c[arg-arg-Arg-Arg-naI-Trp-naI] | 3.1 | 3.1 | 6.2 | 6.2 | 25 | 12.5 | 1.5 | 3.1 |
| IFX-137 | c[Arg-Arg-arg-arg-naI-trp-naI] | 3.1 | 3.1 | 6.2 | 6.2 | 25 | 12.5 | 1.5 | 3.1 |
| IFX-138 | c[arg-arg-Arg-Arg-naI-trp-naI] | 3.1 | 6.2 | 6.2 | 6.2 | 50 | 25 | 3.1 | 6.2 |
| IFX-139 | c[Arg-Arg-arg-arg-NaI-trp-NaI] | 3.1 | 6.2 | 6.2 | 6.2 | 25 | 12.5 | 1.5 | 3.1 |
| IFX-140 | c[arg-arg-Arg-Arg-NaI-trp-NaI] | 3.1 | 6.2 | 6.2 | 6.2 | 25 | 12.5 | 1.5 | 3.1 |
| IFX-141 | c[arg-arg-arg-arg-NaI-trp-NaI] | 6.2 | 6.2 | 6.2 | 12.5 | 50 | 12.5 | 1.5 | 3.1 |
| IFX-142 | c[arg-arg-arg-arg-naI-trp-naI] | 6.2 | 3.1 | 6.2 | 12.5 | 50 | 25 | 1.5 | 3.1 |
| IFX-143 | c[Arg-Arg-Arg-Arg-naI-trp-naI] | 3.1 | 3.1 | 6.2 | 12.5 | 50 | 12.5 | 3.1 | 6.2 |

TABLE 16
Broad-spectrum activity of peptides in this invention against Gram-Negative bacteria.

| | | E. coli | K. pneumonia | K. pneumonia | A. baumannii | P. aeruginosa | P. aeruginosa |
|---|---|---|---|---|---|---|---|
| Peptide | | (ATCC BAA- | (ATCC | (ATCC | (ATCC | (ATCC | (ATCC |
| Code | Peptide Sequence | 2452) | 13883) | BAA-1705) | BAA-1605) | 10145) | BAA-1744) |
| IFX-002 | c[Arg-Arg-Arg-Dip-Dip-Dip] | 25 | 50 | 12.5 | 25 | 50 | 25 |
| IFX-008 | c[Arg-Arg-ArgArg-Dip-Dip-Dip] | 6.2 | 50 | 12.5 | 12.5 | 25 | 12.5 |
| IFX-009 | c[Arg-Arg-ArgArg-NaI-NaI-NaI] | 6.2 | 50 | 12.5 | 12.5 | 25 | 12.5 |
| IFX-016 | c[Arg-Arg-Arg-Arg-Trp-Dip-Dip] | 12.5 | 50 | 25 | 12.5 | 25 | 12.5 |
| IFX-017 | c[Arg-Arg-Arg-Arg-Dip-Trp-Dip] | 12.5 | 50 | 25 | 12.5 | 25 | 25 |
| IFX-018 | c[Arg-Arg-Arg-Arg-Dip-Dip-Trp] | 12.5 | 50 | 25 | 6.2 | 50 | 25 |
| IFX-019 | c[Arg-Arg-Arg-Arg-Trp-NaI-NaI] | 6.2 | 50 | 25 | 12.5 | 25 | 12.5 |
| IFX-020 | c[Arg-Arg-Arg-Arg-NaI-Trp-NaI] | 6.2 | 50 | 12.5 | 25 | 25 | 12.5 |
| IFX-021 | c[Arg-Arg-Arg-Arg-NaI-NaI-Trp] | 12.5 | >50 | 12.5 | 25 | 50 | 25 |
| IFX-060 | c[Arg-Arg-Arg-Arg-Dip-Dip-dip] | 12.5 | 50 | 12.5 | 6.2 | 25 | 12.5 |
| IFX-061 | c[Arg-Arg-Arg-Arg-Dip-dip-Dip] | 12.5 | 50 | 25 | 6.2 | 25 | 12.5 |
| IFX-062 | c[Arg-Arg-Arg-Arg-dip-Dip-Dip] | 6.2 | 50 | 12.5 | 6.2 | 25 | 12.5 |
| IFX-063 | c[Arg-Arg-Arg-arg-Dip-Dip-Dip] | 12.5 | 50 | 12.5 | 6.2 | 25 | 25 |
| IFX-064 | c[Arg-Arg-arg-Arg-Dip-Dip-Dip] | 6.2 | 50 | 12.5 | 6.2 | 25 | 25 |
| IFX-065 | c[Arg-arg-Arg-Arg-Dip-Dip-Dip] | 6.2 | 50 | 12.5 | 6.2 | 12.5 | 12.5 |
| IFX-066 | c[arg-Arg-Arg-Arg-Dip-Dip-Dip] | 12.5 | 50 | 12.5 | 6.2 | 25 | 12.5 |
| IFX-067 | c[arg-arg-arg-arg-dip-dip-dip] | 12.5 | >50 | 25 | 12.5 | 50 | 25 |
| IFX-068 | c[arg-Arg-arg-Arg-dip-dip-dip] | 25 | 50 | 25 | 6.2 | 25 | 25 |
| IFX-069 | c[arg-Arg-arg-Arg-dip-Dip-dip] | 12.5 | 50 | 12.5 | 6.2 | 12.5 | 25 |
| IFX-070 | c[Arg-arg-Arg-arg-dip-dip-dip] | 12.5 | 50 | 25 | 12.5 | >50 | 25 |
| IFX-071 | c[Arg-arg-Arg-arg-Dip-dip-Dip] | 12.5 | 50 | 12.5 | 6.2 | 25 | 25 |
| IFX-072 | c[Arg-Arg-Arg-Arg-dip-dip-dip] | 12.5 | 50 | 25 | 12.5 | 50 | 25 |
| IFX-073 | c[arg-arg-arg-arg-Dip-Dip-Dip] | 12.5 | 50 | 25 | 12.5 | 50 | 25 |
| IFX-074 | c[Arg-Arg-Arg-Arg-NaI-NaI-naI] | 12.5 | 50 | 12.5 | 12.5 | 25 | 25 |
| IFX-075 | c[Arg-Arg-Arg-Arg-NaI-naI-NaI] | 6.2 | 50 | 25 | 12.5 | 25 | 12.5 |
| IFX-076 | c[Arg-Arg-Arg-Arg-naI-NaI-NaI] | 12.5 | 50 | 25 | 12.5 | 25 | 12.5 |
| IFX-077 | c[Arg-Arg-Arg-arg-NaI-NaI-NaI] | 12.5 | 50 | 12.5 | 6.2 | 25 | 12.5 |
| IFX-078 | c[Arg-Arg-arg-Arg-NaI-NaI-NaI] | 12.5 | 50 | 25 | 25 | 25 | 12.5 |
| IFX-079 | c[Arg-arg-Arg-Arg-NaI-NaI-NaI] | 12.5 | 50 | 25 | 12.5 | 25 | 25 |
| IFX-080 | c[arg-Arg-Arg-Arg-NaI-NaI-NaI] | 12.5 | 50 | 25 | 12.5 | 12.5 | 12.5 |
| IFX-081 | c[arg-arg-arg-arg-naI-naI-naI] | 25 | >50 | 50 | 25 | 50 | 50 |
| IFX-082 | c[arg-Arg-arg-Arg-naI-naI-naI] | 12.5 | 25 | 25 | 25 | 12.5 | 12.5 |
| IFX-083 | c[arg-Arg-arg-Arg-naI-NaI-naI] | 12.5 | 50 | 25 | 12.5 | 25 | 25 |
| IFX-084 | c[Arg-arg-Arg-arg-naI-naI-naI] | 25 | >50 | 25 | 25 | 25 | 25 |
| IFX-085 | c[Arg-arg-Arg-arg-NaI-naI-NaI] | 12.5 | 50 | 25 | 12.5 | 25 | 12.5 |
| IFX-086 | c[Arg-Arg-Arg-Arg-naI-naI-naI] | 12.5 | 50 | 12.5 | 25 | 25 | 12.5 |
| IFX-087 | c[arg-arg-arg-arg-NaI-NaI-NaI] | 12.5 | 50 | 25 | 12.5 | 25 | 12.5 |
| IFX-088 | c[Arg-Arg-Arg-Arg-Dip-Trp-dip] | 12.5 | 50 | 25 | 12.5 | 25 | 50 |
| IFX-089 | c[Arg-Arg-Arg-Arg-Dip-trp-Dip] | 12.5 | 50 | 25 | 12.5 | 25 | 25 |
| IFX-090 | c[Arg-Arg-Arg-Arg-dip-Trp-Dip] | 12.5 | 50 | 25 | 12.5 | 25 | 25 |
| IFX-091 | c[Arg-Arg-Arg-arg-Dip-Trp-Dip] | 12.5 | 50 | 12.5 | 12.5 | 25 | 25 |
| IFX-092 | c[Arg-Arg-arg-Arg-Dip-Trp-Dip] | 12.5 | 50 | 25 | 12.5 | 25 | 25 |
| IFX-093 | c[Arg-arg-Arg-Arg-Dip-Trp-Dip] | 6.2 | 50 | 12.5 | 12.5 | 25 | 25 |
| IFX-094 | c[arg-Arg-Arg-Arg-Dip-Trp-Dip] | 12.5 | 50 | 12.5 | 12.5 | 50 | 25 |
| IFX-095 | c[arg-arg-arg-arg-dip-Trp-dip] | 25 | 50 | 12.5 | 12.5 | 25 | 25 |
| IFX-096 | c[arg-Arg-arg-Arg-dip-Trp-dip] | 12.5 | 50 | 25 | 25 | 50 | 50 |
| IFX-097 | c[Arg-arg-Arg-arg-dip-Trp-dip] | 25 | >50 | 25 | 12.5 | 50 | 50 |
| IFX-098 | c[Arg-Arg-Arg-Arg-dip-Trp-dip] | 25 | 50 | 25 | 12.5 | 25 | 25 |
| IFX-099 | c[arg-arg-arg-arg-Dip-Trp-Dip] | 25 | 50 | 25 | 12.5 | 25 | 25 |
| IFX-100 | c[Arg-Arg-Arg-Arg-dip-trp-dip] | 25 | >50 | 25 | 12.5 | 50 | 25 |
| IFX-101 | c[arg-arg-arg-arg-dip-trp-dip] | 25 | >50 | 25 | 12.5 | 50 | 25 |
| IFX-102 | c[Arg-Arg-Arg-Arg-Trp-NaI-naI] | 12.5 | 50 | 25 | 25 | 25 | 25 |
| IFX-103 | c[Arg-Arg-Arg-Arg-Trp-naI-NaI] | 12.5 | 50 | 25 | 6.2 | 25 | 25 |
| IFX-104 | c[Arg-Arg-Arg-Arg-trp-NaI-NaI] | 12.5 | 50 | 25 | 12.5 | 25 | 25 |
| IFX-105 | c[Arg-Arg-Arg-arg-Trp-NaI-NaI] | 12.5 | 50 | 25 | 25 | 25 | 25 |
| IFX-106 | c[Arg-Arg-arg-Arg-Trp-NaI-NaI] | 12.5 | 50 | 25 | 25 | 25 | 25 |
| IFX-107 | c[Arg-arg-Arg-Arg-Trp-NaI-NaI] | 6.2 | 50 | 25 | 12.5 | 25 | 12.5 |
| IFX-108 | c[arg-Arg-Arg-Arg-Trp-NaI-NaI] | 12.5 | 50 | 25 | 12.5 | 25 | 12.5 |
| IFX-109 | c[arg-arg-arg-arg-Trp-naI-naI] | 12.5 | >50 | 25 | 25 | >50 | 25 |
| IFX-110 | c[arg-Arg-arg-Arg-Trp-naI-naI] | 12.5 | >50 | 25 | 25 | 50 | 25 |
| IFX-111 | c[Arg-arg-Arg-arg-Trp-naI-naI] | 6.2 | 50 | 25 | 6.2 | 25 | 12.5 |
| IFX-112 | c[Arg-Arg-Arg-Arg-Trp-naI-naI] | 12.5 | >50 | 25 | 25 | 50 | 25 |
| IFX-113 | c[arg-arg-arg-arg-Trp-NaI-NaI] | 12.5 | >50 | 25 | 25 | >50 | 25 |
| IFX-114 | c[Arg-Arg-Arg-Arg-trp-naI-naI] | 12.5 | 50 | 12.5 | 12.5 | 25 | 12.5 |
| IFX-115 | c[arg-arg-arg-arg-trp-naI-naI] | 12.5 | >50 | 25 | 25 | >50 | 25 |
| IFX-116 | c[Arg-Arg-arg-arg-Trp-naI-naI] | 12.5 | 50 | 50 | 25 | 25 | 25 |
| IFX-117 | c[arg-arg-Arg-Arg-Trp-naI-naI] | 6.2 | 50 | 25 | 12.5 | 25 | 12.5 |
| IFX-118 | c[Arg-Arg-arg-arg-trp-naI-naI] | 12.5 | 50 | 25 | 12.5 | 25 | 12.5 |
| IFX-119 | c[arg-arg-Arg-Arg-trp-naI-naI] | 12.5 | >50 | 12.5 | 25 | >50 | 25 |
| IFX-120 | c[Arg-Arg-arg-arg-trp-NaI-NaI] | 12.5 | >50 | >50 | 25 | 50 | 25 |
| IFX-121 | c[arg-arg-Arg-Arg-trp-NaI-NaI] | 6.2 | 50 | 50 | 6.2 | 25 | 12.5 |
| IFX-122 | c[arg-arg-arg-arg-trp-NaI-NaI] | 12.5 | >50 | >50 | 25 | 50 | 25 |
| IFX-123 | c[arg-arg-arg-arg-arg-trp-NaI-NaI] | 12.5 | >50 | >50 | 25 | >50 | 25 |
| IFX-124 | c[arg-arg-arg-arg-arg-trp-naI-naI] | 12.5 | 50 | 25 | 12.5 | 25 | 12.5 |
| IFX-125 | c[Arg-Arg-Arg-arg-arg-Trp-naI-naI] | 12.5 | >50 | 50 | 25 | >50 | 25 |
| IFX-135 | c[Arg-Arg-arg-arg-naI-Trp-naI] | 12.5 | 50 | 12.5 | 12.5 | 25 | 12.5 |
| IFX-136 | c[arg-arg-Arg-Arg-naI-Trp-naI] | 12.5 | 50 | 25 | 12.5 | 50 | 25 |
| IFX-137 | c[Arg-Arg-arg-arg-naI-trp-naI] | 25 | 50 | 50 | 12.5 | 25 | 25 |
| IFX-138 | c[arg-arg-Arg-Arg-naI-trp-naI] | 12.5 | 50 | 50 | 25 | 50 | 50 |
| IFX-139 | c[Arg-Arg-arg-arg-NaI-trp-NaI] | 25 | >50 | 25 | 12.5 | 50 | 50 |
| IFX-140 | c[arg-arg-Arg-Arg-NaI-trp-NaI] | 25 | 50 | >50 | 12.5 | 25 | 25 |
| IFX-141 | c[arg-arg-arg-arg-NaI-trp-NaI] | 25 | 50 | >50 | 12.5 | 25 | 25 |
| IFX-142 | c[arg-arg-arg-arg-naI-trp-naI] | 25 | >50 | >50 | 12.5 | 50 | 25 |
| IFX-143 | c[Arg-Arg-Arg-Arg-naI-trp-naI] | 25 | >50 | 25 | 12.5 | 50 | 25 |

TABLE 17
Minimum inhibitory concentration (MIC) determination
of three compounds against Clostridium difficile.

| | Clostridium difficile | | | Clostridium difficile | | |
|---|---|---|---|---|---|---|
| | (ATCC 700057) | | | (NAP1/027) | | |
| Compound | MIC₉₀ | MIC₉₅ | MIC₉₉ | MIC₉₀ | MIC₉₅ | MIC₉₉ |
| IFX-031 | 12.5 | 12.5 | 25 | 15. | 25 | 25 |
| IFX-031-1 | 25 | 25 | 25 | 25 | >50 | 25 |
| IFX-111 | 12.5 | 12.5 | 12.5 | 6.25 | 6.25 | 6.25 |
| Vancomycin | 0.63 | 0.63 | 0.63 | 0.31 | 0.31 | 0.31 |
| Ciprofloxacin | | | | 50 | >50 | >50 |

TABLE 18
Antibacterial activities of IFX-111 and IFX-135 in the
presence of Serum and various physiologically relevant
salts (The MICs were measured in MH broth supplemented with
various salt ions (150 mM NaCl, 4.5 mM KCl, 6 mM NH₄Cl,
1 mM MgCl₂, and 2 mM CaCl₂) or FBS (25%).

| | MH | | | | | | |
|---|---|---|---|---|---|---|---|
| Peptide | Media | NaCl | KCl | NH₄Cl | MgCl₂ | CaCl₂ | FBS |
| | MIC (μg/mL) Methicillin-resistant | | | | | | |
| | Staphylococcus aureus (MRSA) (ATCC BAA-1556) | | | | | | |
| IFX-111 | 1.5 | 1.5 | 1.5 | 1.5 | 1.5 | 1.5 | 1.5 |
| IFX-135 | 1.5 | 1.5 | 1.5 | 1.5 | 1.5 | 1.5 | 3.2 |
| Daptomycin | 0.7 | 0.7 | 0.7 | 0.7 | 0.7 | 0.7 | 1.5 |
| Polymyxin B | NA | NA | NA | NA | NA | NA | NA |
| | MIC (μg/mL) Escherichia coli (ATCC 25922) | | | | | | |
| IFX-111 | 12.5 | 12.5 | 12.5 | 12.5 | 25 | 12.5 | 12.5 |
| IFX-135 | 6.2 | 6.2 | 6.2 | 6.2 | 12.5 | 6.2 | 12.5 |
| Daptomycin | NA | NA | NA | NA | NA | NA | NA |
| Polymyxin B | 0.7 | 0.7 | 0.7 | 0.7 | 1.5 | 0.7 | 0.7 |

TABLE 19
Antibacterial activity of cyclic peptides
contining arginine and tryptophan residues.

| | | MIC μg/mL | | | |
|---|---|---|---|---|---|
| | | MRSA | K. pneumoniae | | E. coli |
| | | (ATCC | (ATCC | P. aeruginosa | (ATCC |
| Sequence | | BAA-1556) | BAA-1705) | ATCC 27883 | 25922) |
| IFX-300 | [R₂W₃] | 32 | 256 | 256 | 256 |
| IFX-301 | [R₅W₄] | 4 | 32 | 32 | 16 |
| IFX-302 | [R₃W₃] | 16 | 64 | 128 | 32 |
| IFX-303 | [R₃W₄] | 8 | 64 | 256 | 32 |
| IFX-304 | [R₃W₅] | 128 | 128 | 64 | 128 |
| IFX-305 | [R₃W₆] | 64 | 128 | 256 | 64 |
| IFX-306 | [R₃W₇] | 256 | 64 | 256 | 256 |
| IFX-307 | [dR₃W₇] | 64 | 256 | 256 | 256 |
| IFX-308 | [W_{Me4}R₄] | 8 | NT | NT | 16 |
| IFX-309 | [dR₄W₄] | 8 | NT | NT | 16 |
| IFX-310 | [R₄W₅] | 8 | 128 | 128 | 64 |
| IFX-311 | [R₄W₆] | 128 | 256 | 128 | 128 |
| IFX-312 | [R₄W₇] | 256 | 256 | >256 | 128 |
| IFX-313 | [R₂W₄] | 64 | 256 | 256 | 256 |
| IFX-314 | [R₅W₅] | 4 | 32 | 32 | 16 |
| IFX-315 | [R₅W₄K] | 4 | 32 | 16 | 16 |
| IFX-316 | [R₅W₆] | >256 | >256 | >256 | >256 |
| IFX-317 | [R₅W₇] | >256 | >256 | >256 | >256 |
| IFX-318 | [R₆W₄] | 4 | 64 | 8 | 32 |
| IFX-319 | [R₆W₅] | 8 | 64 | 16 | 32 |
| IFX-320 | [R₆W₆] | 16 | 64 | 128 | 64 |
| IFX-321 | [R₆W₇] | 64 | 256 | >256 | 128 |
| IFX-322 | [R₇W₄] | 16 | 64 | 32 | 32 |
| IFX-323 | [R₇W₅] | 8 | NT | NT | 64 |
| IFX-324 | [R₇W₆] | 32 | NT | NT | 64 |
| IFX-325 | [R₇W₇] | 16 | NT | NT | 32 |
| IFX-326 | [R₄W₄] | 4 | 32 | 64 | 16 |
| | Meropenem | 2 | 16 | 1 | 1 |
| | Vancomycin | 0.5 | >256 | >256 | >256 |

TABLE 20
MBC (μg/mL) of selected cyclic peptides.

| | | MBC μg/mL | | | |
|---|---|---|---|---|---|
| | | MRSA | K. pneumoniae | | E. coli |
| | | (ATCC | (ATCC | P. aeruginosa | (ATCC |
| Code | Sequence | BAA-1556) | BAA-1705) | (ATCC 27883) | 25922) |
| IFX-301 | [R₅W₄] | 8 | 64 | 32 | 16 |
| IFX-314 | [R₅W₅] | 16 | 32 | 32 | 64 |
| IFX-318 | [R₆W₄] | 16 | 128 | 16 | 32 |
| IFX-319 | [R₆W₅] | 16 | 64 | 32 | 128 |
| IFX-326 | [R₄W₄] | 32 | 64 | 128 | 32 |

TABLE 21
MIC (μg/mL) of cyclic and linear peptides containing
arginine, tryptophan, and cysteine residues

| | | MRSA | KPC | PSA | E. coli | |
|---|---|---|---|---|---|---|
| | | (ATCC | (ATCC | (ATCC | (ATCC | |
| | | BAA-1556) | BAA-1705) | 27883 | 25922) | SEQ |
| | | MIC | MIC | MIC | MIC | ID |
| Code | Sequence | (μg/mL) | (μg/mL) | (μg/mL) | (μg/mL) | NO.: |
| IFX-327 | [CR₄W₄C] disulfide | 32 | 256 | 512 | 256 | |
| | cyclization | | | | | |
| IFX-328 | CR₄W₄C- linear | 64 | 16 | 512 | 256 | 1 |
| | (SEQ ID NO. 1) | | | | | |
| IFX-329 | [R₃CW₄CR] amide | 8 | 32 | 128 | 32 | |
| | cyclization | | | | | |
| IFX-330 | [R₃CW₄CR] amide + | 8 | 32 | 128 | 32 | |
| | disulfide cyclization | | | | | |
| IFX-331 | R₄CW₄C | 16 | 64 | 128 | 32 | 2 |
| | (SEQ ID NO. 2) | | | | | |
| IFX-332 | W₄CR₄C | 64 | 512 | 256 | 128 | 3 |
| | (SEQ ID NO. 3) | | | | | |
| IFX-333 | R₄[CW₄C] disulfide | 16 | 32 | 128 | 16 | |
| | cyclization | | | | | |
| IFX-334 | W₄[CR₄C] disulfide | 32 | 256 | 256 | 32 | |
| | cyclization | | | | | |
| IFX-335 | R₄C₄ | 512 | 512 | 512 | 256 | 4 |
| | (SEQ ID NO. 4) | | | | | |
| IFX-336 | [R₄C₄] amide | 512 | 512 | 256 | 256 | |
| | cyclization | | | | | |
| IFX-337 | R₄W₄C₄ | 512 | >512 | >512 | 512 | 5 |
| | (SEQ ID NO. 5) | | | | | |
| IFX-338 | [R₄W₄C₄] amide | 64 | 512 | 512 | 256 | |
| | cyclization | | | | | |
| IFX-326 | [R₄W₄] | 4 | 32 | 64 | 16 | |
| | Meropenem | 2 | 16 | 1 | 1 | |
| | Vancomycin | 0.5 | >512 | >512 | >512 | |

TABLE 22
MBC μg/mL of of cyclic and linear peptides containing
arginine, tryptophan, and cysteine residues

| | | MRSA | KPC | PSA | E. coli | |
|---|---|---|---|---|---|---|
| | | (ATCC | (ATCC | (ATCC | (ATCC | |
| | | BAA-1556) | BAA-1705) | 27883) | 25922) | SEQ |
| | | MBC | MBC | MBC | MBC | ID |
| Code | Sequence | (μg/mL) | (μg/mL) | (μg/mL) | (μg/mL) | NO.: |
| IFX-327 | | 64 | 512 | 512 | 512 | |
| IFX-328 | [CR₄W₄C] disulfide | 128 | 32 | 512 | 512 | |
| | cyclization | | | | | |
| IFX-329 | CR₄W₄C- linear | 32 | 32 | 128 | 64 | 1 |
| | (SEQ ID NO. 1) | | | | | |
| IFX-330 | [R₃CW₄CR] amide | 32 | 128 | 256 | 64 | |
| | cyclization | | | | | |
| IFX-331 | [R₃CW₄CR] amide + | 32 | 128 | 265 | 32 | |
| | disulfide cyclization | | | | | |
| IFX-332 | R₄CW₄C | 128 | NT | NT | 256 | 2 |
| | (SEQ ID NO. 2) | | | | | |
| IFX-333 | W₄CR₄C | 32 | 64 | 128 | 32 | 3 |
| | (SEQ ID NO. 3) | | | | | |
| IFX-334 | R₄[CW₄C] disulfide | 64 | NT | 256 | 64 | |
| | cyclization | | | | | |
| IFX-335 | W₄[CR₄C] disulfide | NT | NT | 512 | NT | |
| | cyclization | | | | | |
| IFX-336 | R₄C₄ | NT | NT | 512 | NT | 4 |
| | (SEQ ID NO. 4) | | | | | |
| IFX-337 | [R₄C₄] amide | NT | NT | NT | NT | |
| | cyclization | | | | | |
| IFX-338 | R₄W₄C₄ | NT | NT | NT | NT | 5 |
| | (SEQ ID NO. 5) | | | | | |
| IFX-326 | [R₄W₄C₄] amide | 32 | 64 | 128 | 32 | |
| | cyclization | | | | | |

TABLE 23
MIC values of peptides in the presence of salts and serum.

| | NaCl | KCl | MgCl₂ | CaCl₂ | NH₄Cl | FeCl₃ | FBS | Absence |
|---|---|---|---|---|---|---|---|---|
| Peptide | 150 mM | 4.5 mM | 1 mM | 2 mM | 6 mM | 8 μM | 25% | of salts |

MICs μg/ml against MRSA (ATCC BAA-1556)

| | | | | | | | | |
|---|---|---|---|---|---|---|---|---|
| IFX-301 [R₅W₄] | 4 | 4 | 2 | 4 | 2 | 4 | 4 | 4 |
| IFX-318 [R₆W₄] | 8 | 8 | 4 | 8 | 4 | 8 | 8 | 8 |

MICs μg/ml against KPC (ATCC BAA-1705)

| | | | | | | | | |
|---|---|---|---|---|---|---|---|---|
| IFX-301 [R₅W₄] | 32 | 32 | 16 | 16 | 16 | 32 | 32 | 32 |
| IFX-318 [R₆W₄] | 64 | 64 | 32 | 64 | 32 | 64 | 64 | 64 |

MICs μg/ml against PSA (ATCC 27883)

| | | | | | | | | |
|---|---|---|---|---|---|---|---|---|
| IFX-301 [R₅W₄] | 32 | 32 | 16 | 16 | 16 | 32 | 32 | 32 |
| IFX-318 [R₆W₄] | 16 | 16 | 8 | 16 | 8 | 16 | 16 | 16 |

MICs μg/ml against E. coli (ATCC 25922)

| | | | | | | | | |
|---|---|---|---|---|---|---|---|---|
| IFX-301 [R₅W₄] | 16 | 16 | 8 | 16 | 8 | 16 | 16 | 16 |
| IFX-318 [R₆W₄] | 32 | 32 | 16 | 32 | 16 | 32 | 32 | 32 |

TABLE 24
Combination studies of IFX-318 [R₆W₄] with antibiotics.

| | | | | | | |
|---|---|---|---|---|---|---|
| | | MIC of | MIC of | FIC antibiotic/ | FIC | |
| Peptide | Antibiotic | peptide | antibiotic | peptides | index | Result |

MRSA (ATCC BAA-1556)

| | | | | | | |
|---|---|---|---|---|---|---|
| IFX-318 | Tetracycline | 8 | 0.250 | 0.0625/2 | 0.5 | Synergy |
| [R₆W₄] | Tobramycin | 8 | 0.5 | 0.0625/2 | 0.375 | Synergy |
| MIC = 8 μg/ml | Clindamycin | 8 | 0.125 | 0.031/2 | 0.498 | Synergy |
| FIC = 2 μg/ml | | | | | | |

Pseudomonas aeruginosa (ATCC 27883)

| | | | | | | |
|---|---|---|---|---|---|---|
| IFX-318 | Tetracycline | 16 | 32 | 8/4 | 0.5 | Synergy |
| [R₆W₄] | Meropenem | 16 | 1 | 0.250/2 | 0.5 | Synergy |
| MIC = 16 ug/ml | Ciprofloxacin | 16 | 0.5 | 0.125/2 | 0.5 | Synergy |
| FIC = 4 ug/ml | | | | | | |

Escherichia coli (ATCC 25922)

| | | | | | | |
|---|---|---|---|---|---|---|
| IFX-318 | Tetracycline | 32 | 8 | 0.5/8 | 0.313 | Synergy |
| [R₆W₄] | Meropenem | 32 | 1 | 0.125/8 | 0.375 | Synergy |
| MIC = 32 μg/ml | Ciprofloxacin | 32 | 64 | 8/8 | 0.375 | Synergy |
| FIC = 8 μg/ml | Clindamycin | 32 | 64 | 4/8 | 0.313 | Synergy |
| | Polymyxin | 32 | 2 | 0.125/8 | 0.313 | Synergy |
| | Levofloxacin | 32 | 64 | 8/8 | 0.375 | Synergy |
| | Kanamycin | 32 | 32 | 4/8 | 0.375 | Synergy |
| | Daptomycin | 32 | >256 | 128/8 | 0. | Synergy |

Klebsiella pneumoniae (ATCC BAA-1705)

| | | | | | | |
|---|---|---|---|---|---|---|
| IFX-318 | Tetracycline | 64 | 16 | 2/16 | 0.375 | Synergy |
| [R₆W₄] | Ciprofloxacin | 64 | 256 | 16/16 | 0.313 | Synergy |
| MIC = 64 μg/ml | Clindamycin | 64 | >256 | 8/16 | 0. | Synergy |
| FIC = 16 μg/ml | Polymyxin | 64 | 1 | 0.031/16 | 0.281 | Synergy |
| | Levofloxacin | 64 | 64 | 8/16 | 0.375 | Synergy |
| | Kanamycin | 64 | 64 | 16/16 | 0.5 | Synergy |
| | Daptomycin | 64 | >256 | 64/16 | 0. | Synergy |
| | Vancomycin | 64 | >256 | 32/16 | 0. | Synergy |

TABLE 25
Combination studies of IFX-301 [R₅W₄] with antibiotics.
MRSA (ATCC BAA-1556)

| | | | FIC | | | |
|---|---|---|---|---|---|---|
| | | MIC of | antibiotic/ | | | |
| | | antibiotic | peptides | Phy-Mix | FIC | |
| Peptide | Antibiotic | μg/ml | ug/ml | μg/ml | index | Result |
| [R₅W₄] | Tetracycline | 0.250 | .065/1 | 0.065 | 0.375 | Synergy |
| MIC = 4 ug/ml | Tobramycin | 0.5 | 0.125/1 | 0.125 | 0.5 | Synergy |
| FIC = 1 ug/ml | Clindamycin | 0.125 | 0.033/1 | .033 | 0.313 | Synergy |
| | Levofloxacin | 4 | 1/1 | 1 | 0.375 | Synergy |

Pseudomonas aeruginosa (ATCC 27883)

| | | | FIC | | | |
|---|---|---|---|---|---|---|
| | | MIC of | antibiotic/ | | | |
| | | antibiotic | peptides | Phy-Mix | FIC | |
| Peptide | Antibiotic | μg/ml | μg/ml | μg/ml | index | Result |
| [R₅W₄] | Tetracycline | 32 | 4/8 | 2 | 0.375 | Synergy |
| MIC = 32 ug/ml | Tobramycin | 0.5 | 0.125/8 | 0.125 | 0.5 | Synergy |
| FIC = 8 ug/ml | Ciprofloxacin | 0.5 | 0.125/8 | 0.125 | 0.5 | Synergy |
| | Clindamycin | >256 | 8/8 | 4 | 0. | Synergy |
| | Polymyxin | 1 | 0.125/8 | 0.250 | 0.375 | Synergy |
| | Levofloxacin | 1 | 0.125/8 | 0.250 | 0.375 | Synergy |
| | Kanamycin | 256 | 8/8 | 8 | 0.281 | Synergy |
| | Meropenem | 1 | 0.125/8 | 0.125 | 0.375 | Synergy |
| | Vancomycin | >256 | 16/8 | | 0. | Synergy |
| | Daptomycin | >256 | 32/8 | | 0. | Synergy |

Escherichia coli (ATCC 25922)

| | | | FIC | | | |
|---|---|---|---|---|---|---|
| | | MIC of | antibiotic/ | | | |
| | | antibiotic | peptides | Phy-Mix | FIC | |
| Peptide | Antibiotic | μg/ml | μg/ml | ug/ml | index | Result |
| [R₅W₄] | Tetracycline | 8 | 1/4 | 1 | 0.375 | Synergy |
| MIC = 16 ug/ml | Tobramycin | 8 | 2/4 | 2 | 0.5 | Synergy |
| FIC = 4 ug/ml | Clindamycin | 64 | 4/4 | 4 | 0.313 | Synergy |
| | Polymyxin | 2 | 0.250/4 | 0.5 | 0.375 | Synergy |
| | Levofloxacin | 64 | 8/4 | 8 | 0.375 | Synergy |
| | Kanamycin | 32 | 8/4 | 4 | 0.5 | Synergy |

Klebsiella pneumoniae (ATCC BAA-1705)

| | | | FIC | | | |
|---|---|---|---|---|---|---|
| | | MIC of | antibiotic/ | | | |
| | | antibiotic | peptides | Phy-Mix | FIC | |
| Peptide | Antibiotic | ug/ml | (μg/ml) | ug/ml | index | Result |
| [R₅W₄] | Tetracycline | 16 | 2/8 | 2 | 0.375 | Synergy |
| MIC = 32 ug/ml | Tobramycin | 16 | 2/8 | 4 | 0.375 | Synergy |
| FIC = 8 ug/ml | Ciprofloxacin | 256 | 16/8 | 8 | 0.313 | Synergy |
| | Clindamycin | >256 | 2/8 | 2 | 0. | Synergy |
| | Polymyxin | 1 | 0.125/8 | 0.5 | 0.375 | Synergy |
| | Levofloxacin | 64 | 8/8 | 8 | 0.375 | Synergy |
| | Kanamycin | 64 | 8/8 | 8 | 0.375 | Synergy |
| | Meropenem | 16 | 4/8 | 2 | 0.5 | Synergy |
| | Vancomycin | >256 | 64/8 | | 0. | Synergy |

TABLE 26
Combination studies of IFX-315 [R₅W₄K] with antibiotics.
MRSA (ATCC BAA-1556)

| | | | FIC | | |
|---|---|---|---|---|---|
| | | MIC of | antibiotic/ | | |
| | | antibiotic | peptide | FIC | |
| Peptide | Antibiotic | μg/ml | μg/ml | index | Result |
| IFX-315 | Daptomycin | 2 | 0.5/2 | 0.5 | Synergy |
| [R₅W₄K] | Clindamycin | 0.125 | 0.031/2 | 0.5 | Synergy |
| MIC = 8 ug/ml | Levofloxacin | 4 | 1/2 | 0.5 | Synergy |
| FIC = 2 ug/ml | | | | | |

| | | | | | | |
|---|---|---|---|---|---|---|
| | | | FIC | | | |
| | | MIC of | antibiotic/ | | | |
| | | antibiotic | peptide | Phy-Mix | FIC | |
| Peptide | Antibiotic | ug/ml | ug/ml | ug/ml | index | Result |

Pseudomonas aeruginosa (ATCC 27883)

| | | | | | | |
|---|---|---|---|---|---|---|
| IFX-315 | Tetracycline | 32 | 4/8 | 4 | 0.375 | Synergy |
| [R₅W₄K] | Tobramycin | 0.5 | 0.125/8 | 0.125 | 0.375 | Synergy |
| MIC = 16 ug/ml | Polymyxin | 1 | 0.125/8 | 0.250 | 0.375 | Synergy |
| FIC = 4 ug/ml | Levofloxacin | 1 | 0.125/8 | 0.250 | 0.375 | Synergy |
| | Meropenem | 1 | 0.250/8 | 0.125 | 0.5 | Synergy |

Escherichia coli (ATCC 25922)

| | | | | | | |
|---|---|---|---|---|---|---|
| IFX-315 | Tetracycline | 8 | 2/4 | 2 | 0.5 | Synergy |
| [R₅W₄K] | Polymyxin | 2 | 0.125/4 | 0.5 | 0.313 | Synergy |
| MIC = 16 ug/ml | Levofloxacin | 64 | 8/4 | 4 | 0.375 | Synergy |
| FIC = 4 ug/ml | Kanamycin | 32 | 4/4 | 4 | 0.375 | Synergy |

Klebsiella pneumoniae (ATCC BAA-1705)

| | | | FIC | | |
|---|---|---|---|---|---|
| | | MIC of | antibiotic/ | | |
| | | antibiotics | peptide | FIC | |
| Peptide | Antibiotics | ug/ml | ug/ml | index | Result |
| IFX-315 | Tetracycline | 16 | 1/16 | 0.313 | Synergy |
| [R₅W₄K] | Tobramycin | 16 | 2/16 | 0.375 | Synergy |
| MIC = 64 ug/ml | Polymyxin | 1 | 0.063/16 | 0.313 | Synergy |
| FIC = 16 ug/ml | Clindamycin | >256 | 2/16 | 0.0 | Synergy |
| | Kanamycin | 64 | 8/16 | 0.375 | Synergy |
| | Daptomycin | >256 | 32/16 | 0.0 | |

TABLE 27
Combination studies of IFX-315 [R₅W₄K] with antibiotics.
MRSA (ATCC BAA-1556)

| Clindamycin | IFX135 | FIC | FIC | | FIC |
|---|---|---|---|---|---|
| MIC | MIC | Clindamycin | Peptide | PHY-MIX | indx |
| 0.125 μg/ml | 2 μg/ml | 0.063 μg/ml | 0.5 μg/ml | 0.065 μg/ml | 0.77 |

Escherichia coli (ATCC 25922)

| Polymyxin | IFX135 | FIC | FIC | | FIC |
|---|---|---|---|---|---|
| MIC | MIC | Polymyxin | Peptide | PHY-MIX | indx |
| 2 μg/ml | 8 μg/ml | 0.250 μg/ml | 0.5 μg/ml | 2 μg/ml | 0.5 |

Klebsiella pneumoniae (ATCC BAA-1705)

| Polymyxin | IFX135 | FIC | FIC | | FIC |
|---|---|---|---|---|---|
| MIC | MIC | Polymyxin | Peptide | PHY-MIX | indx |
| 0.5 μg/ml | 16 μg/ml | 0.063 μg/ml | 4 μg/ml | 0.125 μg/ml | 0.376 |

Pseudomonas aeruginosa (ATCC 27883)

| Tetracycline | IFX135 | FIC | FIC | | FIC |
|---|---|---|---|---|---|
| MIC | MIC | Tetracycline | Peptide | PHY-MIX | indx |
| 32 | 32 | 8 | 8 | 8 | 0.5 |
| Tobramycin | IFX135 | FIC | FIC | | FIC |
| MIC | MIC | Tobramycin | Peptide | PHY-MIX | indx |
| 0.5 | 32 | 0.063 | 8 | 0.063 | 0.376 |
| Meropenem | IFX135 | FIC | FIC | | FIC |
| MIC | MIC | Meropenem | Peptide | PHY-MIX | indx |
| 1 | 32 | 0.250 | 8 | 0.250 | 0.5 |
| Polymyxin | IFX135 | FIC | FIC | | FIC |
| MIC | MIC | Polymyxin | Peptide | PHY-MIX | indx |
| 1 | 32 | 0.250 | 8 | 0.250 | 0.5 |

TABLE 28
Antibacterial activity of IFX-031 in combination with antibiotics.

| | | | Pseudomonas | Klebsiella |
|---|---|---|---|---|
| | MRSA | Escherichia | aeruginosa | pneumoniae |
| | (ATCC | coli (ATCC | (ATCC | (ATCC |
| | BAA-1556) | 25922) | 27883) | BAA-1705) |
| | MIC | MIC | MIC | MIC |
| Sequence | μg/mL | μg/mL | μg/mL | μg/mL |
| IFX-031 | 2 | 16 | 16 | 32 |
| Tetracycline | 0.250 | 16 | 32 | 8 |
| PHY-MIX of IFX-031 with | 0.065 | 2 | 4 | 4 |
| tetracycline (1:1 w/w) | | | | |
| Tobramycin | 0.5 | 8 | 0.5 | 16 |
| PHY-MIX of IFX-031 with | 0.125 | 2 | 0.250 | 4 |
| tobramycin (1:1 w/w) | | | | |
| Levofloxacin | 4 | 16 | 1 | 32 |
| PHY-MIX of IFX-031 with | 1 | 4 | 0.125 | 16 |
| levofloxacin (1:1 w/w) | | | | |
| PHY-MIX of IFX-031 with | 1 | 8 | 0.125 | 16 |
| Ciprofloxacin (1:1 w/w) | | | | |
| Metronidazole | >32 | >32 | 32 | >32 |
| PHY-MIX of IFX-031 with | 2 | 16 | 32 | 16 |
| metronidazole (1:1 w/w) | | | | |
| Clindamycin | 0.125 | 64 | >64 | >256 |
| PHY-MIX of IFX-031 with | 0.065 | 4 | 16 | 8 |
| clindamycin (1:1 w/w) | | | | |
| Daptomycin | 2 | >256 | >64 | >256 |
| PHY-MIX of IFX-031 with | 1 | 16 | 8 | 16 |
| daptomycin (1:1 w/w) | | | | |
| Polymyxin | 64 | 2 | 1 | 1 |
| PHY-MIX of IFX-031 with | 1 | 0.250 | .5 | 1 |
| polymyxin (1:1 w/w) | | | | |
| Kanamycin | >64 | 32 | >64 | 64 |
| PHY-MIX of IFX-031 with | 1 | 4 | 16 | 8 |
| kanamycin (1:1 w/w) | | | | |
| Meropenem | 2 | 1 | 1 | 16 |
| PHY-MIX of IFX-031 with | 0.250 | 2 | 0.5 | 4 |
| meropenem (1:1 w/w) | | | | |
| Vancomycin | 1 | >64 | >64 | >256 |
| PHY-MIX of IFX-031 with | 0.250 | 8 | 16 | 16 |
| vancomycin (1:1 w/w) | | | | |

TABLE 29
Antibacterial activity of [R₆W₄] (IFX-318) in combination with antibiotics.

| | | Klebsiella | Pseudomonas | |
|---|---|---|---|---|
| | MRSA | pneumoniae | aeruginosa | Escherichia |
| | (ATCC | (ATCC | (ATCC | coli (ATCC |
| | BAA-1556) | BAA- | 27883) | 25922) |
| | MIC | 1705)MIC | MIC | MIC |
| Sequence | μg/mL | μg/mL | μg/mL | μg/mL |
| [R₆W₄] | 8 | 64 | 16 | 32 |
| Tetracycline | 0.250 | 16 | 32 | 8 |
| PHY-MIX of IFX-318 with | 0.065 | 4 | 4 | 2 |
| tetracycline (1:1 w/w) | | | | |
| Tobramycin | 0.5 | 16 | 0.5 | 8 |
| PHY-MIX of IFX-318 with | 0.125 | 4 | 0.250 | 4 |
| tobramycin (1:1 w/w) | | | | |
| Levofloxacin | 4 | 64 | 1 | 64 |
| PHY-MIX of IFX-318 with | 2 | 16 | 0.5 | 8 |
| levofloxacin (1:1 w/w) | | | | |
| PHY-MIX of IFX-318 with | 4 | 16 | 0.125 | 8 |
| Ciprofloxacin (1:1 w/w) | | | | |
| Metronidazole | 32 | >32 | >32 | >32 |
| PHY-MIX of IFX-318 with | 2 | 16 | 4 | 16 |
| metronidazole (1:1 w/w) | | | | |
| Clindamycin | 0.125 | 64 | >64 | >256 |
| PHY-MIX of IFX-318 with | 0.033 | 8 | 8 | 8 |
| clindamycin (1:1 w/w) | | | | |
| Daptomycin | 2 | >256 | >64 | >256 |
| PHY-MIX of IFX-318 with | 1 | 16 | 8 | 8 |
| daptomycin (1:1 w/w) | | | | |
| Polymyxin | 64 | 1 | 1 | 2 |
| PHY-MIX of IFX-318 with | 4 | 0.250 | 0.5 | 0.5 |
| polymyxin (1:1 w/w) | | | | |
| Kanamycin | >64 | 64 | >64 | 32 |
| PHY-MIX of IFX-318 with | 4 | 8 | 4 | 4 |
| kanamycin (1:1 w/w) | | | | |
| Meropenem | 2 | 16 | 1 | 1 |
| PHY-MIX of IFX-318 with | 1 | 4 | 0.125 | 0.065 |
| meropenem (1:1 w/w) | | | | |
| Vancomycin | 1 | >256 | >64 | >64 |
| PHY-MIX of IFX-318 with | 0.250 | 16 | 8 | 16 |
| vancomycin (1:1 w/w) | | | | |

TABLE 30
Antibacterial activity of [R₅W₄K] (IFX-315) in combination with antibiotics.

| | | Klebsiella | Pseudomonas | |
|---|---|---|---|---|
| | MRSA | pneumoniae | aeruginosa | Escherichia |
| | (ATCC | (ATCC | (ATCC | coli (ATCC |
| | BAA-1556) | BAA- | 27883) | 25922) |
| | MIC | 1705)MIC | MIC | MIC |
| Sequence | μg/mL | μg/mL | μg/mL | μg/mL |
| [R₅W₄K] | 4 | 32 | 16 | 16 |
| Tetracycline | 0.250 | 16 | 32 | 8 |
| PHY-MIX of IFX-315 with | 0.125 | 4 | 4 | 2 |
| tetracycline (1:1 w/w) | | | | |
| Tobramycin | 0.5 | 16 | 0.5 | 8 |
| PHY-MIX of IFX-315 with | 0.125 | 4 | 0.125 | 4 |
| tobramycin (1:1 w/w) | | | | |
| Levofloxacin | 4 | 64 | 1 | 64 |
| PHY-MIX of IFX-315 with | 1 | 16 | 0.250 | 4 |
| levofloxacin (1:1 w/w) | | | | |
| PHY-MIX of IFX-315 with | 2 | 16 | 0.250 | 8 |
| Ciprofloxacin (1:1 w/w) | | | | |
| Metronidazole | 32 | >32 | >32 | >32 |
| PHY-MIX of IFX-315 with | 8 | 16 | 16 | 16 |
| metronidazole (1:1 w/w) | | | | |
| Clindamycin | 0.125 | >256 | >64 | 64 |
| PHY-MIX of IFX-315 with | 0.033 | 8 | 8 | 8 |
| clindamycin (1:1 w/w) | | | | |
| Daptomycin | 2 | >256 | >64 | >256 |
| PHY-MIX of IFX-315 with | 0.5 | 8 | 16 | 8 |
| daptomycin (1:1 w/w) | | | | |
| Polymyxin | 64 | 1 | 1 | 2 |
| PHY-MIX of IFX-315 with | 4 | 0.250 | 0.250 | .5 |
| polymyxin (1:1 w/w) | | | | |
| Kanamycin | >64 | 64 | >64 | 32 |
| PHY-MIX of IFX-315 with | 4 | 8 | 8 | 4 |
| kanamycin (1:1 w/w) | | | | |
| Meropenem | 2 | 16 | 1 | 1 |
| PHY-MIX of IFX-315 with | 1 | 8 | .125 | .5 |
| meropenem (1:1 w/w) | | | | |
| Vancomycin | 1 | >256 | >64 | >64 |
| PHY-MIX of IFX-315 with | 0.5 | 16 | 16 | 8 |
| vancomycin (1:1 w/w) | | | | |

TABLE 31
Antibacterial activity of [R₅W₄] (IFX-301) in combination with antibiotics.

| | | Klebsiella | Pseudomonas | |
|---|---|---|---|---|
| | MRSA | pneumoniae | aeruginosa | Escherichia |
| | (ATCC | (ATCC | (ATCC | coli (ATCC |
| | BAA-1556) | BAA- | 27883) | 25922) |
| | MIC | 1705)MIC | MIC | MIC |
| Sequence | μg/mL | μg/mL | μg/mL | μg/mL |
| [R₅W₄] | 4 | 32 | 32 | 16 |
| Tetracycline | 0.250 | 16 | 32 | 8 |
| PHY-MIX of IFX-301 with | 0.065 | 2 | 2 | 1 |
| tetracycline (1:1 w/w) | | | | |
| Tobramycin | 0.5 | 16 | 0.5 | 8 |
| PHY-MIX of IFX-301 with | 0.125 | 4 | 0.125 | 2 |
| tobramycin (1:1 w/w) | | | | |
| Levofloxacin | 4 | 64 | 1 | 64 |
| PHY-MIX of IFX-301 with | 1 | 8 | 0.250 | 8 |
| levofloxacin (1:1 w/w) | | | | |
| PHY-MIX of IFX-301 with | 2 | 8 | 0.125 | 8 |
| Ciprofloxacin (1:1 w/w) | | | | |
| Metronidazole | >32 | >32 | 32 | >32 |
| PHY-MIX of IFX-301 with | 2 | 8 | 8 | 16 |
| metronidazole (1:1 w/w) | | | | |
| Clindamycin | 0.125 | >256 | >64 | 64 |
| PHY-MIX of IFX-301 with | 0.033 | 2 | 4 | 4 |
| clindamycin (1:1 w/w) | | | | |
| Daptomycin | 2 | >256 | >64 | >256 |
| PHY-MIX of IFX-3 01 with | 1 | 16 | 4 | 8 |
| daptomycin (1:1 w/w) | | | | |
| Polymyxin | 64 | 1 | 1 | 2 |
| PHY-MIX of IFX-3 01 with | 2 | 0.5 | 0.250 | 0.5 |
| polymyxin (1:1 w/w) | | | | |
| Kanamycin | >64 | 64 | >64 | 32 |
| PHY-MIX of IFX-301 with | 4 | 8 | 8 | 4 |
| kanamycin (1:1 w/w) | | | | |
| Meropenem | 2 | 16 | 1 | 1 |
| PHY-MIX of IFX-301 with | 1 | 2 | 0.125 | 0.5 |
| meropenem (1:1 w/w) | | | | |
| Vancomycin | 1 | >64 | >64 | >64 |
| PHY-MIX of IFX-301 with | 0.5 | 4 | 8 | 8 |
| vancomycin (1:1 w/w) | | | | |

TABLE 32
Antibacterial activity of conjugate of meropenem with IFX-315.

| | | Klebsiella | Pseudomonas | |
|---|---|---|---|---|
| | MRSA | pneumoniae | aeruginosa | Escherichia |
| | (ATCC | (ATCC | (ATCC | coli (ATCC |
| | BAA-1556) | BAA- | 27883) | 25922) |
| | MIC | 1705)MIC | MIC | MIC |
| Sequence | μg/mL | μg/mL | μg/mL | μg/mL |
| Meropenem | 2 | 16 | 1 | 1 |
| [R₄W₅K] (IFX-315) | 4 | 32 | 16 | 16 |
| [R₅W₄K-Mero. Conjugate] | 4 | 16 | 16 | 8 |
| Phy-Mix 1:1 | 1 | 8 | .125 | .5 |

TABLE 33
MIC of peptides and Peptide-capped Au-NPs against Gram-positive bacteria.

| | MIC | MIC | MIC | MIC | |
|---|---|---|---|---|---|
| | μg/ml | μg/ml | μg/ml | μg/ml | |
| | [R₅W₄] | [R₅W₄] | [R₆W₄] | [R₆W₄] | MIC μg/ml |
| Bacterial strain | (IFX-301) | Au-NP | (IFX-318) | Au-NP | Daptomycin |
| Staphylococcus aureus | 4 | 4 | 8 | 16 | 1 |
| (ATCC 29213) | | | | | |
| Enterococcus faecium | 8 | 8 | 8 | 16 | 4 |
| (ATCC 27270) | | | | | |
| Enterococcus faecium | 4 | 8 | 4 | 8 | 2 |
| (ATCC 700221) | | | | | |
| Enterococcus faecalis | 8 | 16 | 16 | 16 | 16 |
| (ATCC 29212) | | | | | |
| Enterococcus faecalis | 16 | 32 | 32 | 64 | 4 |
| (ATCC 51575) | | | | | |
| Staphylococcus | 2 | 1 | 1 | 2 | 4 |
| pneumonia (ATCC | | | | | |
| 49619) | | | | | |
| Staphylococcus | 2 | 2 | 2 | 4 | 8 |
| pneumonia (ATCC | | | | | |
| 51938) | | | | | |
| Bacillus subtilis | 4 | 4 | 1 | 4 | 0.5 |
| (ATCC-6633) | | | | | |
| Bacillus cereus | 16 | 8 | 16 | 32 | 2 |
| (ATCC-13061) | | | | | |
| MRSA | 4 | 8 | 8 | 16 | 2 |

TABLE 34
MIC of peptides and Peptide-capped Au-NPs against Gram-positive bacteria.

| | | MIC ug/ml | MIC ug/ml | MIC ug/ml | |
|---|---|---|---|---|---|
| | MIC ug/ml | IFX-135- | [R₅W₄K] | [R₃CW₄CR] | MIC ug/ml |
| Bacterial strain | IFX-135 | Au-NP | (IFX-315) | (IFX-330) | Meropenem |
| Staphylococcus aureus | 2 | 4 | 8 | 16 | 0.250 |
| (ATCC 29213) | | | | | |
| Enterococcus faecium | 4 | 16 | 8 | 8 | 0.5 |
| (ATCC 27270) | | | | | |
| Enterococcus faecium | 4 | 16 | 8 | 8 | 64 |
| (ATCC 700221) | | | | | |
| Enterococcus faecalis | 8 | 64 | 16 | 32 | 2 |
| (ATCC 29212) | | | | | |
| Enterococcus faecalis | 16 | 32 | 32 | 32 | 8 |
| (ATCC 51575) | | | | | |
| Staphylococcus | 16 | 8 | 2 | 2 | 0.250 |
| pneumonia (ATCC | | | | | |
| 49619) | | | | | |
| Staphylococcus | 16 | 8 | 2 | 1 | 0.250 |
| pneumonia (ATCC | | | | | |
| 51938) | | | | | |
| Bacillus subtilis (ATCC- | 2 | 16 | 8 | 8 | 0.250 |
| 6633) | | | | | |
| Bacillus cereus (ATCC- | 8 | 64 | 16 | 16 | 4 |
| 13061) | | | | | |
| MRSA | 2 | 16 | 8 | 8 | 2 |

TABLE 35
MIC of peptides and Peptide-capped Au-NPs against Gram-negative bacteria.

| | MIC | MIC | MIC | MIC | |
|---|---|---|---|---|---|
| | μg/ml | μg/ml | μg/ml | μg/ml | |
| | [R₅W₄] | [R₅W₄] | [R₆W₄] | [R₆W₄] | MIC μg/ml |
| Bacterial strain | IFX-301 | Au-NP | IFX-301 | Au-Np | Polymyxin |
| Escherichia coli | 16 | 16 | 16 | 16 | 1 |
| (ATCC BAA-2452) | | | | | |
| Klebsiella pneumonia | 32 | 32 | 64 | 32 | 1 |
| (ATCC 13883) | | | | | |
| Acinetobacter | 32 | 32 | 32 | 32 | 1 |
| baumannii (ATCC | | | | | |
| BAA-1605) | | | | | |
| Pseudomonas | 32 | 8 | 32 | 32 | 2 |
| aeruginosa (ATCC | | | | | |
| 10145) | | | | | |
| Pseudomonas | 32 | 8 | 16 | 16 | 2 |
| aeruginosa (ATCC | | | | | |
| BAA-1744) | | | | | |
| Klebsiella pneumoniae | 32 | 16 | 64 | 32 | 0.5 |
| (ATCC BAA-1705) | | | | | |
| P. aeruginosa | 32 | 16 | 16 | 8 | 1 |
| ATCC 27883 | | | | | |
| E. coli | 16 | 16 | 32 | 16 | 2 |
| ATCC 25922 | | | | | |

TABLE 36
MIC of peptides and Peptide-capped Au-NPs against Gram-negative bacteria.

| | | MIC μg/ml | MIC μg/ml | MIC μg/ml | |
|---|---|---|---|---|---|
| | MIC μg/ml | IFX-135 | [R₅W₄K] | [R₃CW₄CR] | MIC μg/ml |
| Bacterial strain | IFX-135 | Au-NP | (IFX-315) | (IFX-330) | Polymyxin |
| Escherichia coli | 16 | 8 | 25 | 32 | 1 |
| (ATCC BAA-2452) | | | | | |
| Klebsiella | 64 | 32 | 8 | 32 | 1 |
| pneumonia (ATCC | | | | | |
| 13883) | | | | | |
| Acinetobacter | 16 | 16 | 32 | 32 | 1 |
| baumannii (ATCC | | | | | |
| BAA-1605) | | | | | |
| Pseudomonas | 32 | 16 | 32 | 64 | 2 |
| aeruginosa (ATCC | | | | | |
| 10145) | | | | | |
| Pseudomonas | 16 | 8 | 16 | 32 | 2 |
| aeruginosa (ATCC | | | | | |
| BAA-1744) | | | | | |
| Klebsiella | 16 | 32 | 32 | 32 | 0.5 |
| pneumoniae (ATCC | | | | | |
| BAA-1705) | | | | | |
| P. aeruginosa | 32 | 64 | 16 | 128 | 1 |
| ATCC 27883 | | | | | |
| E. coli | 8 | 32 | 16 | 32 | 2 |
| ATCC 25922 | | | | | |

TABLE 37
Antibacterial activity of peptides and peptide-capped gold nanoparticles.

| | MIC μg/mL | | | |
|---|---|---|---|---|
| | | Klebsiella | Pseudomonas | |
| | MRSA | pneumoniae | aeruginosa | Escherichia |
| | (ATCC | (ATCC | (ATCC | coli (ATCC |
| Sequence | BAA-1556) | BAA-1705) | 27883) | 25922) |
| [R₃W₃] (IFX-302) | 16 | 64 | 128 | 32 |
| [R₃W₃]AUNP | 32 | 64 | 64 | 32 |
| [R₄W₄] (IFX-326) | 4 | 32 | 64 | 16 |
| [R₄W₄]AUNP | 8 | 32 | 16 | 32 |
| [R₅W₄] (IFX-301) | 4 | 32 | 32 | 16 |
| [R₅W₄]AUNP | 8 | 16 | 16 | 16 |
| [R₅W₅] (IFX-314) | 4 | 32 | 32 | 16 |
| [R₅W₅]AUNP | 16 | 32 | 32 | 64 |
| [R₆W₄] (IFX-318) | 4 | 64 | 16 | 32 |
| [R₆W₄]AUNP | 4 | 32 | 8 | 32 |
| [R₇W₄] (IFX-322) | 16 | 64 | 32 | 32 |
| [R₇W₄]AUNP | 8 | 32 | 8 | 16 |
| Linear R₅W₄K | 8 | 64 | 32 | 32 |
| (SEQ ID NO. 36) | | | | |
| Linear R₅W₄K-AUNP | 32 | 64 | 32 | 32 |
| (SEQ ID NO. 37) | | | | |
| Meropenem | 4 | 8 | 0.5 | 0.125 |
| Vancomycin | 0.5 | No effect | No effect | No effect |

TABLE 38
In-vivo toxicity study of IFX301.

| | | | | | | | | | | | |
|---|---|---|---|---|---|---|---|---|---|---|---|
| | Dose Level | | | | | | | | | | |
| Compound | & Volume | Mouse ID | Gender | Day 1 | Day 2 | Day 3 | Day 4 | Day 5 | Day 6 | Day 7 | Day 8 |

Body Weight (g) of ICR MTD Study (IFX301, IP Route)

| | | | | | | | | | | | |
|---|---|---|---|---|---|---|---|---|---|---|---|
| IFX301 | Vehicle | Mouse#31 | Male | 23 | 24 | 25 | 27 | 28 | 29 | 30 | 30 |
| | & 5 mL/kg | Mouse#32 | | 23 | 25 | 27 | 28 | 29 | 30 | 30 | 31 |
| | | Mouse#33 | | 22 | 25 | 26 | 28 | 30 | 30 | 31 | 31 |
| | | Mouse#34 | Female | 22 | 23 | 24 | 25 | 26 | 26 | 26 | 27 |
| | | Mouse#35 | | 21 | 22 | 21 | 23 | 23 | 23 | 23 | 23 |
| | | Mouse#36 | | 22 | 22 | 22 | 23 | 23 | 24 | 24 | 24 |
| | 10 mg/kg | Mouse#37 | Male | 22 | 23 | 24 | 24 | 25 | 26 | 26 | 26 |
| | & 5 mL/kg | Mouse#38 | | 22 | 22 | 23 | 24 | 26 | 27 | 28 | 28 |
| | | Mouse#39 | | 21 | 24 | 24 | 26 | 26 | 27 | 27 | 28 |
| | | Mouse#40 | Female | 20 | 20 | 21 | 23 | 24 | 24 | 24 | 25 |
| | | Mouse#41 | | 21 | 23 | 23 | 23 | 24 | 24 | 24 | 25 |
| | | Mouse#42 | | 21 | 21 | 21 | 23 | 24 | 24 | 25 | 25 |
| | 50 mg/kg | Mouse#43 | Male | 22 | 23 | 23 | 23 | 23 | 23 | 25 | 25 |
| | & 5 mL/kg | Mouse#44 | | 22 | 21 | 22 | 22 | 24 | 25 | 26 | 26 |
| | | Mouse#45 | | 22 | 22 | 23 | 24 | 25 | 25 | 26 | 27 |
| | | Mouse#46 | Female | 20 | 20 | 20 | 20 | 21 | 22 | 22 | 22 |
| | | Mouse#47 | | 20 | 19 | 19 | 18 | 18 | 20 | 20 | 20 |
| | | Mouse#48 | | 20 | 19 | 19 | 20 | 21 | 21 | 22 | 22 |
| | 25 mg/kg | Mouse#49 | Male | 23 | 23 | 24 | 24 | 25 | 25 | 25 | 26 |
| | & 5 mL/kg | Mouse#50 | | 22 | 23 | 24 | 25 | 26 | 26 | 26 | 26 |
| | | Mouse#51 | | 21 | 22 | 22 | 23 | 24 | 25 | 26 | 26 |
| | | Mouse#52 | Female | 20 | 21 | 20 | 21 | 21 | 22 | 22 | 23 |
| | | Mouse#53 | | 20 | 19 | 21 | 22 | 22 | 23 | 23 | 23 |
| | | Mouse#54 | | 20 | 21 | 20 | 21 | 21 | 22 | 22 | 23 |

Average Body Weight (g) of ICR MTD Study (IFX301, IP Route)

| | | | | | | | | | | | |
|---|---|---|---|---|---|---|---|---|---|---|---|
| IFX301 | Vehicle | M31~M33 | Male | 23 | 25 | 26 | 28 | 29 | 30 | 30 | 31 |
| | & 5 mL/kg | M34~M36 | Female | 22 | 22 | 22 | 24 | 24 | 24 | 24 | 25 |
| | 10 mg/kg | M37~M39 | Male | 22 | 23 | 24 | 25 | 26 | 27 | 27 | 27 |
| | & 5 mL/kg | M40~M42 | Female | 21 | 21 | 22 | 23 | 24 | 24 | 24 | 25 |
| | 50 mg/kg | M43~M45 | Male | 22 | 22 | 23 | 23 | 24 | 24 | 26 | 26 |
| | & 5 mL/kg | M46~M48 | Female | 20 | 19 | 19 | 19 | 20 | 21 | 21 | 21 |
| | 25 mg/kg | M49~M51 | Male | 22 | 23 | 23 | 24 | 25 | 25 | 26 | 26 |
| | & 5 mL/kg | M52~M54 | Female | 20 | 20 | 20 | 21 | 21 | 22 | 22 | 23 |

Body Weight Growth Rate of ICR MTD Study (IFX301, IP Route)

| | | | | | | | | | | | |
|---|---|---|---|---|---|---|---|---|---|---|---|
| IFX301 | Vehicle | Mouse #31 | Male | NA | 4.35% | 8.70% | 17.39% | 21.74% | 26.09% | 30.43% | 30.43% |
| | & 5 mL/kg | Mouse #32 | | NA | 8.70% | 17.39% | 21.74% | 26.09% | 30.43% | 30.43% | 34.78% |
| | | Mouse #33 | | NA | 13.64% | 18.18% | 27.27% | 36.36% | 36.36% | 40.91% | 40.91% |
| | | Mouse #34 | Female | NA | 4.55% | 9.09% | 13.64% | 18.18% | 18.18% | 18.18% | 22.73% |
| | | Mouse #35 | | NA | 4.76% | 0.00% | 9.52% | 9.52% | 9.52% | 9.52% | 9.52% |
| | | Mouse #36 | | NA | 0.00% | 0.00% | 4.55% | 4.55% | 9.09% | 9.09% | 9.09% |
| | 10 mg/kg | Mouse #37 | Male | NA | 4.55% | 9.09% | 9.09% | 13.64% | 18.18% | 18.18% | 18.18% |
| | & 5 mL/kg | Mouse #38 | | NA | 0.00% | 4.55% | 9.09% | 18.18% | 22.73% | 27.27% | 27.27% |
| | | Mouse #39 | | NA | 14.29% | 14.29% | 23.81% | 23.81% | 28.57% | 28.57% | 33.33% |
| | | Mouse #40 | Female | NA | 0.00% | 5.00% | 15.00% | 20.00% | 20.00% | 20.00% | 25.00% |
| | | Mouse #41 | | NA | 9.52% | 9.52% | 9.52% | 14.29% | 14.29% | 14.29% | 19.05% |
| | | Mouse #42 | | NA | 0.00% | 0.00% | 9.52% | 14.29% | 14.29% | 19.05% | 19.05% |
| | 50 mg/kg | Mouse #43 | Male | NA | 4.55% | 4.55% | 4.55% | 4.55% | 4.55% | 13.64% | 13.64% |
| | & 5 mL/kg | Mouse #44 | | NA | −4.55% | 0.00% | 0.00% | 9.09% | 13.64% | 18.18% | 18.18% |
| | | Mouse #45 | | NA | 0.00% | 4.55% | 9.09% | 13.64% | 13.64% | 18.18% | 22.73% |
| | | Mouse #46 | Female | NA | 0.00% | 0.00% | 0.00% | 5.00% | 10.00% | 10.00% | 10.00% |
| | | Mouse #47 | | NA | −5.00% | −5.00% | −10.00% | −10.00% | 0.00% | 0.00% | 0.00% |
| | | Mouse #48 | | NA | −5.00% | −5.00% | 0.00% | 5.00% | 5.00% | 10.00% | 10.00% |
| | 25 mg/kg | Mouse #49 | Male | NA | 0.00% | 4.35% | 4.35% | 8.70% | 8.70% | 8.70% | 13.04% |
| | & 5 mL/kg | Mouse #50 | | NA | 4.55% | 9.09% | 13.64% | 18.18% | 18.18% | 18.18% | 18.18% |
| | | Mouse #51 | | NA | 4.76% | 4.76% | 9.52% | 14.29% | 19.05% | 23.81% | 23.81% |
| | | Mouse #52 | Female | NA | 5.00% | 0.00% | 5.00% | 5.00% | 10.00% | 10.00% | 15.00% |
| | | Mouse #53 | | NA | −5.00% | 5.00% | 10.00% | 10.00% | 15.00% | 15.00% | 15.00% |
| | | Mouse #54 | | NA | 5.00% | 0.00% | 5.00% | 5.00% | 10.00% | 10.00% | 15.00% |

Average Body Weight Growth Rate of ICR MTD Study (IFX301, IP Route)

| | | | | | | | | | | | |
|---|---|---|---|---|---|---|---|---|---|---|---|
| IFX301 | Vehicle | M31~M33 | Male | NA | 8.82% | 14.71% | 22.06% | 27.94% | 30.88% | 33.82% | 35.29% |
| | & 5 mL/kg | M34~M36 | Female | NA | 3.08% | 3.08% | 9.23% | 10.77% | 12.31% | 12.31% | 13.85% |
| | 10 mg/kg | M37~M39 | Male | NA | 6.15% | 9.23% | 13.85% | 18.46% | 23.08% | 24.62% | 26.15% |
| | & 5 mL/kg | M40~M42 | Female | NA | 3.23% | 4.84% | 11.29% | 16.13% | 16.13% | 17.74% | 20.97% |
| | 50 mg/kg | M43~M45 | Male | NA | 0.00% | 3.03% | 4.55% | 9.09% | 10.61% | 16.67% | 18.18% |
| | & 5 mL/kg | M46~M48 | Female | NA | 3.33% | 3.33% | 3.33% | 0.00% | 5.00% | 6.67% | 6.67% |
| | 25 mg/kg | M49~M51 | Male | NA | 3.03% | 6.06% | 9.09% | 13.64% | 15.15% | 16.67% | 18.18% |
| | & 5 mL/kg | M52~M54 | Female | NA | 1.67% | 1.67% | 6.67% | 6.67% | 11.67% | 11.67% | 15.00% |

DMPK
Organ/Body Coefficient for MID Study
Organ Weight of Male ICR MTD Study (IFX301, IP Route)

| | | | | Body | | | | | | | | | |
|---|---|---|---|---|---|---|---|---|---|---|---|---|---|
| | | | | Weight | | | | | | | | | |
| | Dose | | | before | Adrenal | | | | | | | | |
| | Level & | | | Necropsy | glands | Brain | Heart | Kidneys | Liver | Spleen | Thymus | Lung | Testes |
| Compound | Volume | Mouse ID | Gender | (g) | (g) | (g) | (g) | (g) | (g) | (g) | (g) | (g) | (g) |
| IFX301 | Vehicle | Mouse#31 | Male | 30 | 0.011 | 0.465 | 0.150 | 0.540 | 2.068 | 0.097 | 0.086 | 0.195 | 0.189 |
| | & 5 mL/kg | Mouse#32 | | 31 | 0.011 | 0.480 | 0.173 | 0.578 | 2.144 | 0.155 | 0.097 | 0.277 | 0.199 |
| | | Mouse#33 | | 31 | 0.010 | 0.456 | 0.162 | 0.522 | 2.190 | 0.124 | 0.116 | 0.196 | 0.170 |
| | 10 mg/kg | Mouse#37 | Male | 26 | 0.004 | 0.457 | 0.132 | 0.465 | 1.764 | 0.117 | 0.077 | 0.199 | 0.140 |
| | & 5 mL/kg | Mouse#38 | | 28 | 0.016 | 0.441 | 0.130 | 0.456 | 1.849 | 0.137 | 0.067 | 0.226 | 0.176 |
| | | Mouse#39 | | 28 | 0.010 | 0.441 | 0.167 | 0.457 | 2.108 | 0.149 | 0.104 | 0.206 | 0.166 |
| | 50 mg/kg | Mouse#43 | Male | 25 | 0.010 | 0.430 | 0.112 | 0.471 | 2.005 | 0.157 | 0.056 | 0.175 | 0.193 |
| | & 5 mL/kg | Mouse#44 | | 26 | 0.003 | 0.454 | 0.124 | 0.525 | 2.023 | 0.174 | 0.074 | 0.174 | 0.179 |
| | | Mouse#45 | | 27 | 0.011 | 0.457 | 0.147 | 0.614 | 2.422 | 0.203 | 0.064 | 0.210 | 0.164 |
| | 25 mg/kg | Mouse#49 | Male | 26 | 0.004 | 0.452 | 0.125 | 0.505 | 2.221 | 0.087 | 0.082 | 0.299 | 0.170 |
| | & 5 mL/kg | Mouse#50 | | 26 | 0.015 | 0.434 | 0.132 | 0.527 | 2.259 | 0.086 | 0.083 | 0.196 | 0.189 |
| | | Mouse#51 | | 26 | 0.006 | 0.416 | 0.184 | 0.647 | 2.261 | 0.188 | 0.075 | 0.282 | 0.128 |

Organ Weight of Female ICR MTD Study (IFX301, IP Route)

| | | | | Body | | | | | | | | | |
|---|---|---|---|---|---|---|---|---|---|---|---|---|---|
| | | | | Weight | | | | | | | | | |
| | Dose | | | before | Adrenal | | | | | | | | |
| | Level & | | | Necropsy | glands | Brain | Heart | Kidneys | Liver | Spleen | Thymus | Lung | Ovaries |
| Compound | Volume | Mouse ID | Gender | (g) | (g) | (g) | (g) | (g) | (g) | (g) | (g) | (g) | (g) |
| IFX301 | Vehicle | Mouse#34 | Female | 27 | 0.017 | 0.427 | 0.136 | 0.366 | 1.915 | 0.111 | 0.107 | 0.178 | 0.022 |
| | & 5 mL/kg | Mouse#35 | | 23 | 0.011 | 0.437 | 0.148 | 0.375 | 1.493 | 0.110 | 0.111 | 0.183 | 0.018 |
| | | Mouse#36 | | 24 | 0.009 | 0.437 | 0.113 | 0.327 | 1.587 | 0.080 | 0.088 | 0.196 | 0.014 |
| | 10 mg/kg | Mouse#40 | Female | 25 | 0.011 | 0.449 | 0.128 | 0.399 | 1.563 | 0.160 | 0.122 | 0.218 | 0.028 |
| | & 5 mL/kg | Mouse#41 | | 25 | 0.009 | 0.465 | 0.125 | 0.345 | 1.706 | 0.113 | 0.102 | 0.223 | 0.024 |
| | | Mouse#42 | | 25 | 0.010 | 0.414 | 0.126 | 0.352 | 1.610 | 0.174 | 0.137 | 0.228 | 0.006 |
| | 50 mg/kg | Mouse#46 | Female | 22 | 0.009 | 0.433 | 0.107 | 0.362 | 1.798 | 0.185 | 0.105 | 0.194 | 0.022 |
| | & 5 mL/kg | Mouse#47 | | 20 | 0.006 | 0.389 | 0.087 | 0.426 | 1.585 | 0.173 | 0.058 | 0.127 | 0.015 |
| | | Mouse#48 | | 22 | 0.007 | 0.439 | 0.119 | 0.391 | 1.623 | 0.160 | 0.058 | 0.160 | 0.016 |
| | 25 mg/kg | Mouse#52 | Female | 23 | 0.012 | 0.430 | 0.125 | 0.385 | 1.694 | 0.137 | 0.106 | 0.252 | 0.019 |
| | & 5 mL/kg | Mouse#53 | | 23 | 0.012 | 0.450 | 0.130 | 0.401 | 1.953 | 0.157 | 0.081 | 0.223 | 0.022 |
| | | Mouse#54 | | 23 | 0.011 | 0.469 | 0.109 | 0.409 | 1.772 | 0.151 | 0.151 | 0.307 | 0.018 |

| | | | | | | | | | | | | | |
|---|---|---|---|---|---|---|---|---|---|---|---|---|---|
| | | | | | Adrenal | | | | | | | | |
| | | | | Body | glands/ | Brain/ | Heart/ | Kidneys/ | Liver/ | Spleen/ | Thymus/ | Lung/ | Testes/ |
| | | | | Weight | Body | Body | Body | Body | Body | Body | Body | Body | Body |
| | Dose | | | before | Coeffi- | Coeffi- | Coeffi- | Coeffi- | Coeffi- | Coeffi- | Coeffi- | Coeffi- | Coeffi- |
| | Level & | | | Necropsy | cient | cient | cient | cient | cient | cient | cient | cient | cient |
| Compound | Volume | Mouse ID | Gender | (g) | (%) | (%) | (%) | (%) | (%) | (%) | (%) | (%) | (%) |

Organ/Body Coefficient of Male ICR MTD Study (IFX301, IP Route)

| | | | | | | | | | | | | | |
|---|---|---|---|---|---|---|---|---|---|---|---|---|---|
| IFX301 | Vehicle | Mouse#31 | Male | 30 | 0.04% | 1.55% | 0.50% | 1.80% | 6.89% | 0.32% | 0.29% | 0.65% | 0.63% |
| | & 5 mL/kg | Mouse#32 | | 31 | 0.04% | 1.55% | 0.56% | 1.86% | 6.92% | 0.50% | 0.31% | 0.89% | 0.64% |
| | | Mouse#33 | | 31 | 0.03% | 1.47% | 0.52% | 1.68% | 7.06% | 0.40% | 0.37% | 0.63% | 0.55% |
| | 10 mg/kg | Mouse#37 | Male | 26 | 0.02% | 1.76% | 0.51% | 1.79% | 6.78% | 0.45% | 0.30% | 0.77% | 0.54% |
| | & 5 mL/kg | Mouse#38 | | 28 | 0.06% | 1.58% | 0.46% | 1.63% | 6.60% | 0.49% | 0.24% | 0.81% | 0.63% |
| | | Mouse#39 | | 28 | 0.04% | 1.58% | 0.60% | 1.63% | 7.53% | 0.53% | 0.37% | 0.74% | 0.59% |
| | 50 mg/kg | Mouse#43 | Male | 25 | 0.04% | 1.72% | 0.45% | 1.88% | 8.02% | 0.63% | 0.22% | 0.70% | 0.77% |
| | & 5 mL/kg | Mouse#44 | | 26 | 0.01% | 1.75% | 0.48% | 2.02% | 7.78% | 0.67% | 0.28% | 0.67% | 0.69% |
| | | Mouse#45 | | 27 | 0.04% | 1.69% | 0.54% | 2.27% | 8.97% | 0.75% | 0.24% | 0.78% | 0.61% |
| | 25 mg/kg | Mouse#49 | Male | 26 | 0.02% | 1.74% | 0.48% | 1.94% | 8.54% | 0.33% | 0.32% | 1.15% | 0.65% |
| | & 5 mL/kg | Mouse#50 | | 26 | 0.06% | 1.67% | 0.51% | 2.03% | 8.69% | 0.33% | 0.32% | 0.75% | 0.73% |
| | | Mouse#51 | | 26 | 0.02% | 1.60% | 0.71% | 2.49% | 8.70% | 0.72% | 0.29% | 1.08% | 0.49% |

Organ/Body Coefficient of Female ICR MTD Study (IFX301, IP Route)

| | | | | | | | | | | | | | |
|---|---|---|---|---|---|---|---|---|---|---|---|---|---|
| IFX301 | Vehicle | Mouse#34 | Female | 27 | 0.06% | 1.58% | 0.50% | 1.36% | 7.09% | 0.41% | 0.40% | 0.66% | 0.08% |
| | & 5 mL/kg | Mouse#35 | | 23 | 0.05% | 1.90% | 0.64% | 1.63% | 6.49% | 0.48% | 0.48% | 0.80% | 0.08% |
| | | Mouse#36 | | 24 | 0.04% | 1.82% | 0.47% | 1.36% | 6.61% | 0.33% | 0.37% | 0.82% | 0.06% |
| | 10 mg/kg | Mouse#40 | Female | 25 | 0.04% | 1.80% | 0.51% | 1.60% | 6.25% | 0.64% | 0.49% | 0.87% | 0.11% |
| | & 5 mL/kg | Mouse#41 | | 25 | 0.04% | 1.86% | 0.50% | 1.38% | 6.82% | 0.45% | 0.41% | 0.89% | 0.10% |
| | | Mouse#42 | | 25 | 0.04% | 1.66% | 0.50% | 1.41% | 6.44% | 0.70% | 0.55% | 0.91% | 0.02% |
| | 50 mg/kg | Mouse#46 | Female | 22 | 0.04% | 1.97% | 0.49% | 1.65% | 8.17% | 0.84% | 0.48% | 0.88% | 0.10% |
| | & 5 mL/kg | Mouse#47 | | 20 | 0.03% | 1.95% | 0.44% | 2.13% | 7.93% | 0.87% | 0.29% | 0.64% | 0.08% |
| | | Mouse#48 | | 22 | 0.03% | 2.00% | 0.54% | 1.78% | 7.38% | 0.73% | 0.26% | 0.73% | 0.07% |
| | 25 mg/kg | Mouse#52 | Female | 23 | 0.05% | 1.87% | 0.54% | 1.67% | 7.37% | 0.60% | 0.46% | 1.10% | 0.08% |
| | & 5 mL/kg | Mouse#53 | | 23 | 0.05% | 1.96% | 0.57% | 1.74% | 8.49% | 0.68% | 0.35% | 0.97% | 0.10% |
| | | Mouse#54 | | 23 | 0.05% | 2.04% | 0.47% | 1.78% | 7.70% | 0.66% | 0.66% | 1.33% | 0.08% |

Average Organ/Body Coefficient of Male ICR MTD Study (IFX301, IP Route)

| | | | | Average | Adrenal | | | | | | | | |
|---|---|---|---|---|---|---|---|---|---|---|---|---|---|
| | | | | Body | glands/ | Brain/ | Heart/ | Kidneys/ | Liver/ | Spleen/ | Thymus/ | Lung/ | Testes/ |
| | | | | Weight | Body | Body | Body | Body | Body | Body | Body | Body | Body |
| | Dose | | | before | Coeffi- | Coeffi- | Coeffi- | Coeffi- | Coeffi- | Coeffi- | Coeffi- | Coeffi- | Coeffi- |
| | Level & | | | Necropsy | cient | cient | cient | cient | cient | cient | cient | cient | cient |
| Compound | Volume | Mouse ID | Gender | (g) | (%) | (%) | (%) | (%) | (%) | (%) | (%) | (%) | (%) |
| IFX301 | Vehicle | M31~M33 | Male | 31 | 0.03% | 1.52% | 0.53% | 1.78% | 6.96% | 0.41% | 0.33% | 0.73% | 0.61% |
| | & 5 mL/kg | | | | | | | | | | | | |
| | 10 mg/kg | M37~M39 | Male | 27 | 0.04% | 1.63% | 0.52% | 1.68% | 6.98% | 0.49% | 0.30% | 0.77% | 0.59% |
| | & 5 mL/kg | | | | | | | | | | | | |
| | 50 mg/kg | M43~M45 | Male | 26 | 0.03% | 1.72% | 0.49% | 2.06% | 8.27% | 0.68% | 0.25% | 0.72% | 0.69% |
| | & 5 mL/kg | | | | | | | | | | | | |
| | 25 mg/kg | M49~M51 | Male | 26 | 0.03% | 1.67% | 0.57% | 2.15% | 8.64% | 0.46% | 0.31% | 1.00% | 0.62% |
| | & 5 mL/kg | | | | | | | | | | | | |

Average Organ/Body Coefficient of Female ICR MTD Study (IFX301, IP Route)

| | | | | Average | Adrenal | | | | | | | | |
|---|---|---|---|---|---|---|---|---|---|---|---|---|---|
| | | | | Body | glands/ | Brain/ | Heart/ | Kidneys/ | Liver/ | Spleen/ | Thymus/ | Lung/ | Ovaries/ |
| | | | | Weight | Body | Body | Body | Body | Body | Body | Body | Body | Body |
| | Dose | | | before | Coeffi- | Coeffi- | Coeffi- | Coeffi- | Coeffi- | Coeffi- | Coeffi- | Coeffi- | Coeffi- |
| | Level & | | | Necropsy | cient | cient | cient | cient | cient | cient | cient | cient | cient |
| Compound | Volume | Mouse ID | Gender | (g) | (%) | (%) | (%) | (%) | (%) | (%) | (%) | (%) | (%) |
| IFX301 | Vehicle | M34~M36 | Female | 25 | 0.05% | 1.76% | 0.54% | 1.44% | 6.75% | 0.41% | 0.41% | 0.75% | 0.07% |
| | & 5 mL/kg | | | | | | | | | | | | |
| | 10 mg/kg | M40~M42 | Female | 25 | 0.04% | 1.77% | 0.51% | 1.46% | 6.51% | 0.60% | 0.48% | 0.89% | 0.08% |
| | & 5 mL/kg | | | | | | | | | | | | |
| | 50 mg/kg | M46~M48 | Female | 21 | 0.03% | 1.97% | 0.49% | 1.84% | 7.82% | 0.81% | 0.35% | 0.75% | 0.08% |
| | & 5 mL/kg | | | | | | | | | | | | |
| | 25 mg/kg | M52~M54 | Female | 23 | 0.05% | 1.96% | 0.53% | 1.73% | 7.85% | 0.64% | 0.49% | 1.13% | 0.09% |
| | & 5 mL/kg | | | | | | | | | | | | |

### REFERENCES

Ageitos J M, Sánchez-Perez A, Calo-Mata P, Villa T G. 2017. Antimicrobial peptides (AMPs): Ancient compounds that represent a novel weapons in the fight of bacteria. Biochem. Pharmacol. 133: 177-138.
Domhan C, Uhl P, Meinhardt A, Zimmermann S, Kleist C, Linder T, Leotta K, Mier W, Wink M. 2018. A novel tool against multi-resistant bacterial pathogens lipopeptide. Modification of the natural antimicrobial peptide renalexin for enhanced antimicrobial activity and improved pharmacokinetics. Int J Antimicrob Agents. 52(1): 52-62.
De Smet K, Contreras R. 2005. Human antimicrobial peptides: defensins, cathelicidins and histatins. Biotechnol Lett 27(18): 1337-1347.
El-Mahallawy H A, Hassan S S, El-Wakil M, Moneer M M. 2016. Bacteremia due to ESKAPE Pathogens. An emerging problem in cancer patients. J. Egypt Natl. Canc. Inst. 28: 157-162.
Knappe D, Henklein P, Hilpert K. 2010. Easy strategy to protect antimicrobial peptides from fast degradation in serum. Antimicrob Agents Chemother. 54(9): 4003-4005.
Nordström R, Malmsten M. 2017. Delivery systems of antimicrobial peptides. Adv. Colloid Interface Sci. 242: 17-34).
Oh D, Sun J., Nasrolahi Shirazi A., LaPlante K. L., Rowley D. C., Parang K. 2014. Antibacterial activities of amphiphilic cyclic cell-penetrating peptides against multidrug resistant pathogens. Mol. Pharmaceutics 11, 3528-3536.
Kumar P, Kizhakkedathu J N, Straus S K. 2018. Antimicrobial peptides: diversity, mechanism of action and strategies to improve the activity and biocompatibility in vivo. Biomolecules. 8(1): 4
Robert S, Rothenburger M, Graninger W, Joukhadar C. 2008. Daptomycin: A review 4 Years after first approval. Pharmacology. 81(2): 79-91.
Seo M.-D. Won H.-S., Kim, J. H., Mishig-Ochir T, Lee B. J. 2012. Antimicrobial peptides for therapeutic applications: A review. Molecules. 17: 12276-12286.

What is claimed is:

1. A synthetic peptide comprising a sequence of amino acids XₙYₘ, wherein X represents positively charged amino acid, Y represents hydrophobic amino acid, and both n and m are greater than 2.

2. The synthetic peptide of claim 1, comprising a linear arrangement of amino acids as (XₙYₘ).

3. The synthetic peptide of claim 1 or 2, comprising a cyclic arrangement of amino acids as [XₙYₘ].

4. The synthetic peptide of claim 1, comprising a plurality of cyclic arrangements of amino acids as hybrid peptides (cyclic-linear) [Xₙ]Yₘ, or Xₙ[Yₘ], with cyclic peptides contain positively-charged residues or hydrophobic residues attached to linear hydrophobic or positively-charged residues

5. The synthetic peptide of claim 1, comprising a plurality of cyclic peptides, wherein at least two of the plurality of cyclic peptides are coupled by a linker to form a cyclic peptide couplet [X]ₙ[Y]ₘ.

6. The synthetic peptide of claim 5, wherein the cyclic peptide couplet comprises a first cyclic peptide comprising positively charged amino acids and a second cyclic peptide comprising hydrophobic amino acids.

7. The synthetic peptide of any one of claims 1 to 6, wherein both n and m range from 2 to 9.

8. The synthetic peptide of any one of claims 1 to 7, comprising a positively charged region and a hydrophobic region.

9. The synthetic peptide of any one of claims 1 to 8, further comprising one or more additional amino acids positioned between a positively charged amino acid and a hydrophobic amino acid.

10. The synthetic peptide of any one of claims 1 to 9, wherein positively charged amino acids of the peptide are identical species.

11. The synthetic peptide of any one of claims 1 to 9, wherein positively charged amino acids of the peptide are different species.

12. The synthetic peptide of any one of claims 1 to 9, wherein hydrophobic amino acids of the peptide are identical species.

13. The synthetic peptide of any one of claims 1 to 9, wherein hydrophobic amino acids of the peptide are different species.

14. The synthetic peptide of any one of claims 1 to 13, wherein positively charged amino acids are L- or D-optical isomers of naturally occurring positively charged amino acids.

15. The synthetic peptide of claim 14, wherein positively charged amino acids are selected from the group consisting of arginine, lysine, histidine, and ornithine.

16. The synthetic peptide of any one of claims 1 to 13, wherein positively charged amino acids are selected from L- or D-isomers of non-naturally occurring positively-charged amino acids.

17. The synthetic peptide of claim 16, wherein non-naturally occurring positively-charged amino acids are selected from the group consisting of an arginine with a modified side chain, C3-arginine (Agp), C4-arginine (Agb), lysine with a modified side chain, an ornithine with a modified side chain, a histidine with a modified side chain, diaminopropionic acid (Dap), diaminobutyric acid (Dab), an amino acid having a free side chain amino group, and an amino acid having a free side chain guanidine group.

18. The synthetic peptide of any one of claims 1 to 17, wherein hydrophobic amino acids are L- or D-optical isomers of naturally occurring hydrophobic amino acids.

19. The synthetic peptide of claim 18, wherein hydrophobic amino acids are selected from the group consisting of tryptophan, phenylalanine, leucine, and isoleucine.

20. The synthetic peptide of any one of claims 1 to 17, wherein hydrophobic amino acids are L- or D-optical isomers of non-naturally occurring hydrophobic amino acids.

21. The synthetic peptide of claim 20, wherein hydrophobic amino acids are selected from the group consisting of p-phenyl-L-phenylalanine (Bip), 3,3-diphenyl-L-alanine (Dip), 3,3-diphenyl-D-alanine (dip), 3(2-naphthyl)-L-alanine (NaI), 3(2-naphthyl)-D-alanine (naI), 6-amino-2-naphthoic acid, 3-amino-2-naphthoic acid, 1,2,3,4-tetrahydronorharmane-3-carboxylic acid, 1,2,3,4-tetrahydro-3-isoquinolinecarboxylic acid (Tic-OH), 1,2,3,4-tetrahydro-3-isoquinolinecarboxylic acid, modified D- or L-tryptophan; N-alkyl tryptophan, N-aryl tryptophan, 5-hydroxy-L-tryptophan, 5-methoxy-L-tryptophan, 6-chloro-L-tryptophan), fatty amino acids NH₂—(CH₂)ₓ—COOH (x=1-20), and an N-heteroaromatic amino acid.

22. A use of a synthetic peptide of any one of claims 1 to 21, wherein the synthetic peptide is used in combination with an antibiotic compound.

23. A use of a synthetic peptide of any one of claims 1 to 21, wherein the synthetic peptide is used in conjugation with an antibiotic compound.

24. A use of a synthetic peptide of any one of claims 1 to 21, wherein the synthetic peptide is used in combination with an antiviral compound.

25. A use of a synthetic peptide of any one of claims 1 to 21, wherein the synthetic peptide is used in conjugation with an antiviral compound.

26. An antimicrobial composition comprising:
a synthetic peptide of any one of claims 1 to 21; and
a nanoparticle,
wherein the synthetic peptide is combined with a nanoparticle.

27. The antimicrobial composition of claim 26, wherein the synthetic peptide is noncovalently coated onto the nanoparticle.

28. The antimicrobial composition of claim 26, wherein the synthetic peptide is covalently coupled to the nanoparticle.

29. The antimicrobial composition of any one of claims 26 to 28, wherein the nanoparticle is a gold nanoparticle or a silver nanoparticle.

30. The antimicrobial composition of any one of claims 26 to 29, further comprising an antimicrobial compound.

31. Use of a synthetic peptide of any one of claims 1 to 21 or the antimicrobial composition of any one of claims 26 to 30 for the treatment of an infection.

32. The use of claim 31, wherein the infection is selected from the group consisting of a bacterial infection, a Gram-positive bacterial infection, a Gram-negative bacterial infection, a fungal infection, and a viral infection

33. The use of claim 31 or 32, wherein the infection is caused by a multidrug-resistant bacteria.

34. The use of one of claims 31 to 32, wherein the infection is caused by an organism selected from the group consisting of methicillin-resistant Staphylococcus aureus (MRSA), Acinetobacter baumannii, Enterococcus faecalis, Clostridium difficile, Klebsiella pneumonia, Escherichia coli, Streptococcus pneumoniae, Pseudomonas aeruginosa, Mycobacterium tuberculosis, Carbapenem-resistant Enterobacteriaceae (CRE) gut bacteria, Neisseria gonorrhea, Candida albicans, and Cryptococcus neoformans.

35. The use of claim 31 or 32, wherein the infection is caused by an organism selected from the group consisting of suitable DNA viruses include (but are not limited to) Herpesviruses, Poxviruses, Hepadnaviruses, and Asfarviridae. Suitable RNA viruses include (but are not limited to) Flavivirus, Alphavirus, Togavirus, Coronavirus, Hepatitis D, Orthomyxovirus, Paramyxovirus, Rhabdovirus, Bunyavirus, Filovirus, Retroviruses, and Retroviruses.

36. A method of inhibiting or halting microbial growth, comprising applying a synthetic peptide of any one of claims 1 to 21 or the antimicrobial composition of any one of claims 26 to 30 in an amount effective to inhibit or halt microbial growth.

37. The method of claim 36, comprising a step of applying the synthetic peptide to an animal, wherein the amount is selected to effective to inhibit or halt microbial growth in the animal.

38. The method of claim 36, comprising a step of applying the synthetic peptide to a biofilm, wherein the amount is selected to effective to inhibit or halt microbial growth in the biofilm.

39. The method of claim 38, wherein the biofilm comprises a Gram-positive bacteria.

40. The method of claim 38, wherein the biofilm comprises a Gram-negative bacteria.

41. The method of claim 36, comprising a step of applying the synthetic peptide to a food or food product, wherein the amount is selected to effective to inhibit or halt microbial growth in the food or food product.

42. The method of claim 36, comprising a step of applying the synthetic peptide to an aqueous solution, wherein the amount is selected to effective to inhibit or halt microbial growth in the aqueous solution.

43. The method of claim 42, wherein the aqueous solution is a water supply.

44. The method of claim 42, wherein the aqueous solution is selected from the group consisting of an eyedrop, a contact lens solution, and an eye wash solution.

45. The method of claim 36, comprising a step of applying the synthetic peptide to an article, and wherein the amount is selected to be effective to inhibit or halt microbial growth on or within the article.

46. The method of claim 45, wherein the article is a personal article selected from the group consisting of a contact lens, a personal wipe, a baby wipe, a diaper, a wound dressing. a towelette, and a cosmetic product.

47. The method of claim 45, wherein the article is a medical device.

48. A composition comprising a synthetic peptide of any one of claims 1 to 21 or the antimicrobial composition of any one of claims 26 to 30, wherein the synthetic peptide comprises a non-peptide bond coupling two adjacent amino acids of the peptide.

49. The composition of claim 48, wherein the non-peptide bond is selected from the group consisting of a thioamide, an N-methyl group, and a CH₂—NH group.

50. Use of a synthetic peptide of one of claims 1 to 21 or the antimicrobial composition of any one of claims 26 to 30 to inhibit or halt microbial growth in or on an animal.

51. A kit comprising:
a synthetic peptide of any one of claims 1 to 21 or the antimicrobial composition of any one of claims 26 to 30; and
instructions for applying the synthetic peptide in a manner effective to inhibit or halt microbial growth.

52. A pharmaceutical formulation comprising:
a synthetic peptide of any one of claims 1 to 21 or the antimicrobial composition of any one of claims 26 to 30; and
a pharmaceutically acceptable vehicle.

53. The pharmaceutical formulation of claim 52, wherein the pharmaceutically acceptable vehicle is selected to provide at least one of topical, oral, inhalation, injection, rectal, vaginal, intravenous infusion, and nasal administration of the synthetic peptide.

54. A method of increasing transport of a compound across a cell membrane, comprising application of a peptide of any one of claims 1 to 21 or the antimicrobial composition of any one of claims 26 to 30 to the cell membrane.

55. The method of claim 54, wherein the compound is an antibiotic, an antifungal, or antiviral.

56. The method of claim 54 or 55, wherein the cell membrane is a bacterial cell membrane, viral cell membrane, or fungal cell membrane.

57. A pegylated form of a synthetic peptide of any one of claims 1 to 21.

58. A pharmaceutical composition comprising a compound of any one of claims 1 to 21 or the antimicrobial composition of any one of claims 26 to 30, in an amount that is effective against a coronavirus.

59. The pharmaceutical composition of claim 58 comprising a carrier or excipient.

60. The pharmaceutical composition of claim 58, wherein the pharmaceutical composition is formulated as an injectable, a solid or semi-solid form, a tablet, a film, a gel, a cream, an ointment, a spray, a solution, a suspension, a micellar suspension, powder or granule, an encapsulated granule, an atomized mist, or a pessary.

61. A method of treating a bacterial disease, comprising:
obtaining a pharmaceutical formulation comprising a compound of any one of claims 1 to 21; and
applying an effective amount of the pharmaceutical formulation to an individual in need of treatment.

62. The method of claim 61, wherein application comprises topical application of the pharmaceutical formulation to a body surface.

63. The method of claim 62, wherein the body surface is a mucus membrane.

64. The method of claim 61, wherein application comprises injection of the pharmaceutical formulation.

65. The method of claim 64, wherein injection is selected from the group consisting of subcutaneous injection, intramuscular injection, intraocular injection, intravenous injection, and infusion.

66. The method of claim 61, wherein application comprises inhalation of the pharmaceutical formulation.

67. The method of any one of claims 61 to 66, wherein treatment is prophylactic.

68. The method of any one of claims 61 to 66, wherein the individual has an active bacterial infection but is asymptomatic.

69. The method of any one of claims 61 to 68, wherein the pharmaceutical formulation comprises a second active compound.

70. The method of claim 69, wherein the second active compound is selected from the group consisting of an antiviral compound, an antibacterial compound, an antifungal compound, an anti-inflammatory compound, and a bronchodilator.

71. The method of claim 70, wherein the bacterial compound is a second compound as in one of claims 1 to 26.


SEQUENCE LISTING (36 sequences)

SEQ ID NO: 1 - protein, 10 aa, Artificial Sequence
  Synthetic Construct
CRRRRWWWWC

SEQ ID NO: 2 - protein, 10 aa, Artificial Sequence
  Synthetic Construct
RRRRCWWWWC

SEQ ID NO: 3 - protein, 10 aa, Artificial Sequence
  Synthetic Construct
WWWWCRRRRC

SEQ ID NO: 4 - protein, 8 aa, Artificial Sequence
  Synthetic Construct
RRRRCCCC

SEQ ID NO: 5 - protein, 12 aa, Artificial Sequence
  Synthetic Construct
RRRRWWWWCCCC

SEQ ID NO: 6 - protein, 6 aa, Artificial Sequence
  Synthetic Construct
  MISC_FEATURE (4)..(6): p-phenyl-L-phenylalanine
RRRXXX

SEQ ID NO: 7 - protein, 6 aa, Artificial Sequence
  Synthetic Construct
  MISC_FEATURE (4)..(6): 3,3-diphenyl-L-alanine
RRRXXX

SEQ ID NO: 8 - protein, 6 aa, Artificial Sequence
  Synthetic Construct
  MISC_FEATURE (4)..(6): Naphthylalanine
RRRXXX

SEQ ID NO: 9 - protein, 8 aa, Artificial Sequence
  Synthetic Construct
  MISC_FEATURE (5)..(8): p-phenyl-L-phenylalanine
RRRRXXXX

SEQ ID NO: 10 - protein, 8 aa, Artificial Sequence
  Synthetic Construct
  MISC_FEATURE (5)..(8): 3,3-diphenyl-L-alanine
RRRRXXXX

SEQ ID NO: 11 - protein, 8 aa, Artificial Sequence
  Synthetic Construct
  MISC_FEATURE (5)..(8): Naphthylalanine
RRRRXXXX

SEQ ID NO: 12 - protein, 7 aa, Artificial Sequence
  Synthetic Construct
  MISC_FEATURE (5)..(7): p-phenyl-L-phenylalanine
RRRRXXX

SEQ ID NO: 13 - protein, 7 aa, Artificial Sequence
  Synthetic Construct
  MISC_FEATURE (5)..(7): 3,3-diphenyl-L-alanine
RRRRXXX

SEQ ID NO: 14 - protein, 7 aa, Artificial Sequence
  Synthetic Construct
  MISC_FEATURE (5)..(7): Naphthylalanine
RRRRXXX

SEQ ID NO: 15 - protein, 6 aa, Artificial Sequence
  Synthetic Construct
  MISC_FEATURE (5)..(6): 3,3-diphenyl-L-alanine
RRRRXX

SEQ ID NO: 16 - protein, 6 aa, Artificial Sequence
  Synthetic Construct
  MISC_FEATURE (5)..(6): Naphthylalanine
RRRRXX

SEQ ID NO: 17 - protein, 8 aa, Artificial Sequence
  Synthetic Construct
  MISC_FEATURE (6)..(8): 3,3-diphenyl-L-alanine
RRRRRXXX

SEQ ID NO: 18 - protein, 8 aa, Artificial Sequence
  Synthetic Construct
  MISC_FEATURE (6)..(8): Naphthylalanine
RRRRRXXX

SEQ ID NO: 19 - protein, 7 aa, Artificial Sequence
  Synthetic Construct
  MISC_FEATURE (2)..(2): 3,3-diphenyl-L-alanine
  MISC_FEATURE (5)..(6): 3,3-diphenyl-L-alanine
RXRRXXR

SEQ ID NO: 20 - protein, 7 aa, Artificial Sequence
  Synthetic Construct
  MISC_FEATURE (3)..(4): 3,3-diphenyl-L-alanine
  MISC_FEATURE (7)..(7): 3,3-diphenyl-L-alanine
RRXXRRX

SEQ ID NO: 21 - protein, 7 aa, Artificial Sequence
  Synthetic Construct
  MISC_FEATURE (2)..(2): Naphthylalanine
  MISC_FEATURE (5)..(6): Naphthylalanine
RXRRXXR

SEQ ID NO: 22 - protein, 7 aa, Artificial Sequence
  Synthetic Construct
  MISC_FEATURE (3)..(4): Naphthylalanine
  MISC_FEATURE (7)..(7): Naphthylalanine
RRXXRRX

SEQ ID NO: 23 - protein, 6 aa, Artificial Sequence
  Synthetic Construct
  MISC_FEATURE (5)..(6): 3,3-diphenyl-L-alanine
RRRWXX

SEQ ID NO: 24 - protein, 6 aa, Artificial Sequence
  Synthetic Construct
  MISC_FEATURE (4)..(4): 3,3-diphenyl-L-alanine
  MISC_FEATURE (6)..(6): 3,3-diphenyl-L-alanine
RRRXWX

SEQ ID NO: 25 - protein, 6 aa, Artificial Sequence
  Synthetic Construct
  MISC_FEATURE (4)..(5): 3,3-diphenyl-L-alanine
RRRXXW

SEQ ID NO: 26 - protein, 6 aa, Artificial Sequence
  Synthetic Construct
  MISC_FEATURE (5)..(6): Naphthylalanine
RRRWXX

SEQ ID NO: 27 - protein, 6 aa, Artificial Sequence
  Synthetic Construct
  MISC_FEATURE (4)..(4): Naphthylalanine
  MISC_FEATURE (6)..(6): Naphthylalanine
RRRXWX

SEQ ID NO: 28 - protein, 6 aa, Artificial Sequence
  Synthetic Construct
  MISC_FEATURE (4)..(5): Naphthylalanine
RRRXXW

SEQ ID NO: 29 - protein, 7 aa, Artificial Sequence
  Synthetic Construct
  MISC_FEATURE (6)..(7): 3,3-diphenyl-L-alanine
RRRRWXX

SEQ ID NO: 30 - protein, 7 aa, Artificial Sequence
  Synthetic Construct
  MISC_FEATURE (5)..(5): 3,3-diphenyl-L-alanine
  MISC_FEATURE (7)..(7): 3,3-diphenyl-L-alanine
RRRRXWX

SEQ ID NO: 31 - protein, 7 aa, Artificial Sequence
  Synthetic Construct
  MISC_FEATURE (5)..(6): 3,3-diphenyl-L-alanine
RRRRXXW

SEQ ID NO: 32 - protein, 7 aa, Artificial Sequence
  Synthetic Construct
  MISC_FEATURE (6)..(7): Naphthylalanine
RRRRWXX

SEQ ID NO: 33 - protein, 7 aa, Artificial Sequence
  Synthetic Construct
  MISC_FEATURE (5)..(5): Naphthylalanine
  MISC_FEATURE (7)..(7): Naphthylalanine
RRRRXWX

SEQ ID NO: 34 - protein, 7 aa, Artificial Sequence
  Synthetic Construct
  MISC_FEATURE (5)..(6): Naphthylalanine
RRRRXXW

SEQ ID NO: 35 - protein, 10 aa, Artificial Sequence
  Synthetic Construct
RRRRRWWWWK

SEQ ID NO: 36 - protein, 10 aa, Artificial Sequence
  Synthetic Construct
RRRRRWWWWK